(19) United States
(12) Patent Application Publication
(10) Pub. No.: US 2021/0393765 A1
(43) Pub. Date: Dec. 23, 2021

(54) ROTAVIRUS VP7 FUSION PROTEINS AND ROTAVIRUS-LIKE PARTICLES COMPRISING THEM

(71) Applicants: MEDICAGO INC., Québec (CA); MITSUBISHI TANABE PHARMA CORPORATION, Chuo-ku, Osaka (JP)

(72) Inventors: Marc-Andre D AOUST, Québec (CA); Pierre-Olivier LAVOIE, Québec (CA)

(73) Assignees: MEDICAGO INC., Québec (CA); MITSUBISHI TANABE PHARMA CORPORATION, Chuo-ku, Osaka (JP)

(21) Appl. No.: 17/431,987

(22) PCT Filed: Feb. 19, 2020
(86) PCT No.: PCT/CA2020/050212

§ 371 Date: Aug. 18, 2021

Related U.S. Application Data
(60) Provisional application No. 62/807,389, filed on Feb. 19, 2019.

(51) Int. Cl. A61K 39/12; C12N 15/82

(52) U.S. Cl. CPC A61K 39/12; C12N 15/8257; A61K 38/00; C07K 2319/00; C12N 2720/12323

(57) ABSTRACT

Nucleic acids encoding rotavirus VP7 fusion proteins and rotavirus-like particle (RLPs) comprising the rotavirus VP7 fusion proteins are provided. Methods for rotavirus VP7 fusion protein and RLP production in plants are also described. The VP7 fusion protein comprises a first sequence encoding a 7-1a subdomain, a second sequence encoding a 7-2 domain and a third sequence encoding a 7-1b subdomain; wherein the sequence of the 7-2 domain is derived from a first rotavirus strain and the sequence of the 7-1a subdomain, the sequence of the 7-1b subdomain or the sequence of the 7-1a subdomain and the sequence of the 7-1b subdomain are derived from a second rotavirus strain.

### FIELD OF INVENTION

This disclosure relates to rotavirus fusion proteins, Rotavirus-like Particles comprising rotavirus fusion proteins, and methods of producing the same.

### BACKGROUND OF THE INVENTION

Acute gastroenteritis has been demonstrated as a major cause of morbidity and mortality of children in both developed and developing countries. It has been well established that virtually every child becomes infected with a rotavirus at least once by 3 years of age. The rotaviruses, which comprise a genus in the family Reoviridae, are spherical in appearance and measure about 80 nm in diameter.

The structures of infectious and sub-viral rotavirus particles have been solved using X-ray crystallography and single-particle reconstructions of cryo-EM images (McClain B, Settembre E, Temple B R, Bellamy A R, Harrison S C J Mol Biol. 2010; 397:587-599). At approximately 80 nm in diameter, the infectious triple-layered particle (TLP) is relatively large compared to many other non-enveloped, icosahedral viruses. The innermost layer of the TLP, is referred to as the core shell and immediately surrounds the viral dsRNA genome. The core shell is composed of 60 dimer of VP2 (102 kDa). Surrounding the rotavirus VP2 shell are two additional protein layers (Trask et al. Nat Rev Microbiol. 2012 Jan. 23; 10(3): 165-177). The intermediate layer is relatively thick compared to the other two layers and is made up of 260 trimers of VP6 (monomer, 45 kDa). Binding of VP6 to VP2 results in a dramatic stabilization of the very fragile core and the formation of the non-infectious double layer virus particle (DLP). VP6 also serves as an adaptor for the rotavirus outer capsid proteins, which are critical for attachment and entry into a host cell. Specifically, 260 trimers of the glycoprotein VP7 (monomer, 37 kDa) sit directly on top of the VP6 trimers and form a continuous, perforated shell. VP7 trimers are dependent on bound calcium ions for stability; two calcium ions are held at each subunit interface, requiring six total bound ions per trimer (Aoki S T, et al. Science. 2009; 324:1444-1447). Protruding through the VP7 layer on the rotavirus virion are 60 trimeric spikes 120 Å in length that emanate from the peripentonal channels of the VP6 layer. These spikes are formed by the viral attachment protein, VP4 (88 kDa). The outer capsid proteins VP7 and VP4 (including the VP4 cleavage products, VP5* and VP8*) are the primary targets of rotavirus-neutralizing antibodies. The VP7 glycoprotein (G-antigen) and the protease-sensitive spike protein, VP4 (P-antigen), are used to classify rotavirus strain serotype based on sequence comparison and reactivity with neutralizing antibodies.

Rotaviruses are divided into 7 groups (A-G) and four subgroups (I, II, I+II and Non I/II) in group A which are based on the antigenic properties of VP6. The 2 outer capsid proteins define the dual serotype classification of the viruses, with VP4 (protease-sensitive) defining the P serotype, and VP7 (a glycoprotein) the G serotype.

Groups A, B, and C have been found in both humans and animals, and groups D, E, and F have been found only in animals. Rotaviral group A is a common cause of rotavirus diarrhea in humans, and it is the first-choice candidate for vaccine development.

Despite high diversity among the subgroups, VP6 protein is stringently conserved and very immunogenic among all group A rotaviruses. However, capsid proteins VP7 and VP4 have very high diversity among strains and are the main target for neutralizing antibodies.

Due to the lack of proper immunological reagents and the increasing ease of sequencing, serotyping is being complemented with genotyping, which is based on identities between sequences of cognate rotavirus gene segments. So far, 15 G genotypes (14 G serotypes) have been identified, and out of 27 different P genotypes, 14 P serotypes (1A, 1B, and 2 to 14) have been identified with available VP4-specific antibodies. (Matthijnssens J. et al. J. Virol. April 2008 vol. 82 no. 7 3204-3219).

Traditionally, a cutoff value of 89% VP7 amino acid sequence identity has been used to classify G genotypes, yielding a nearly complete concordance with the different G serotypes (Estes, M. K., and A. Z. Kapikian. 2007. Rotaviruses and their replication, p. 1917-1974. In B. N. Fields, D. M. Knipe, P. M. Howley, D. E. Griffin, R. A. Lamb, M. A. Martin, B. Roizman, and S. E. Straus (ed.), Fields virology, 5th ed. Lippincott, Williams and Wilkins, Philadelphia, Pa.). In contrast, the 89% amino acid identity cutoff value for VP4, established by Gorziglia and colleagues (Gorziglia, M., G. Larralde, A. Z. Kapikian, and R. M. Chanock. 1990. Antigenic relationships among human rotaviruses as determined by outer capsid protein VP4. Proc. Natl. Acad. Sci. USA 87:7155-7159.), does not result in an absolute concordance between different P genotypes and P serotypes. Specifically, P serotypes have not been defined for approximately half of the P genotypes, which are designated by an Arabic numeral between square brackets (Estes, M. K., and A. Z. Kapikian. 2007. Rotaviruses and their replication, p. 1917-1974. In B. N. Fields, D. M. Knipe, P. M. Howley, D. E. Griffin, R. A. Lamb, M. A. Martin, B. Roizman, and S. E. Straus (ed.), Fields virology, 5th ed. Lippincott, Williams and Wilkins, Philadelphia, Pa.).

The nomenclatures for G genotypes and serotypes are identical (G followed by a number), but the numbers indicating P genotypes are enclosed in brackets, while those for serotypes are not. Since the VP4 and VP7 genes are independently segregated, different G and P combinations have been observed in natural infections. Based on global epidemiology data, G1P[8], G2P[4], G3P[8], G4P[8], G9P[6], and G9P[8] are the most prevalent genotypic combinations found in humans. Other genotypes are often found in animals, although transmission to humans is possible, and the spectrum of genotypes appearing in humans is increasing.

Molecular analyses of VP6 is limited to only a 379-bp fragment of VP6, which results in two broad genogroups that do not correlate with the SG specificities. The classification of rotavirus nonstructural proteins is limited to NSP4, and six genotypes (A to F) have been recognized based on clustering patterns in amino-acid-based phylogenetic dendrograms (Ito, H., M. Sugiyama, K. Masubuchi, Y. Mori, and N. Minamoto. 2001. Complete nucleotide sequence of a group A avian rotavirus genome and a comparison with its counterparts of mammalian rotaviruses. Virus Res. 75:123-138). More recently a classification system that encompasses all 11 RV gene segments has been introduced, and two major genotype (non-G, non-P) constellations termed Wa-like and DS-1-like have been identified.

The rotavirus glycoprotein VP7 defines the G serotypes VP7, or G-protein. VP7 is a Rossmann-fold domain with N- and C-terminal extensions (arms) and with a β-jelly-roll domain inserted into a loop of the Rossmann fold. A Ca2+-stabilized VP7 trimer caps each VP6 trimer in the DLP.

The VP7 trimer contains two structurally-defined antigenic regions, (also referred to herein as domains): 7-1 and 7-2. The 7-2 antigenic region or domain encompasses amino acids 161 to amino acid 255 of the rotavirus VP7 protein. Region 7-1 spans the intersubunit boundary, and is further divided into two subregions (or subdomains): 7-1a, on one side of the interface, and 7-1b on the other. The 7-1a subregion or subdomain spans amino acids 78 to amino acid 160 and the 7-1b subregion or subdomain spans amino acids 256 to 311 of the rotavirus VP7 protein. Each region or domain includes several “epitopes” that have previously been identified and designated by letter and the 7-1 region is the “immunodominant” region that is recognized by 58 out of 68 tested antibodies (Aoki et al. Science, 2009 Jun. 12; 324(5933):1444-1447).

A variety of different approaches have been taken to generate a rotavirus vaccine suitable to protect human populations from the various serotypes of rotavirus. These approaches include various Jennerian approaches, use of live attenuated viruses, use of virus-like particles (VLPs), nucleic acid vaccines and viral sub-units as immunogens. At present there are two oral vaccines available on the market, however, these have low efficacy in some developing countries due to strain variation and presence of other pathogens.

Departing from traditional methods of vaccine generation, advances in the field of molecular biology have permitted the expression of individual rotavirus proteins and the production of Rotavirus-like particles (RLPs).

RLPs are highly organized multimeric protein complexes that self-assemble from viral structural protein(s) and mimic the morphological structure of the corresponding native virus particles without the viral genome, and non-structural viral protein(s). They are produced by the recombinant expression of structural protein(s) in different heterologous expression host cells from bacterial expression systems to various mammalian cell lines. RLPs can be safe and effective alternative candidate vaccines to live-attenuated oral vaccines.

Crawford et al. (J Virol. 1994 September; 68(9): 5945-5952) cloned VP2, VP4, VP6, and VP7 coding for the major capsid protein into the baculovirus expression system and expressed each protein in insect cells. Co-expression of different combinations of the rotavirus major structural proteins resulted in the formation of stable virus-like particles (VLPs). The co-expression of VP2 and VP6 alone or with VP4 resulted in the production of VP2/6 or VP2/4/6 RLPs, which were similar to double-layered rotavirus particles. Co-expression of VP2, VP6, and VP7, with or without VP4, produced triple-layered VP2/6/7 or VP2/4/6/7 RLPs, which were similar to native infectious rotavirus particles. The RLPs maintained the structural and functional characteristics of native particles, as determined by electron microscopic examination of the particles, the presence of non-neutralizing and neutralizing epitopes on VP4 and VP7, and hemagglutination activity of the VP2/4/6/7 RLPs.

While many researchers have successfully produced and purified RV VLPs in insect cells; the efficiency of assembly of RV VLPs in insect cells is very low, with only about 15% of the total virus capsid proteins produced in the insect cells participating in the formation of RV VLPs (Vieira H L A, Alves P M, et al. Intracellular dynamics in rotavirus-like particles production: Evaluation of multigene and monocistronic infection strategies. Proc Biochem, 2006, 41: 2188-2199).

The self-assembly efficiency of 2/6-RV VLPs produced in transgenic plants was even lower. Saldana et al. expressed VP2 and VP6 in the cytoplasm of tomato plants using a cauliflower mosaic virus (CaMV) 35S promoter and recombinant A. tumefaciens (Saldana et al., 2006). Electron microscopy studies showed that a small proportion of the particles had assembled into 2/6 VLPs. A protective immune response was detected in mice and this may have to some extent been contributed by the non-assembled VPs. (Saldana S, Esquivel Guadarrama F, Olivera Flores T de J, et al. Production of rotavirus-like particles in tomato (Lycopersicon esculentum L.) fruit by expression of capsid proteins VP2 and VP6 and immunological studies. Viral immunol, 2006, 19: 42-53).

U.S. Pat. No. 6,867,353 discloses the expression of recombinant rotavirus structural protein VP2, VP4 and VP7 in stable transformed tomato plants. VP7 protein of serotype G1, G2, G3 and G4 were expressed. However, U.S. Pat. No. 6,867,353 does not show the production of rotavirus-like particle.

Choi et al. also expressed a VP7-cholera toxin B fusion protein (CTB::VP7 fusion) in potato. VP7 from simian rotavirus SA11, was fused to the carboxyl terminus of the cholera toxin B subunit and expressed in potato tuber tissue. However ELISA results showed that the CTB::VP7 fusion protein made up only about 0.01% of the total soluble tuber protein (Choi, N W., Estes, M. K. & Langridge, W. H. R. Mol Biotechnol (2005) 31: 193).

Wu et al., 2003 expressed human group A rotavirus serotype G1 VP7 in transgenic potato. Mice immunized with the transformed tubers successfully elicited serum IgG and mucosal IgA specific for VP7. However, the neutralizing activity against rotavirus of VP7 mainly depended on antibodies IgA but not IgG, since the mucosal IgA titer was as high as 1000, while serum IgG titer was only 600 (Wu et al. 2003 “Oral immunization with rotavirus VP7 expressed in transgenic potatoes induced high titers of mucosal neutralizing IgA” Virology, 313 (2003), pp. 337-342).

Another study using transgenic potato plants to express human group A rotavirus serotype G1 VP7 showed that the VP7 gene was stable over 50 generations in the transformed plants. VP7 protein from the 50th generation induced both protective and neutralizing antibodies in adult mice (Li et al. 2006 “Immunogenicity of a plant-derived edible rotavirus subunit vaccine transformed over fifty generations, Virology, Volume 356, Issues 1-2, 5-20 Dec. 2006, Pages 171-178.)

Yang et al. 2011 (Science China Life Sciences January 2011, Volume 54, Issue 1, pp 82-89) co-expressed three rotavirus capsid proteins VP2, VP6 and VP7 of group A RV (P[8]G1) in tobacco plants and expression levels of these proteins, as well as formation of rotavirus-like particles and immunogenicity were studied. VLPs were purified from transgenic tobacco plants and analyzed by electron microscopy and Western blot. Yang et al. results indicate that the plant derived VP2, VP6 and VP7 protein self-assembled into 2/6 or 2/6/7 rotavirus like particle with a diameter of 60-80 nm. However, only a small portion of the expressed rotavirus capsid proteins produced in transgenic tobacco plants assembled into RV VLPs. Yang et al. 2011 found that VP7 was under-expressed in their plants and speculated that VP7 could be the limiting protein during the assembly of triple-layered particles.

Difficulties in expressing recombinant VP7 have been previously described for E. coli and eukaryote cell expression systems, where VP7 was shown to be toxic for the cells (Emslie K R, Miller J M, Slade M B, Dormitzer P R, Greenberg H B, Williams K L. Expression of the rotavirus SA11 protein VP7 in the simple eukaryote Dictyostelium discoideum. J Virol. 1995; 69(3):1747-54, McCrae M A, Corquodale J G. Expression of a major bovine rotavirus neutralization antigen (VP7c) in Escherichia coli. Gene. 1987; 55:9-18.)

Pera et al. 2015 (Virology Journal 201512:205) transiently expressed VP2 and VP6 from human G9P[6] (RVA/Human-wt/ZAF/GR10924/1999/G9P[6]) strain in Nicotiana benthamiana. Pera et al. also attempted the expression of the rotavirus glycoprotein VP7 and the spike protein VP4. However, VP7 expression caused plant wilting during the course of the time trial and expression could never be detected for either protein.

WO 2013/166609 expressed rotavirus capsid proteins VP2, VP6, VP4 and VP7 in plants and the rotavirus proteins auto-assembled into rotavirus-like particles. The VP7 protein had a truncated signal peptide or a non-native signal peptide to increase expression and/or yield of the VP7 protein.

### SUMMARY OF THE INVENTION

The present disclosure relates to producing rotavirus structural proteins in plants. More specifically, the present invention also relates to producing virus-like particles comprising rotavirus structural protein in plants.

According to the present invention there is provided a nucleic acid comprising a nucleotide sequence encoding a rotavirus VP7 fusion protein, the sequence comprising a first sequence encoding a 7-1a subdomain, a second sequence encoding a 7-2 domain and a third sequence encoding a 7-1b subdomain; wherein the sequence of the 7-2 domain is derived from a first rotavirus strain and the sequence of the 7-1a subdomain, the sequence of the 7-1b subdomain or the sequence of the 7-1a subdomain and the sequence of the 7-1b subdomain are derived from a second rotavirus strain, wherein the first rotavirus strain is a different rotavirus strain than the second rotavirus strain.

The 7-2 domain and the 7-1b subdomain may be derived from a first rotavirus strain and the 7-1a subdomain is derived from a second rotavirus strain. The 7-2 domain and the 7-1a subdomain may be derived from a first rotavirus strain and the 7-1b subdomain is derived from a second rotavirus strain. The 7-2 domain may be derived from a first rotavirus strain and the 7-1a subdomain and the 7-1b subdomain are derived from a second rotavirus strain. Furthermore, the first rotavirus strain and the second rotavirus strain may be selected from any one of rotavirus strain having genotype G1 to G19. For example the first rotavirus strain may be a rotavirus strain of genotype G12. In addition the second rotavirus strain or the first rotavirus strain and the second rotavirus strain may not be a rotavirus strain of genotype G4.

The nucleic acid of the current disclosure may further encode a leader peptide (also termed signal peptide) and a grip arm, wherein the leader peptide and a grip arm are derived from the first rotavirus strain.

In another aspect the present disclosure further provides a rotavirus VP7 fusion protein encoded by the nucleic acid as described above.

In a further aspect it is provided a rotavirus like particle (RLP) comprising the rotavirus VP7 fusion protein as described above. The RLP may further comprising rotavirus protein VP2 and VP6 and may be triple-layered. The RLP may comprise a ratio of VP7:VP6 from 0.2 to 0.85. The RLP may further comprise rotavirus structural protein VP2, VP6 and VP7 fusion protein, wherein 5% to 38% of the total structural protein mass of the RLP is VP7 fusion protein.

In another aspect the present disclosure provides a method of producing a rotavirus VP7 fusion protein in a plant, portion of a plant, or a plant cell comprising, providing a plant, portion of a plant, or a plant cell comprising the nucleic acids as described above, and incubating the plant, the portion of a plant, or the plant cell under conditions that permit the expression and production of the rotavirus VP7 fusion protein. Furthermore rotavirus VP7 fusion protein produced by the method as described above are also provided.

In another aspect it is provided a method of producing a rotavirus VP7 fusion protein in a plant, portion of a plant, or a plant cell comprising, introducing the nucleic acid as described above into the plant, the portion of a plant, or the plant cell, and incubating the plant, the portion of the plant, or the plant cell under conditions that permit the expression and production of the rotavirus VP7 fusion protein. Furthermore rotavirus VP7 fusion protein produced by the method as described above are also provided.

In another aspect the present disclosure provides a method (A) of producing a rotavirus like particle (RLP) in a plant, portion of a plant or plant cell comprising:
a) providing a plant, portion of a plant or plant cell comprising a first nucleic acid comprising a first regulatory region active in the plant operatively linked to a first nucleotide sequence encoding the rotavirus VP7 fusion protein of the present disclosure, a second nucleic acid comprising a second regulatory region active in the plant operatively linked to a second nucleotide sequence encoding rotavirus VP2 protein and a third nucleic acid comprising a third regulatory region active in the plant operatively linked to a third nucleotide sequence encoding rotavirus VP6 protein; and
b) incubating the plant, portion of a plant or plant cell under conditions that permit the expression of the first, second and third nucleic acid, thereby producing the RLP.

In the method above the plant, portion of the plant or plant cell may further optionally comprises a fourth nucleic acid comprising a fourth regulatory region active in the plant and operatively linked to a fourth nucleotide sequence encoding rotavirus NSP4 protein or rotavirus VP4 protein. In the method above the plant, portion of the plant or plant cell may further optionally comprises a fifth nucleic acid comprising a fifth regulatory region active in the plant and operatively linked to a fifth nucleotide sequence encoding rotavirus NSP4 protein or rotavirus VP4 protein.

In yet another aspect it is provided a method (B) of producing a rotavirus like particle (RLP) in a plant, portion of a plant or plant cell, comprising:
a) introducing into the plant, portion of a plant or plant cell
   a first nucleic acid comprising a first regulatory region active in the plant operatively linked to a first nucleotide sequence encoding a first rotavirus structural protein selected from one of VP2, VP6 and the rotavirus VP7 fusion protein as described in the present disclosure,
   a second nucleic acid comprising a second regulatory region active in the plant operatively linked to a second nucleotide sequence encoding a second rotavirus structural protein selected from one of VP2, VP6 and the rotavirus VP7 fusion protein, and
   a third nucleic acid comprising a third regulatory region active in the plant operatively linked to a third nucleotide sequence encoding a third rotavirus structural protein selected from one of VP2, VP6 and the rotavirus VP7 fusion protein,
b) incubating the plant, portion of a plant or plant cell under conditions that permit the expression of the first, second and third nucleic acid, thereby producing the RLP comprising VP2, VP6 and the VP7 fusion protein.

In Method (B) a fourth nucleic acid comprising a fourth regulatory region active in the plant and operatively linked to a fourth nucleotide sequence encoding rotavirus protein NSP4 or rotavirus protein VP4 may further be introduced into the plant, portion of the plant or the plant cell in step a), and may be expressed when incubating the plant, portion of the plant or the plant cell in step b) to produce RLP. Furthermore a fifth nucleic acid comprising a fifth regulatory region active in the plant and operatively linked to a fifth nucleotide sequence encoding rotavirus protein NSP4 or rotavirus protein VP4 may further be introduced into the plant, portion of the plant or the plant cell in step a), and may be expressed when incubating the plant, portion of the plant or the plant cell in step b) to produce RLP.

The methods (A) and (B) as described above may further comprise the steps of c) harvesting the plant, portion of a plant or plant cell, and d) extracting and purifying the RLPs from the plant, portion of a plant or plant cell. In the method (A) or (B) the first, second and third nucleic acid may be transiently or stably expressed in the plant, portion of the plant or plant cell.

In a further aspect it is provide RLP produced by methods (A) or (B) as described above. The RLP may comprise a ratio of VP7:VP6 from 0.2 to 0.85. 5% to 38% of the total structural protein mass of the RLP may be VP7 fusion protein.

In another aspect it is provided an antibody or antibody fragment prepared using the rotavirus VP7 fusion protein as described above. The antibody or antibody fragment may recognize an epitope of the 7-1a subdomain.

In another aspect it is provided an antibody or antibody fragment prepared using the RLP as described above. The antibody or antibody fragment may recognize an epitope of the 7-1a subdomain.

In another aspect it is provided a method of producing an antibody or an antibody fragment comprising, administering the rotavirus VP7 fusion protein or the RLP as described above to a subject in need thereof, or a host animal, thereby producing the antibody or the antibody fragment. A composition for inducing an immune response comprising, an effective dose of the rotavirus VP7 fusion protein of or the RLP, and a pharmaceutically acceptable carrier, adjuvant, vehicle or excipient are also provided. In addition a vaccine comprising an effective dose of the rotavirus VP7 fusion protein or the RLP for inducing an immune response are provided.

In another aspect it is provided a method for inducing immunity to a rotavirus infection in a subject, the method comprising administering the rotavirus VP7 fusion protein or the RLP as described herewith to the subject.

In a further aspect a plant, portion of the plant, or the plant cell or a plant extract comprising the nucleic acid, the rotavirus VP7 fusion protein or the RLP as described herewith are provided.

In yet another aspect the disclosure provides a method of increasing yield of production of a rotavirus VP7 fusion protein in a plant, portion of a plant, or a plant cell, comprising:
a) introducing the nucleic acid as described herewith into the plant, portion of the plant, or plant cell; or providing a plant, portion of a plant, or plant cell comprising the nucleic acid as described herewith; and
b) incubating the plant, portion of the plant, or plant cell under conditions that permit expression of the rotavirus VP7 fusion protein encoded by the nucleic acid, thereby producing the rotavirus VP7 fusion protein at a higher yield compared to a plant, portion of the plant, or plant cell expressing native rotavirus VP7 protein.

In method may further comprise step c), harvesting the plant, portion of the plant, or plant cell, and purifying the rotavirus VP7 fusion protein.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1A shows a ribbon diagram of the trimer, viewed along its threefold axis (i.e., as if looking onto the surface of the virion) with one subunit shown in dark grey and the other two subunits in light gray. The 7-1 domain (Rossmann-fold domain; domain I), which comprise 7-1a and 7-1b subdomains and the 7-2 domain (β-barrel domain: domain II), are indicated.

FIG. 1B shows a schematic diagram of the VP7 primary structure, including the leader sequence, also referred to as signal peptide (Sp) (residues 1-50, light gray), the grip arm (residues 51-77), the 7-1a subdomain (residues 78-160, grey), the 7-2 domain (residues 161-255, black), the 7-1b subdomain (residues 256-311, grey) and the C-terminal end (residues 312-326). The pattern of intrasubunit disulfide bonds is also shown, with numbers corresponding to the positions of the cysteine residues.

FIG. 2A shows a schematic diagram of the domain structure of a native VP7 protein from a first rotavirus genotype (e.g. genotype G1) and a second genotype and exemplary VP7 fusion proteins. The VP7 proteins comprise a signal peptide (Sp), a grip arm domain (not marked), a 7-1a subdomain, a 7-2 domain, a 7-1b subdomain and a C-terminal end (not marked). The rotavirus 7-1a VP7 fusion protein (7-1a₂--7-2₁--7-1b₁) comprises a signal peptide, a grip arm, a 7-2 domain, a 7-1b subdomain and a C-terminal end from a first rotavirus genotype and a 7-1a subdomain from a second rotavirus genotype. The rotavirus 7-1b VP7 fusion protein (7-1a₁--7-2₁--7-1b₂) comprises a signal peptide, a grip arm, a 7-1a subdomain, a 7-2 domain, and a C-terminal end from a first rotavirus genotype and a 7-1b subdomain from a second rotavirus genotype. The rotavirus 7-1a+7-1b VP7 fusion protein (7-1a₂--7-2₁--7-1b₂) comprises a signal peptide, a grip arm, a 7-2 domain, and a C-terminal end from a first rotavirus genotype and a 7-1a subdomain and a 7-1b subdomain from a second rotavirus genotype. FIG. 2B shows alignments of the amino acid sequence of the boundary sequences of VP7 domains and subdomains of rotavirus of genotype G1P8 (G1P8_Rtx; H2E8G2; RVA/Vaccine/USA/Rotarix-A41CB052A/1988/G1P1A[8]; Genbank: AEX30682), G2P5 (G2P5_SC2-9; RVA/Vaccine/USA/RotaTeq-SC2-9/1992/G2P7[5], GenBank: ADK27036), G3P5 (G3P6_WI78-8; RVA/Vaccine/USA/RotaTeq-WI78-8/1992/G3P7[5]; GenBank: ADK27037), G4P5 (G4P5_BrB-9; RVA/Vaccine/USA/RotaTeq-BrB-9/1996/G4P7[5]; GenBank: ADK27038), G9P8 (G9P8_WI61;A Hu/WI61/1983/G9P1A[8]; UniProtKB/Swiss-Prot: B3SRX9) and G12P8 (G12P8_KDH651; RVA/Human-tc/KEN/KDH651/2010/G12P[8]; GenBank: BAO74145). Conserved amino acids are shaded in dark grey. Numbers corresponding to the amino acid position in the sequence of the VP7 protein sequence are indicated.

FIG. 3A upper panel shows Coomassie-stained SDS-PAGE analysis of crude protein extracts prepared from N. benthamiana leaves having been transformed with constructs encoding the following VP7 protein: human codon optimized native G1 VP7 (‘VP7 Rtx’, USA/Rotarix-A41CB052A/1988/G1P1A[8], Genbank: AEX30682, construct 1199, SEQ ID NO: 20); human codon optimized native G2P5 VP7 (‘VP7 G2P5’, RVA/Vaccine/USA/RotaTeq-SC2-9/1992/G2P7[5], GenBank: ADK27036, construct 3463, SEQ ID NO: 26); human codon optimized fusion VP7 RVA(Rtx G1) VP7 (7-1a G2) (‘VP7(Rtx)+(7-1a)G2, construct 4540, SEQ ID NO: 28); human codon optimized fusion VP7 RVA(Rtx G1) VP7 (7-1b G2) (VP7(Rtx)+(7-1b)G2, construct 4541, SEQ ID NO: 31); human codon optimized fusion VP7 RVA(Rtx G1) VP7 (7-1a-1b G2) (‘VP7(Rtx)+(7-1a-1b)G2, construct 4542, SEQ ID NO: 33); human codon optimized native G9P8 VP7 (Hu/WI61/1983/G9P1A[8], UniProtKB/Swiss-Prot: B3SRX9, construct 3481, SEQ ID NO: 37); human codon optimized fusion VP7 RVA(Rtx G1) VP7 (7-1a G9) (‘VP7(Rtx)+(7-1a)G9, construct 4546, SEQ ID NO: 39); human codon optimized fusion VP7 RVA(Rtx G1) VP7 (7-1b-G9) (‘VP7(Rtx)+(7-1b)G9, construct 4547, SEQ ID NO: 41 and human codon optimized fusion VP7 RVA(Rtx G1) VP7 (7-1a-1b G9) (‘VP7(Rtx)+(7-1a-1b’)G9, construct 4548, SEQ ID NO: 43). Lower panel shows Western blot analysis with anti-VP7 antibodies of the gel shown in the upper panel.

FIG. 3B upper panel shows Coomassie-stained SDS-PAGE analysis of crude protein extracts prepared from N. benthamiana leaves having been transformed with constructs encoding the following VP7 protein: human codon optimized native G1 VP7 (‘VP7 Rtx’, USA/Rotarix-A41CB052A/1988/G1P1A[8], Genbank: AEX30682, construct 1199, SEQ ID NO: 20); human codon optimized native G3P5 VP7 (‘VP7 G3P5’, RVA/Vaccine/USA/RotaTeq-WI78-8/1992/G3P7[5], GenBank: ADK27037, construct 3469, SEQ ID NO: 47); human codon optimized fusion VP7 RVA(Rtx G1) VP7 (7-1b G3) (‘VP7(rtx)+7-1b)G3, construct 4551, SEQ ID NO: 50); human codon optimized fusion VP7 RVA(Rtx G1) VP7 (7-1a-1b G3) (‘VP7(rtx)+7-1a-1b)G3, construct 4552, SEQ ID NO: 52); human codon optimized native G12P8 VP7 (‘VP7 G12P8’, RVA/Human-tc/KEN/KDH651/2010/G12P[8], GenBank: BAO74145, construct 3487, SEQ ID NO: 56); human codon optimized fusion VP7 RVA(Rtx G1) VP7 (7-1b G12) (‘VP7(Rtx)+7-1b)G12, construct 4553, SEQ ID NO: 59) and human codon optimized fusion VP7 RVA(Rtx G1) VP7 (7-1a-1b G12) (‘VP7(Rtx)+7-1a-1b)G12, construct 4554, SEQ ID NO: 61). Lower panel shows Western blot analysis with anti-VP7 antibodies of the gel shown in the upper panel.

FIG. 4A shows electron micrographs of rotavirus RLPs produced in plants, the RLPs comprise native VP7 from rotavirus strain G9P[6] (Hu/BEL/BE2001/2009/G9P[6], GenBank: AFJ11215). FIG. 4B shows electron micrographs of rotavirus RLPs produced in plants, the RLPs comprise human codon optimized fusion VP7 RVA(Rtx G1) VP7 (7-1a-1b G9) (construct 4548, SEQ ID NO: 43).

FIG. 5 shows a neutralizing activity of native G9-RLP (G9-RLP AFJ11215) and G9-RLP comprising VP7 fusion protein (G9-RLP chimera) against WI61 strain (G9P[8]).

FIG. 6a shows a schematic representation of vector 1710 (RVA(WA) VP2(opt)); FIG. 6b shows a schematic representation of vector 1191 (C5-1 gamma); FIG. 6c shows a schematic representation of vector 1713 (RVA(WA) VP6(opt)); FIG. 6d shows a schematic representation of vector 1706 (RVA(WA) NSP4); FIG. 6e shows a schematic representation of vector 1708 (RVA(WA) VP6(opt) and RVA(WA) VP2(opt)); FIG. 6f shows a schematic representation of vector 2252 (RVA(WA) VP6(opt), RVA(WA) VP2(opt) and RVA(WA) NSP4); FIG. 6g shows a schematic representation of vector 1190 (C5-1 gamma); FIG. 6h shows a schematic representation of vector 1199 (RVA(Rtx G1) VP7(opt)); FIG. 6i shows a schematic representation of vector 3463 (RVA(Sc2-9 G2) VP7(opt)); FIG. 6j shows a schematic representation of vector 4540 (RVA(Rtx G1) VP7 (7-1a G2)) FIG. 6k shows a schematic representation of vector 4541 (RVA(Rtx G1) VP7 (7-1b G2)); FIG. 6l shows a schematic representation of vector 4542 (RVA(Rtx G1) VP7 (7-1a-1b G2)); FIG. 6m shows a schematic representation of vector 3481 (RVA(WI61 G9) VP7(opt)); FIG. 6n shows a schematic representation of vector 4546 (RVA(Rtx G1) VP7 (7-1a G9)); FIG. 6o shows a schematic representation of vector 4547 (RVA(Rtx G1) VP7 (7-1b-G9)); FIG. 6p shows a schematic representation of vector 4548 (RVA(Rtx G1) VP7 (7-1a-1b G9)); FIG. 6q shows a schematic representation of vector 3469 (RVA(WI78-8 G3) VP7(opt)); FIG. 6r shows a schematic representation of vector 4551 (RVA(Rtx G1) VP7 (7-1b G3)); FIG. 6s shows a schematic representation of vector 4552 (RVA(Rtx G1) VP7 (7-1a-1b G3)); FIG. 6t shows a schematic representation of vector 3487 (RVA(KDH651 G12) VP7(opt)); FIG. 6u shows a schematic representation of vector 4553 (RVA(Rtx G1) VP7 (7-1b G12)); FIG. 6v shows a schematic representation of vector 4554 (RVA(Rtx G1) VP7 (7-1a-1b G12)).

FIG. 7a shows a schematic representation of vector 6026 (RVA (G3 HCR3) VP7 (opt)). FIG. 7b shows a schematic representation of vector 6501 (RVA (G3 HCR3) VP7 (7-1a-1b G1 Rtx) (opt); FIG. 7c shows a schematic representation of vector 6502 (RVA (G3 HCR3) VP7 (7-1a-1b G2 Sc2-9) (opt); FIG. 7d shows a schematic representation of vector 6503 (RVA (G3 HCR3) VP7 (7-1a-1b G4 BrB-9) (opt); FIG. 7e shows a schematic representation of vector 6504 RVA (G3 HCR3) VP7 (7-1a-1b G9 BE2001) (opt); FIG. 7f shows a schematic representation of vector 6505 (RVA (G3 HCR3) VP7 (7-1a-1b G12 K12) (opt)); FIG. 7g shows a schematic representation of vector 3475 (RVA (G4 BrB-9) VP7 (opt); FIG. 7h shows a schematic representation of vector 6506 (RVA (G4 BrB-9) VP7 (7-1a-1b G1 Rtx) (opt)); FIG. 7i shows a schematic representation of vector 6507 (RVA (G4 BrB-9) VP7 (7-1a-1b G2 Sc2-9) (opt)); FIG. 7j shows a schematic representation of vector 6508 (RVA (G4 BrB-9) VP7 (7-1a-1b G3 HCR3) (opt); FIG. 7k shows a schematic representation of vector 6509 (RVA (G4 BrB-9) VP7 (7-1a-1b G9 BE2001) (opt); FIG. 7l shows a schematic representation of vector 6510 (RVA (G4 BrB-9) VP7 (7-1a-1b G12 K12) (opt); FIG. 7m shows a schematic representation of vector 4568 (RVA (G9 BE2001) VP7 (opt) (RVA(WI61 G9) VP7(opt)); FIG. 7n shows a schematic representation of vector 6511 (RVA (G9 BE2001) VP7 (7-1a-1b G1 Rtx) (opt)); FIG. 7o shows a schematic representation of vector 6512 (RVA (G9 BE2001) VP7 (7-1a-1b G2 Sc2-9) (opt)); FIG. 7p shows a schematic representation of vector 6513 (RVA (G9 BE2001) VP7 (7-1a-1b G3 HCR3) (opt)); FIG. 7q shows a schematic representation of vector 6514 (RVA (G9 BE2001) VP7 (7-1a-1b G4 BrB-9) (opt)); FIG. 7r shows a schematic representation of vector 6515 (RVA (G9 BE2001) VP7 (7-1a-1b G12 K12) (opt)); FIG. 7s shows a schematic representation of vector 6042 (RVA (G12 K12) VP7 (opt)); FIG. 7t shows a schematic representation of vector 6516 (RVA (G12 K12) VP7 (7-1a-1b G1 Rtx) (opt)); FIG. 7u shows a schematic representation of vector 6517 (RVA (G12 K12) VP7 (7-1a-1b G2 Sc2-9) (opt)); FIG. 7v shows a schematic representation of vector 6518 (RVA (G12 K12) VP7 (7-1a-1b G3 HCR3) (opt)). FIG. 7w shows a schematic representation of vector 6519 (RVA (G12 K12) VP7 (7-1a-1b G4 BrB-9) (opt)); FIG. 7x shows a schematic representation of vector 6520 (RVA (G12 K12) VP7 (7-1a-1b G9 BE2001) (opt).

### DETAILED DESCRIPTION

The following description is of a preferred embodiment.

The present invention has been described with regard to one or more embodiments. However, it will be apparent to persons skilled in the art that a number of variations and modifications can be made without departing from the scope of the invention as defined in the claims.

As used herein, the terms “comprising,” “having,” “including” and “containing,” and grammatical variations thereof, are inclusive or open-ended and do not exclude additional, un-recited elements and/or method steps. The term “consisting essentially of” when used herein in connection with a use or method, denotes that additional elements and/or method steps may be present, but that these additions do not materially affect the manner in which the recited method or use functions. The term “consisting of” when used herein in connection with a use or method, excludes the presence of additional elements and/or method steps. A use or method described herein as comprising certain elements and/or steps may also, in certain embodiments, consist essentially of those elements and/or steps, and in other embodiments consist of those elements and/or steps, whether or not these embodiments are specifically referred to. In addition, the use of the singular includes the plural, and “or” means “and/or” unless otherwise stated. The term “plurality” as used herein means more than one, for example, two or more, three or more, four or more, and the like. Unless otherwise defined herein, all technical and scientific terms used herein have the same meaning as commonly understood by one of ordinary skill in the art. As used herein, the term “about” refers to an approximately +/−10% variation from a given value. It is to be understood that such a variation is always included in any given value provided herein, whether or not it is specifically referred to. The use of the word “a” or “an” when used herein in conjunction with the term “comprising” may mean “one,” but it is also consistent with the meaning of “one or more,” “at least one” and “one or more than one.”

Rotavirus VP7 fusion proteins and methods of producing rotavirus VP7 fusion proteins in a plants, portion of a plants or plant cells are described herein. The rotavirus VP7 fusion protein may have one or more than one domain or subdomain from a first rotavirus fused to one or more than one domain or subdomain from a second rotavirus. It has been observed that expression of the VP7 fusion protein in a plant, portion of a plant or plant cell increases the yield of the VP7 fusion protein, when compared to the yield of a wildtype or native VP7 protein expressed in the same type of plant, portion of a plant or plant cell and under the same conditions.

Furthermore, methods of producing rotavirus like particle (RLP) comprising rotavirus VP7 fusion proteins in a plants, portion of a plants or plant cells are also described. It has been observed that when RLPs are produced that comprise rotavirus VP7 fusion proteins as described herein, the yield of RLP production is increased compared to the yield of RLPs comprising wildtype or native VP7 produced in the same type of plant, portion of a plant or plant cell and under the same conditions.

It has also been observed that when RLPs are produced that comprise rotavirus VP7 fusion proteins as described herein, the RLP comprise a higher content, higher amount or higher incorporation of rotavirus VP7 fusion proteins compared to RLP that comprise wildtype of native VP7. Methods of increasing rotavirus VP7 incorporation into RLP produced in a plants, portion of a plants or plant cells and RLPs with increased rotavirus VP7 protein incorporation that have been produced in a plants, portion of a plants or plant cells are, therefore, also provided.

The higher content, higher amount or higher incorporation of rotavirus VP7 fusion protein, may be expressed for example as ratio of rotavirus VP7 fusion protein (VP7) to rotavirus VP6 protein (VP6). Accordingly, it is further provided RLPs that may comprise a higher ratio of VP7:VP6, when compared to RLP that comprise wildtype or native VP7.

### Rotavirus Strains

The term “rotavirus”, as used herein, refers to multi-layered, non-enveloped viral strain of the genus rotavirus of the family Reoviridae. The mature particle consists of a triple-layered capsid consisting of the outer, intermediate, and inner layers. The outer capsid or layer contains the VP4 and VP7 protein, whereas the intermediate layer is formed by VP6, and the inner by VP2 which encloses two other proteins VP1 and VP3, as well as the viral genome consisting of 11 segments of double-stranded RNA, the latter encoding six structural and six nonstructural proteins. The inner layer of the capsid is a thin shell made up of 120 polypeptides of VP2, which form 60 asymmetric dimers which are, in turn, arranged with T=1 icosahedral symmetry. The outer layer of the double-layered particle (DLP) i.e., the middle layer of the mature particle is composed of 780 VP6 polypeptides, which are distributed as 260 trimers. The outer layer of the virion is composed of 260 trimers of the 37-kDa glycoprotein VP7, the most abundant external protein, which constitute the smooth surface of the virion, and 60 dimeric spikes of the 88-kDa protein VP4. Because of the segmented nature of the rotavirus genome, genetic reassortment occurs at high frequency during mixed infection.

Rotaviruses can be serotyped by neutralization assays with panels of antisera and genotyped by sequence analysis of different gene segments. Although there is a close relationship between the two classification systems, it has recently been proposed that the term serotype should be reserved for serological analysis and that the term genotype should be used for genetic classification and comparative sequence analysis. Generally it is accepted that strains that have more than 89% amino acid identity are considered to be of the same genotype (Estes, M. K. 2001. Rotaviruses and their replication, p. 1747-1786. In P. M. Howley (ed.), Fields virology, vol. 2., 4th ed. Lippincott Williams & Wilkins, Philadelphia, Pa.). However based on more recent phylogenetic analyses, appropriate identity cut-off values were determined for each gene. For the VP7 gene, a nucleotide identity cut-off value of 80% largely coincided with the established G genotypes, but identified four additional distinct genotypes comprised of murine or avian rotavirus strains. (J Virol. 2008 April; 82(7):3204-19).

Accordingly, for the present application it is considered that two or more rotaviruses belong to the same “rotavirus strain” or the same “rotavirus genotype”, when the amino acid sequences of the VP7 protein from the rotaviruses have at least 89% amino acid identity or when the nucleotide sequences encoding the VP7 protein from the rotaviruses share at least 80% sequence similarity. Conversely, two or more rotavirus are considered to belong to different “rotavirus strains” or different “rotavirus genotypes” when the amino acid sequences of the VP7 protein from the rotaviruses have less than 89% amino acid identity or when the nucleotide sequences encoding the VP7 protein from the rotaviruses share less than 80% sequence similarity.

Methods for determining sequence identity or sequence similarity are well-known in the art and may be determined using a nucleotide sequence comparison program, such as that provided within DNASIS (using, for example but not limited to, the following parameters: GAP penalty 5, #of top diagonals 5, fixed GAP penalty 10, k tuple 2, floating gap 10, and window size 5). However, other methods of alignment of sequences comparison and determination of sequence identity or similarity are well-known in the art for example the algorithms of Smith & Waterman (1981, Adv. Appl. Math. 2:482), Needleman & Wunsch (J. Mol. Biol. 48:443, 1970), Pearson & Lipman (1988, Proc. Nat'l. Acad. Sci. USA 85:2444), and by computerized implementations of these algorithms (GAP, BESTFIT, FASTA, and BLAST, available through the NIH.), or by manual alignment and visual inspection (see, e.g., Current Protocols in Molecular Biology, Ausubel et al., eds. 1995 supplement), or using Southern or Northern hybridization under stringent conditions (see Maniatis et al., in Molecular Cloning (A Laboratory Manual), Cold Spring Harbor Laboratory, 1982).

### VP7 Fusion Protein

Rotavirus VP7 fusion proteins and methods of producing rotavirus VP7 fusion proteins in plants are described herein. The rotavirus VP7 fusion protein (also referred to a ‘VP7 fusion’ or ‘fusion VP7’) may have one or more than one domain or subdomain from a first rotavirus fused to one or more than one domain or subdomain from a second rotavirus.

The rotavirus VP7 fusion protein may have a 7-1a subdomain, a 7-2 domain and a 7-1b subdomain; wherein the sequence of the 7-2 domain is derived from a first rotavirus strain and the sequence of the 7-1a subdomain, the sequence of the 7-1b subdomain or the sequence of the 7-1a subdomain and the sequence of the 7-1b subdomain are derived from a second rotavirus strain. The first rotavirus strain is a different rotavirus strain than the second rotavirus strain.

The expression “first rotavirus” or “first rotavirus strain” refers to a rotavirus that has a first genotype or a first serotype based on genotyping or serotyping of the rotavirus VP7 protein of the first rotavirus. The expression “second rotavirus” or “second rotavirus strain” refers to one or more rotavirus that have a second genotype or second serotype based on the genotyping or serotyping of the rotavirus VP7 protein of the second rotavirus, wherein the second genotype or second serotype differs from the first genotype or first serotype. A first rotavirus strain differs from a second rotavirus strain in genotype, serotype or genotype and serotype of the VP7 protein.

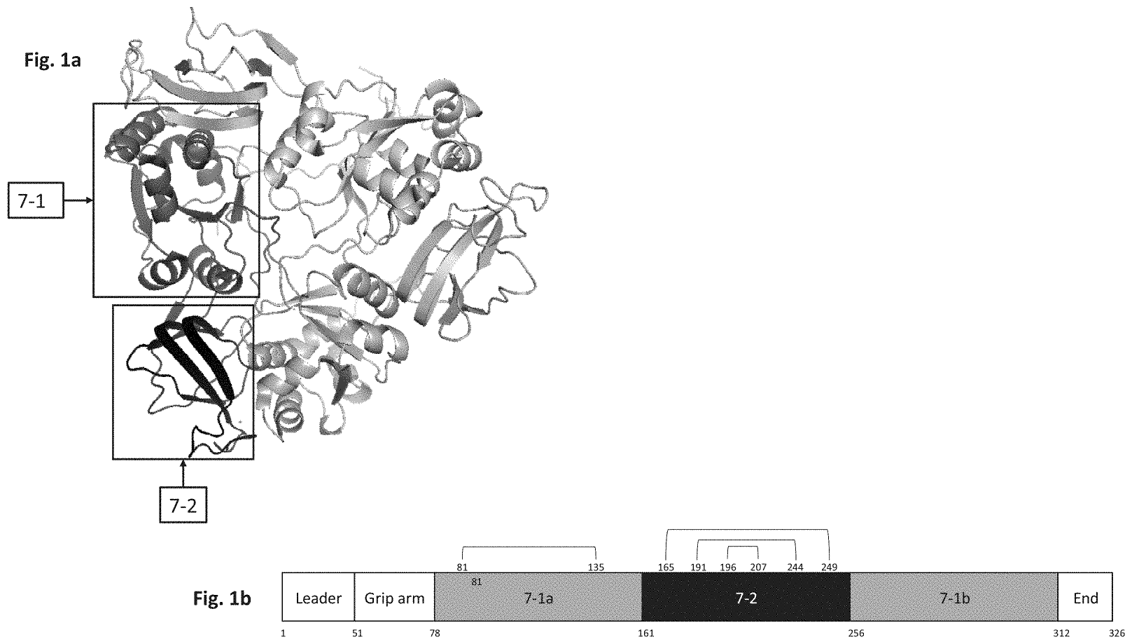

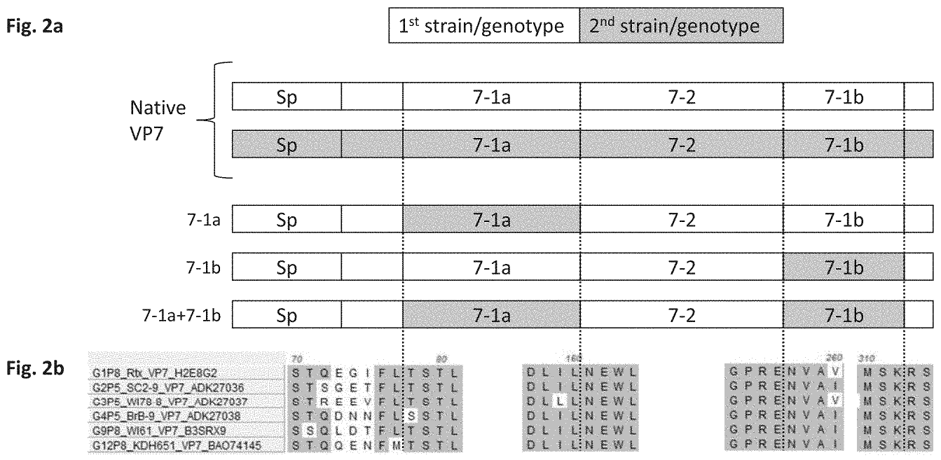

The domain organization of rotavirus VP7 is illustrated in FIGS. 1B and 2A. The rotavirus VP7 primary structure includes a leader sequence, also referred to as signal peptide (Sp) (residues 1-50), a grip arm (residues 51-77), a 7-1a subdomain (residues 78-160), a 7-2 domain (residues 161-255), a 7-1b subdomain (residues 256-311) and a C-terminal end (residues 312-326).

The corresponding amino acid positions in rotavirus strains of various genotypes may be determined by alignment to known sequences of the rotavirus VP7 protein. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

As described above although VP7 protein has a high diversity among strains, multiple nucleotide sequences, or corresponding polypeptide sequences of rotavirus VP7, may be aligned to determine a “consensus” or “consensus sequence” of the VP7 protein.

The amino acid sequences adjacent to the boundaries of the VP7 domains and subdomains of rotavirus VP7 are well conserved (see FIG. 2b) and comprise of the following exemplary consensus sequences:

a. Non-limiting example of a boundary sequence between the grip arm domain and the 7-1a subdomain of rotavirus VP7

(SEQ ID NO: 118)
. . . StqXXXFlM∥tSTL . . . ,

where “∥” indicates the boundary between the grip arm domain and the 7-1a subdomain. The boundary sequence may comprise amino acids from position 70 to position 81 within the rotavirus VP7 protein and the boundary between the grip arm domain and the 7-1a subdomain may be located between amino acid 77 and 78 of the rotavirus VP7 protein.

b. Non-limiting example of a boundary sequence between the 7-1a subdomain and the 7-2 domain of rotavirus VP7

(SEQ ID NO: 119)
. . . DLiL∥NEWL . . . ,

where “∥” indicates the boundary between the 7-1a subdomain and 7-2 domain. The boundary sequence may comprise amino acids from position 157 to position 164 within the rotavirus VP7 protein and the boundary between the 7-1a subdomain and the 7-2 domain may be located between amino acid 160 and 161 of the rotavirus VP7 protein.

c. Non-limiting example of a boundary sequence between the 7-2 domain and the 7-1b subdomain of rotavirus VP7

(SEQ ID NO: 120)
. . . GPRE∥NVAi . . . ,

where “∥” indicates the boundary between the 7-2 domain and the 7-1b subdomain. The boundary sequence may comprise amino acids from position 253 to position 260 within the rotavirus VP7 protein and the boundary between the 7-2 domain and the 7-1b subdomain may be located between amino acid 256 and 257 of the rotavirus VP7 protein.

d. Boundary sequence between the 7-1b subdomain and the C-terminal end of rotavirus VP7

(SEQ ID NO: 121)
. . . MSK∥RS . . . ,

where “∥” indicates the boundary between the 7-1b subdomain and the C-terminal end. The boundary sequence may comprise amino acids from position 310 to position 314 within the rotavirus VP7 protein and the boundary between the 7-1b subdomain and the C-terminal end may be located between amino acid 312 and 313 of the rotavirus VP7 protein.

The rotavirus VP7 fusion protein may comprise a 7-2 domain derived a first rotavirus genotype or strain and a 7-1a subdomain, a 7-1b subdomain or a 7-1a subdomain and a 7-1b subdomain derived from a second rotavirus genotype or strain. Accordingly, by “VP7 fusion protein” or “chimeric VP7 protein” it is meant, a protein comprising a 7-2 domain derived from a first rotavirus genotype fused to the 7-1a, 7-1b or 7-1a and 7-1b subdomains derived from a second rotavirus genotype, wherein the VP7 fusion protein comprises at least one domain or subdomain from a first rotavirus genotype or strain and at least one or more domain or subdomain from a second rotavirus genotype or strain. The VP7 fusion protein may comprise a 7-2 domain derived from a first rotavirus genotype or strain and one or more than one of the 7-1a and 7-1b subdomain may be derived from a second rotavirus genotype or strain:
7-1a_{1st or 2nd strain}--7-2_{1st strain}--7-1b_{1st or 2nd strain} (7-1a_{1/2}-7-2₁--7-1b_{1/2}).

The sequence encoding the VP7 fusion protein may be optimized for human codon usage, for having an increased GC content, or a combination thereof

The rotavirus structural protein described herewith may comprise a truncated, native or a non-native signal peptide (SP). The signal peptide (SP) may be native to the rotavirus structural protein such as for example VP7 fusion, VP2, VP4, VP6 or NSP4. For example, in the VP7 fusion protein the signal peptide may be from a first or a second rotavirus genotype or strain. The signal peptide may also be heterologous in the sense that the signal peptide may be from a third rotavirus genotype or strain which is different from the first or second rotavirus genotype or strain. For example the signal peptide may be heterologous with respect to the first or a second rotavirus genotype or strain in the VP7 fusion protein. The native signal peptide of rotavirus structural protein may be used to express the rotavirus structural protein in host or host cell such for example a plant system, plant, portion of a plant or plant cell.

A signal peptide may also be non-native, for example, from a protein, viral protein or native structural protein of a virus other than rotavirus protein, or from a plant, animal or bacterial polypeptide. A non-limiting example of a signal peptide that may be used is disulfide isomerase signal (PDI) peptide for example alfalfa protein disulfide isomerase (nucleotides 32-103 of Accession No. Z11499). Furthermore, the signal peptide may be completely deleted or truncated. By truncation or truncated it is meant that 1%, 5%, 10%, 15%, 20%, 25%, 30%, 35%, 40%, 45%, 50%, 55%, 60%, 65%, 70%, 75%, 80%, 85%, 90%, 95%, 100% or any amount therebetween of amino acid residues are deleted from the signal peptide. Accordingly, the truncated signal peptide may have 1-50 amino acids or any amount therebetween deleted. For example the truncated signal peptide may have 1, 2, 3, 4, 5, 6, 7, 8, 9, 10, 11, 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49 or 50 amino acids deleted from its original sequence. Preferably, the truncated amino acid residues are continuous, and the truncation occurs from the second methionine onward.

The VP7 fusion protein is heterologous (or chimeric) in that the fusion protein comprises a 7-2 domain from a first VP7 protein (from a first rotavirus strain or genotype) and a 7-1a subdomain, a 7-1b subdomain or a 7-1a subdomain and a 7-1b subdomain from a second VP7 protein (from a second rotavirus strain or genotype). The heterologous VP7 fusion protein may comprise a 7-2 domain, a 7-1a subdomain and a 7-1b subdomain with an amino acid sequence that falls within, or the amino acid sequence is found within (or maps against) the 7-2 domain, the 7-1a subdomain, the 7-1b subdomain consensus sequence of the VP7 sequence and wherein the 7-1a subdomain sequence is between the Grip arm∥7-1a boundary (SEQ ID NO: 62) and the 7-1a∥7-2 boundary (SEQ ID NO: 63), the 7-2 domain sequence is between the 7-1a∥7-2 boundary (SEQ ID NO: 63) and the 7-2∥7-1b boundary (SEQ ID NO: 64) and the 7-1b subdomain sequence is between the 7-2∥7-1b boundary (SEQ ID NO: 64) and the 7-1b∥C-terminal end boundary (SEQ ID NO: 65), as shown in FIGS. 2a and 2b and provided that the 7-2 domain of the fusion protein is heterologous to the 7-1a subdomain, the 7-1b subdomain or the 7-1a subdomain and the 7-1b subdomain and that the VP7 fusion protein induces immunity to rotavirus in a subject, when the VP7 protein is administered to the subject. The induced immunity might be against the second rotavirus strain or genotype, the first rotavirus strain or genotype or both.

A. 7-1a₂--7-2₁--7-1b₁; 7-1a

For example, the rotavirus VP7 fusion protein, and methods of producing the rotavirus VP7 fusion protein, may include a rotavirus VP7 fusion protein comprising a 7-2 domain and a 7-1b subdomain derived from a first rotavirus genotype or strain and a 7-1a subdomain from a second rotavirus genotype or strain. The 7-1a subdomain from a second rotavirus genotype or strain is being fused to the 7-2 domain and the 7-1b subdomain, wherein the 7-2 domain and the 7-1b subdomain both are derived from a first rotavirus genotype or strain:
7-1a_{2nd stain}--7-2_{1st strain}--7-1b_{1st strain} (7-1a₂--7-2₁--7-1b₁; 7-1a).

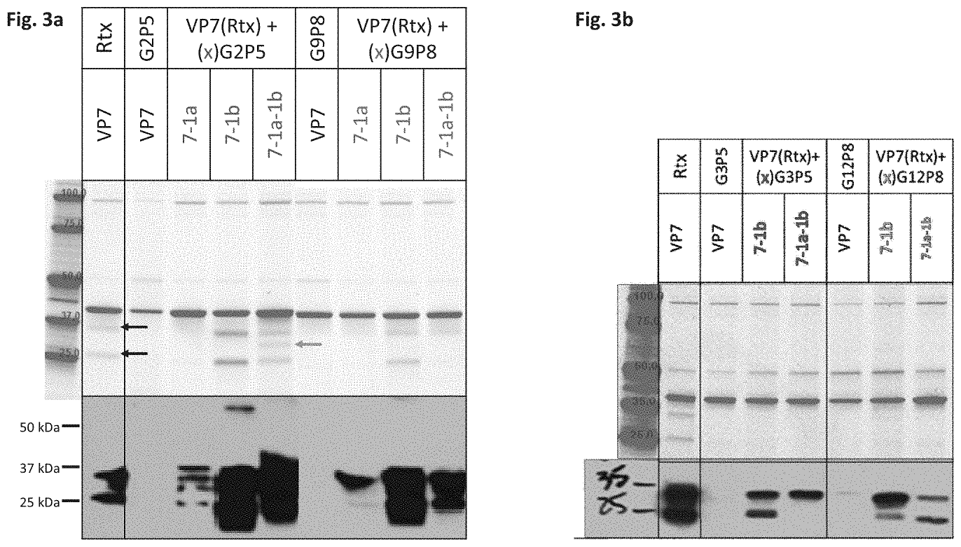

It has been observed that expression of the VP7 fusion protein (7-1a₂--7-2₁--7-1b₁) comprising a 7-2 domain and a 7-1b subdomain derived from a first rotavirus genotype or strain and a 7-1a subdomain from a second rotavirus genotype or strain increases the yield of the VP7 fusion protein, when compared to the yield of a wildtype or native VP7 protein comprising a 7-2 domain and a 7-1 domain (7-1a and 7-1b subdomains) from the same second rotavirus genotype or strain, when expressed in the same type of plant and under the same conditions, as a native or wildtype VP7 protein (see for example FIG. 3a, third and seventh lane (7-1a), compared to the second (VP7 G2P5) and sixth lane (VP7 G9P8), respectively).

Examples of VP7 fusion protein of the form 7-1a2-7-2₁--7-1b₁ (7-1a) include, but are not limited to: VP7(Rtx)+(7-1a)G2P5 [RVA(Rtx G1) VP7 (7-1a G2); SEQ ID NO: 28] comprising a 7-2 domain and a 7-1b subdomain from USA/Rotarix-A41CB052A/1988/G1P1A[8] and a 7-1a subdomain from rotavirus strain G2P5 [RVA/Vaccine/USA/RotaTeq-SC2-9/1992/G2P7[5]] or VP7(Rtx)+(7-1a)G9P8 [RVA(Rtx G1) VP7 (7-1a G9); SEQ ID NO: 39] comprising a 7-2 domain and a 7-1b subdomain from USA/Rotarix-A41CB052A/1988/G1P1A[8] and a 7-1a subdomain from rotavirus strain G9P8 [RVA/Hu/WI61/1983/G9P1A[8]], or a sequence that exhibits from about 59-100% or any amount therebetween, sequence similarity or identity with the amino acid sequence of the 7-1a2-7-2₁--7-1b₁ (7-1a) fusion amino acid sequence of SEQ ID NO: 28 or 39, for example from about 59, 60, 62, 64, 66, 68, 70, 72, 74, 76, 77, 78, 79, 80, 81, 82, 83, 84, 85, 86, 87, 88, 89, 90, 91, 92, 93, 94, 95, 96, 97, 98, 99, 100% or any amount therebetween, sequence similarity or identity with the amino acid sequence of 7-1a2-7-2₁--7-1b₁ (7-1a) fusion amino acid sequence of SEQ ID NO: 28 or 39 provided that the VP7 fusion protein induces immunity to rotavirus in a subject, when the VP7 protein is administered to the subject.

B. 7-1a₁--7-2₁--7-1b₂; 7-1b

For example, the rotavirus VP7 fusion protein, and methods of producing the rotavirus VP7 fusion protein, may include a rotavirus VP7 fusion protein comprising a 7-2 domain and a 7-1a subdomain derived from a first rotavirus genotype or strain and a 7-1b subdomain from a second rotavirus genotype or strain. The 7-1a subdomain and 7-2 domain are both derived from a first rotavirus genotype or strain and are being fused to the 7-1b subdomain derived from a second rotavirus genotype or strain:
7-1a_{1nd strain}--7-2_{1st strain}--7-1b_{2st strain} (7-1a₁-7-2₁--7-1b₂; 7-1b).

It has been observed that expression of the VP7 fusion protein (7-1a₁-7-2₁--7-1b₂) comprising a 7-2 domain and a 7-1a subdomain derived from a first rotavirus genotype or strain and a 7-1b subdomain from a second rotavirus genotype or strain increases the yield of the VP7 fusion protein, when compared to the yield of a wildtype or native VP7 protein comprising a 7-2 domain and a 7-1 domain (7-1a and 7-1b subdomains) from the same second rotavirus genotype or strain, when expressed in the same type of plant and under the same conditions, as a native or wildtype VP7 protein (see for example FIG. 3a, fourth and eight lane (7-1b), compared to the second (VP7 G2P5) and sixth lane (VP7 G9P8), respectively and FIG. 3b, third and sixth lane (7-1b) compared to the second (VP7 G3P5) and fifth (VP7 G12P8) lane, respectively).

Examples of VP7 fusion protein of the form: 7-1a₁-7-2₁--7-1b₂ (7-1b) include, but are not limited to: VP7(Rtx)+(7-1b)G2P5 [RVA(Rtx G1) VP7 (7-1b G2); SEQ ID NO: 31] comprising a 7-1a subdomain and a 7-2 domain from USA/Rotarix-A41CB052A/1988/G1P1A[8] and a 7-1b subdomain from rotavirus strain G2P5 [RVA/Vaccine/USA/RotaTeq-SC2-9/1992/G2P7[5]], VP7(Rtx)+(7-1b)G9P8 [RVA(Rtx G1) VP7 G9); SEQ ID NO: 41] comprising a 7-1a subdomain and a 7-2 domain from USA/Rotarix-A41CB052A/1988/G1P1A[8] and a 7-1b subdomain from rotavirus strain G9P8 [RVA/Hu/WI61/1983/G9P1A[8]], VP7(Rtx)+(7-1b)G3P5 [RVA(Rtx G1) VP7 (7-1b G3); SEQ ID NO: 50] comprising a 7-1a subdomain and a 7-2 domain from USA/Rotarix-A41CB052A/1988/G1P1A[8] and a 7-1b subdomain from rotavirus strain G3P5 [RVA/Vaccine/USA/RotaTeq-WI78-8/1992/G3P7[5]], and VP7(Rtx)+(7-1b)G12P8 [RVA(Rtx G1) VP7 (7-1b G12); SEQ ID NO: 59] comprising a 7-1a subdomain and a 7-2 domain from USA/Rotarix-A41CB052A/1988/G1P1A[8] and a 7-1b subdomain from rotavirus strain G12P8 [RVA/Human-tc/KEN/KDH651/2010/G12P[8]], or a sequence that exhibits from about 59-100% or any amount therebetween, sequence similarity or identity with the amino acid sequence of the 7-1a₁--7-2₁--7-1b₂ (7-1b) fusion amino acid sequence of SEQ ID NO: 31, 41, 50 or 59, for example from about 59, 60, 62, 64, 66, 68, 70, 72, 74, 76, 77, 78, 79, 80, 81, 82, 83, 84, 85, 86, 87, 88, 89, 90, 91, 92, 93, 94, 95, 96, 97, 98, 99, 100% or any amount therebetween, sequence similarity or identity with the amino acid sequence of 7-1a₁--7-2₁--7-1b₂(7-1b) fusion amino acid sequence of SEQ ID NO: 31, 41, 50 or 59 provided that the VP7 fusion protein induces immunity to rotavirus in a subject, when the VP7 protein is administered to the subject.

C. 7-1a₂--7-2₁--7-1b₂; 7-1a-1b

For example, the rotavirus VP7 fusion protein, and methods of producing the rotavirus VP7 fusion protein, may include a rotavirus VP7 fusion protein comprising a 7-2 domain derived from a first rotavirus genotype or strain and a 7-1a subdomain and a 7-1b subdomain from a second rotavirus genotype or strain. The 7-1a subdomain is derived from a second rotavirus genotype or strain and fused to the 7-2 domain which is derived from a first rotavirus genotype or strain, which in turn is fused to the 7-1b subdomain which is derived from a second rotavirus genotype or strain:
7-1a_{2nd strain}--7-2_{1st strain}--7-1b_{2st strain} (7-1a₂--7-2₁--7-1b₂; 7-1a-1b).

It has been observed that expression of the VP7 fusion protein (7-1a₂--7-2₁--7-1b₂; 7-1a-1b) comprising a 7-2 domain derived from a first rotavirus genotype or strain and a 7-1a subdomain and a 7-1b subdomain from a second rotavirus genotype or strain increases the yield of the VP7 fusion protein, when compared to the yield of a wildtype or native VP7 protein comprising a 7-2 domain and a 7-1 domain (7-1a and 7-1b subdomains) from the same second rotavirus genotype or strain, when expressed in the same type of plant and under the same conditions, as a native or wildtype VP7 protein (see for example FIG. 3a, fifth and ninth lane (7-1a-1b), compared to the second (VP7 G2P5) and sixth lane (VP7 G9P8), respectively and FIG. 3b, fourth and seventh lane (7-1a-1b) compared to the second (VP7 G3P5) and fifth (VP7 G12P8) lane, respectively).

Examples of VP7 fusion protein of the form: 7-1a₂--7-2₁--7-1b₂ (7-1a-1b) include, but are not limited to: VP7(Rtx)+(7-1a-1b)G2P5 [RVA(Rtx G1) VP7 (7-1a-1b G2); SEQ ID NO: 33) comprising a 7-2 domain from USA/Rotarix-A41CB052A/1988/G1P1A[8] and a 7-1a and 7-1b subdomain from rotavirus strain G2P5 [RVA/Vaccine/USA/RotaTeq-SC2-9/1992/G2P7[5]], VP7(Rtx)+(7-1a-1b)G9P8 [RVA(Rtx G1) VP7 (7-1a-1b G9); SEQ ID NO: 43] comprising a 7-2 domain from USA/Rotarix-A41CB052A/1988/G1P1A[8] and a 7-1a and 7-1b subdomain from rotavirus strain G9P8 [RVA/Hu/WI61/1983/G9P1A[8]], VP7(Rtx)+(7-1a-1b)G3P5 [RVA(Rtx G1) VP7 (7-1a-1b G3); SEQ ID NO: 52) comprising a 7-2 domain from USA/Rotarix-A41CB052A/1988/G1P1A[8] and a 7-1a and 7-1b subdomain from rotavirus strain G3P5 [RVA/Vaccine/USA/RotaTeq-WI78-8/1992/G3P7[5]], and VP7(Rtx)+(7-1a-1b)G12P8 [RVA(Rtx G1) VP7 (7-1a-1b G12); SEQ ID NO: 61] comprising a 7-2 domain from USA/Rotarix-A41CB052A/1988/G1P1A[8] and a 7-1a and 7-1b subdomain from rotavirus strain G12P8 [RVA/Human-tc/KEN/KDH651/2010/G12P[8]], or a sequence that exhibits from about 59-100% or any amount therebetween, sequence similarity or identity with the amino acid sequence of the 7-1a₂--7-2₁--7-1b₂ (7-1a-1b) fusion amino acid sequence of SEQ ID NO: 33, 43, 52, or 61, for example from about 59, 60, 62, 64, 66, 68, 70, 72, 74, 76, 77, 78, 79, 80, 81, 82, 83, 84, 85, 86, 87, 88, 89, 90, 91, 92, 93, 94, 95, 96, 97, 98, 99, 100% or any amount therebetween, sequence similarity or identity with the amino acid sequence of 77-1a₂--7-2₁--7-1b₂ (7-1a-1b) fusion amino acid sequence of SEQ ID NO: 33, 43, 52, or 61 provided that the VP7 fusion protein induces immunity to rotavirus in a subject, when the VP7 protein is administered to the subject.

The rotavirus VP7 fusion protein may comprise one or more than one domain or subdomain derived from any rotavirus strain having a genotype of any combinations of G- and P-types from G1 to G27 and from P1 to P34, and more preferably from G1 to G19 and from P1 to P27, including, but not limited to G1P[8], G2P[4], G2P[8], G2P[5], G3P[5], G3P[8], G4P[5], G4P[8], G9P[6], G9P[8], G12P[8], rotavirus A WA strain, rotavirus USA/Rotarix-A41CB052A/1988/G1P1A[8] strain, rotavirus SA11 strain, human rotavirus HCR3 (GenBank: AAA18522), porcine-like human G9P[6] rotavirus strain (A Hu/BEL/BE2001/2009/G9P[6]; GenBank: AFJ11215.1), rotavirus G12 (GenBank: BAD89095); rotavirus strain G4 BrB-9, RVA/Vaccine/USA/RotaTeq-SC2-9/1992/G2P7[5] (GenBank: ADK27036), RVA/Vaccine/USA/RotaTeq-WI78-8/1992/G3P7[5]; (GenBan; ADK27037), RVA/Vaccine/USA/RotaTeq-BrB-9/1996/G4P7[5] (GenBank: ADK27038), RVA Hu/WI61/1983/G9P1A[8] (UniProtKB/Swiss-Prot: B3SRX9) RVA/Human-tc/KEN/KDH651/2010/G12P[8] (GenBank: BAO74145), RVA/Vaccine/USA/Rotarix-A41CB052A/1988/G1P1A[8] (GenBank: JN849114.1), RVA/Vaccine/USA/RotaTeq-WI79-9/1992/G1P7[5](GenBank: GU565057), RVA/Human-wt/BEL/BE1520/2009/G1P[8] (GenBank: JN849152), RVA/Human-wt/BEL/BE1175/2009/G1P[8] (GenBank: JN849154), RVA/Human-wt/BEL/BE1280/2009/G1P[8] (GenBank: JN849150), RVA/Human-wt/BEL/BE1001a/2008/G1P[8] (GenBank: JN849126), RVA/Human-wt/BEL/BE0253/2008/G1P[8] (GenBank: JN849120), RVA/Human-wt/BEL/BE1023/2008/G1P[8] (GenBank: JN849122), RVA/Human-wt/BEL/BE1286/2009/G1P[8] (GenBank: JN849148), RVA/Human-wt/BEL/BE1128/2009/G1P[8] (GenBank: JN849136), RVA/Vaccine/USA/RotaTeq-SC2-9/1992/G2P7[5] (GenBank: GU565068), RVA/Human-wt/BEL/BE1248/2009/G2P[4] (GenBank: JN849130), RVA/Human-wt/BEL/BE1141/2009/G2P[4] (GenBank: JN849156), RVA/Human-wt/BEL/BE1058/2008/G2P[4] (GenBank: JN849124), RVA/Human-wt/BEL/BE1251/2009/G2P[4] (GenBank: JN849144), RVA/Vaccine/USA/RotaTeq-WI78-8/1992/G3P7[5] (GenBank: GU565079), RVA/Human-wt/BEL/BE1322/2009/G3P[6] (GenBank: JF460828), RVA/Human-wt/BEL/BE1214/2009/G3P[8] (GenBank: JN849140), RVA/Human-wt/BEL/BE1259/2009/G3P[8] (GenBank: JN8491460), RVA/Vaccine/USA/RotaTeq-BrB-9/1996/G4P7[5] (GenBank: GU565090), RVA/Human-wt/BEL/BE1129/2009/G4P[8] (GenBank: JN849138), RVA/Human-wt/BEL/BE1113/2009/G4P[8] (GenBank: JN849134), RVA/Vaccine/USA/RotaTeq-WI79-4/1992/G6P1A[8](GenBank: GU565046), RVA/Human-wt/BEL/BE1242/2009/G9P[8] (GenBank: JN849142), RVA/Human-wt/BEL/BE1119/2009/G9P[8] (GenBank: JN849132), RVA/Human-wt/BEL/BE1032/2008/G9P[8] (GenBank: JN849128), RVA/Human-wt/BEL/BE0258/2008/G12P[8] (GenBank: JN849118), RVA/Human-wt/BEL/BE0085/2008/G12P[8] (GenBank: JN849116), a sequence that exhibits from about 59-100% or any amount therebetween, sequence similarity or identity with the amino acid sequence of the sequence of GenBank: AAA18522, GenBank: AFJ11215.1, GenBank: BAD89095, GenBank: ADK27036, GenBan; ADK27037, GenBank: ADK27038, UniProtKB/Swiss-Prot: B3SRX9, GenBank: BAO74145, GenBank: JN849114.1, GenBank: GU565057, GenBank: JN849152, GenBank: JN849154, GenBank: JN849150, GenBank: JN849126, GenBank: JN849120, GenBank: JN849122, GenBank: JN849148, GenBank: JN849136, GenBank: GU565068, GenBank: JN849130, GenBank: JN849156, GenBank: JN849124, GenBank: JN849144, GenBank: GU565079, GenBank: JF460828, GenBank: JN849140, GenBank: JN8491460, GenBank: GU565090, GenBank: JN849138, GenBank: JN849134, GenBank: GU565046, GenBank: JN849142, GenBank: JN849132, GenBank: JN849128, GenBank: JN849118, GenBank: JN849116, for example from about 59, 60, 62, 64, 66, 68, 70, 72, 74, 76, 77, 78, 79, 80, 81, 82, 83, 84, 85, 86, 87, 88, 89, 90, 91, 92, 93, 94, 95, 96, 97, 98, 99, 100% or any amount therebetween, sequence similarity or identity with the amino acid sequence of the sequence of GenBank: AAA18522, GenBank: AFJ11215.1, GenBank: BAD89095, GenBank: ADK27036, GenBan; ADK27037, GenBank: ADK27038, UniProtKB/Swiss-Prot: B3SRX9, GenBank: BAO74145, GenBank: JN849114.1, GenBank: GU565057, GenBank: JN849152, GenBank: JN849154, GenBank: JN849150, GenBank: JN849126, GenBank: JN849120, GenBank: JN849122, GenBank: JN849148, GenBank: JN849136, GenBank: GU565068, GenBank: JN849130, GenBank: JN849156, GenBank: JN849124, GenBank: JN849144, GenBank: GU565079, GenBank: JF460828, GenBank: JN849140, GenBank: JN8491460, GenBank: GU565090, GenBank: JN849138, GenBank: JN849134, GenBank: GU565046, GenBank: JN849142, GenBank: JN849132, GenBank: JN849128, GenBank: JN849118, GenBank: JN849116, provided that the VP7 fusion protein induces immunity to rotavirus in a subject, when the VP7 protein is administered to the subject.

Rotavirus strains or genotypes as disclosed herein include, any known rotavirus strain or genotype, but also modifications to known rotavirus strains that are known to develop on a regular basis over time (see for example Kirkwood C D The Journal of Infectious Diseases, 2010, Volume 202 (Supplement 1)). Accordingly, the first rotavirus strain or genotype or the second rotavirus strain or genotype may for example be derived from any rotavirus strain having a genotype of any combinations of G- and P-types from G1 to G27 and from P1 to P34, and more preferably from G1 to G19 and from P1 to P27, including, but not limited to G1P[8], G2P[4], G2P[8], G2P[5], G3P[5], G3P[8], G4P[5], G4P[8], G9P[6], G9P[8], G12P[8], rotavirus A WA strain, rotavirus USA/Rotarix-A41CB052A/1988/G1P1A[8] strain, rotavirus SA11 strain, human rotavirus HCR3 (GenBank: AAA18522), porcine-like human G9P[6] rotavirus strain (A Hu/BEL/BE2001/2009/G9P[6]; GenBank: AFJ11215.1), rotavirus G12 (GenBank: BAD89095); rotavirus strain G4 BrB-9, RVA/Vaccine/USA/RotaTeq-SC2-9/1992/G2P7[5] (GenBank: ADK27036), RVA/Vaccine/USA/RotaTeq-WI78-8/1992/G3P7[5]; (GenBan; ADK27037), RVA/Vaccine/USA/RotaTeq-BrB-9/1996/G4P7[5] (GenBank: ADK27038), RVA Hu/WI61/1983/G9P1A[8] (UniProtKB/Swiss-Prot: B3SRX9) RVA/Human-tc/KEN/KDH651/2010/G12P[8] (GenBank: BAO74145), RVA/Vaccine/USA/Rotarix-A41CB052A/1988/G1P1A[8] (GenBank: JN849114.1), RVA/Vaccine/USA/RotaTeq-WI79-9/1992/G1P7[5](GenBank: GU565057), RVA/Human-wt/BEL/BE1520/2009/G1P[8] (GenBank: JN849152), RVA/Human-wt/BEL/BE1175/2009/G1P[8] (GenBank: JN849154), RVA/Human-wt/BEL/BE1280/2009/G1P[8] (GenBank: JN849150), RVA/Human-wt/BEL/BE1001 a/2008/G1P[8] (GenBank: JN849126), RVA/Human-wt/BEL/BE0253/2008/G1P[8] (GenBank: JN849120), RVA/Human-wt/BEL/BE1023/2008/G1P[8] (GenBank: JN849122), RVA/Human-wt/BEL/BE1286/2009/G1P[8] (GenBank: JN849148), RVA/Human-wt/BEL/BE1128/2009/G1P[8] (GenBank: JN849136), RVA/Vaccine/USA/RotaTeq-SC2-9/1992/G2P7[5] (GenBank: GU565068), RVA/Human-wt/BEL/BE1248/2009/G2P[4] (GenBank: JN849130), RVA/Human-wt/BEL/BE1141/2009/G2P[4] (GenBank: JN849156), RVA/Human-wt/BEL/BE1058/2008/G2P[4] (GenBank: JN849124), RVA/Human-wt/BEL/BE1251/2009/G2P[4] (GenBank: JN849144), RVA/Vaccine/USA/RotaTeq-WI78-8/1992/G3P7[5] (GenBank: GU565079), RVA/Human-wt/BEL/BE1322/2009/G3P[6] (GenBank: JF460828), RVA/Human-wt/BEL/BE1214/2009/G3P[8] (GenBank: JN849140), RVA/Human-wt/BEL/BE1259/2009/G3P[8] (GenBank: JN8491460), RVA/Vaccine/USA/RotaTeq-BrB-9/1996/G4P7[5] (GenBank: GU565090), RVA/Human-wt/BEL/BE1129/2009/G4P[8] (GenBank: JN849138), RVA/Human-wt/BEL/BE1113/2009/G4P[8] (GenBank: JN849134), RVA/Vaccine/USA/RotaTeq-WI79-4/1992/G6P1A[8](GenBank: GU565046), RVA/Human-wt/BEL/BE1242/2009/G9P[8] (GenBank: JN849142), RVA/Human-wt/BEL/BE1119/2009/G9P[8] (GenBank: JN849132), RVA/Human-wt/BEL/BE1032/2008/G9P[8] (GenBank: JN849128), RVA/Human-wt/BEL/BE0258/2008/G12P[8] (GenBank: JN849118), RVA/Human-wt/BEL/BE0085/2008/G12P[8] (GenBank: JN849116), a sequence that exhibits from about 59-100% or any amount therebetween, sequence similarity or identity with the amino acid sequence of the sequence of GenBank: AAA18522, GenBank: AFJ11215.1, GenBank: BAD89095, GenBank: ADK27036, GenBan; ADK27037, GenBank: ADK27038, UniProtKB/Swiss-Prot: B3SRX9, GenBank: BAO74145, GenBank: JN849114.1, GenBank: GU565057, GenBank: JN849152, GenBank: JN849154, GenBank: JN849150, GenBank: JN849126, GenBank: JN849120, GenBank: JN849122, GenBank: JN849148, GenBank: JN849136, GenBank: GU565068, GenBank: JN849130, GenBank: JN849156, GenBank: JN849124, GenBank: JN849144, GenBank: GU565079, GenBank: JF460828, GenBank: JN849140, GenBank: JN8491460, GenBank: GU565090, GenBank: JN849138, GenBank: JN849134, GenBank: GU565046, GenBank: JN849142, GenBank: JN849132, GenBank: JN849128, GenBank: JN849118, GenBank: JN849116, for example from about 59, 60, 62, 64, 66, 68, 70, 72, 74, 76, 77, 78, 79, 80, 81, 82, 83, 84, 85, 86, 87, 88, 89, 90, 91, 92, 93, 94, 95, 96, 97, 98, 99, 100% or any amount therebetween, sequence similarity or identity with the amino acid sequence of the sequence of GenBank: AAA18522, GenBank: AFJ11215.1, GenBank: BAD89095, GenBank: ADK27036, GenBan; ADK27037, GenBank: ADK27038, UniProtKB/Swiss-Prot: B3SRX9, GenBank: BAO74145, GenBank: JN849114.1, GenBank: GU565057, GenBank: JN849152, GenBank: JN849154, GenBank: JN849150, GenBank: JN849126, GenBank: JN849120, GenBank: JN849122, GenBank: JN849148, GenBank: JN849136, GenBank: GU565068, GenBank: JN849130, GenBank: JN849156, GenBank: JN849124, GenBank: JN849144, GenBank: GU565079, GenBank: JF460828, GenBank: JN849140, GenBank: JN8491460, GenBank: GU565090, GenBank: JN849138, GenBank: JN849134, GenBank: GU565046, GenBank: JN849142, GenBank: JN849132, GenBank: JN849128, GenBank: JN849118, GenBank: JN849116, provided that the VP7 fusion protein induces immunity to rotavirus in a subject, when the VP7 protein is administered to the subject.

### Rotavirus-Like Particle (RLP) Comprising Fusion Protein

Also provided herewith are methods of producing virus-like particle (VLP) also referred to as rotavirus-like particle (RLP) or methods of increasing production of VLP comprising rotavirus VP7 fusion protein in plants, portion of a plant, or a plant cell.

VLPs may also be referred to as “rotavirus VLP”, “rotavirus-like particle (RVLP)”, “rotavirus-like particle (RLP)”, “rotavirus-like particle”, “RVLP”, “RLP” or “fusion RLP” that comprise the VP7 fusion protein. VLPs or RLPs are structures that self-assemble and comprise one or more rotavirus native structural proteins, one or more rotavirus fusion protein or a combination thereof. For example the RLP may comprise one or more than one of rotavirus structural protein VP2, VP4 and/or VP6 and/or one or more than one VP7 fusion protein. In a non-limiting example the RLP comprises structural protein VP2, VP6 and fusion protein VP7. In another non-limiting example the RLP may comprise structural protein VP2, VP4, VP6 and fusion protein VP7. VLPs or RLPs are generally morphologically and antigenically similar to virions produced in an infection, but lack genetic information sufficient to replicate and thus are non-infectious.

RLPs or VLPs comprising VP2 protein, VP6 protein and VP7 fusion protein, or VP2 protein, VP6 protein, VP4 protein and VP7 fusion protein, are of the size from about 50 nm to 120 nm or any amount therebetween, for example 55, 60, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 nm, or any amount therebetween. For example, RLPs may be from about 75 to about 110 nm.

VLP or RLP comprising rotavirus VP7 fusion protein produced by the method as described herein may comprise a higher ratio of VP7:VP6, when compared to RLP that comprise wild type of native VP7 (see Example 3).

The mature rotavirion is a T=13 icosahedron consisting of three concentric layers (shells) of protein and a genome of eleven segments of ds RNA (Patton J. T. Journal of General Virology (1995), 76, 2633-264). The outer most layer is made up of 780 copies of the glycoprotein, VP7 (37 kDa), and 60 spikes formed from dimers of the viral attachment protein, VP4 (87 kDa) (Patton J. T. Journal of General Virology (1995), 76, 2633-264). The intermediate shell or layer consists of 260 trimers of VP6 (45 kDa) arranged in a T=13 lattice. The innermost layer is a T=1 structure made up of 60 dimers of the RNA-binding protein VP2 (102 kDa) (Patton J. T. Journal of General Virology (1995), 76, 2633-264). The stoichiometric mass ratio of VP2:VP6:VP7 in the mature rotavirion is approximately: 1:2.8:2.4. Therefore, in the mature rotavirion the mass ratio of VP7:VP6 is approximately 0.85.

Yang et al. 2011 (Science China Life Sciences January 2011, Volume 54, Issue 1, pp 82-89) co-expressed three rotavirus capsid proteins VP2, VP6 and VP7 of group A RV (P[8]G1) in tobacco plants and expression levels of these proteins, as well as formation of rotavirus-like particles were studied. However only a small portion of the expressed rotavirus capsid proteins produced assembled into RV VLPs and the outer-layer protein, VP7, may only partially exist or be completely absent in some RV VLPs produced by Yang et al. 2011.

Based on the data provided in Yang et. al. 2011 the stoichiometric mass ratio of VP7:VP6 in their RLP is 0.18. in their RLP This ratio can be determined from the data provided in Table 2 of Yang et al., which shows that the VP6:VP2 ratio is 4.26, and the VP7/VP2 ratio is 0.77, which equals a ratio of VP7:VP6 as 0.18 by solving for VP7/VP6=0.77/4.26=0.18.

RLP comprising rotavirus VP7 fusion protein produced by the method as described herein may comprise a higher ratio of VP7:VP6, when compared to RLP that comprise wild type or native VP7.

Also provided herewith are methods of producing RLPs comprising increased amounts or increased incorporation of the rotavirus VP7 fusion protein when compared to RLPs that are produced under the same condition as the RLPs comprising VP7 fusion protein, but wherein the RLPs comprise wildtype or native VP7 proteins.

The amounts or incorporation of the rotavirus VP7 fusion protein in an RLP might be expressed for example as stoichiometric mass ratio of VP7:VP6, as described above. Therefore, methods of increasing the stoichiometric mass ratio of VP7:VP6 in a rotavirus-like particle (RLP) in plants, portion of a plant, or a plant cell are provided. Furthermore, RLP with an increased stoichiometric mass ratio of VP7:VP6 are provided. The stoichiometric mass ratio of VP7:VP6 in the method or RLP is compared to RLPs that comprise wildtype or native VP7 proteins and are produced under the same conditions as the RLP comprising rotavirus VP7 fusion protein.

The stoichiometric mass ratio of VP7:VP6 in the RLP comprising rotavirus VP7 fusion protein may be from about 0.2 to 0.85 or any amount therebetween. For example the stoichiometric mass ratio of VP7:VP6 may be 0.2, 0.25, 0.3, 0.35, 0.4, 0.45, 0.5, 0.55, 0.7, 0.75, 0.8, 0.85 or any amount therebetween.

It is further provided a triple-layered rotavirus like particle (RLP) comprising rotavirus structural protein VP2, VP6 and VP7 fusion protein, wherein the ratio of VP7:VP6 of the RLP, is from 0.2 to 0.85 or any amount therebetween. For example the triple-layered RLP may comprise rotavirus structural protein VP2, VP6 and VP7, wherein the stoichiometric mass ratio of VP7:VP6 is 0.2, 0.25, 0.3, 0.35, 0.4, 0.45, 0.5, 0.55, 0.7, 0.75, 0.8, 0.85 or any amount therebetween. The triple-layered RLP may optionally comprise rotavirus structural protein VP4.

VP7 incorporation or VP7 content in an RLP may be further expressed at % content of VP7 in an RLP (see for example Table 5). In the native rotavirus approximately 37% of total structural protein mass of an RLP (comprising VP2, VP4, VP6 and VP7) is VP7 protein calculated by using theoretical molecular weight and structural protein stoichiometry. The VP7 fusion protein content in the RLP of the present disclosure may be from about 5% to about 38%, or any amount therebetween of total structural protein mass of an RLP comprising VP7 fusion, VP2 and VP6. For example the VP7 fusion protein content in the RLP may be 5%, 10%, 12%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or any amount therebetween of total structural protein mass of an RLP comprising VP7 fusion, VP2 and VP6.

It is further provided a triple-layered rotavirus like particle (RLP) comprising rotavirus structural protein VP2, VP6 and VP7 fusion protein, wherein 5% to 38% or any amount therebetween of the total structural protein mass of the RLP is VP7 protein. For example the triple-layered RLP may comprise rotavirus structural protein VP2, VP6 and VP7, wherein the VP7 fusion protein content in the RLP may be 5%, 10%, 12%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or any amount therebetween of total structural protein mass of the RLP. The triple-layered RLP may optionally comprise rotavirus structural protein VP4.

In the methods of producing RLP or methods of increasing production of RLP comprising rotavirus VP7 fusion protein in plants, portion of a plant, or a plant cell, a nucleic acid encoding a rotavirus VP7 fusion protein as described herein, for example protein 7-1a (7-1a_{2nd strain}--7-2_{1st strain}--7-1b_{1st strain}), protein 7-1 b (7-1a_{1nd strain}--7-2_{1st strain}--7-1b_{2st strain}), protein 7-1a-1b (7-1a_{2nd strain}--7-2_{1st strain}--7-1b_{2st strain}) or a combination thereof is introduced into the plants, portion of the plant, or plant cell. The nucleic acid is expressed under suitable conditions in the plant, portion of a plant, or a plant cell and RLPs comprising the rotavirus VP7 fusion protein are produced. One or more than one type of rotavirus fusion protein may be expressed in a plant, portion of the plant or plant cell in order to produce a RLP comprising one or more than one type of rotavirus fusion protein.

The methods of producing a RLP comprising a VP7 fusion protein may also comprise a step of co-expressing a nucleic acid sequence encoding a rotavirus VP2 structural protein, a rotavirus VP6 structural protein and rotavirus VP4 structural protein in the plant or portion of the plant. The rotavirus VP2, VP6 and/or VP4 structural protein may be from the first rotavirus strain from which the domain or subdomain of the VP7 fusion are derived. Furthermore, the rotavirus VP2, VP6 and/or VP4 structural protein may be derived from the second rotavirus strain from which the domain or subdomain of the VP7 fusion is derived. Furthermore, the rotavirus VP2, VP6 and/or VP4 structural protein may be derived from a third rotavirus strain, wherein the third rotavirus strain is a different rotavirus strain than the first or the second rotavirus strain.

For example rotavirus structural protein VP2, VP6 or both VP2 and VP6 may be derived from any rotavirus strain having a genotype of any combinations of G- and P-types from G1 to G27 and from P1 to P34, and more preferably from G1 to G19 and from P1 to P27, including, but not limited to G1P[8], G2P[4], G2P[8], G2P[5], G3P[5], G3P[8], G4P[5], G4P[8], G9P[6], G9P[8] and G12P[8]. Therefore, rotavirus structural protein VP2, VP6 or both VP2 and VP6 may be derived from any rotavirus strain having a genotype of G1, G2, G3, G4, G5, G6, G7, G8, G9, G10, G11, G12, G13, G14, G15, G16, G17, G18, G19, G20, G21, G22, G23, G24, G25 or G27.

Furthermore, the methods of producing a RLP comprising a VP7 fusion protein as described above may further comprise a step of expressing a nucleic acid sequence encoding a rotavirus non-structural protein, for example NSP4.

It has been found that by introducing and co-expressing rotavirus structural protein and rotavirus non-structural protein in the host, such as a plant or portion of the plant that the yield of the VLP or RLP produced may be modulated. In particular, it has been found that by co-expressing rotavirus structural proteins along with a rotavirus non-structural protein NSP4 in the host, such as a plant, portion of the plant, or plant cell, that the incorporation of structural protein VP7 into the RLP may be increased, when compared to the level of VP7 produced by a second host, such as a second plant, portion of a second plant, or second plant cell that expresses the same rotavirus structural proteins but that does not express the rotavirus non-structural protein, under the same conditions (see for example WO 2016/115630, which is herein incorporated by reference).

Accordingly, the methods of producing a RLP comprising a VP7 fusion in a host or host cell may comprise providing a host or host cell such as a plant, portion of a plant or plant cell, comprising one or more nucleic acid comprising a first nucleotide sequence encoding the VP7 fusion, a second nucleotide sequence encoding VP2 and a third nucleotide sequence encoding VP6, and a fourth nucleotide sequence encoding NSP4. The first, second, third and fourth nucleotide sequence are being operatively linked to one or more regulatory region active in the host or host cell, such as a plant, portion of a plant or plant cell. The host or host cell is incubated under conditions that permit the expression of the one or more nucleic acid, so that each of VP7 fusion, VP2, VP6 and NSP4 are expressed. The VLPs (or RLPs) produced by the method comprise rotavirus structural proteins VP7 fusion, VP2 and VP6. The RLPs do not contain rotavirus protein NSP4.

The host or host cell, such as a plant, portion of a plant or plant cell may further comprise a fifth nucleotide sequence encoding rotavirus structural protein VP4 and upon expression VP4 is produced within the host or host cell. The VLPs (or RLPs) produced by this method comprise rotavirus structural proteins VP7 fusion, VP2, VP6 and optionally VP4.

The rotavirus VP2, VP6, VP4, NSP4 protein or a combination thereof may be from the first rotavirus strain from which the domain or subdomain of the VP7 fusion are derived. Furthermore, the rotavirus VP2, VP6, VP4, NSP4 protein or a combination thereof may be derived from the second rotavirus strain from which the domain or subdomain of the VP7 fusion is derived. Furthermore, the rotavirus VP2, VP6, VP4, NSP4 protein or a combination thereof may be derived from a third rotavirus strain, wherein the third rotavirus strain is a different rotavirus strain than the first or the second rotavirus strain. In addition, VP2, VP6, VP4, NSP4 protein or a combination thereof may be from a fourth rotavirus strain, wherein the third rotavirus strain is a different rotavirus strain than the first, the second or the fourth rotavirus strain. As indicated in table 1, any one of VP2, VP6, VP4 and NSP4 from one strain maybe combined with any one of VP2, VP6, VP4 and NSP4 from one or more than one other stains, as long as at least one VP2 and one VP6 are being used. Rotavirus protein VP4 and NSP4 may be used optionally in the described methods.

TABLE 1
Combination of rotavirus proteins and strains

| | Protein | | | |
|---|---|---|---|---|
| | Rotavirus | Rotavirus VP4 | Rotavirus | Rotavirus NSP4 |
| Strain | VP2 | (optionally) | VP6 | (optionally) |
| First (1) | VP2 (1) | VP4 (1) | VP6 (1) | NSP4 (1) |
| Second (2) | VP2 (2) | VP4 (2) | VP6 (2) | NSP4 (2) |
| Third (3) | VP2 (3) | VP4 (3) | VP6 (3) | NSP4 (3) |
| Fourth (4) | VP2 (4) | VP4 (4) | VP6 (4) | NSP4 (4) |

*(_) indicate the different rotavirus strains Any one of the first, second, third and fourth rotavirus strain is a different strain.

For Example, the following combinations of rotavirus protein VP2 and VP6 may be used/co-expressed with the VP7 fusion protein described herewith: VP2(1), VP6(1); VP2(1), VP6(2); VP2(1), VP6(3); VP2(1), VP6(4); VP2(2), VP6(1); VP2(2), VP6(2); VP2(2), VP6(3); VP2(2), VP6(4); VP2(3), VP6(1); VP2(3), VP6(2); VP2(3), VP6(3); VP2(3), VP6(4); VP2(4), VP6(1); VP2(4), VP6(2); VP2(4), VP6(3) or VP2(4), VP6(4).

When one or more than one type of the rotavirus VP7 fusion protein is co-expressed with one or more than one rotavirus structural protein, for example VP2, VP6 and/or VP4 protein in the plant, portion of the plant or the plant cell, the one or more than one type of VP7 fusion proteins and the one or more than one rotavirus structural protein auto-assemble into RLPs. The plant or portion of the plant may be harvested under suitable extraction and purification conditions to maintain the integrity of the RLP, and the RLP comprising the one or more than one type of VP7 fusion protein may be purified. The one or more than one VP7 fusion protein may also be co-expressed with a nucleotide sequence encoding rotavirus VP2 protein (VP2) and a nucleotide sequence encoding rotavirus VP6 protein (VP6) so that the RLP may comprise VP7 fusion protein, VP2 and VP6 protein. The one or more than one VP7 fusion protein may also be co-expressed with a nucleotide sequence encoding VP2, a nucleotide sequence encoding VP6 a nucleotide sequence encoding rotavirus VP4 protein (VP4) so that the RLP may comprise VP7 fusion protein, VP2, VP6 and VP4 protein. The present disclosure also provides for the production of one or more than one type of VP7 fusion protein as described herein within a plant, portion of a plant, or plant cell, and the extraction and purification of the one or more than one type of VP7 fusion protein from the plant, the portion of the plant, or the plant cell to produce plant matter, a plant extract, or a protein extract, comprising the VP7 fusion protein.

The plant matter, plant extract, or protein extract may be used to induce immunity to rotavirus infection in a subject. Alternatively, the VP7 fusion protein, or the RLP comprising the VP7 fusion protein, may be purified or partially purified, and the purified or partially purified preparation may be used in inducing immunity to rotavirus infection in a subject.

The present disclosure also provides a composition comprising an effective dose of one or more than one type of rotavirus VP7 fusion protein, or RLPs comprising one or more than one type of rotavirus VP7 fusion protein, for inducing an immune response, and a pharmaceutically acceptable carrier, adjuvant, vehicle, or excipient.

Also provided herein are methods of inducing immunity to a rotavirus infection in a subject comprising of administering one or more than one type of rotavirus VP7 fusion protein or RLPs comprising one or more than one types of rotavirus VP7 fusion proteins to a subject orally, intranasally, intramuscularly, intraperitoneally, intravenously, or subcutaneously.

The present disclosure also provides for a method of producing RLPs in a plant, wherein a first nucleic acid encoding the rotavirus VP7 fusion protein is co-expressed with a second nucleic acid encoding a second rotavirus structural protein, for example a VP2 protein and a third nucleic acid encoding a third rotavirus structural protein, for example VP6, so that the first, the second, and the third nucleic acids are co-expressed in the plant. The first nucleic acid, second nucleic acid, and third nucleic acid may be introduced into the plant in the same step, or may be introduced to the plant sequentially.

Furthermore, a plant that expresses a first nucleic acid encoding a VP7 fusion protein, a second nucleic acid encoding a second rotavirus structural protein for example VP2 protein and a third nucleic acid encoding a third rotavirus structural protein for example VP6 protein may be further transformed with a fourth nucleic acid encoding a fourth rotavirus structural protein, for example VP4 protein, so that the first, the second nucleic acids, third and fourth nucleic acids are co-expressed in the plant.

Furthermore, a first plant expressing the first nucleic acid encoding a VP7 fusion, may be crossed with a second plant expressing the second nucleic acid encoding one or more rotavirus structural protein for example but not limited to VP6 or VP2 protein, to produce a progeny plant (third plant) that co-expresses the first and second nucleic acids encoding VP7 fusion protein and VP6 protein or VP7 fusion protein and VP2 protein, respectively. Furthermore, the third plant expressing the first and second nucleic acids encoding VP7 fusion protein and VP6 protein or VP7 fusion protein and VP2 protein, respectively, may be crossed with a fourth plant expressing the third nucleic acid encoding one or more rotavirus structural protein for example but not limited to VP6 or VP2 protein, to produce a further progeny plant (fifth plant) that co-expresses the first, second and third nucleic acids encoding VP7 fusion protein, VP2 protein or VP6 protein and VP6 or VP2 protein, so that VP7 fusion protein, VP2 protein and VP6 protein are expressed within the fifth plant.

The fifth plant expressing VP7 fusion protein, VP2 protein and VP6 protein may be further crossed with a sixth plant expressing a fourth nucleic acid encoding one or more rotavirus structural protein for example but not limited to VP4 to produce a seventh plant that expresses VP7 fusion protein, VP2 protein, VP6 protein and VP4 protein.

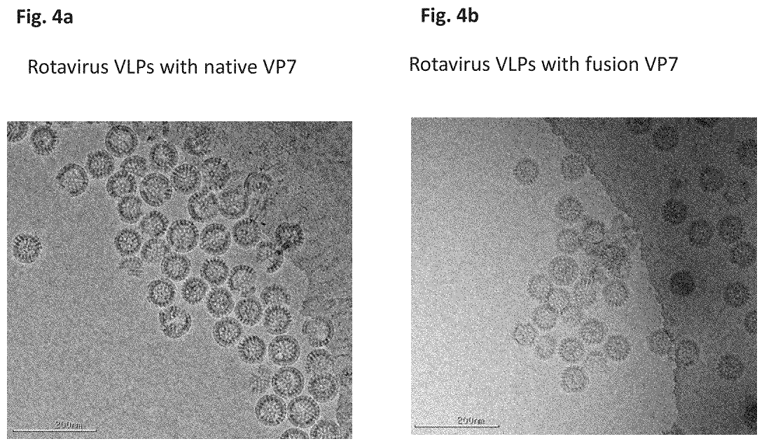

As seen in FIG. 4b, rotavirus VP7 fusion proteins self-assemble with rotavirus structural protein VP2 and VP6 into RLPs in plants. The isolated RLPs exhibit a structural conformation similar to that of RLPs with wildtype VP7 protein (FIG. 4a).

### Nucleic Acid

The present disclosure further provides a nucleic acid comprising a nucleotide sequence encoding a rotavirus VP7 fusion protein as described herewith. Accordingly the nucleic acid may comprise a nucleotide sequence encoding a VP7 fusion protein that may comprise a 7-2 domain derived from a first rotavirus genotype or strain and a 7-1a subdomain, a 7-1b subdomain or a 7-1a subdomain and a 7-1b subdomain derived from a second rotavirus genotype or strain (7-1a_{1st or 2nd strain}--7-2_{1st strain}--7-1b_{1st or 2nd strain} (7-1a_{1/2}-7-2₁-7-1b_{1/2}).

It is further provided a nucleic acid comprising a nucleotide sequence encoding a rotavirus VP7 fusion protein, the sequence comprising a first sequence encoding a 7-1a subdomain, a second sequence encoding a 7-2 domain and a third sequence encoding a 7-1b subdomain; wherein the sequence of the 7-2 domain is derived from a first rotavirus strain and the sequence of the 7-1a subdomain, the sequence of the 7-1b subdomain or the sequence of the 7-1a subdomain and the sequence of the 7-1b subdomain are derived from a second rotavirus strain, wherein the first rotavirus strain is a different rotavirus strain than the second rotavirus strain.

A nucleic acid encoding a rotavirus fusion protein may be described as a “rotavirus VP7 fusion nucleic acid”, a “rotavirus VP7 fusion nucleotide sequence”. Non-limiting examples of such nucleic acid are the sequences disclosed in SEQ ID NO: 27, 30, 32, 38, 40, 42, 49, 51, 58 or 60, or a sequence that exhibits from about 59-100% or any amount therebetween sequence similarity or identity with the sequences in SEQ ID NO: 27, 30, 32, 38, 40, 42, 49, 51, 58, 60, for example from about 59, 60, 62, 64, 66, 68, 70, 72, 74, 76, 77, 78, 79, 80, 81, 82, 83, 84, 85, 86, 87, 88, 89, 90, 91, 92, 93, 94, 95, 96, 97, 98, 99, 100% or any amount therebetween, sequence similarity or identity with the sequence of SEQ ID NO: 27, 30, 32, 38, 40, 42, 49, 51, 58 or 60.

The nucleotide sequence may be optimized for example for human codon usage or plant codon usage. Furthermore the nucleotide sequence encoding the VP7 fusion protein may be operatively linked to one or more than one amplification elements. In addition, the nucleotide sequence encoding the rotavirus protein such for example rotavirus protein VP2, VP4, VP6, VP7 fusion protein or NSP4 may be operatively linked to one or more than one enhancer sequence. For example the expression enhancer may be an enhancer derived from Cowpea Mosaic Virus (CPMV), referred to as CPMV enhancer element.

The term “CPMV enhancer element”, as used herein, refers to a nucleotide sequence encoding the 5′UTR regulating the Cowpea Mosaic Virus (CPMV) RNA2 polypeptide or a modified CPMV sequence as is known in the art. For example, a CPMV enhancer element or a CPMV expression enhancer, includes a nucleotide sequence as described in WO2015/14367; WO2015/103704; WO2007/135480; WO2009/087391; Sainsbury F., and Lomonossoff G. P., (2008, Plant Physiol. 148: pp. 1212-1218), each of which is incorporated herein by reference. A CPMV enhancer sequence can enhance expression of a downstream heterologous open reading frame (ORF) to which they are attached. The CPMV expression enhancer may include CPMV HT, CPMVX, CPMVX+, CPMV-HT+, CPMV HT+[WT115], or CPMV HT+[511] (WO2015/14367; WO2015/103704 which are incorporated herein by reference). The CPMV expression enhancer may be used within a plant expression system comprising a regulatory region that is operatively linked with the CPMV expression enhancer sequence and a nucleotide sequence of interest. The term “5′UTR” or “5′ untranslated region” or “5′ leader sequence” refers to regions of an mRNA that are not translated. The 5′UTR typically begins at the transcription start site and ends just before the translation initiation site or start codon of the coding region. The 5″ UTR may modulate the stability and/or translation of an mRNA transcript.

The one or more than one enhancer sequence operatively linked to the rotavirus protein such for example rotavirus protein VP2, VP4, VP6, VP7 fusion protein or NSP4 may further be a plant expression enhancer as described in U.S. Application No. 62/643,053 (which is herein incorporated by reference). Accordingly, non-limiting example of expression enhancer that may be used include:

nbGT61
(SEQ ID NO: 122)
ATCCAGAAGTAGGAATTCTTCAGTATAATCTA
GGGTTTTTTGAAAAGCAAATTGATCGAAA;
nbATL75
(SEQ ID NO: 123)
ATCTCCACCACCAAAAACCCTAATCGCCTCTC
CGTTTCTTCATCAGATTCTCGGTTCTCTTCTT
CTACAGCAACA;
nbDJ46
(SEQ ID NO: 124)
ACTCACCAAGAAAATAAACAAATTAAAGAATT
TTAAGAAAAACAAG;
nbCHP79
(SEQ ID NO: 125)
ATTCTGCCCTCAGTTAACTAAATTATCTCTCT
GATTAACAGTACTTTCTGATTTTCTGTGATTT
CTACAAATCTGAGAC;
nbEN42
(SEQ ID NO: 126)
ACTTTTGTATAGCTCCATTGAAATAGAGAAAA
GAAAATAGCC;
atHSP69
(SEQ ID NO: 127)
AAATTCAAAATTTAACACACAAACACAAACAC
ACACACCAAAAAAAACACAGACCTTAAAAAAA
TAAAA;
atGRP62
(SEQ ID NO: 128)
ATAACAAAACAAGATTTTGAAGTAAAACATAA
AAGAAAATAAACCCTAAGAATATATCGAAA;
atPK65
(SEQ ID NO: 129)
GCAAAAACAAAAATAAAAAAAACATCGCACAA
GAAAATAAAAGATTTGTAGAATCAA
CTAAGAAA;
atRP46
(SEQ ID NO: 130)
AGAAACAAAAAGAATTAAAAAAAAAAAAAAAA
AAAAGAATAAAGAA;
nb30572
(SEQ ID NO: 131)
ATCTTTCCCTCAAAACCCTAGCCGCAGTCACT
TCCGTAGGTGCTTACTTCGCTGTTAGTGCAAT
TCCAAACC;
nbMT78
(SEQ ID NO: 132)
(AC)ACAATTTGCTTTAGTGATTAAACTTTC
TTTTACAACAAATTAAAGGTCTATTATCTCCC
AACAACATAAGAAAACA;
nbPV55
(SEQ ID NO: 133)
AATTAAAGATCAATTCACTGTATCCCTCTTCT
CCAAAAAAAACTCTGCTGTAGTC;
nbPPI43
(SEQ ID NO: 134)
(AGC) ACAAATCGTACACAGCGAAAACCTCA
CTGAAATATTTAGAGAG;
nbPM64
(SEQ ID NO: 135)
(GTTC)AGAAAGATTTGTTTCCTCTGAAATA
GTTTTACAGAGCCAGAAGAAGAAAAAGAAGAA
GAGAGCA;
and
nbH2A86
(SEQ ID NO: 136)
ACTCAACACTCAAATCGCAATCCAAAAGCTT
CAATTTTTCCTAATACTTCTCTGTATTCAAG
CTTCGTAAACTTTCATTCACATCA.

A nucleic acid sequence referred to in the present disclosure, may be “substantially homologous”, “substantially similar” or “substantially identical” to a sequence, or a compliment of the sequence if the nucleic acid sequence hybridise to one or more than one nucleotide sequence or a compliment of the nucleic acid sequence as defined herein under stringent hybridisation conditions. Sequences are “substantially homologous” “substantially similar” “substantially identical” when at least about 70%, or between 70 to 100%, or any amount therebetween, for example 70, 72, 74, 76, 78, 80, 82, 84, 86, 88, 90, 92, 94, 96, 98, 100%, or any amount therebetween, of the nucleotides match over a defined length of the nucleotide sequence providing that such homologous sequences exhibit one or more than one of the properties of the sequence, or the encoded product as described herein.

Such a sequence similarity or identity may be determined using a nucleotide sequence comparison program, such as that provided within DNASIS (using, for example but not limited to, the following parameters: GAP penalty 5, #of top diagonals 5, fixed GAP penalty 10, k-tuple 2, floating gap 10, and window size 5). However, other methods of alignment of sequences for comparison are well-known in the art for example the algorithms of Smith & Waterman (1981, Adv. Appl. Math. 2:482), Needleman & Wunsch (J. Mol. Biol. 48:443, 1970), Pearson & Lipman (1988, Proc. Nat'l. Acad. Sci. USA 85:2444), and by computerized implementations of these algorithms (GAP, BESTFIT, FASTA, and BLAST, available through the NIH.), or by manual alignment and visual inspection (see, e.g., Current Protocols in Molecular Biology, Ausubel et al., eds. 1995 supplement), or using Southern or Northern hybridization under stringent conditions (see Maniatis et al., in Molecular Cloning (A Laboratory Manual), Cold Spring Harbor Laboratory, 1982). Preferably, sequences that are substantially homologous exhibit at least about 80% and most preferably at least about 90% sequence similarity over a defined length of the molecule.

An example of one such stringent hybridization conditions may be overnight (from about 16-20 hours) hybridization in 4×SSC at 65° C., followed by washing in 0.1×SSC at 65° C. for an hour, or 2 washes in 0.1×SSC at 65° C. each for 20 or 30 minutes. Alternatively an exemplary stringent hybridization condition could be overnight (16-20 hours) in 50% formamide, 4×SSC at 42° C., followed by washing in 0.1×SSC at 65° C. for an hour, or 2 washes in 0.1×SSC at 65° C. each for 20 or 30 minutes, or overnight (16-20 hours), or hybridization in Church aqueous phosphate buffer (7% SDS; 0.5M NaPO₄ buffer pH 7.2; 10 mM EDTA) at 65° C., with 2 washes either at 50° C. in 0.1×SSC, 0.1% SDS for 20 or 30 minutes each, or 2 washes at 65° C. in 2×SSC, 0.1% SDS for 20 or 30 minutes each for unique sequence regions.

### Codon Optimization

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. The process of optimizing the nucleotide sequence coding for a heterologously expressed protein can be an important step for improving expression yields. The optimization requirements may include steps to improve the ability of the host to produce the foreign protein.

“Codon optimization” is defined as modifying a nucleic acid sequence for enhanced expression in cells of interest by replacing at least one, more than one, or a significant number, of codons of the native sequence with codons that may be more frequently or most frequently used in the genes of another organism or species. Various species exhibit particular bias for certain codons of a particular amino acid.

The present disclosure includes synthetic polynucleotide sequences that have been codon optimized for example the sequences have been optimized for human codon usage or plant codon usage. The codon optimized polynucleotide sequences may then be expressed in plants. More specifically the sequences optimized for human codon usage or plant codon usage may be expressed in plants. Without wishing to be bound by theory, it is believed that the sequences optimized for human codon increases the guanine-cytosine content (GC content) of the sequence and improves expression yields in plants.

There are different codon-optimization techniques known in the art for improving, the translational kinetics of translationally inefficient protein coding regions. These techniques mainly rely on identifying the codon usage for a certain host organism. If a certain gene or sequence should be expressed in this organism, the coding sequence of such genes and sequences will then be modified such that one will replace codons of the sequence of interest by more frequently used codons of the host organism.

### Induction of Immunity Against Rotavirus Infection

An “immune response” generally refers to a response of the adaptive immune system of a subject. The adaptive immune system generally comprises a humoral response, and a cell-mediated response. The humoral response is the aspect of immunity that is mediated by secreted antibodies, produced in the cells of the B lymphocyte lineage (B cell). Secreted antibodies bind to antigens on the surfaces of invading microbes (such as viruses or bacteria), which flags them for destruction. Humoral immunity is used generally to refer to antibody production and the processes that accompany it, as well as the effector functions of antibodies, including Th2 cell activation and cytokine production, memory cell generation, opsonin promotion of phagocytosis, pathogen elimination and the like. The terms “modulate” or “modulation” or the like refer to an increase or decrease in a particular response or parameter, as determined by any of several assays generally known or used, some of which are exemplified herein.

A cell-mediated response is an immune response that does not involve antibodies but rather involves the activation of macrophages, natural killer cells (NK), antigen-specific cytotoxic T-lymphocytes, and the release of various cytokines in response to an antigen. Cell-mediated immunity is used generally to refer to some Th cell activation, Tc cell activation and T-cell mediated responses. Cell mediated immunity may be of particular importance in responding to viral infections.

For example, the induction of antigen specific CD8 positive T lymphocytes may be measured using an ELISPOT assay; stimulation of CD4 positive T-lymphocytes may be measured using a proliferation assay. Anti-rotavirus antibody titres may be quantified using an ELISA assay; isotypes of antigen-specific or cross reactive antibodies may also be measured using anti-isotype antibodies (e.g. anti-IgG, IgA, IgE or IgM). Methods and techniques for performing such assays are well-known in the art.

Cytokine presence or levels may also be quantified. For example a T-helper cell response (Th1/Th2) will be characterized by the measurement of IFN-γ and IL-4 secreting cells using by ELISA (e.g. BD Biosciences OptEIA kits). Peripheral blood mononuclear cells (PBMC) or splenocytes obtained from a subject may be cultured, and the supernatant analyzed. T lymphocytes may also be quantified by fluorescence-activated cell sorting (FACS), using marker specific fluorescent labels and methods as are known in the art.

A microneutralization assay may also be conducted to characterize an immune response in a subject, see for example the methods of Rowe et al., 1973. Virus neutralization titers may be quantified in a number of ways, including: enumeration of lysis plaques (plaque assay) following crystal violent fixation/coloration of cells; microscopic observation of cell lysis in in vitro culture; and 2) ELISA and spectrophotometric detection of rotavirus.

The term “epitope” or “epitopes”, as used herein, refers to a structural part of an antigen to which an antibody specifically binds.

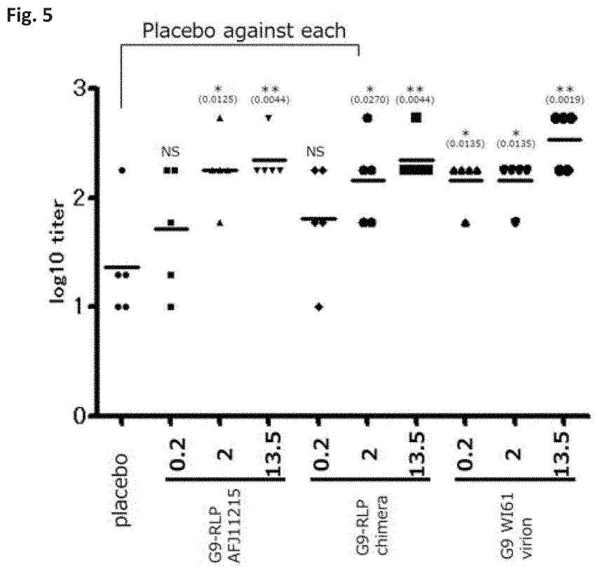

Immune responses elicited in response to administration of a VP7 fusion protein or a plant-produced RLP comprising a VP7 fusion protein may for example be observed in Balb/C mice. Serum samples from blood collected from animals may be analyzed by ELISA for VP7-specific total IgG and IgA antibodies. Mice immunized with VP7 fusion protein or a plant-produced RLP comprising a VP7 fusion protein may exhibit rotavirus VP7-specific IgG antibody titers in sera for each treatment group (see FIG. 5). FIG. 5 shows the microneutralization titers of cell cultures infected with rotavirus G9 strain in presence of G9-mice immunized serum. As seen in the figure, native and RLP comprising VP7 fusion protect against G9 virus infection as well as immunization that was obtained with the G9 virus.

A method of producing an antibody or antibody fragment is provided, the method comprising administering the VP7 fusion protein or RLP comprising the VP7 fusion protein as described herewith to a subject, or a host animal, thereby producing the antibody or the antibody fragment. Antibodies or the antibody fragments produced by the method are also provided.

The present disclosure therefore also provides the use of a VP7 fusion protein or RLP comprising the VP7 fusion protein, as described herein, for inducing immunity to a rotavirus infection in a subject. Also disclosed herein is an antibody or antibody fragment, prepared by administering the VP7 fusion protein or RLP comprising the VP7 fusion protein, to a subject or a host animal. Further provided is a composition comprising an effective dose of VP7 fusion protein or RLP comprising the VP7 fusion protein, as described herein, and a pharmaceutically acceptable carrier, adjuvant, vehicle, or excipient, for inducing an immune response in a subject. Also provided is a vaccine for inducing an immune response in a subject, wherein the vaccine comprises an effective dose of the VP7 fusion protein or RLP comprising the VP7 fusion protein.

For rotavirus VP7 protein, the 7-1a subdomain is immune-dominant. Accordingly, the 7-1a subdomain or 7-1a epitope may produce the dominant immune response in a subject. Accordingly, in one aspect the VP7 fusion protein or RLP comprising the VP7 fusion protein may induce immunity in a subject to the rotavirus genotype or strain from which the 7-1a subdomain is derived. In another aspect the VP7 fusion protein or RLP comprising the VP7 fusion protein may induce immunity in a subject to the rotavirus genotype or strain from which the 7-1a subdomain and the 7-1b subdomain are derived.

The rotavirus VP7 fusion protein may comprise a first sequence encoding a 7-2 domain derived from a first rotavirus strain and a second sequence encoding a 7-1a domain, a 7-1b subdomain or a 7-1a subdomain and a 7-1b subdomain derived from a second rotavirus strain. The VP7 fusion protein or a RLP comprising the VP7 fusion protein may induce immunity in a subject to the second rotavirus strain.

### Plant Expression

The constructs of the present disclosure can be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants In Plant Metabolism, 2d Ed. D T. Dennis, D H Turpin, D D Lefebvre, D B Layzell (eds), Addison Wesly, Langmans Ltd. London, pp. 561-579 (1997). Other methods include direct DNA uptake, the use of liposomes, electroporation, for example using protoplasts, micro-injection, microprojectiles or whiskers, and vacuum infiltration. See, for example, Bilang, et al. (1991, Gene 100: 247-250), Scheid et al. (1991, Mol. Gen. Genet. 228: 104-112), Guerche et al. (1987, Plant Science 52: 111-116), Neuhause et al. (1987, Theor. Appl Genet. 75: 30-36), Klein et al. (2987, Nature 327: 70-73); Freeman et al. (1984, Plant Cell Physiol. 29: 1353), Howell et al. (1980, Science 208: 1265), Horsch et al. (1985, Science 227: 1229-1231), DeBlock et al. (1989, Plant Physiology 91: 694-701), Methods for Plant Molecular Biology (Weissbach and Weissbach, eds., Academic Press Inc., 1988), Methods in Plant Molecular Biology (Schuler and Zielinski, eds., Academic Press Inc., 1989), WO 92/09696, WO 94/00583, EP 331083, EP 175966, Liu and Lomonossoff (2002, J Virol Meth, 105:343-348), EP 290395; WO 8706614; U.S. Pat. Nos. 4,945,050; 5,036,006; and 5,100,792, U.S. patent application Ser. No. 08/438,666, filed May 10, 1995, and Ser. No. 07/951,715, filed Sep. 25, 1992, (all of which are hereby incorporated by reference).

Transient expression methods may be used to express the constructs of the present disclosure (see D'Aoust et al., 2009, Methods in molecular biology, Vol 483, pages 41-50; Liu and Lomonossoff, 2002, Journal of Virological Methods, 105:343-348; which is incorporated herein by reference). Alternatively, a vacuum-based transient expression method, as described by Kapila et al. (1997, Plant Sci. 122, 101-108; which is incorporated herein by reference), or WO 00/063400, WO 00/037663 (which are incorporated herein by reference) may be used. These methods may include, for example, but are not limited to, a method of Agro-inoculation or Agro-infiltration, syringe infiltration, however, other transient methods may also be used as noted above. With Agro-inoculation, Agro-infiltration, or syringe infiltration, a mixture of Agrobacteria comprising the desired nucleic acid enter the intercellular spaces of a tissue, for example the leaves, aerial portion of the plant (including stem, leaves and flower), other portion of the plant (stem, root, flower), or the whole plant. After crossing the epidermis the Agrobacteria infect and transfer t-DNA copies into the cells. The t-DNA is episomally transcribed and the mRNA translated, leading to the production of the protein of interest in infected cells, however, the passage of t-DNA inside the nucleus is transient.

Also considered part of this disclosure are transgenic plants, plant cells or seeds containing the gene construct of the present disclosure that may be used as a platform plant suitable for transient protein expression described herein. Methods of regenerating whole plants from plant cells are also known in the art (for example see Guerineau and Mullineaux (1993, Plant transformation and expression vectors. In: Plant Molecular Biology Labfax (Croy R R D ed) Oxford, BIOS Scientific Publishers, pp 121-148). In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques.

Transgenic plants can also be generated without using tissue culture. Methods for stable transformation, and regeneration of these organisms are established in the art and known to one of skill in the art. Available techniques are reviewed in Vasil et al. (Cell Culture and Somatic Cell Genetics of Plants, VoI I, II and III, Laboratory Procedures and Their Applications, Academic Press, 1984), and Weissbach and Weissbach (Methods for Plant Molecular Biology, Academic Press, 1989). The method of obtaining transformed and regenerated plants is not critical to the present disclosure.

If plants, plant portions or plant cells are to be transformed or co-transformed by two or more nucleic acid constructs, the nucleic acid construct may be introduced into the Agrobacterium in a single transfection event so that the nucleic acids are pooled, and the bacterial cells transfected. Alternatively, the constructs may be introduced serially. In this case, a first construct is introduced into the Agrobacterium as described, the cells are grown under selective conditions (e.g. in the presence of an antibiotic) where only the singly transformed bacteria can grow. Following this first selection step, a second nucleic acid construct is introduced into the Agrobacterium as described, and the cells are grown under doubly-selective conditions, where only the doubly-transformed bacteria can grow. The doubly-transformed bacteria may then be used to transform a plant, plant portion or plant cell as described herein, or may be subjected to a further transformation step to accommodate a third nucleic acid construct.

Alternatively, if plants, plant portions, or plant cells are to be transformed or co-transformed by two or more nucleic acid constructs, the nucleic acid construct may be introduced into the plant by co-infiltrating a mixture of Agrobacterium cells with the plant, plant portion, or plant cell, each Agrobacterium cell may comprise one or more constructs to be introduced within the plant. In order to vary the relative expression levels within the plant, plant portion or plant cell, of a nucleotide sequence of interest within a construct, during the step of infiltration, the concentration of the various Agrobacteria populations comprising the desired constructs may be varied.

The term “plant”, “portion of a plant”, “plant portion”, “plant matter”, “plant biomass”, “plant material”, plant extract”, or “plant leaves”, as used herein, may comprise an entire plant, tissue, cells, or any fraction thereof, intracellular plant components, extracellular plant components, liquid or solid extracts of plants, or a combination thereof, that are capable of providing the transcriptional, translational, and post-translational machinery for expression of one or more than one nucleic acids described herein, and/or from which an expressed protein or RLP may be extracted and purified. Plants may include, but are not limited to, agricultural crops including for example canola, Brassica spp., maize, Nicotiana spp., (tobacco) for example, Nicotiana benthamiana, Nicotiana rustica, Nicotiana, tabacum, Nicotiana alata, Arabidopsis thaliana, alfalfa, potato, sweet potato (Ipomoea batatus), ginseng, pea, oat, rice, soybean, wheat, barley, sunflower, cotton, corn, rye (Secale cereale), sorghum (Sorghum bicolor, Sorghum vulgare), safflower (Carthamus tinctorius).

The term “plant portion”, as used herein, refers to any part of the plant including but not limited to leaves, stem, root, flowers, fruits, a plant cell obtained from leaves, stem, root, flowers, fruits, a plant extract obtained from leaves, stem, root, flowers, fruits, or a combination thereof. The term “plant extract”, as used herein, refers to a plant-derived product that is obtained following treating a plant, a portion of a plant, a plant cell, or a combination thereof, physically (for example by freezing followed by extraction in a suitable buffer), mechanically (for example by grinding or homogenizing the plant or portion of the plant followed by extraction in a suitable buffer), enzymatically (for example using cell wall degrading enzymes), chemically (for example using one or more chelators or buffers), or a combination thereof A plant extract may be further processed to remove undesired plant components for example cell wall debris. A plant extract may be obtained to assist in the recovery of one or more components from the plant, portion of the plant or plant cell, for example a protein (including protein complexes, protein suprastructures and/or RLPs), a nucleic acid, a lipid, a carbohydrate, or a combination thereof from the plant, portion of the plant, or plant cell. If the plant extract comprises proteins, then it may be referred to as a protein extract. A protein extract may be a crude plant extract, a partially purified plant or protein extract, or a purified product, that comprises one or more proteins, protein complexes, protein suprastructures, and/or VLPs, from the plant tissue. If desired a protein extract, or a plant extract, may be partially purified using techniques known to one of skill in the art, for example, the extract may be subjected to salt or pH precipitation, centrifugation, gradient density centrifugation, filtration, chromatography, for example, size exclusion chromatography, ion exchange chromatography, affinity chromatography, or a combination thereof. A protein extract may also be purified, using techniques that are known to one of skill in the art.

The term nucleic acid segment as used herein refers to a sequence of nucleic acids that encodes a protein of interest. In addition to the sequence of nucleic acids, the nucleic acid segment comprise a regulatory region and a terminator that are operatively linked to the sequence of nucleic acids. The regulatory region may for example comprise a promoter, and optionally, an enhancer element operatively linked to the promoter.

The rotavirus proteins of the present description, for example the rotavirus VP7 fusion protein as described herewith may be expressed in an expression system comprising a viral based, DNA or RNA, expression system, for example but not limited to, a comovirus-based expression cassette.

The expression system as described herein may comprise an expression cassette based on a bipartite virus, or a virus with a bipartite genome. For example, the bipartite viruses may be of the Comoviridae family. Genera of the Comoviridae family include Comovirus, Nepovirus, Fabavirus, Cheravirus and Sadwavirus. Comoviruses include Cowpea mosaic virus (CPMV), Cowpea severe mosaic virus (CPSMV), Squash mosaic virus (SqMV), Red clover mottle virus (RCMV), Bean pod mottle virus (BPMV), Turnip ringspot virus (TuRSV), Broad bean true mosaic virus (BBtMV), Broad bean stain virus (BBSV), Radish mosaic virus (RaMV). Examples of comovirus RNA-2 sequences comprising enhancer elements that may be useful for various aspects of the invention include, but are not limited to: CPMV RNA-2 (GenBank Accession No. NC_003550), RCMV RNA-2 (GenBank Accession No. NC_003738), BPMV RNA-2 (GenBank Accession No. NC_003495), CPSMV RNA-2 (GenBank Accession No. NC_003544), SqMV RNA-2 (GenBank Accession No. NC_003800), TuRSV RNA-2 (GenBank Accession No. NC_013219.1). BBtMV RNA-2 (GenBank Accession No. GU810904), BBSV RNA2 (GenBank Accession No. FJ028650), RaMV (GenBank Accession No. NC_003800).

Segments of the bipartite comoviral RNA genome are referred to as RNA-1 and RNA-2. RNA-1 encodes the proteins involved in replication while RNA-2 encodes the proteins necessary for cell-to-cell movement and the two capsid proteins. Any suitable comovirus-based cassette may be used including CPMV, CPSMV, SqMV, RCMV, or BPMV, for example, the expression cassette may be based on CPMV.

“Expression cassette” refers to a nucleotide sequence comprising a nucleic acid of interest under the control of, and operably (or operatively) linked to, an appropriate promoter or other regulatory elements for transcription of the nucleic acid of interest in a host cell.

The term “nucleic acid complex” as used herein refers to a combination of two or more than two nucleic acid segments. The two or more than two nucleic acid segments may be present in a single nucleic acid, so that the nucleic acid complex comprises two, or more than two nucleic acid segments, with each nucleic acid segment under the control of a regulatory region and a terminator. Alternatively, the nucleic acid complex may comprise two or more separate nucleic acids, each of the nucleic acids comprising one or more than one nucleic acid segment, where each nucleic acid segment is under the control of a regulatory region and a terminator. For example a nucleic acid complex may comprise one nucleic acid that comprises two nucleic acid segments, a nucleic acid complex may comprise two nucleic acids, each nucleic acid comprising one nucleic acid segment, or a nucleic acid complex may comprise two or more than two nucleic acids, with each nucleic acid comprising one or more than one nucleic acid segment.

The term “vector” or “expression vector”, as used herein, refers to a recombinant nucleic acid for transferring exogenous nucleic acid sequences into host cells (e.g. plant cells) and directing expression of the exogenous nucleic acid sequences in the host cells. “Expression cassette” refers to a nucleotide sequence comprising a nucleic acid of interest under the control of, and operably (or operatively) linked to, an appropriate promoter or other regulatory elements for transcription of the nucleic acid of interest in a host cell. As one of skill in the art would appreciate, the expression cassette may comprise a termination (terminator) sequence that is any sequence that is active the plant host. For example the termination sequence may be derived from the RNA-2 genome segment of a bipartite RNA virus, e.g. a comovirus, the termination sequence may be a NOS terminator, or terminator sequence may be obtained from the 3′UTR of the alfalfa plastocyanin gene.

The constructs of the present disclosure may further comprise a 3′ untranslated region (UTR). A 3′ untranslated region contains a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon. Non-limiting examples of suitable 3′ regions are the 3′ transcribed non-translated regions containing a polyadenylation signal of Agrobacterium tumor inducing (Ti) plasmid genes, such as the nopaline synthase (Nos gene) and plant genes such as the soybean storage protein genes, the small subunit of the ribulose-1, 5-bisphosphate carboxylase gene (ssRUBISCO; U.S. Pat. No. 4,962,028; which is incorporated herein by reference), the promoter used in regulating plastocyanin expression.

By “regulatory region” “regulatory element” or “promoter” it is meant a portion of nucleic acid typically, but not always, upstream of the protein coding region of a gene, which may be comprised of either DNA or RNA, or both DNA and RNA. When a regulatory region is active, and in operative association, or operatively linked, with a nucleotide sequence of interest, this may result in expression of the nucleotide sequence of interest. A regulatory element may be capable of mediating organ specificity, or controlling developmental or temporal gene activation. A “regulatory region” includes promoter elements, core promoter elements exhibiting a basal promoter activity, elements that are inducible in response to an external stimulus, elements that mediate promoter activity such as negative regulatory elements or transcriptional enhancers. “Regulatory region”, as used herein, also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element may comprise a basal promoter element, responsible for the initiation of transcription, as well as other regulatory elements that modify gene expression.

There are several types of regulatory regions, including those that are developmentally regulated, inducible or constitutive. A regulatory region that is developmentally regulated or controls the differential expression of a gene under its control, is activated within certain organs or tissues of an organ at specific times during the development of that organ or tissue. However, some regulatory regions that are developmentally regulated may preferentially be active within certain organs or tissues at specific developmental stages, they may also be active in a developmentally regulated manner, or at a basal level in other organs or tissues within the plant as well. Examples of tissue-specific regulatory regions, for example see-specific a regulatory region, include the napin promoter, and the cruciferin promoter (Rask et al., 1998, J. Plant Physiol. 152: 595-599; Bilodeau et al., 1994, Plant Cell 14: 125-130). An example of a leaf-specific promoter includes the plastocyanin promoter (see U.S. Pat. No. 7,125,978, which is incorporated herein by reference).

An inducible regulatory region is one that is capable of directly or indirectly activating transcription of one or more DNA sequences or genes in response to an inducer. In the absence of an inducer the DNA sequences or genes will not be transcribed. Typically, the protein factor that binds specifically to an inducible regulatory region to activate transcription may be present in an inactive form, which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory region may be exposed to an inducer by externally applying the inducer to the cell or plant such as by spraying, watering, heating or similar methods. Inducible regulatory elements may be derived from either plant or non-plant genes (e.g. Gatz, C. and Lenk, I. R. P., 1998, Trends Plant Sci. 3, 352-358). Examples, of potential inducible promoters include, but not limited to, tetracycline-inducible promoter (Gatz, C., 1997, Ann. Rev. Plant Physiol. Plant Mol. Biol. 48, 89-108), steroid inducible promoter (Aoyama, T. and Chua, N. H., 1997, Plant J. 2, 397-404) and ethanol-inducible promoter (Salter, M. G., et al, 1998, Plant Journal 16, 127-132; Caddick, M. X., et al, 1998, Nature Biotech. 16, 177-180) cytokinin inducible IB6 and CKI1 genes (Brandstatter, I. and Kieber, J. J., 1998, Plant Cell 10, 1009-1019; Kakimoto, T., 1996, Science 274, 982-985) and the auxin inducible element, DR5 (Ulmasov, T., et al., 1997, Plant Cell 9, 1963-1971).

A constitutive regulatory region directs the expression of a gene throughout the various parts of a plant and continuously throughout plant development. Examples of known constitutive regulatory elements include promoters associated with the CaMV 35S transcript. (p35S; Odell et al., 1985, Nature, 313: 810-812; which is incorporated herein by reference), the rice actin 1 (Zhang et al, 1991, Plant Cell, 3: 1155-1165), actin 2 (An et al., 1996, Plant J., 10: 107-121), or tms 2 (U.S. Pat. No. 5,428,147), and triosephosphate isomerase 1 (Xu et. al., 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646), the tobacco translational initiation factor 4A gene (Mandel et al, 1995 Plant Mol. Biol. 29: 995-1004). the Cassava Vein Mosaic Virus promoter, pCAS, (Verdaguer et al., 1996); the promoter of the small subunit of ribulose biphosphate carboxylase, pRbcS: (Outchkourov et al., 2003), the pUbi (for monocots and dicots).

The term “constitutive” as used herein does not necessarily indicate that a nucleotide sequence under control of the constitutive regulatory region is expressed at the same level in all cell types, but that the sequence is expressed in a wide range of cell types even though variation in abundance is often observed.

The expression constructs as described above may be present in a vector. The vector may comprise border sequences which permit the transfer and integration of the expression cassette into the genome of the organism or host. The construct may be a plant binary vector, for example a binary transformation vector based on pPZP (Hajdukiewicz, et al. 1994). Other example constructs include pBin19 (see Frisch, D. A., L. W. Harris-Haller, et al. 1995, Plant Molecular Biology 27: 405-409).

The term “native”, “native protein” or “native domain”, as used herein, refers to a protein or domain having a primary amino acid sequence identical to wildtype. Native proteins or domains may be encoded by nucleotide sequences having 100% sequence similarity to the wildtype sequence. A native amino acid sequence may also be encoded by a human codon (hCod) optimized nucleotide sequence or a nucleotide sequence comprising an increased GC content when compared to the wild type nucleotide sequence provided that the amino acid sequence encoded by the hCod-nucleotide sequence exhibits 100% sequence identity with the native amino acid sequence.

By a nucleotide sequence that is “human codon optimized” or a “hCod” nucleotide sequence, it is meant the selection of appropriate DNA nucleotides for the synthesis of an oligonucleotide sequence or fragment thereof that approaches the codon usage generally found within an oligonucleotide sequence of a human nucleotide sequence. By “increased GC content” it is meant the selection of appropriate DNA nucleotides for the synthesis of an oligonucleotide sequence or fragment thereof in order to approach codon usage that, when compared to the corresponding native oligonucleotide sequence, comprises an increase of GC content, for example, from about 1 to about 30%, or any amount therebetween, over the length of the coding portion of the oligonucleotide sequence. For example, from about 1, 2, 4, 6, 8, 10, 12, 14, 16, 18, 20, 22, 24, 26, 28, 30%, or any amount therebetween, over the length of the coding portion of the oligonucleotide sequence. As described below, a human codon optimized nucleotide sequence, or a nucleotide sequence comprising an increased GC contact (when compared to the wild type nucleotide sequence) exhibits increased expression within a plant, portion of a plant, or a plant cell, when compared to expression of the non-human optimized (or lower GC content) nucleotide sequence.

TABLE 2
SEQ ID NOs and Description of Sequences

| SEQ ID NO: | Description of Sequence | SEQ ID NO: | Description of Sequence |
|---|---|---|---|
| SEQ ID NO: 1 | IF-WA_VP2 (opt).s1 + 3c | SEQ ID NO: 69 | G3HCR3 + 7-1a-1b_ |
| | | | G4Brb-9_VP7_AA |
| SEQ ID NO: 2 | IF-WA_VP2 (opt).s1 − 4r | SEQ ID NO: 70 | G3HCR3 + 7-1a-1b_ |
| | | | G9BE2001_VP7_DNA_opt |
| SEQ ID NO: 3 | Wa_VP2_DNA_Opt | SEQ ID NO: 71 | G3HCR3 + 7-1a-1b_ |
| | | | G9BE2001_VP7_AA |
| SEQ ID NO: 4 | Wa_VP2_AA | SEQ ID NO: 72 | G3HCR3 + 7-1a-1b_ |
| | | | G12K12_VP7_DNA_opt |
| SEQ ID NO: 5 | Cloning vector 1191 | SEQ ID NO: 73 | G3HCR3 + 7-1a-1b_ |
| | from left to right T-DNA | | Gl2K12_VP7_AA |
| SEQ ID NO: 6 | Construct 1710 from | SEQ ID NO: 74 | IF-(160) RVA |
| | 2X35S to NOS | | (G4P5BrB-9) VP7.c |
| SEQ ID NO: 7 | IF-WA_VP6 (opt).s1 + 3c | SEQ ID NO: 75 | IF-RVA (G4P5BrB-9) VP7.r |
| SEQ ID NO: 8 | IF-WA_VP6 (opt).s1 − 4r | SEQ ID NO: 76 | G4BrB-9_VP7_DNA_opt |
| SEQ ID NO: 9 | Wa_VP6_DNA_Opt | SEQ ID NO: 77 | G3HCR3_VP7_AA |
| SEQ ID NO: 10 | Wa_VP6_AA | SEQ ID NO: 78 | G4BrB-9_VP7_AA |
| SEQ ID NO: 11 | Construct 1713 from | SEQ ID NO: 79 | G4BrB-9 + 7-1a-1b_ |
| | 2X35S to NOS | | G1Rtx_VP7_DNA_opt |
| SEQ ID NO: 12 | IF-WA_NSP4.s1 + 3c | SEQ ID NO: 80 | G4BrB-9 + 7-1a-1b_ |
| | | | G1Rtx_VP7_AA |
| SEQ ID NO: 13 | IF-WA_NSP4.s1 − 4r | SEQ ID NO: 81 | G4BrB-9 + 7-1a-1b_ |
| | | | G2Sc2-9_VP7_DNA_opt |
| SEQ ID NO: 14 | Wa_NSP4_DNA | SEQ ID NO: 82 | G4BrB-9 + 7-1a-1b_ |
| | | | G2Sc2-9_VP7_AA |
| SEQ ID NO: 15 | Wa_NSP4_AA | SEQ ID NO: 83 | G4BrB-9 + 7-1a-1b_ |
| | | | G3HCR3_VP7_DNA_opt |
| SEQ ID NO: 16 | Construct 1706 from | SEQ ID NO: 84 | G4BrB-9 + 7-1a-1b_ |
| | 2X35S to NOS | | G3HCR3_VP7_AA |
| SEQ ID NO: 17 | IF (C160)- | SEQ ID NO: 85 | G4BrB-9 + 7-1a-1b_ |
| | TrSP + Rtx_VP7 (opt).c | | G9BE2001_VP7_DNA_opt |
| SEQ ID NO: 18 | IF-Rtx_VP7 (opt).s1 − 4r | SEQ ID NO: 86 | G4BrB-9 + 7-1a-1b_ |
| | | | G9BE2001_VP7_AA |
| SEQ ID NO: 19 | Rtx_VP7_DNA_Opt | SEQ ID NO: 87 | G4BrB-9 + 7-1a-1b_ |
| | | | G12K12_VP7_DNA_opt |
| SEQ ID NO: 20 | Rtx_VP7_AA | SEQ ID NO: 88 | G4BrB-9 + 7-1a-1b_ |
| | | | G12K12_VP7_AA |
| SEQ ID NO: 21 | Cloning vector 1190 | SEQ ID NO: 89 | IF-VP7 |
| | from left to right T-DNA | | (G9AFJ11215) (opt).c |
| SEQ ID NO: 22 | Construct 1199 from | SEQ ID NO: 90 | IF-VP7 |
| | 2X35S to NOS | | (G9AFJ11215) (opt).r |
| SEQ ID NO: 23 | IF-(160) RVA | SEQ ID NO: 91 | G9BE2001_VP7_DNA_opt |
| | (G2P5SC2-9) VP7.c | | |
| SEQ ID NO: 24 | IF-RVA | SEQ ID NO: 92 | G9BE2001_VP7_AA |
| | (G2P5SC2-9) VP7.r | | |
| SEQ ID NO: 25 | Sc2-9_VP7_DNA_Opt | SEQ ID NO: 93 | G9BE2001 + 7-1a-1b_ |
| | | | G1Rtx_VP7_DNA_opt |
| SEQ ID NO: 26 | Sc2-9_VP7_AA | SEQ ID NO: 94 | G9BE2001 + 7-1a-1b_ |
| | | | G1Rtx_VP7_AA |
| SEQ ID NO: 27 | 7-1a_Sc2-9_ | SEQ ID NO: 95 | G9BE2001 + 7-1a-1b_ |
| | VP7_DNA_Opt | | G2Sc2-9_VP7_DNA_opt |
| SEQ ID NO: 28 | 7-la_Sc2-9_VP7_AA | SEQ ID NO: 96 | G9BE2001 + 7-1a-1b_ |
| | | | G2Sc2-9_VP7_AA |
| SEQ ID NO: 29 | IF-VP7 (3End) Rtx + | SEQ ID NO: 97 | G9BE2001 + 7-1a-1b_ |
| | VP7-1b (G2SC2-9).r | | G3HCR3_VP7_DNA_opt |
| SEQ ID NO: 30 | 7-1b_Sc2-9_ | SEQ ID NO: 98 | G9BE2001 + 7-1a-1b_ |
| | VP7_DNA_Opt | | G3HCR3_VP7_AA |
| SEQ ID NO: 31 | 7-1b_Sc2-9_VP7_AA | SEQ ID NO: 99 | G9BE2001 + 7-1a-1b_ |
| | | | G4BrB-9_VP7_DNA_opt |
| SEQ ID NO: 32 | 7-1a + 1b_Sc2-9_ | SEQ ID NO: 100 | G9BE2001 + 7-1a-1b_ |
| | VP7_DNA_Opt | | G4BrB-9_VP7_AA |
| SEQ ID NO: 33 | 7-1a + 1b_Sc2-9_ | SEQ ID NO: 101 | G9BE2001 + 7-1a-1b_ |
| | VP7_AA | | G12K12_VP7_DNA_opt |
| SEQ ID NO: 34 | IF-(160) RVA | SEQ ID NO: 102 | G9BE2001 + 7-1a-1b_ |
| | (G9P8WI61) VP7.c | | Gl2K12_VP7_AA |
| SEQ ID NO: 35 | IF-RVA | SEQ ID NO: 103 | IF-VP7 |
| | (G9P8WI61) VP7.r | | (G12BAD89095).c |
| SEQ ID NO: 36 | WI61_VP7_DNA_Opt | SEQ ID NO: 104 | IF-VP7 |
| | | | (G12BAD89095).r |
| SEQ ID NO: 37 | WI61_VP7_AA | SEQ ID NO: 105 | G12K12_VP7_DNA_opt |
| SEQ ID NO: 38 | 7-1a_WI6l_VP7_ | SEQ ID NO: 106 | G12K12_VP7_AA |
| | DNA_Opt | | |
| SEQ ID NO: 39 | 7-1a_WI6l_ | SEQ ID NO: 107 | G12K12 + 7-1a-1b_ |
| | VP7_AA | | G1Rtx_VP7_DNA_opt |
| SEQ ID NO: 40 | 7-1b_WI61_VP7_ | SEQ ID NO: 108 | G12K12 + 7-1a-1b_ |
| | DNA_Opt | | G1Rtx_VP7_AA |
| SEQ ID NO: 41 | 7-1b_WI61_ | SEQ ID NO: 109 | Gl2K12 + 7-1a-1b_ |
| | VP7_AA | | G2Sc2-9_VP7_DNA_opt |
| SEQ ID NO: 42 | 7-1a + 1b_WI61_ | SEQ ID NO: 110 | G12K12 + 7-1a-1b_ |
| | VP7_DNA_Opt | | G2Sc2-9_VP7_AA |
| SEQ ID NO: 43 | 7-1a + 1b_WI61_ | SEQ ID NO: 111 | G12K12 + 7-1a-1b_ |
| | VP7_AA | | G3HCR3_VP7_DNA_opt |
| SEQ ID NO: 44 | IF-(160) RVA | SEQ ID NO: 112 | G12K12 + 7-1a-1b_ |
| | (G3P5WI78-8) VP7.r | | G3HCR3_VP7_AA |
| SEQ ID NO: 45 | IF-RVA | SEQ ID NO: 113 | G12K12 + 7-1a-1b_ |
| | (G3P5WI78-8) VP7.r | | G4BrB-9_VP7_DNA_opt |
| SEQ ID NO: 46 | WI78-8_VP7_DNA_Opt | SEQ ID NO: 114 | Gl2K12 + 7-1a-1b_ |
| | | | G4BrB-9_VP7_AA |
| SEQ ID NO: 47 | WI78-8_VP7_AA | SEQ ID NO: 115 | G12K12 + 7-1a-1b_ |
| | | | G9BE2001_VP7_DNA_opt |
| SEQ ID NO: 48 | IF-VP7 (3End) Rtx + | SEQ ID NO: 116 | G12K12 + 7-1a-1b_ |
| | VP7-1b (G3P5).r | | G9BE2001_VP7_AA |
| SEQ ID NO: 49 | 7-1b_WI78-8_ | SEQ ID NO: 117 | G3HCR3_VP7_DNA_opt |
| | VP7_DNA_Opt | | |
| SEQ ID NO: 50 | 7-1b_WI78-8_VP7_AA | SEQ ID NO: 118 | Boundary Sequence |
| SEQ ID NO: 51 | 7-1a + 1b_WI78-8_ | SEQ ID NO:119 | Boundary Sequence |
| | VP7_DNA_Opt | | |
| SEQ ID NO: 52 | 7-1a + 1b_WI78-8_ | SEQ ID NO: 120 | Boundary Sequence |
| | VP7_AA | | |
| SEQ ID NO: 53 | IF-(160) RVA | | |
| | (G12P8KDH651) VP7.c | SEQ ID NO: 121 | Boundary Sequence |
| SEQ ID NO: 54 | IF-RVA | SEQ ID NO: 122 | Enhancer nbGT61 |
| | (G12P8KDH651) VP7.r | | |
| SEQ ID NO: 55 | KDH651_VP7_DNA_Opt | SEQ ID NO: 1231 | Enhancer nbATL75 |
| SEQ ID NO: 56 | KDH651_VP7_AA | SEQ ID NO: 124 | Enhancer nbDJ46 |
| SEQ ID NO: 57 | IF-VP7(3End)Rtx + | SEQ ID NO: 125 | Enhancer nbCHP79 |
| | VP7-1b(G12P8).r | | |
| SEQ ID NO: 58 | 7-1b_KDH651_ | SEQ ID NO: 126 | Enhancer nbEN42 |
| | VP7_DNA_Opt | | |
| SEQ ID NO: 59 | 7-1b_KDH651_ | SEQ ID NO: 127 | Enhancer atHSP69 |
| | VP7_AA | | |
| SEQ ID NO: 60 | 7-la + 1b_KDH651_ | SEQ ID NO: 128 | Enhancer atGRP62 |
| | VP7_DNA_Opt | | |
| SEQ ID NO: 61 | 7-1a + 1b_KDH651_ | SEQ ID NO: 129 | Enhancer atPK65 |
| | VP7_AA | | |
| SEQ ID NO: 62 | IF-VP7 (G3AAA18522).c | SEQ ID NO: 130 | Enhancer atRP46 |
| SEQ ID NO: 63 | IF-VP7 (G3AAA18522).r | SEQ ID NO: 131 | Enhancer nb30S72 |
| SEQ ID NO: 64 | G3HCR3 + 7-1a-1b_ | SEQ ID NO: 132 | Enhancer nbMT78 |
| | G1Rtx_VP7_DNA_opt | | |
| SEQ ID NO: 65 | G3HCR3 + 7-1a-1b_ | SEQ ID NO: 133 | Enhancer nbPV55 |
| | G1Rtx_VP7_AA | | |
| SEQ ID NO: 66 | G3HCR3 + 7-1a-1b_ | SEQ ID NO: 134 | Enhancer nbPPI43 |
| | G2Sc2-9_VP7_DNA_opt | | |
| SEQ ID NO: 67 | G3HCR3 + 7-1a-1b_ | SEQ ID NO: 135 | Enhancer nbPM64 |
| | G2Sc2-9_VP7_AA | | |
| SEQ ID NO: 68 | G3HCR3 + 7-1a-1b_ | SEQ ID NO: 136 | Enhancer nbH2A86 |
| | G4Brb-9_VP7_DNA_opt | | |

TABLE 3
Rotavirus strains and constructs.

| | | SEQ ID | Constr. | Fig. |
|---|---|---|---|---|
| Trivial name | Strains | NO: | # | # |
| VP7 Fusions | | | | |
| VP7 (G1) + 7-1a G2 (aa) | RVA (Rtx G1) + VP7 (7-1a G2) | 28 | — | — |
| VP7 (G1) + 7-1a G2 (nt) | RVA (Rtx G1) + VP7 (7-1a G2) | 27 | 4540 | 6j |
| VP7 (G1) + 7-1b G2 (aa) | RVA (Rtx G1) VP7 (7-1b G2) | 31 | — | — |
| VP7 (G1) + 7-1b G2 (nt) | RVA (Rtx G1) VP7 (7-1b G2) | 30 | 4541 | 6k |
| VP7 (G1) + 7-1a-1b G2 (aa) | RVA (Rtx G1) VP7 (7-1a-1b G2) | 33 | — | — |
| VP7 (G1) + 7-1a-1b G2 (nt) | RVA (Rtx G1) VP7 (7-1a-1b G2) | 32 | 4542 | 6l |
| VP7 (G1) + 7-1a G9 (aa) | RVA (Rtx G1) VP7 (7-1a G9) | 39 | — | — |
| VP7 (G1) + 7-1a G9 (nt) | RVA (Rtx G1) VP7 (7-1a G9) | 38 | 4546 | 6n |
| VP7 (G1) + 7-1b G9 (aa) | RVA (Rtx G1) VP7 (7-1b- G9) | 41 | — | |
| VP7 (G1) + 7-1b G9 (nt) | RVA (Rtx G1) VP7 (7-1b- G9) | 40 | 4547 | 6o |
| VP7 (G1) + 7-1a-1b G9 (aa) | RVA (Rtx G1) VP7 (7-1a-1b G9) | 43 | — | — |
| VP7 (G1) + 7-1a-1b G9 (nt) | RVA (Rtx G1) VP7 (7-1a-1b G9) | 42 | 4548 | 6p |
| VP7 (G1) + 7-1b G3 (aa) | RVA (Rtx G1) VP7 (7-1b G3) | 50 | — | — |
| VP7 (G1) + 7-1b G3 (nt) | RVA (Rtx G1) VP7 (7-1b G3) | 49 | 4551 | 6r |
| VP7 (G1) + 7-1a-1b G3 (aa) | RVA (Rtx G1) VP7 (7-1a-1b G3) | 52 | — | — |
| VP7 (G1) + 7-1a-1b G3 (nt) | RVA (Rtx G1) VP7 (7-1a-1b G3) | 51 | 4552 | 6s |
| VP7 (G1) + 7-1b G12 (aa) | RVA (Rtx G1) VP7 (7-1b G12) | 59 | — | — |
| VP7 (G1) + 7-1b G12 (nt) | RVA (Rtx G1) VP7 (7-1b G12) | 58 | 4553 | 6u |
| VP7 (G1) + 7-1a-1b G12 (aa) | RVA (Rtx G1) VP7 (7-1a-1b G12) | 61 | — | — |
| VP7 (G1) + 7-1a-1b G12 (nt) | RVA (Rtx G1) VP7 (7-1a-1b G12) | 60 | 4554 | 6v |
| VP7 (G3 HCR3)-native (aa) | VP7 (G3 HCR3) | 77 | — | — |
| VP7 (G3 HCR3)-native (nt) | VP7 (G3 HCR3) | 117 | 6026 | 7a |
| VP7 (G3) + 7-1a-1b G1 (aa) | RVA (G3 HCR3) VP7 (7-1a-1b Rtx G1) | 65 | — | — |
| VP7 (G3) + 7-1a-1b G1 (nt) | RVA (G3 HCR3) VP7 (7-1a-1b Rtx G1) | 64 | 6501 | 7b |
| VP7 (G3) + 7-1a-1b G2 (aa) | RVA (G3 HCR3) VP7 (7-1a-1b G2) | 67 | — | — |
| VP7 (G3) + 7-1a-1b G2 (nt) | RVA (G3 HCR3) VP7 (7-1a-1b G2) | 66 | 6502 | 7c |
| VP7 (G3) + 7-1a-1b G4 (aa) | RVA (G3 HCR3) VP7 (7-1a-1b G4) | 69 | — | — |
| VP7 (G3) + 7-1a-1b G4 (nt) | RVA (G3 HCR3) VP7 (7-1a-1b G4) | 68 | 6503 | 7d |
| VP7 (G3) + 7-1a-1b G9 (aa) | RVA (G3 HCR3) VP7 (7-1a-1b G9) | 71 | — | — |
| VP7 (G3) + 7-1a-1b G9 (nt) | RVA (G3 HCR3) VP7 (7-1a-1b G9) | 70 | 6504 | 7e |
| VP7 (G3) + 7-1a-1b G12 (aa) | RVA (G3 HCR3) VP7 (7-1a-1b G12) | 73 | — | — |
| VP7 (G3) + 7-1a-1b G12 (nt) | RVA (G3 HCR3) VP7 (7-1a-1b G12) | 72 | 6505 | 7f |
| VP7 (G4 BrB9)-native (aa) | VP7 (G4 BrB9) | 78 | — | — |
| VP7 (G4 BrB9)-native (nt) | VP7 (G4 BrB9) | 76 | 3475 | 7g |
| VP7 (G4) + 7-1a-1b G1 (aa) | RVA (G4 BrB9) VP7 (7-1a-1b Rtx G1) | 80 | — | — |
| VP7 (G4) + 7-1a-1b G1 (nt) | RVA (G4 BrB9) VP7 (7-1a-1b Rtx G1) | 79 | 6506 | 7h |
| VP7 (G4) + 7-1a-1b G2 (aa) | RVA (G4 BrB9) VP7 (7-1a-1b G2) | 82 | — | — |
| VP7 (G4) + 7-1a-1b G2 (nt) | RVA (G4 BrB9) VP7 (7-1a-1b G2) | 81 | 6507 | 7i |
| VP7 (G4) + 7-1a-1b G3 (aa) | RVA (G4 BrB9) VP7 (7-1a-1b G3) | 84 | — | — |
| VP7 (G4) + 7-1a-1b G3 (nt) | RVA (G4 BrB9) VP7 (7-1a-1b G3) | 83 | 6508 | 7j |
| VP7 (G4) + 7-1a-1b G9 (aa) | RVA (G4 BrB9) VP7 (7-1a-1b G9) | 86 | — | — |
| VP7 (G4) + 7-1a-1b G9 (nt) | RVA (G4 BrB9) VP7 (7-1a-1b G9) | 85 | 6509 | 7k |
| VP7 (G4) + 7-1a-1b G12 (aa) | RVA (G4 BrB9) VP7 (7-1a-1b G12) | 87 | — | — |
| VP7 (G4) + 7-1a-1b G12 (nt) | RVA (G4 BrB9) VP7 (7-1a-1b G12) | 88 | 6510 | 7l |
| VP7 (G9 BE2001)-native (aa) | VP7 (G9 BE2001) | 92 | — | — |
| VP7 (G9 BE2001)-native (nt) | VP7 (G9 BE2001) | 91 | 4568 | 7m |
| VP7 (G9) + 7-1a-1b G1 (aa) | RVA (G9 BE2001) VP7 (7-1a-1b Rtx G1) | 94 | — | — |
| VP7 (G9) + 7-1a-1b G1 (nt) | RVA (G9 BE2001) VP7 (7-1a-1b Rtx G1) | 93 | 6511 | 7n |
| VP7 (G9) + 7-1a-1b G2 (aa) | RVA (G9 BE2001) VP7 (7-1a-1b G2) | 96 | — | — |
| VP7 (G9) + 7-1a-1b G2 (nt) | RVA (G9 BE2001) VP7 (7-1a-1b G2) | 95 | 6512 | 7o |
| VP7 (G9) + 7-1a-1b G3 (aa) | RVA (G9 BE2001) VP7 (7-1a-1b G3) | 97 | — | — |
| VP7 (G9) + 7-1a-1b G3 (nt) | RVA (G9 BE2001) VP7 (7-1a-1b G3) | 98 | 6513 | 7p |
| VP7 (G9) + 7-1a-1b G4 (aa) | RVA (G9 BE2001) VP7 (7-1a-1b G4) | 100 | — | — |
| VP7 (G9) + 7-1a-1b G4 (nt) | RVA (G9 BE2001) VP7 (7-1a-1b G4) | 99 | 6514 | 7q |
| VP7 (G9) + 7-1a-1b G12 (aa) | RVA (G9 BE2001) VP7 (7-1a-1b G12) | 102 | — | — |
| VP7 (G9) + 7-1a-1b G12 (nt) | RVA (G9 BE2001) VP7 (7-1a-1b G12) | 101 | 6515 | 7r |
| VP7 (G12 K12)-native (aa) | VP7 (G12 K12) | 105 | — | — |
| VP7 (G12 K12)-native (nt) | VP7 (G12 K12) | 106 | 6042 | 7s |
| VP7 (G12) + 7-1a-1b G1 (aa) | RVA (G12 K12) VP7 (7-1a-1b Rtx G1) | 108 | — | — |
| VP7 (G12) + 7-1a-1b G1 (nt) | RVA (G12 K12) VP7 (7-1a-1b Rtx G1) | 107 | 6516 | 7t |
| VP7 (G12) + 7-1a-1b G2 (aa) | RVA (G12 K12) VP7 (7-1a-1b G2) | 110 | — | — |
| VP7 (G12) + 7-1a-1b G2 (nt) | RVA (G12 K12) VP7 (7-1a-1b G2) | 109 | 6517 | 7u |
| VP7 (G12) + 7-1a-1b G3 (aa) | RVA (G12 K12) VP7 (7-1a-1b G3) | 112 | — | — |
| VP7 (G12) + 7-1a-1b G3 (nt) | RVA (G12 K12) VP7 (7-1a-1b G3) | 111 | 6518 | 7v |
| VP7 (G12) + 7-1a-1b G4 (aa) | RVA (G12 K12) VP7 (7-1a-1b G4) | 114 | — | — |
| VP7 (G12) + 7-1a-1b G4 (nt) | RVA (G12 K12) VP7 (7-1a-1b G4) | 113 | 6519 | 7w |
| VP7 (G12) + 7-1a-1b G9 (aa) | RVA (G12 K12) VP7 (7-1a-1b G9) | 116 | — | — |
| VP7 (G12) + 7-1a-1b G9 (nt) | RVA (G12 K12) VP7 (7-1a-1b G9) | 115 | 6520 | 7x |

The present invention will be further illustrated in the following examples.

Expression of native rotavirus G2P5 VP7 protein and native G3P5 VP7 protein has proven to be challenging and VP7 protein production is below detectable levels in Western Blots analysis (see FIGS. 3a and 3b). However, as shown in FIGS. 3a and 3b, VP7 fusion proteins comprising 7-1a₂₋₇-2₁--7-1b₁ (7-1a), 7-1a₁--7-2₁--7-1b₂ (7-1b) or 7-1a₂--7-2₁--7-1b₂ (7-1a-1b) resulted in VP7 fusion protein production in plants (as determined using a SDS-Comassie stained gel, or Western analysis) that was similar to or greater than the yield of the native VP7 protein that comprised the corresponding domains or subdomains either exclusively from the first (Rtx) or second rotavirus strain (G2P5 or G3P5), as determined using a SDS-Comassie stained gel, or Western analysis. As can be seen in FIG. 3a, when expressed in plants, VP7 fusion proteins VP7(Rtx)+(7-1a)G2P5, VP7(Rtx)+(7-1b)G2P5 and VP7(Rtx)+(7-1a-1b)G2P5 showed higher expression levels than the native VP7 G2P5 protein. Furthermore, VP7 fusion proteins VP7(Rtx)+(7-1a)G9P8, VP7(Rtx)+(7-1b) G9P8 and VP7(Rtx)+(7-1a-1b) G9P8 showed higher expression levels than the native VP7 G9P8 protein.

Furthermore, as shown in FIG. 3b, when expressed in plants, VP7 fusion proteins VP7(Rtx)+(7-1b)G3P5 and VP7(Rtx)+(7-1a-1b) G3P5 showed higher expression levels than the native VP7 G3P5 protein. Similarly, VP7 fusion proteins VP7(Rtx)+(7-1b)G12P8 and VP7(Rtx)+(7-1a-1b) G12P8 showed higher expression levels than the native VP7 G12P8 protein.

### Example 1: Rotavirus VP7 Constructs

### 2X35S/CPMV-HT/RVA(WA) VP2(Opt)/NOS (Construct Number 1710)

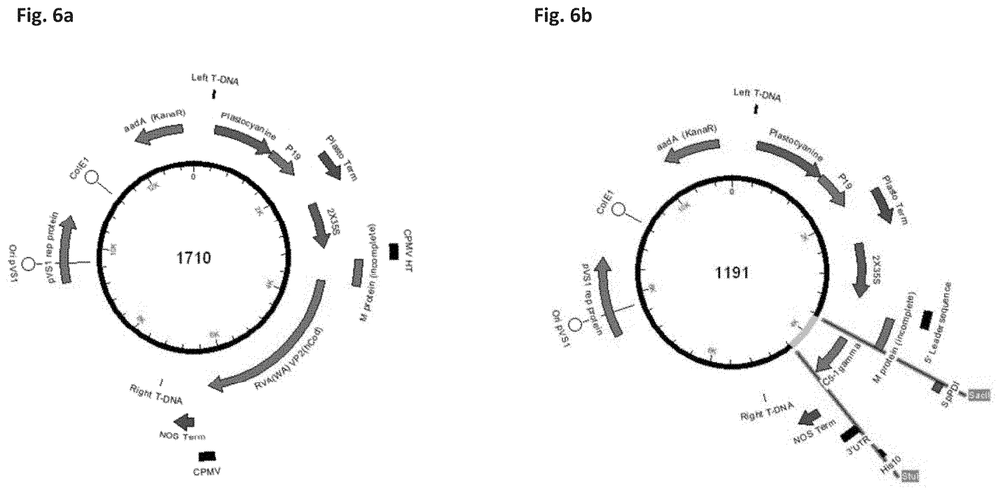

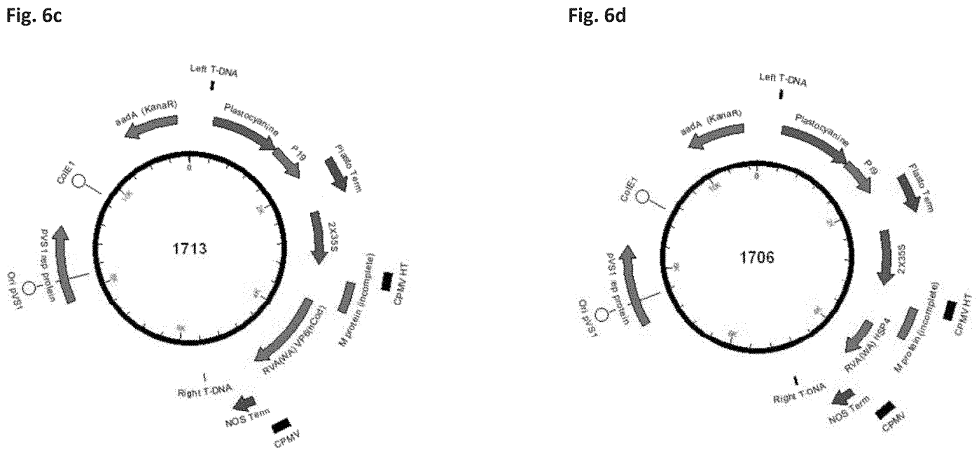

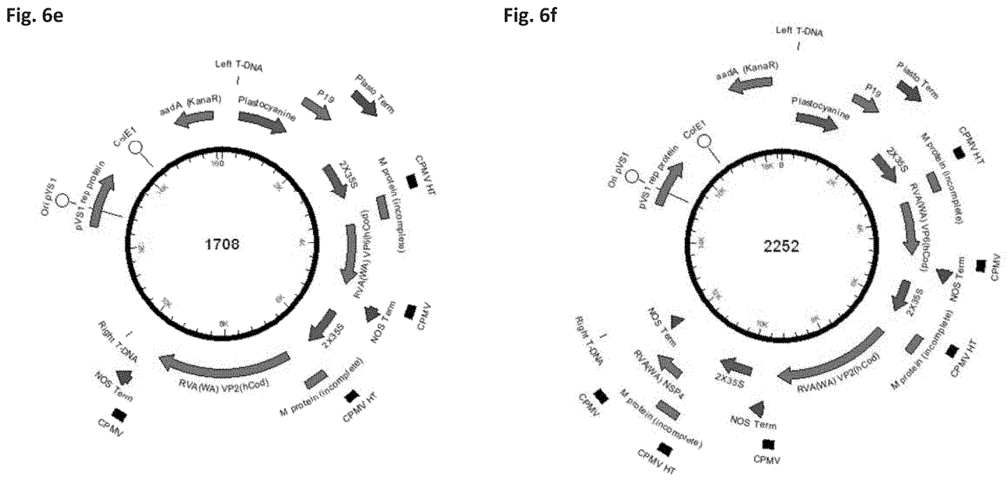

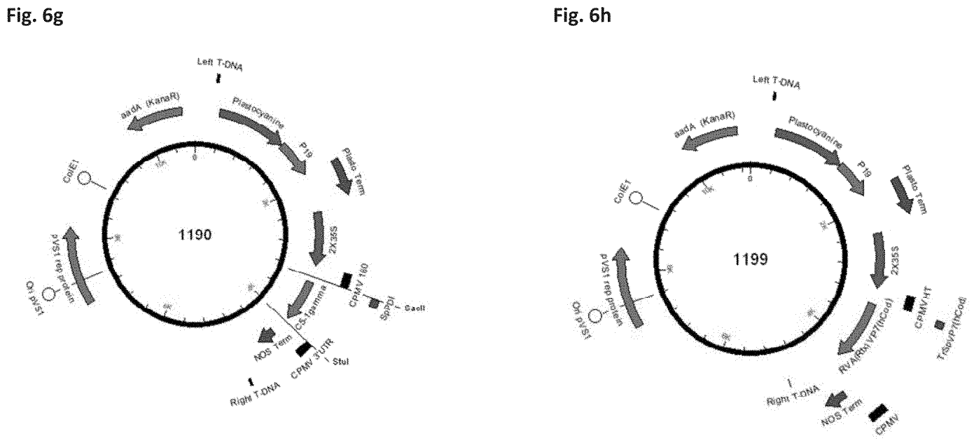

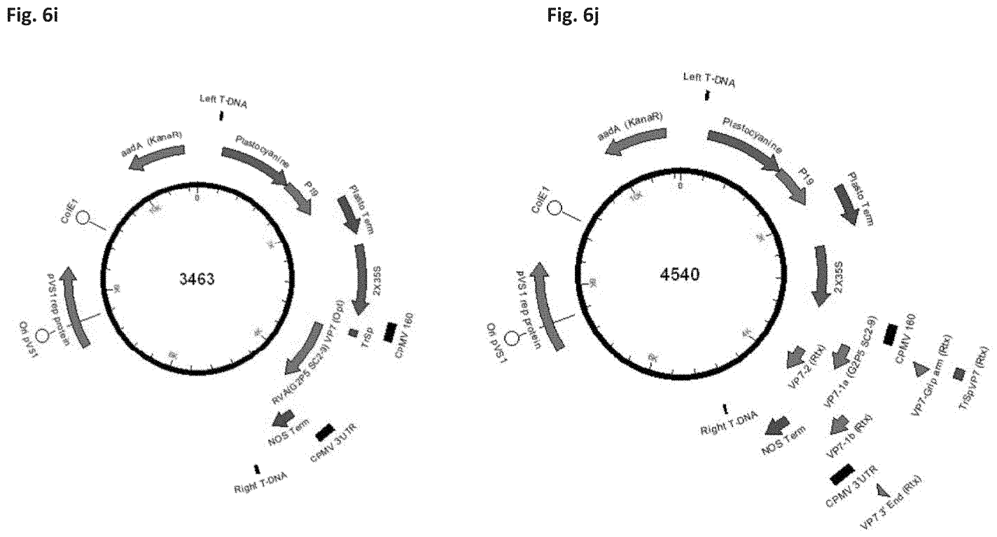

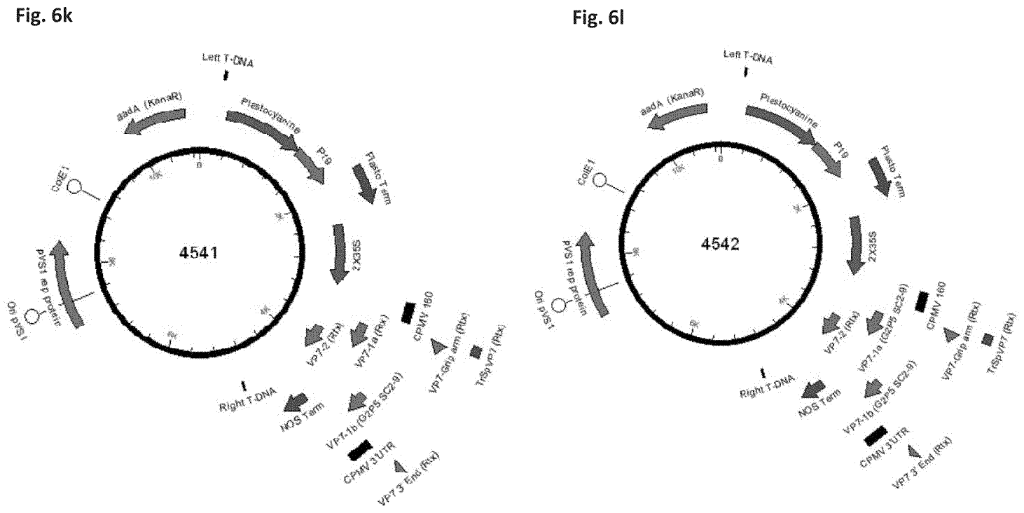

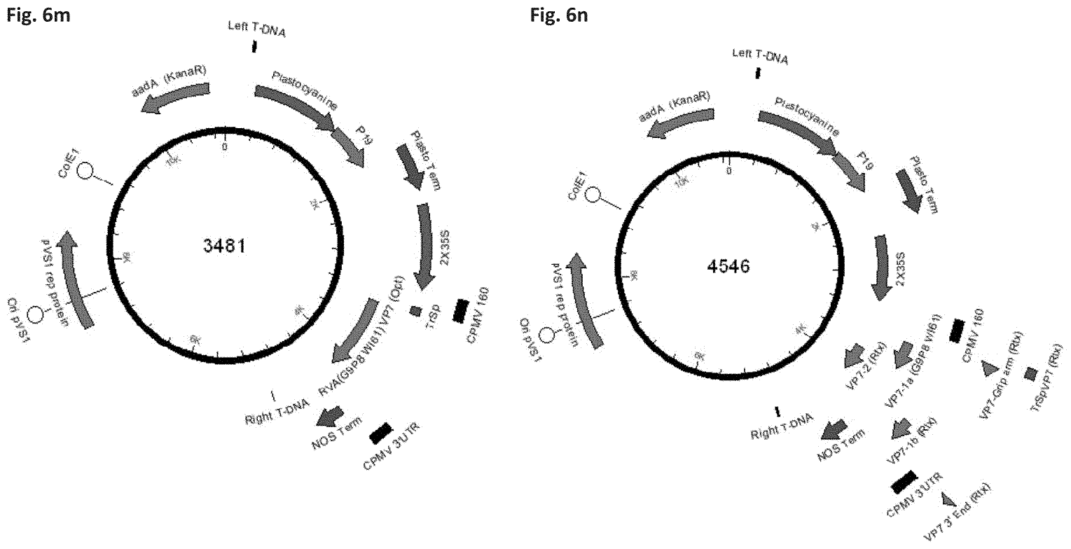

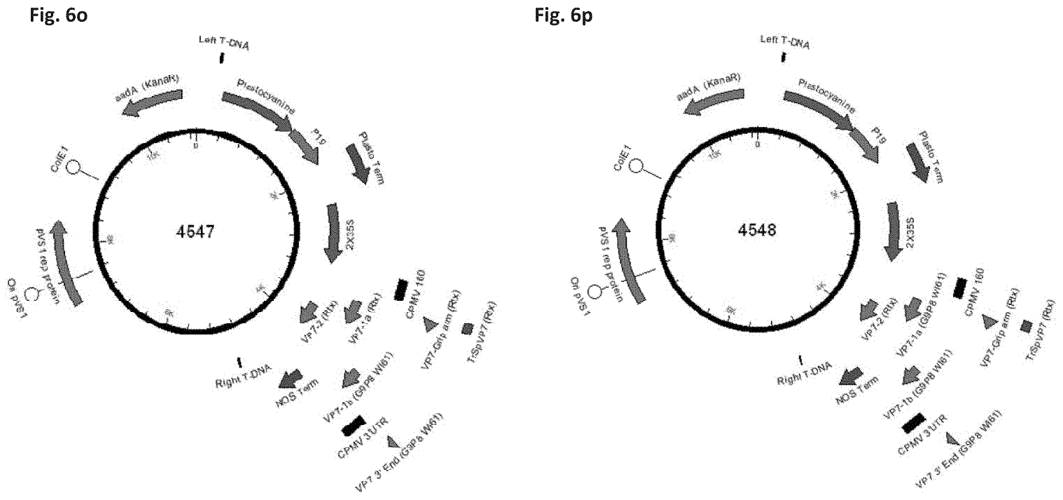

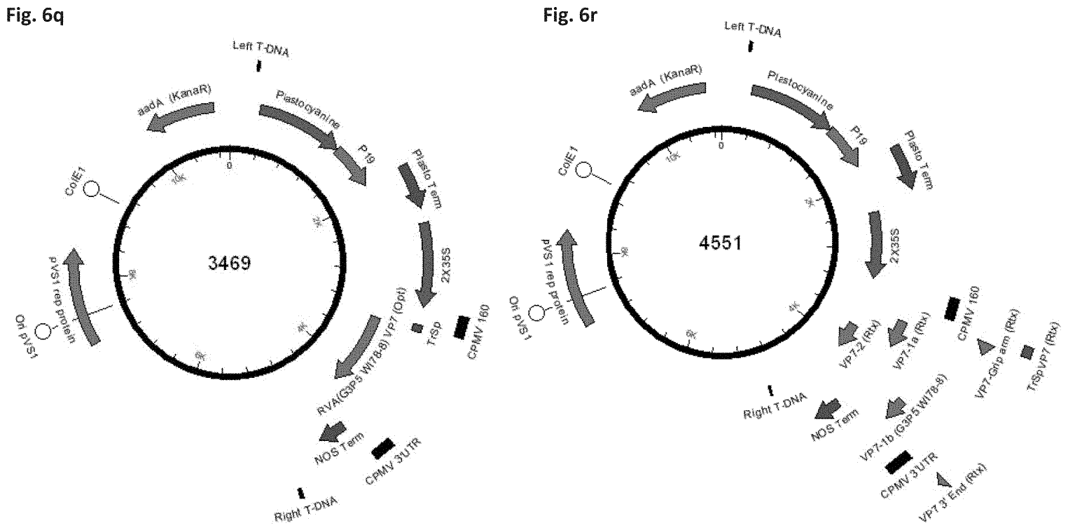

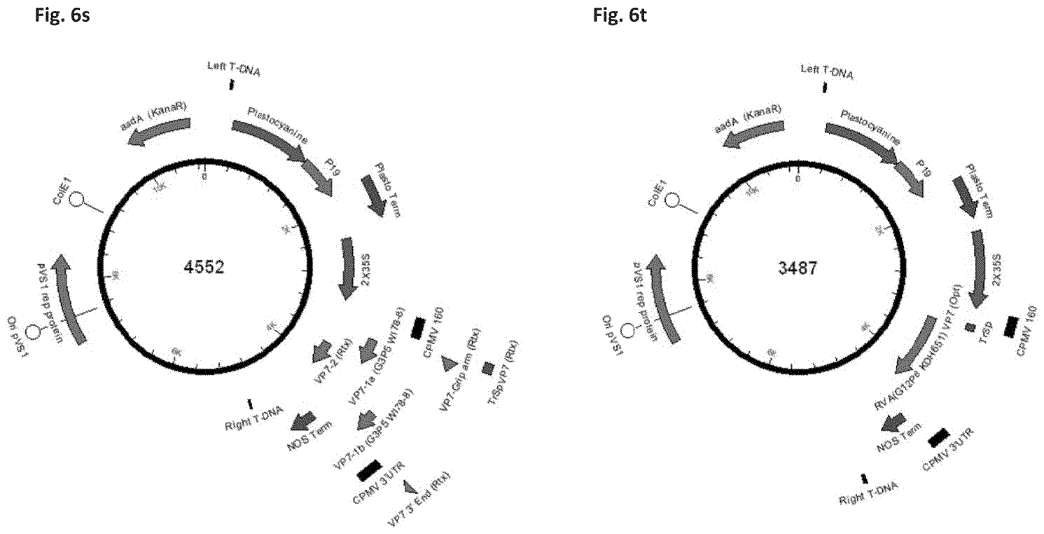

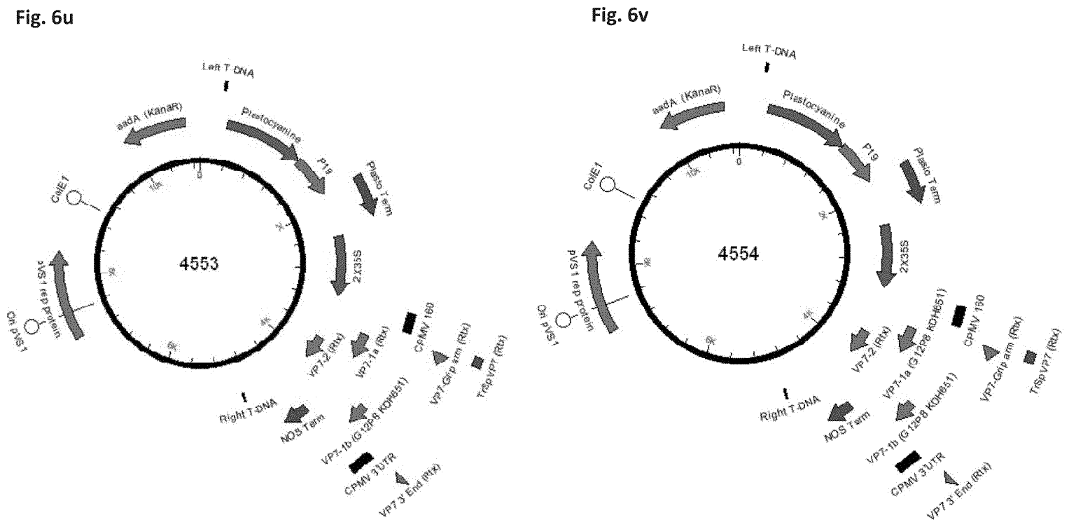

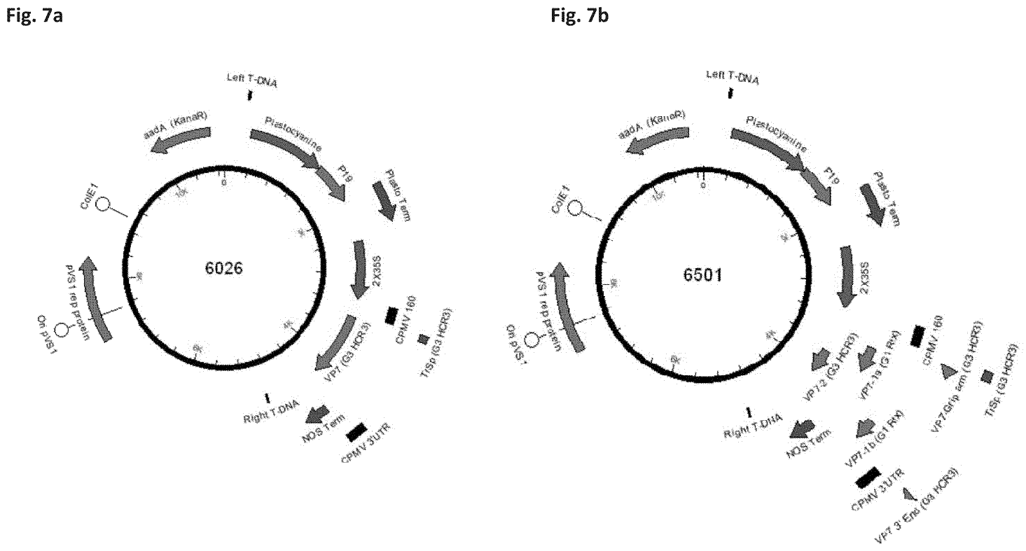

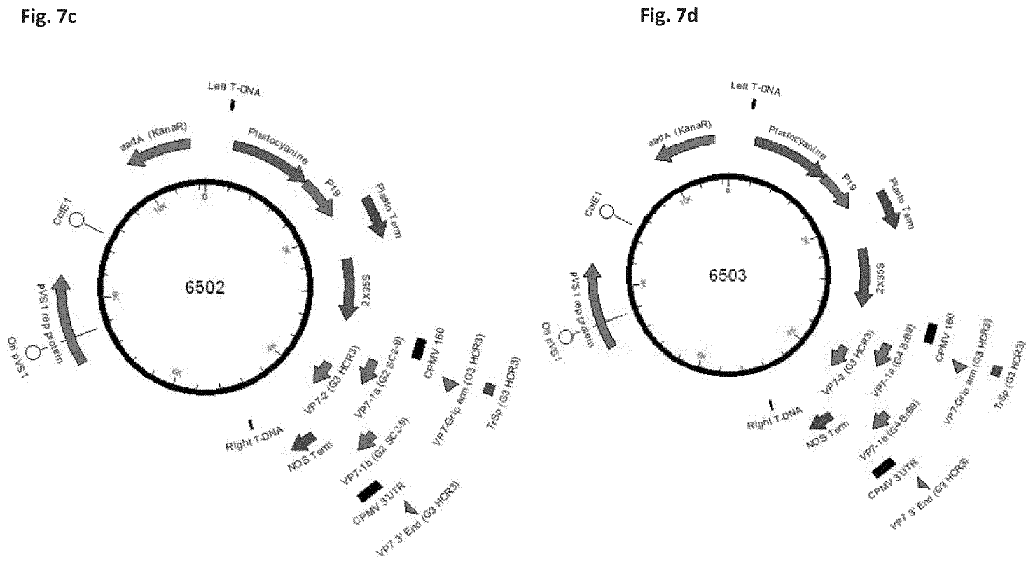

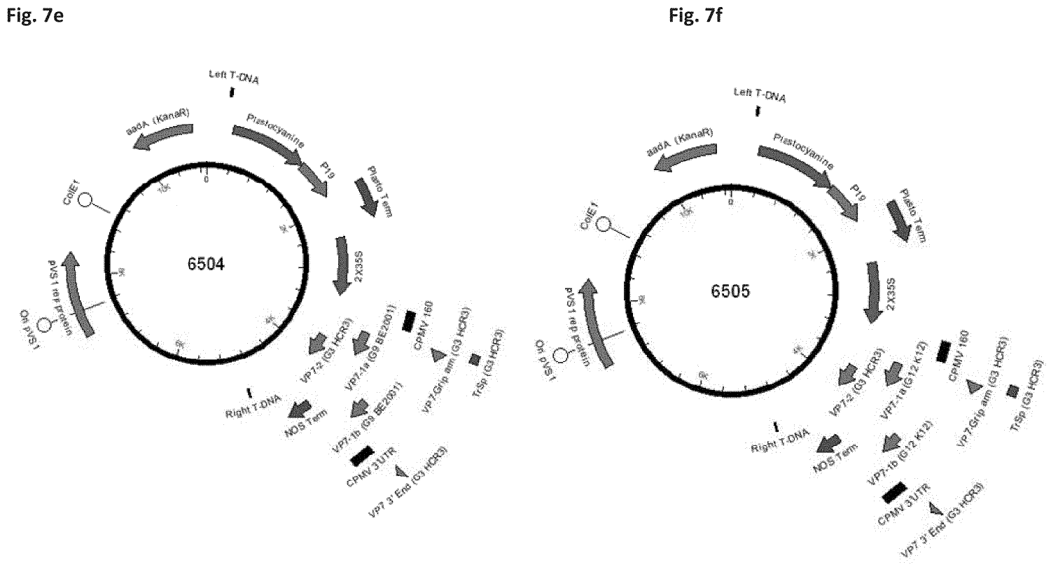

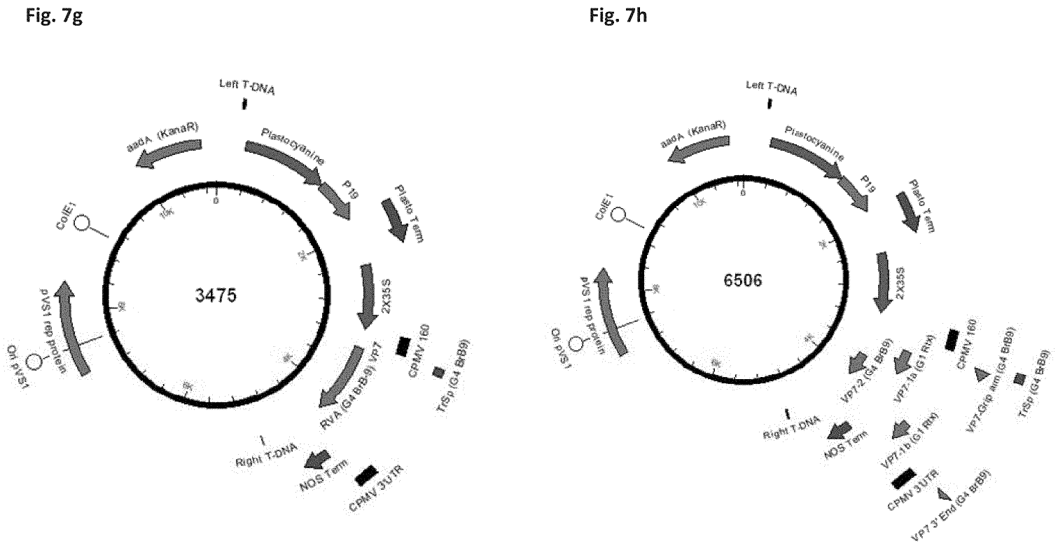

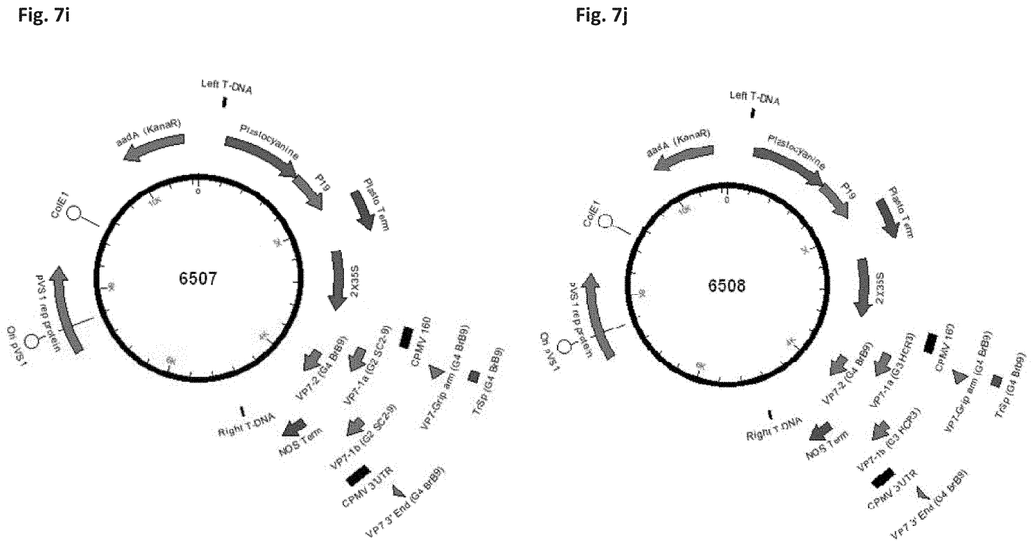

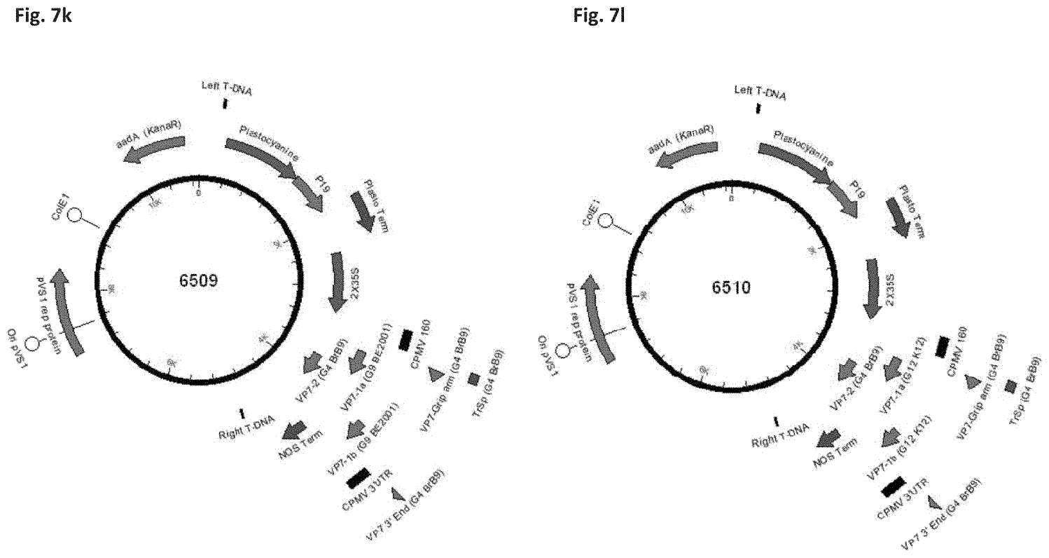

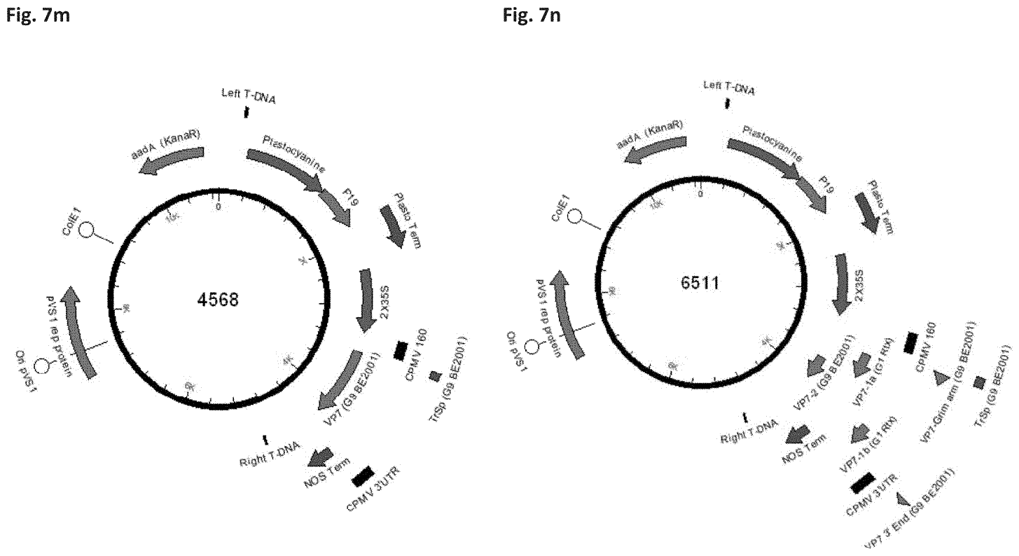

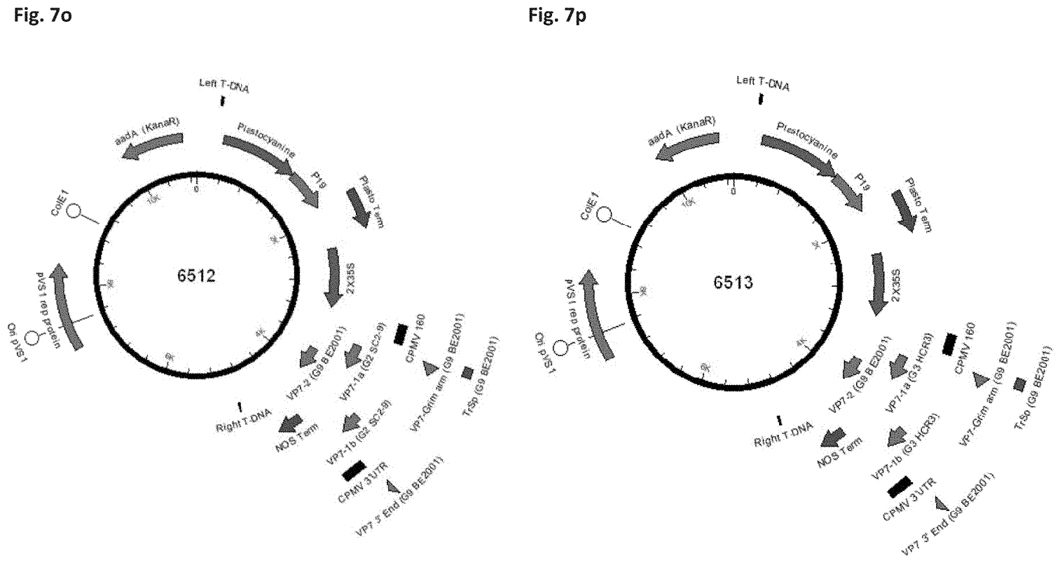

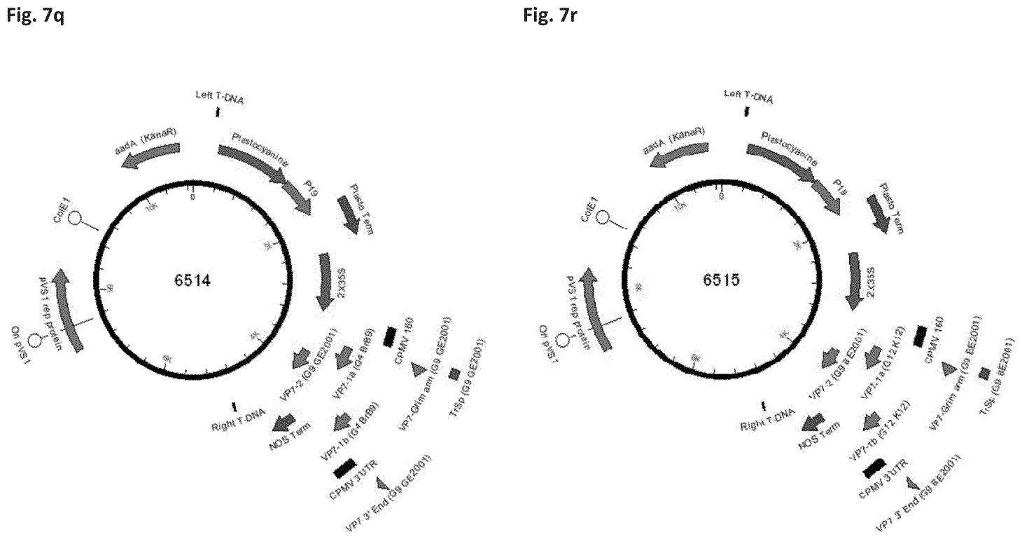

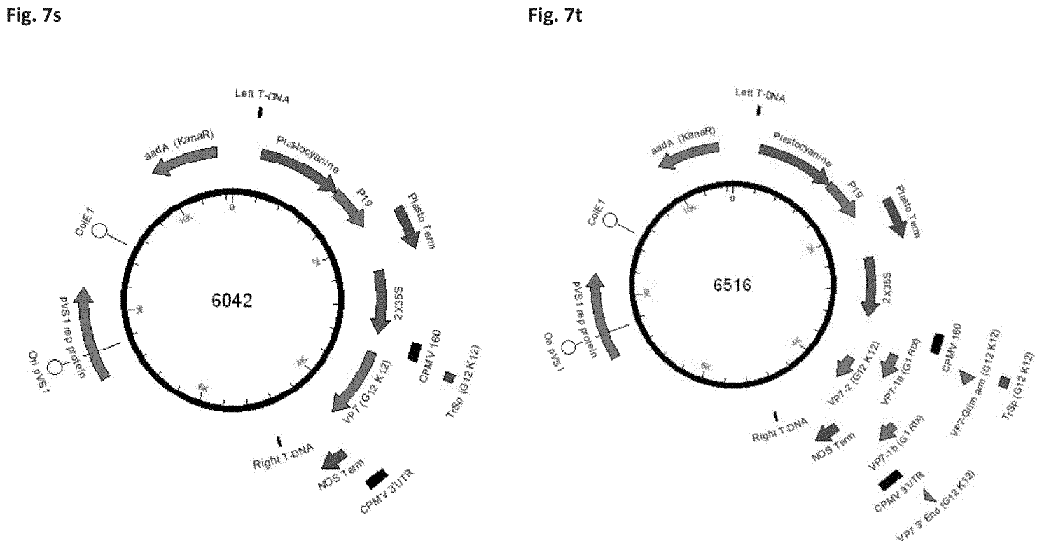

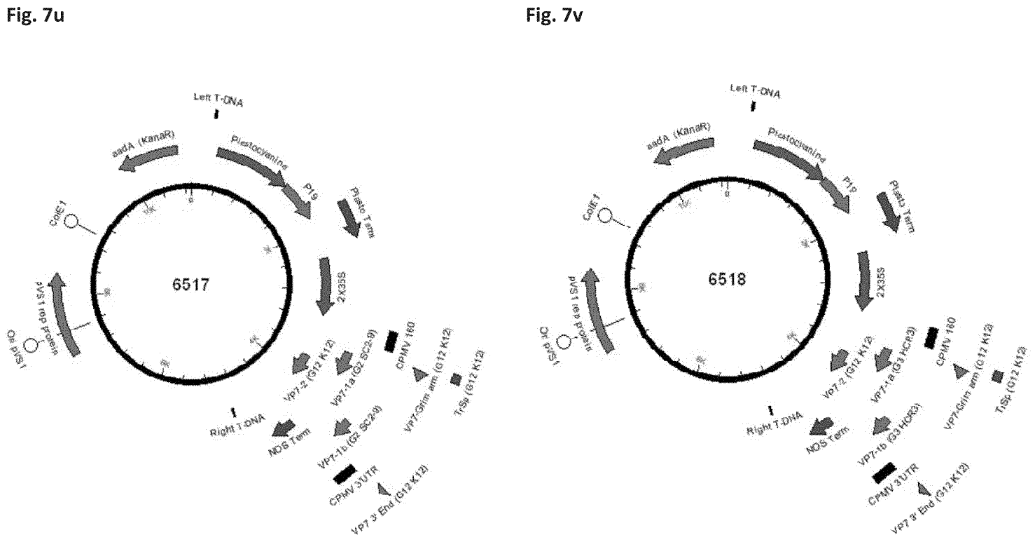

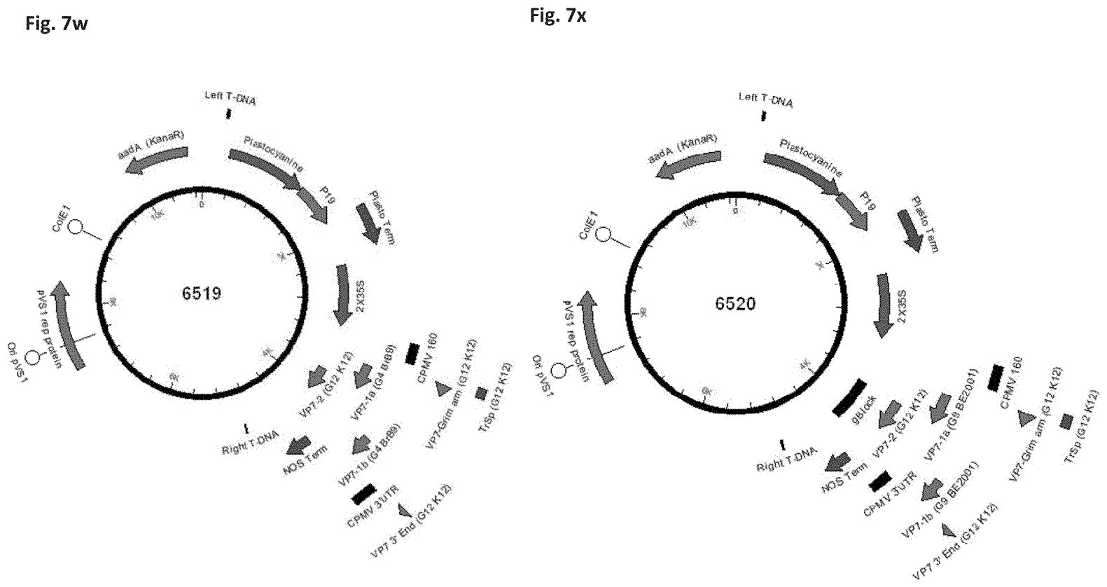

An optimized sequence encoding VP2 from Rotavirus A WA strain was cloned into 2X35S-CPMV-HT-NOS expression system in a plasmid containing Plasto_pro/P19/Plasto_ter expression cassette using the following PCR-based method. A fragment containing the VP2 coding sequence was amplified using primers IF-WA_VP2(opt).s1+3c (SEQ ID NO: 1) and IF-WA_VP2(opt).s1-4r (SEQ ID NO: 2), using optimized VP2 gene sequence (SEQ ID NO:3) as template. For sequence optimization, VP2 protein sequence (Genbank accession number CAA33074) was backtranslated and optimized for human codon usage, GC content and mRNA structure. The PCR product was cloned in 2X35S/CPMV-HT/NOS expression system using In-Fusion cloning system (Clontech, Mountain View, Calif.). Construct number 1191 (FIG. 6b) was digested with SacII and StuI restriction enzyme and the linearized plasmid was used for the In-Fusion assembly reaction. Construct number 1191 is an acceptor plasmid intended for “In Fusion” cloning of genes of interest in a CPMV-HT-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in the sequence of SEQ ID NO: 5. The resulting construct was given number 1710 (FIG. 6a, SEQ ID NO: 6). The amino acid sequence of VP2 from Rotavirus A strain WA is presented in the sequence of SEQ ID NO: 4. A representation of plasmid 1710 is presented in FIG. 6a.

### 2X35S/CPMV-HT/RVA(WA) VP6(Opt)/NOS (Construct Number 1713)

An optimized sequence encoding VP6 from Rotavirus A WA strain was cloned into 2X35S-CPMV-HT-NOS expression system in a plasmid containing Plasto_pro/P19/Plasto_ter expression cassette using the following PCR-based method. A fragment containing the VP6 coding sequence was amplified using primers IF-WA_VP6(opt).s1+3c (SEQ ID NO: 7) and IF-WA_VP6(opt).s1-4r (SEQ ID NO: 8), using optimized VP6 gene sequence (SEQ ID NO:9) as template. For sequence optimization, VP6 protein sequence (Genbank accession number AAA47311) was backtranslated and optimized for human codon usage, GC content and mRNA structure. The PCR product was cloned in 2X35S/CPMV-HT/NOS expression system using In-Fusion cloning system (Clontech, Mountain View, Calif.). Construct number 1191 (FIG. 6b) was digested with SacII and StuI restriction enzyme and the linearized plasmid was used for the In-Fusion assembly reaction. Construct number 1191 is an acceptor plasmid intended for “In Fusion” cloning of genes of interest in a CPMV-HT-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in the sequence of SEQ ID NO: 5). The resulting construct was given number 1713 (FIG. 6c, SEQ ID NO: 11). The amino acid sequence of VP6 from Rotavirus A strain WA is presented in the sequence of SEQ ID NO: 10. A representation of plasmid 1713 is presented in FIG. 6c.

### 2X35S/CPMV-HT/RVA(WA) NSP4/NOS (Construct Number 1706)

A sequence encoding NSP4 from Rotavirus A WA strain was cloned into 2X35S-CPMV-HT-NOS expression system in a plasmid containing Plasto_pro/P19/Plasto_ter expression cassette using the following PCR-based method. A fragment containing the NSP4 coding sequence was amplified using primers IF-WA_NSP4.s1+3c (SEQ ID NO: 12) and IF-WA_NSP4.s1-4r (SEQ ID NO: 13), using synthesized NSP4 gene (corresponding to nt 42-569 from GenBank accession number K02032) (SEQ ID NO:14) as template. The PCR product was cloned in 2X35S/CPMV-HT/NOS expression system using In-Fusion cloning system (Clontech, Mountain View, Calif.). Construct number 1191 (FIG. 6b) was digested with SacII and StuI restriction enzyme and the linearized plasmid was used for the In-Fusion assembly reaction. Construct number 1191 is an acceptor plasmid intended for “In Fusion” cloning of genes of interest in a CPMV-HT-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in the sequence of SEQ ID NO: 5. The resulting construct was given number 1706 (FIG. 6d, SEQ ID NO: 16). The amino acid sequence of NSP4 from Rotavirus A strain WA is presented in the sequence of SEQ ID NO: 15. A representation of plasmid 1706 is presented in FIG. 6d.

### Double Gene Construct for the Expression of VP6 and VP2 Under CPMV-HT Expression Cassette (Construct Number 1708)

A single vector for the co-expression of VP6 from Rotavirus A WA strain and VP2 from Rotavirus A WA strain under the control of CPMV-HT expression system was assembled using the following restriction enzyme/ligase-based method. Donor plasmid DNA (construct number 1710; 2X35S/CPMV-HT/RVA(WA) VP2(opt)/NOS)(FIG. 6a, SEQ ID NO: 6) was digested with AvrII (located before the 2X35S promoter) and AscI (located after the NOS terminator) restriction enzymes and the fragment corresponding to 2X35S/CPMV-HT/RVA(WA) VP2(opt)/NOS expression cassette was gel-purified. This fragment was then inserted into the acceptor construct number 1713 (2X35S/CPMV-HT/RVA(WA) VP6(opt)/NOS)(FIG. 6c, SEQ ID NO: 11) linearized using XbaI and AscI restriction enzymes (both sites are located after the NOS terminator of VP6 expression cassette). The resulting construct was given number 1708. A representation of plasmid 1708 is presented in FIG. 6e.

### Triple Gene Construct for the Expression of VP6, VP2 and NSP4 Under CPMV-HT Expression Cassette (Construct Number 2252)

A single vector for the co-expression of VP6 from Rotavirus A WA strain, VP2 from Rotavirus A WA strain and NSP4 from Rotavirus WA strain under the control of CPMV-HT expression system was assembled using the following restriction enzyme/ligase-based method. Donor plasmid DNA (construct number 1706; 2X35S/CPMV-HT/RVA(WA) NSP4/NOS)(FIG. 6d, SEQ ID NO: 16) was digested with AvrII (located before the 2X35S promoter) and AscI (located after the NOS terminator) restriction enzymes and the fragment corresponding to 2X35S/CPMV-HT/RVA(WA) NSP4/NOS expression cassette was gel-purified. This fragment was then inserted into the acceptor construct number 1708 (2X35S/CPMV-HT/RVA(WA) VP6(opt)/NOS+2X35S/CPMV-HT/RVA(WA) VP2(opt)/NO)(FIG. 6e) linearized using XbaI and AscI restriction enzymes (both sites are located after the NOS terminator of VP2 expression cassette). The resulting construct was given number 2252. A representation of plasmid 1708 is presented in FIG. 6e.

### 2X35S/CPMV-160/TrSp-RVA(Rtx) VP7(Opt)/NOS (Construct Number 1199)

An optimized sequence encoding VP7 with a truncated version of the native signal peptide from Rotavirus A vaccine USA/Rotarix-A41CB052A/1988/G1P1A[8] strain was cloned into 2X35S/CPMV-160/NOS expression system in a plasmid containing Plasto_pro/P19/Plasto_ter expression cassette using the following PCR-based method. A fragment containing the VP7 coding sequence was amplified using primers IF(C160)-TrSP+Rtx_VP7(opt).c (SEQ ID NO: 17) and IF-Rtx_VP7(opt).s1-4r (SEQ ID NO: 18), using optimized VP7 gene sequence (SEQ ID NO: 19) as template. For sequence optimization, VP7 protein sequence (Genbank accession number AEX30682) was backtranslated and optimized for human codon usage, GC content and mRNA structure. The PCR product was cloned in 2X35S/CPMV-160/NOS expression system using In-Fusion cloning system (Clontech, Mountain View, Calif.). Construct number 1190 (FIG. 6g) was digested with SacII and StuI restriction enzyme and the linearized plasmid was used for the In-Fusion assembly reaction. Construct number 1190 is an acceptor plasmid intended for “In Fusion” cloning of genes of interest in a CPMV-160-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in the sequence of SEQ ID NO: 21. The resulting construct was given number 1199 (7h, SEQ ID NO: 22). The amino acid sequence of VP7 with truncated signal peptide from Rotavirus A vaccine USA/Rotarix-A41CB052A/1988/G1P1A[8] strain is presented in the sequence of SEQ ID NO: 20. A representation of plasmid 1199 is presented in FIG. 6h.

Other constructs were assembled using the same method as construct 1199 (FIG. 6h, SEQ ID NO: 22) using synthesized genes and primers listed in the table 4 below.

TABLE 4
Examples of constructs that have been prepared as described herein. Sequences are provided in Example 5 and the sequence listing.

| | Constr. | Fig. | | | | | |
|---|---|---|---|---|---|---|---|
| Construct Name | # | # | Forward primer | Reverse primer | PCR template | Resulting gene | Resulting protein |
| RVA(WA) VP2(opt) | 1710 | 6a | SEQ ID NO: 1 | SEQ ID NO: 2 | SEQ ID NO: 3 | SEQ ID NO: 3 | SEQ ID NO: 4 |
| RVA(WA) VP6(opt) | 1713 | 6c | SEQ ID NO: 7 | SEQ ID NO: 8 | SEQ ID NO: 9 | SEQ ID NO: 9 | SEQ ID NO: 10 |
| RVA(WA) NSP4 | 1706 | 6d | SEQ ID NO: 12 | SEQ ID NO: 13 | SEQ ID NO: 14 | SEQ ID NO: 14 | SEQ ID NO: 15 |
| RVA(WA) VP6(opt) | 1708 | 6e | N/Ap | | N/Ap | SEQ ID NO: 9 | SEQ ID NO: 10 |
| RVA(WA) VP2(opt) | | | | | | SEQ ID NO: 3 | SEQ ID NO: 4 |
| RVA(WA) VP6(opt) | 2252 | 6f | N/Ap | | N/Ap | SEQ ID NO: 9 | SEQ ID NO: 10 |
| RVA(WA) VP2(opt) | | | | | | SEQ ID NO: 3 | SEQ ID NO: 4 |
| RVA(WA) NSP4 | | | | | | SEQ ID NO: 14 | SEQ ID NO: 15 |
| RVA(Rtx G1) VP7(opt) | 1199 | 6h | SEQ ID NO: 17 | SEQ ID NO: 18 | SEQ ID NO: 19 | SEQ ID NO: 19 | SEQ ID NO: 20 |
| RVA(Sc2-9 G2) VP7(opt) | 3463 | 6i | SEQ ID NO: 23 | SEQ ID NO: 24 | SEQ ID NO: 25 | SEQ ID NO: 25 | SEQ ID NO: 26 |
| RVA(Rtx G1) VP7 (7-1a G2) | 4540 | 6j | SEQ ID NO: 17 | SEQ ID NO: 18 | SEQ ID NO: 27 | SEQ ID NO: 27 | SEQ ID NO: 28 |
| RVA(Rtx G1) VP7 (7-1b G2) | 4541 | 6k | SEQ ID NO: 17 | SEQ ID NO: 29 | SEQ ID NO: 30 | SEQ ID NO: 30 | SEQ ID NO: 31 |
| RVA(Rtx G1) VP7 (7-1a-1b G2) | 4542 | 6l | SEQ ID NO: 17 | SEQ ID NO: 29 | SEQ ID NO: 32 | SEQ ID NO: 32 | SEQ ID NO: 33 |
| RVA(WI61 G9) VP7(opt) | 3481 | 6m | SEQ ID NO: 34 | SEQ ID NO: 35 | SEQ ID NO: 36 | SEQ ID NO: 36 | SEQ ID NO: 37 |
| RVA(Rtx G1) VP7 (7-1a G9) | 4546 | 6n | SEQ ID NO: 17 | SEQ ID NO: 18 | SEQ ID NO: 38 | SEQ ID NO: 38 | SEQ ID NO: 39 |
| RVA(Rtx G1) VP7 (7-1b-G9) | 4547 | 6o | SEQ ID NO: 17 | SEQ ID NO: 35 | SEQ ID NO: 40 | SEQ ID NO: 40 | SEQ ID NO: 41 |
| RVA(Rtx G1) VP7 (7-1a-1b G9) | 4548 | 6p | SEQ ID NO: 17 | SEQ ID NO: 35 | SEQ ID NO: 42 | SEQ ID NO: 42 | SEQ ID NO: 43 |
| RVA(WI78-8 G3) VP7(opt) | 3469 | 6q | SEQ ID NO: 44 | SEQ ID NO: 45 | SEQ ID NO: 46 | SEQ ID NO: 46 | SEQ ID NO: 47 |
| RVA(Rtx G1) VP7 (7-1b G3) | 4551 | 6r | SEQ ID NO: 17 | SEQ ID NO: 48 | SEQ ID NO: 49 | SEQ ID NO: 49 | SEQ ID NO: 50 |
| RVA(Rtx G1) VP7 (7-1a-1b G3) | 4552 | 6s | SEQ ID NO: 17 | SEQ ID NO: 48 | SEQ ID NO: 51 | SEQ ID NO: 51 | SEQ ID NO: 52 |
| RVA(KDH651 G12) VP7(opt) | 3487 | 6t | SEQ ID NO: 53 | SEQ ID NO: 54 | SEQ ID NO: 55 | SEQ ID NO: 55 | SEQ ID NO: 56 |
| RVA(Rtx G1) VP7 (7-1b G12) | 4553 | 6u | SEQ ID NO: 17 | SEQ ID NO: 57 | SEQ ID NO: 58 | SEQ ID NO: 58 | SEQ ID NO: 59 |
| RVA(Rtx G1) VP7 | 4554 | 6v | SEQ ID NO: 17 | SEQ ID NO: 57 | SEQ ID NO: 60 | SEQ ID NO: 60 | SEQ ID NO: 61 |
| (7-1a-1b G12) | | | | | | | |
| RVA (G3 HCR3) VP7 (opt) | 6026 | 7a | SEQ ID NO: 62 | SEQ ID NO: 63 | SEQ ID NO: 117 | SEQ ID NO:117 | SEQ ID NO: 77 |
| RVA (G3 HCR3) VP7 | 6501 | 7b | SEQ ID NO: 62 | SEQ ID NO: 63 | SEQ ID NO: 64 | SEQ ID NO: 64 | SEQ ID NO: 65 |
| (7-1a-1b G1 Rtx) (opt) | | | | | | | |
| RVA (G3 HCR3) VP7 | 6502 | 7c | SEQ ID NO: 62 | SEQ ID NO: 63 | SEQ ID NO: 66 | SEQ ID NO: 66 | SEQ ID NO: 67 |
| (7-1a-1b G2 Sc2-9) (opt) | | | | | | | |
| RVA (G3 HCR3) VP7 | 6503 | 7d | SEQ ID NO: 62 | SEQ ID NO: 63 | SEQ ID NO: 68 | SEQ ID NO: 68 | SEQ ID NO: 69 |
| (7-1a-1b G4 BrB-9) (opt) | | | | | | | |
| RVA (G3 HCR3) VP7 | 6504 | 7e | SEQ ID NO: 62 | SEQ ID NO: 63 | SEQ ID NO: 70 | SEQ ID NO: 70 | SEQ ID NO: 71 |
| (7-1a-1b G9 BE2001) (opt) | | | | | | | |
| RVA (G3 HCR3) VP7 | 6505 | 7f | SEQ ID NO: 62 | SEQ ID NO: 63 | SEQ ID NO: 72 | SEQ ID NO: 72 | SEQ ID NO: 73 |
| (7-1 a-1b G12 K12) (opt) | | | | | | | |
| RVA (G4 BrB-9) VP7 (opt) | 3475 | 7g | SEQ ID NO: 74 | SEQ ID NO: 75 | SEQ ID NO: 76 | SEQ ID NO: 76 | SEQ ID NO: 78 |
| RVA (G4 BrB-9) VP7 | 6506 | 7h | SEQ ID NO: 74 | SEQ ID NO: 75 | SEQ ID NO: 79 | SEQ ID NO: 79 | SEQ ID NO: 80 |
| (7-1a-1b G1 Rtx) (opt) | | | | | | | |
| RVA (G4 BrB-9) VP7 | 6507 | 7i | SEQ ID NO: 74 | SEQ ID NO: 75 | SEQ ID NO: 81 | SEQ ID NO: 81 | SEQ ID NO: 82 |
| (7-1a-1b G2 Sc2-9) (opt) | | | | | | | |
| RVA (G4 BrB-9) VP7 | 6508 | 7j | SEQ ID NO: 74 | SEQ ID NO: 75 | SEQ ID NO: 83 | SEQ ID NO: 83 | SEQ ID NO: 84 |
| (7-1a-1b G3 HCR3) (opt) | | | | | | | |
| RVA (G4 BrB-9) VP7 | 6509 | 7k | SEQ ID NO: 74 | SEQ ID NO: 75 | SEQ ID NO: 85 | SEQ ID NO: 85 | SEQ ID NO: 86 |
| (7-1a-1b G9 BE2001) (opt) | | | | | | | |
| RVA (G4 BrB-9) VP7 | 6510 | 7l | SEQ ID NO: 74 | SEQ ID NO: 75 | SEQ ID NO: 87 | SEQ ID NO: 87 | SEQ ID NO: 88 |
| (7-1a-1b G12 K12) (opt) | | | | | | | |
| RVA (G9 BE2001) VP7 (opt) | 4568 | 7m | SEQ ID NO: 89 | SEQ ID NO: 90 | SEQ ID NO: 91 | SEQ ID NO: 91 | SEQ ID NO: 92 |
| RVA (G9 BE2001) VP7 | 6511 | 7n | SEQ ID NO: 89 | SEQ ID NO: 90 | SEQ ID NO: 93 | SEQ ID NO: 93 | SEQ ID NO: 94 |
| (7-1a-1b G1 Rtx) (opt) | | | | | | | |
| RVA (G9 BE2001) VP7 | 6512 | 7o | SEQ ID NO: 89 | SEQ ID NO: 90 | SEQ ID NO: 95 | SEQ ID NO: 95 | SEQ ID NO:96 |
| (7-1a-1b G2 Sc2-9) (opt) | | | | | | | |
| RVA (G9 BE2001) VP7 | 6513 | 7p | SEQ ID NO: 89 | SEQ ID NO: 90 | SEQ ID NO: 97 | SEQ ID NO: 97 | SEQ ID NO: 98 |
| (7-1a-1b G3 HCR3) (opt) | | | | | | | |
| RVA (G9 BE2001) VP7 | 6514 | 7q | SEQ ID NO: 89 | SEQ ID NO: 90 | SEQ ID NO: 99 | SEQ ID NO: 99 | SEQ ID NO: 100 |
| (7-1a-1b G4 BrB-9) (opt) | | | | | | | |
| RVA (G9 BE2001) VP7 | 6515 | 7r | SEQ ID NO: 89 | SEQ ID NO: 90 | SEQ ID NO: 101 | SEQ ID NO: 101 | SEQ ID NO: 102 |
| (7-1a-1b G12 K12) (opt) | | | | | | | |
| RVA (G12 K12) VP7 (opt) | 6042 | 7s | SEQ ID NO: 103 | SEQ ID NO: 104 | SEQ ID NO: 105 | SEQ ID NO: 105 | SEQ ID NO: 106 |
| RVA (G12 K12) VP7 | 6516 | 7t | SEQ ID NO: 103 | SEQ ID NO: 104 | SEQ ID NO: 107 | SEQ ID NO: 107 | SEQ ID NO: 108 |
| (7-1a-1b G1 Rtx) (opt) | | | | | | | |
| RVA (G12 K12) VP7 | 6517 | 7u | SEQ ID NO: 103 | SEQ ID NO: 104 | SEQ ID NO: 109 | SEQ ID NO: 109 | SEQ ID NO: 110 |
| (7-1a-1b G2 Sc2-9) (opt) | | | | | | | |
| RVA (G12 K12) VP7 | 6518 | 7v | SEQ ID NO: 103 | SEQ ID NO: 104 | SEQ ID NO: 111 | SEQ ID NO: 111 | SEQ ID NO: 112 |
| (7-1a-1b G3 HCR3) (opt) | | | | | | | |
| RVA (G12 K12) VP7 | 6519 | 7w | SEQ ID NO: 103 | SEQ ID NO: 104 | SEQ ID NO: 113 | SEQ ID NO: 113 | SEQ ID NO: 114 |
| (7-1a-1b G4 BrB-9) (opt) | | | | | | | |
| RVA (G12 K12) VP7 | 6520 | 7x | SEQ ID NO: 103 | SEQ ID NO: 104 | SEQ ID NO: 115 | SEQ ID NO: 115 | SEQ ID NO: 116 |
| (7-1a-1b G9 BE2001) (opt) | | | | | | | |

### Example 2: Assembly of Gene Constructs and Agrobacterium Transformation

All plasmids, including plasmids 1710, 1713, 1730 and 1734, were used to transform Agrobacterium tumefaciens (AGL1; ATCC, Manassas, Va. 20108, USA) by electroporation (Mattanovich et al., 1989, Nucleic Acid Res. 17:6747) alternatively, heat shock using CaCl2-prepared competent cells (X U et al., 2008, Plant Methods 4) may be used. The integrity of the plasmids in the A. tumefaciens strains created was confirmed by restriction mapping. The A. tumefaciens strain transformed with a given binary plasmid is named AGL1/“plasmid number”. For example, the A. tumefaciens strain transformed with construct number 1710 is termed “AGL1/1710”.

### Preparation of Plant Biomass, Inoculum, Agroinfiltration, and Harvesting

Nicotiana benthamiana plants were grown from seeds in flats filled with a commercial peat moss substrate. The plants were allowed to grow in the greenhouse under a 16/8 photoperiod and a temperature regime of 25° C. day/20° C. night. Three weeks after seeding, individual plantlets were picked out, transplanted in pots and left to grow in the greenhouse for three additional weeks under the same environmental conditions.

Agrobacteria transfected with each construct were grown in a LB medium from vegetal origin and supplemented with 10 mM 2-(N-morpholino)ethanesulfonic acid (MES) and 50 μg/ml kanamycin pH5.6 until they reached an OD600 between 0.6 and 2.5. Agrobacterium suspensions were mixed to reach appropriate ratio for each construct and brought to 2.5×OD600 with infiltration medium (10 mM MgCl2 and 10 mM MES pH 5.6). A. tumefaciens suspensions were stored overnight at 4° C. On the day of infiltration, culture batches were diluted with infiltration medium in 2.5 suspension volumes and allowed to warm before use. Whole plants of N. benthamiana were placed upside down in the bacterial suspension in an air-tight stainless steel tank under a vacuum of 20-40 Torr for 2-min. Following infiltration, plants were returned to the greenhouse for a 3-12 day incubation period until harvest. Harvested biomass was kept frozen (−80° C.) until use for purification of particles.

### Extraction and Purification of Rotavirus-Like Particles

Proteins were extracted from frozen biomass by mechanical extraction in a blender with 3 volumes of extraction buffer (TNC: 10 mM Tris pH 7.4, 140 mM NaCl, 10 mM CaCl2). The slurry was filtered through a large pore nylon filter to remove large debris and centrifuged 5000 g for 5 min at 4° C. The supernatant was collected and centrifuged again at 5000 g for 30 min (4° C.) to remove additional debris. The supernatant was depth-filtered and ultra-filtered and the filtrate was centrifuged at 75 000 g for 20 min (4° C.) to concentrate the rotavirus-like particles. The pellet containing the particles was resuspended in 1/12 volume of TNC and the insoluble were remove with a centrifugation at 5000 g for 5 minutes. The supernatant was filtered on Miracloth before being loaded on iodixanol density gradients.

Density gradient centrifugation was performed as follows. Tubes containing step gradients from 5% to 45% of iodixanol were prepared and overlaid with the filtered extracts containing the rotavirus-like particles. The gradients were centrifuged at 120 000 g for 4 hours (4° C.). After centrifugation, 1 ml fractions were collected from the bottom to the top and analysed by Coomassie-stained SDS-PAGE and Western blot. To remove iodixanol for the fractions selected for further analysis, selected fractions were centrifuged 75 000 g for 20 min (4° C.) and the pelleted particles were resuspended in fresh TNC buffer.

### SDS-PAGE and Immunoblotting

Protein concentrations were determined by the BCA protein assay (Pierce Biochemicals, Rockport Ill.). Proteins were separated by SDS-PAGE under reducing or non-reducing conditions and stained with Coomassie Blue. Stained gels were scanned and densitometry analysis performed using ImageJ Software (NIH).

For immunoblotting, electrophoresed proteins were electrotransferred onto polyvinylene difluoride (PVDF) membranes (Roche Diagnostics Corporation, Indianapolis, Ind.). Prior to immunoblotting, the membranes were blocked with 5% skim milk and 0.1% Tween-20 in Tris-buffered saline (TBS-T) for 16-18h at 4° C.

Immunoblotting was performed by incubation with a suitable antibody (Table 6), in 2 μg/ml in 2% skim milk in TBS-Tween 20 0.1%. Secondary antibodies used for chemiluminescence detection were as indicated in Table 6, diluted as indicated in 2% skim milk in TBS-Tween 20 0.1% Immunoreactive complexes were detected by chemiluminescence using luminol as the substrate (Roche Diagnostics Corporation). Horseradish peroxidase-enzyme conjugation of human IgG antibody was carried out by using the EZ-Link Plus® Activated Peroxidase conjugation kit (Pierce, Rockford, Ill.).

### Production of Rotavirus-Like Particles Comprising VP2 Protein, VP6 Protein and VP7 Fusion Protein.

Rotavirus-like particles comprising VP2 protein, VP6 protein and VP7 fusion protein were produced by transient expression in Nicotiana benthamiana. Plants were agro-infiltrated with an inoculum of Agrobacteria containing a mixture of the constructs encoding VP2 protein, VP6 protein and VP7 fusion protein (see table 3 for constructs) in a 1:1:1 proportion and incubated for 7 days before harvest. Rotavirus-like particles were purified from the biomass using the methodology described in the materials and methods section. After centrifugation of the clarified extracts on iodixanol density gradient, the first ten fractions from the bottom of the tube were analyzed by Coomassie-stained SDS-PAGE.

### Production of Rotavirus-Like Particles Comprising VP2, VP6 and VP7.

Rotavirus-like particles comprising VP2 protein, VP4 protein, VP6 protein and VP7 fusion protein were produced by transient expression in Nicotiana benthamiana. Plants were agro-infiltrated with an inoculum of Agrobacteria containing a mixture of the constructs encoding VP2 protein, VP4 protein, VP6 protein and VP7 fusion protein (see table 3 for constructs) in a 1:1:1:1 proportion and incubated for 7 days before harvest. Rotavirus-like particles were purified from the biomass using the methodology described in the materials and methods section. After centrifugation of the clarified extracts on iodixanol density gradient, the first ten fractions from the bottom of the tube were analyzed by Coomassie-stained SDS-PAGE.

Purified VP2/VP6/fusion VP7 RLPs were sent for cryo-electron microscopy analysis (NanoImaging Services Inc., La Jolla, Calif.) to confirm the assembly of the four antigens into particles resembling the rotavirus particle. As shown in FIG. 4b the cryoEM images of VP2/VP6/fusion VP7 particles confirmed the correct assembly of the antigens into rotavirus-like particles.

### Example 3: VP7 Content in Rotavirus-Like Particles (RLPs)

VP7 incorporation or VP7 content in the VP2/VP6/fusion VP7 particles was further analyzed. Briefly, iodixanol density gradient fractions (35%) of crude protein extracts prepared from N. benthamiana leaves co-expressing rotavirus VP2, VP6 and VP7 fusion as described above and in Table 5, where analyzed by coomassie-stained SDS PAGE analysis. In brief, a fixed amount of RLP is loaded on SDS-PAGE and coomassie-stained. Band densitometry is determined for each of the structural protein and corrected for RLP purity to determine each structural protein proportion.

As can be seen in Table 5 below, VP2/VP6/fusion VP7 particles (RLPs) comprising a 7-1a₂--7-2₁--7-1b₂ (7-1a-1b) type VP7 fusion protein as described herein, have VP7 fusion content that ranged from 5% to 35% of total structural protein mass of the particle.

For example, the VP7 content or VP7 incorporation increased from between 5%-10% VP7 content in RLPs that include native/wildtype VP7 from a G4 strain, to 25%-35% VP7 content in RLPs that included VP7 fusion VP7 (G3)+7-1a-1b G4, VP7 (G9)+7-1a-1b G4 or VP7 (G12)+7-1a-1b G4 (see table 5, 5t^{h} column, “G4 BrB-9”).

VP2/VP6/fusion VP7 particles RVA (G3 HCR3) VP7 (7-1a-1b G4 BrB-9) (construct #6503), RVA (G9 BE2001) VP7 (7-1a-1b G4 BrB-9) (construct #6514), RVA (G12 K12) VP7 (7-1a-1b G4 BrB-9) (construct #6519) and RVA (G12 K12) VP7 (7-1a-1b G4 G3 HCR3) (construct #6518) had a VP7 fusion content of between about 25% to about 35% of total structural protein mass of the particle.

VP2/VP6/fusion VP7 particles RVA (G12 K12) VP7 (7-1a-1b G1 Rtx) (construct #6516), RVA (G12 K12) VP7 (7-1a-1b G2 Sc2-9) (construct #6517), RVA (G12 K12) VP7 (7-1a-1b G9 BE2001) (construct #6520), RVA (G9 BE2001) VP7 (7-1a-1b G12 K12) (construct #6515) had a VP7 fusion content of between about 15% to about 25% of total structural protein mass of the particle.

VP2/VP6/fusion VP7 particles RVA (G3 HCR3) VP7 (7-1a-1b G1 Rtx) (construct #6501), RVA (G3 HCR3) VP7 (7-1a-1b G2 Sc2-9) (construct #6502), RVA (G3 HCR3) VP7 (7-1a-1b G9 Be2001) (construct #6504), RVA (G4 BrB9) VP7 (7-1a-1b G1 Rtx) (construct #6506), RVA (G4 BrB9) VP7 (7-1a-1b G3 HCR3) (construct #6508), RVA (G4 BrB9) VP7 (7-1a-1b G12K12) (construct #6510), RVA (G9 BE2001) VP7 (7-1a-1b G1 Rtx) (construct #6511) and RVA (G9 BE2001) VP7 (7-1a-1b G2 Sc2-9) (construct #6512) had a VP7 fusion content of between about 10% to about 15% of total structural protein mass of the particle.

### Example 4—Immunogenic Study

Mice were immunized two times (3 weeks apart) with antigens and doses shown in the Table 6. Three weeks after the last dose, mice were sacrificed and serum were collected.

TABLE 6
Immunogenicity study outline

| Group | | Dose | Concentration | Volume/site × 2 | | |
|---|---|---|---|---|---|---|
| No. | Test group | (μg/body) | (μg/mL) | (mL) | Time | Number |
| 1 | Placebo | 0 | 0 | 0.05 × 2 | 2 | 5 |
| 2 | G9-RLP-AFJ11215 | 0.2 | 2 | 0.05 × 2 | 2 | 5 |
| 3 | G9-RLP-AFJ11215 | 2 | 20 | 0.05 × 2 | 2 | 5 |
| 4 | G9-RLP-AFJ11215 | 13.5 | 135 | 0.05 × 2 | 2 | 5 |
| 5 | G9-RLP-WI61-Wa-chimera | 0.2 | 2 | 0.05 × 2 | 2 | 5 |
| 6 | G9-RLP-WI61-Wa-chimera | 2 | 20 | 0.05 × 2 | 2 | 5 |
| 7 | G9-RLP-WI61-Wa-chimera | 13.5 | 135 | 0.05 × 2 | 2 | 5 |
| 8 | WI61 virion | 0.2 | 2 | 0.05 × 2 | 2 | 5 |
| 9 | WI61 virion | 2 | 20 | 0.05 × 2 | 2 | 5 |
| 10 | WI61 virion | 13.5 | 135 | 0.05 × 2 | 2 | 5 |

### Neutralizing Antibodies Against G9 W161 Virus

Neutralizing activity against WI61 strain (G9P[8]) of serum were evaluated according to procedure below. There were no significant differences in neutralizing activity against the WI61(G9P[8]) strain (G9-WI61 virion) and native G9-RLP (G9-RLP AFJ11215) and G9-RLP comprising VP7 fusion protein (G9-RLP chimera—VP7 (G1)+7-1a-1b G9)(see FIG. 5).

Activated and m.o.i. (multiplicity of infection) adjusted rotavirus WI61 (G9P[8]) strain were mixed with equal volume of MEM diluted mouse sera in a tube for an hour at 37° C. Mixtures were infected to MA-104 cells seeded on 96 well plates for an hour in a humidified CO2 incubator (set at 37° C. and 5% CO₂). The supernatant in the plates were removed and 100 μL of MEM were added to each well of the plate. The plates were incubated in a humidified CO2 incubator for about 16 hours. Plates were fixed with final concentration of 2% paraformaldehyde for 30 minutes at room temperature, and cells were permeabilized with 0.2 w/v % TritonX-100 solution for 30 minutes at room temperature. Cells were stained with 200-fold diluted anti-rotavirus antibody with 3% BSA-PBS-T overnight at room temperature and 2000-fold diluted donkey anti-goat IgG (H+L) secondary antibody, alexa fluor 488 conjugate with 3% BSA-PBS-T for an hour at room temperature. Nucleus were stained with 1000-fold diluted Hoechst33258 (final concentration: 1 μg/mL) with DPBS (Dulbecco's Phosphate-Buffered Saline) for 30 minutes at room temperature, and infected cell (alexa fluor 488 stained cell) numbers were counted with Array Scan VTI.

Neutralizing activity against Wa strain (G1P[8]) of serum were evaluated according to procedure below. Native G9-RLP (G9-RLP), G9-RLP comprising VP7 fusion protein (G9-RLP chimera—VP7 (G1)+7-1a-1b G9) and G9 WI61 virion showed neutralizing activity comparable to placebo control.

Activated and m.o.i. (multiplicity of infection) adjusted rotavirus Wa (G1P[8]) strain were mixed with equal volume of MEM diluted mouse sera in a tube for an hour at 37° C. Mixtures were infected to MA-104 cells seeded on 96 well plates for an hour in a humidified CO2 incubator (set at 37° C. and 5% CO2). The supernatant in the plates were removed and 100 μL of MEM were added to each well of the plate. The plates were incubated in a humidified CO₂ incubator for about 16 hours. Plates were fixed with final concentration of 2% paraformaldehyde for 30 minutes at room temperature, and cells were permeabilized with 0.2 w/v % TritonX-100 solution for 30 minutes at room temperature. Cells were stained with 200-fold diluted anti-rotavirus antibody with 3% BSA-PBS-T overnight at room temperature and 2000-fold diluted donkey anti-goat IgG (H+L) secondary antibody, alexa fluor 488 conjugate with 3% BSA-PBS-T for an hour at room temperature. Nucleus were stained with 1000-fold diluted Hoechst33258 (final concentration: 1 μg/mL) with DPBS (Dulbecco's Phosphate-Buffered Saline) for 30 minutes at room temperature, and infected cell (alexa fluor 488 stained cell) numbers were counted with Array Scan VTI.

### Example 5: Sequences

The following sequences were used (also see Table 4):

IF-WA_VP2(opt).s1 + 3c
SEQ ID NO: 1
AAATTTGTCGGGCCCATGGCATACCGGAAGAGAGGAGCAAAGCG
CGAA
IF-WA_VP2(opt).s1-4r
SEQ ID NO: 2
ACTAAAGAAAATAGGCCTTTAAAGCTCGTTCATTATTCGCATAT
TGTCGA
Wa_VP2_DNA_Opt
SEQ ID NO: 3
ATGGCATACCGGAAGAGAGGAGCAAAGCGCGAAAACCTGCCGCA
ACAGAACGAGAGACTGCAAGAAAAAGAGATAGAGAAAGATGTCG
ACGTAACAATGGAAAACAAGAATAACAATAGGAAACAACAGCTG
TCCGACAAAGTTCTGTCCCAGAAGGAGGAAATTATCACTGACGC
CCAGGACGATATTAAAATTGCCGGAGAAATAAAGAAGAGCTCGA
AAGAAGAATCTAAACAGCTGCTCGAAATTCTGAAAACAAAAGAA
GACCATCAGAAAGAGATTCAATATGAAATTTTGCAAAAAACAAT
ACCTACATTTGAGTCCAAAGAAAGTATCCTCAAGAAGCTTGAAG
ACATAAGACCGGAGCAGGCAAAAAAACAGATGAAACTCTTTCGC
ATTTTCGAGCCAAAACAGCTCCCTATATATCGCGCCAATGGCGA
GAAGGAGCTACGCAACCGGTGGTACTGGAAGTTGAAAAAAGACA
CCCTGCCAGATGGAGATTATGACGTCCGGGAGTATTTCCTCAAT
CTCTATGATCAGATCCTCATCGAAATGCCGGACTATCTGCTCCT
CAAGGACATGGCCGTGGAGAACAAAAATAGCAGAGACGCCGGCA
AAGTTGTCGACTCTGAGACTGCCAATATTTGTGATGCCATCTTC
CAGGATGAGGAGACCGAGGGAGTCGTCCGTAGATTCATCGCTGA
TATGCGGCAACAGGTCCAGGCTGATCGTAACATTGTCAATTACC
CTTCCATCCTTCACCCTATTGATCATGCATTCAATGAGTATTTT
CTTAACCACCAGTTGGTGGAGCCGCTGAACAATGAGATAATCTT
CAATTACATACCAGAGAGGATAAGGAATGACGTGAATTACATCC
TGAACATGGATATGAATCTGCCATCTACAGCCAGGTATATCAGG
CCAAACTTGTTGCAGGATAGACTGAATCTTCACGATAATTTTGA
GTCCCTGTGGGATACCATCACAACATCCAACTACATTCTGGCCA
GGTCCGTCGTTCCCGATTTGAAGGAGAAGGAGCTGGTCTCCACC
GAAGCACAGATCCAGAAAATGAGCCAGGACCTGCAGCTGGAGGC
CCTCACTATTCAGAGCGAGACACAGTTTTTAGCCGGGATTAACA
GTCAGGCTGCCAATGATTGTTTCAAGACCCTCATAGCCGCCATG
CTGTCTCAAAGAACCATGTCTTTGGACTTTGTGACCACGAACTA
TATGAGCCTAATCTCCGGAATGTGGCTACTTACAGTGATTCCCA
ACGATATGTTCCTCCGGGAGTCACTAGTGGCCTGTGAGCTGGCG
ATCATCAACACCATCGTGTATCCAGCATTCGGAATGCAGAGAAT
GCATTACCGGAATGGCGACCCTCAGACACCCTTCCAGATCGCAG
AACAGCAGATCCAGAATTTCCAGGTGGCGAACTGGCTCCATTTT
ATTAACAATAACAGATTCAGGCAAGTTGTGATTGATGGAGTTCT
GAATCAGACTCTGAACGACAATATACGGAATGGACAGGTCATCA
ACCAGCTGATGGAAGCATTGATGCAACTCAGCAGACAGCAGTTC
CCCACGATGCCTGTGGATTACAAACGGAGCATCCAACGGGGCAT
TCTGCTTCTCTCCAATAGGCTGGGGCAGCTTGTCGACTTAACCC
GACTGGTCTCCTATAACTACGAGACGCTAATGGCTTGTGTGACC
ATGAACATGCAGCACGTGCAAACCCTGACAACTGAGAAGTTGCA
GCTCACTTCTGTGACTTCGCTTTGTATGTTAATTGGTAACACAA
CCGTGATTCCGTCCCCACAGACACTGTTCCACTACTACAACATC
AACGTGAATTTCCACTCCAATTATAATGAGCGGATCAACGACGC
CGTCGCCATAATTACCGCAGCAAATAGGCTGAATCTTTATCAGA
AAAAAATGAAGTCCATAGTGGAAGACTTTCTGAAACGGCTCCAG
ATTTTCGACGTACCACGAGTGCCTGACGACCAAATGTACAGGCT
GAGGGATCGCCTTCGGCTCTTACCCGTTGAACGGAGACGGCTTG
ACATATTCAACTTGATCCTGATGAATATGGAGCAGATCGAACGC
GCTTCTGATAAGATTGCTCAGGGGGTTATCATCGCATACCGAGA
TATGCAGCTGGAACGCGACGAGATGTACGGATATGTTAATATTG
CACGGAATCTTGATGGCTACCAGCAAATTAACTTGGAGGAACTC
ATGCGCACCGGTGATTACGGACAAATTACGAACATGCTTCTCAA
CAATCAACCCGTTGCCCTTGTGGGTGCATTGCCCTTCGTTACGG
ACTCATCCGTGATCAGTCTAATCGCCAAGCTCGACGCAACCGTC
TTCGCTCAGATAGTGAAGCTCAGGAAAGTTGACACACTGAAGCC
CATACTGTACAAAATAAACTCGGATTCCAATGACTTTTACCTTG
TGGCCAACTACGACTGGATCCCCACAAGTACAACTAAGGTCTAC
AAACAGGTGCCACAACCATTCGACTTTAGAGCCAGCATGCACAT
GCTGACTTCTAACCTTACGTTTACCGTCTACTCTGACCTACTGT
CATTTGTTTCAGCGGACACGGTAGAGCCCATTAACGCAGTCGCA
TTCGACAATATGCGAATAATGAACGAGCTTTAA
Wa_VP2_AA
SEQ ID NO: 4
MAYRKRGAKRENLPQQNERLQEKEIEKDVDVTMENKNNNRKQQL
SDKVLSQKEEIITDAQDDIKIAGEIKKSSKEESKQLLEILKTKE
DHQKEIQYEILQKTIPTFESKESILKKLEDIRPEQAKKQMKLFR
IFEPKQLPIYRANGEKELRNRWYWKLKKDTLPDGDYDVREYELN
LYDQILIEMPDYLLLKDMAVENKNSRDAGKVVDSETANICDAIF
QDEETEGVVRRFIADMRQQVQADRNIVNYPSILHPIDHAFNEYF
LNHQLVEPLNNEIIFNYIPERIRNDVNYILNMDMNLPSTARYIR
PNLLQDRLNLHDNFESLWDTITTSNYILARSVVPDLKEKELVST
EAQIQKMSQDLQLEALTIQSETQFLAGINSQAANDCFKTLIAAM
LSQRTMSLDFVTTNYMSLISGMWLLTVIPNDMFLRESLVACELA
IINTIVYPAFGMQRMHYRNGDPQTPFQIAEQQIQNFQVANWLHF
INNNRFRQVVIDGVLNQTLNDNIRNGQVINQLMEALMQLSRQQF
PTMPVDYKRSIQRGILLLSNRLGQLVDLTRLVSYNYETLMACVT
MNMQHVQTLTTEKLQLTSVTSLCMLIGNTTVIPSPQTLFHYYNI
NVNEHSNYNERINDAVAIITAANRLNLYQKKMKSIVEDFLKRLQ
IEDVPRVPDDQMYRLRDRLRLLPVERRRLDIFNLILMNMEQIER
ASDKIAQGVIIAYRDMQLERDEMYGYVNIARNLDGYQQINLEEL
MRTGDYGQITNMLLNNQPVALVGALPFVTDSSVISLIAKLDATV
FAQIVKLRKVDTLKPILYKINSDSNDFYLVANYDWIPTSTTKVY
KQVPQPFDFRASMHMLTSNLTFTVYSDLLSEVSADTVEPINAVA
FDNMRIMNEL
Cloning vector 1191 from left to right T-DNA
SEQ ID NO: 5
TGGCAGGATATATTGTGGTGTAAACAAATTGACGCTTAGACAAC
TTAATAACACATTGCGGACGTTTTTAATGTACTGAATTAACGCC
GAATCCCGGGCTGGTATATTTATATGTTGTCAAATAACTCAAAA
ACCATAAAAGTTTAAGTTAGCAAGTGTGTACATTTTTACTTGAA
CAAAAATATTCACCTACTACTGTTATAAATCATTATTAAACATT
AGAGTAAAGAAATATGGATGATAAGAACAAGAGTAGTGATATTT
TGACAACAATTTTGTTGCAACATTTGAGAAAATTTTGTTGTTCT
CTCTTTTCATTGGTCAAAAACAATAGAGAGAGAAAAAGGAAGAG
GGAGAATAAAAACATAATGTGAGTATGAGAGAGAAAGTTGTACA
AAAGTTGTACCAAAATAGTTGTACAAATATCATTGAGGAATTTG
ACAAAAGCTACACAAATAAGGGTTAATTGCTGTAAATAAATAAG
GATGACGCATTAGAGAGATGTACCATTAGAGAATTTTTGGCAAG
TCATTAAAAAGAAAGAATAAATTATTTTTAAAATTAAAAGTTGA
GTCATTTGATTAAACATGTGATTATTTAATGAATTGATGAAAGA
GTTGGATTAAAGTTGTATTAGTAATTAGAATTTGGTGTCAAATT
TAATTTGACATTTGATCTTTTCCTATATATTGCCCCATAGAGTC
AGTTAACTCATTTTTATATTTCATAGATCAAATAAGAGAAATAA
CGGTATATTAATCCCTCCAAAAAAAAAAAACGGTATATTTACTA
AAAAATCTAAGCCACGTAGGAGGATAACAGGATCCCCGTAGGAG
GATAACATCCAATCCAACCAATCACAACAATCCTGATGAGATAA
CCCACTTTAAGCCCACGCATCTGTGGCACATCTACATTATCTAA
ATCACACATTCTTCCACACATCTGAGCCACACAAAAACCAATCC
ACATCTTTATCACCCATTCTATAAAAAATCACACTTTGTGAGTC
TACACTTTGATTCCCTTCAAACACATACAAAGAGAAGAGACTAA
TTAATTAATTAATCATCTTGAGAGAAAATGGAACGAGCTATACA
AGGAAACGACGCTAGGGAACAAGCTAACAGTGAACGTTGGGATG
GAGGATCAGGAGGTACCACTTCTCCCTTCAAACTTCCTGACGAA
AGTCCGAGTTGGACTGAGTGGCGGCTACATAACGATGAGACGAA
TTCGAATCAAGATAATCCCCTTGGTTTCAAGGAAAGCTGGGGTT
TCGGGAAAGTTGTATTTAAGAGATATCTCAGATACGACAGGACG
GAAGCTTCACTGCACAGAGTCCTTGGATCTTGGACGGGAGATTC
GGTTAACTATGCAGCATCTCGATTTTTCGGTTTCGACCAGATCG
GATGTACCTATAGTATTCGGTTTCGAGGAGTTAGTATCACCGTT
TCTGGAGGGTCGCGAACTCTTCAGCATCTCTGTGAGATGGCAAT
TCGGTCTAAGCAAGAACTGCTACAGCTTGCCCCAATCGAAGTGG
AAAGTAATGTATCAAGAGGATGCCCTGAAGGTACTCAAACCTTC
GAAAAAGAAAGCGAGTAAGTTAAAATGCTTCTTCGTCTCCTATT
TATAATATGGTTTGTTATTGTTAATTTTGTTCTTGTAGAAGAGC
TTAATTAATCGTTGTTGTTATGAAATACTATTTGTATGAGATGA
ACTGGTGTAATGTAATTCATTTACATAAGTGGAGTCAGAATCAG
AATGTTTCCTCCATAACTAACTAGACATGAAGACCTGCCGCGTA
CAATTGTCTTATATTTGAACAACTAAAATTGAACATCTTTTGCC
ACAACTTTATAAGTGGTTAATATAGCTCAAATATATGGTCAAGT
TCAATAGATTAATAATGGAAATATCAGTTATCGAAATTCATTAA
CAATCAACTTAACGTTATTAACTACTAATTTTATATCATCCCCT
TTGATAAATGATAGTACACCAATTAGGAAGGAGCATGCTCGCCT
AGGAGATTGTCGTTTCCCGCCTTCAGTTTGCAAGCTGCTCTAGC
CGTGTAGCCAATACGCAAACCGCCTCTCCCCGCGCGTTGGGAAT
TACTAGCGCGTGTCGACAAGCTTGCATGCCGGTCAACATGGTGG
AGCACGACACACTTGTCTACTCCAAAAATATCAAAGATACAGTC
TCAGAAGACCAAAGGGCAATTGAGACTTTTCAACAAAGGGTAAT
ATCCGGAAACCTCCTCGGATTCCATTGCCCAGCTATCTGTCACT
TTATTGTGAAGATAGTGGAAAAGGAAGGTGGCTCCTACAAATGC
CATCATTGCGATAAAGGAAAGGCCATCGTTGAAGATGCCTCTGC
CGACAGTGGTCCCAAAGATGGACCCCCACCCACGAGGAGCATCG
TGGAAAAAGAAGACGTTCCAACCACGTCTTCAAAGCAAGTGGAT
TGATGTGATAACATGGTGGAGCACGACACACTTGTCTACTCCAA
AAATATCAAAGATACAGTCTCAGAAGACCAAAGGGCAATTGAGA
CTTTTCAACAAAGGGTAATATCCGGAAACCTCCTCGGATTCCAT
TGCCCAGCTATCTGTCACTTTATTGTGAAGATAGTGGAAAAGGA
AGGTGGCTCCTACAAATGCCATCATTGCGATAAAGGAAAGGCCA
TCGTTGAAGATGCCTCTGCCGACAGTGGTCCCAAAGATGGACCC
CCACCCACGAGGAGCATCGTGGAAAAAGAAGACGTTCCAACCAC
GTCTTCAAAGCAAGTGGATTGATGTGATATCTCCACTGACGTAA
GGGATGACGCACAATCCCACTATCCTTCGCAAGACCCTTCCTCT
ATATAAGGAAGTTCATTTCATTTGGAGAGGTATTAAAATCTTAA
TAGGTTTTGATAAAAGCGAACGTGGGGAAACCCGAACCAAACCT
TCTTCTAAACTCTCTCTCATCTCTCTTAAAGCAAACTTCTCTCT
TGTCTTTCTTGCGTGAGCGATCTTCAACGTTGTCAGATCGTGCT
TCGGCACCAGTACAACGTTTTCTTTCACTGAAGCGAAATCAAAG
ATCTCTTTGTGGACACGTAGTGCGGCGCCATTAAATAACGTGTA
CTTGTCCTATTCTTGTCGGTGTGGTCTTGGGAAAAGAAAGCTTG
CTGGAGGCTGCTGTTCAGCCCCATACATTACTTGTTACGATTCT
GCTGACTTTCGGCGGGTGCAATATCTCTACTTCTGCTTGACGAG
GTATTGTTGCCTGTACTTCTTTCTTCTTCTTCTTGCTGATTGGT
TCTATAAGAAATCTAGTATTTTCTTTGAAACAGAGTTTTCCCGT
GGTTTTCGAACTTGGAGAAAGATTGTTAAGCTTCTGTATATTCT
GCCCAAATTTGTCGGGCCCGCGGATGGCGAAAAACGTTGCGATT
TTCGGCTTATTGTTTTCTCTTCTTGTGTTGGTTCCTTCTCAGAT
CTTCGCCTGCAGGCTCCTCAGCCAAAACGACACCCCCATCTGTC
TATCCACTGGCCCCTGGATCTGCTGCCCAAACTAACTCCATGGT
GACCCTGGGATGCCTGGTCAAGGGCTATTTCCCTGAGCCAGTGA
CAGTGACCTGGAACTCTGGATCCCTGTCCAGCGGTGTGCACACC
TTCCCAGCTGTCCTGCAGTCTGACCTCTACACTCTGAGCAGCTC
AGTGACTGTCCCCTCCAGCACCTGGCCCAGCGAGACCGTCACCT
GCAACGTTGCCCACCCGGCCAGCAGCACCAAGGTGGACAAGAAA
ATTGTGCCCAGGGATTGTGGTTGTAAGCCTTGCATATGTACAGT
CCCAGAAGTATCATCTGTCTTCATCTTCCCCCCAAAGCCCAAGG
ATGTGCTCACCATTACTCTGACTCCTAAGGTCACGTGTGTTGTG
GTAGACATCAGCAAGGATGATCCCGAGGTCCAGTTCAGCTGGTT
TGTAGATGATGTGGAGGTGCACACAGCTCAGACGCAACCCCGGG
AGGAGCAGTTCAACAGCACTTTCCGCTCAGTCAGTGAACTTCCC
ATCATGCACCAGGACTGGCTCAATGGCAAGGAGCGATCGCTCAC
CATCACCATCACCATCACCATCACCATTAAAGGCCTATTTTCTT
TAGTTTGAATTTACTGTTATTCGGTGTGCATTTCTATGTTTGGT
GAGCGGTTTTCTGTGCTCAGAGTGTGTTTATTTTATGTAATTTA
ATTTCTTTGTGAGCTCCTGTTTAGCAGGTCGTCCCTTCAGCAAG
GACACAAAAAGATTTTAATTTTATTAAAAAAAAAAAAAAAAAAG
ACCGGGAATTCGATATCAAGCTTATCGACCTGCAGATCGTTCAA
ACATTTGGCAATAAAGTTTCTTAAGATTGAATCCTGTTGCCGGT
CTTGCGATGATTATCATATAATTTCTGTTGAATTACGTTAAGCA
TGTAATAATTAACATGTAATGCATGACGTTATTTATGAGATGGG
TTTTTATGATTAGAGTCCCGCAATTATACATTTAATACGCGATA
GAAAACAAAATATAGCGCGCAAACTAGGATAAATTATCGCGCGC
GGTGTCATCTATGTTACTAGATCTCTAGAGTCTCAAGCTTGGCG
CGCCCACGTGACTAGTGGCACTGGCCGTCGTTTTACAACGTCGT
GACTGGGAAAACCCTGGCGTTACCCAACTTAATCGCCTTGCAGC
ACATCCCCCTTTCGCCAGCTGGCGTAATAGCGAAGAGGCCCGCA
CCGATCGCCCTTCCCAACAGTTGCGCAGCCTGAATGGCGAATGC
TAGAGCAGCTTGAGCTTGGATCAGATTGTCGTTTCCCGCCTTCA
GTTTAAACTATCAGTGTTTGACAGGATATATTGGCGGGTAAACC
TAAGAGAAAAGAGCGTTTA
Construct 1710 from 2X35S to NOS
SEQ ID NO: 6
GTCAACATGGTGGAGCACGACACACTTGTCTACTCCAAAAATAT
CAAAGATACAGTCTCAGAAGACCAAAGGGCAATTGAGACTTTTC
AACAAAGGGTAATATCCGGAAACCTCCTCGGATTCCATTGCCCA
GCTATCTGTCACTTTATTGTGAAGATAGTGGAAAAGGAAGGTGG
CTCCTACAAATGCCATCATTGCGATAAAGGAAAGGCCATCGTTG
AAGATGCCTCTGCCGACAGTGGTCCCAAAGATGGACCCCCACCC
ACGAGGAGCATCGTGGAAAAAGAAGACGTTCCAACCACGTCTTC
AAAGCAAGTGGATTGATGTGATAACATGGTGGAGCACGACACAC
TTGTCTACTCCAAAAATATCAAAGATACAGTCTCAGAAGACCAA
AGGGCAATTGAGACTTTTCAACAAAGGGTAATATCCGGAAACCT
CCTCGGATTCCATTGCCCAGCTATCTGTCACTTTATTGTGAAGA
TAGTGGAAAAGGAAGGTGGCTCCTACAAATGCCATCATTGCGAT
AAAGGAAAGGCCATCGTTGAAGATGCCTCTGCCGACAGTGGTCC
CAAAGATGGACCCCCACCCACGAGGAGCATCGTGGAAAAAGAAG
ACGTTCCAACCACGTCTTCAAAGCAAGTGGATTGATGTGATATC
TCCACTGACGTAAGGGATGACGCACAATCCCACTATCCTTCGCA
AGACCCTTCCTCTATATAAGGAAGTTCATTTCATTTGGAGAGGT
ATTAAAATCTTAATAGGTTTTGATAAAAGCGAACGTGGGGAAAC
CCGAACCAAACCTTCTTCTAAACTCTCTCTCATCTCTCTTAAAG
CAAACTTCTCTCTTGTCTTTCTTGCGTGAGCGATCTTCAACGTT
GTCAGATCGTGCTTCGGCACCAGTACAACGTTTTCTTTCACTGA
AGCGAAATCAAAGATCTCTTTGTGGACACGTAGTGCGGCGCCAT
TAAATAACGTGTACTTGTCCTATTCTTGTCGGTGTGGTCTTGGG
AAAAGAAAGCTTGCTGGAGGCTGCTGTTCAGCCCCATACATTAC
TTGTTACGATTCTGCTGACTTTCGGCGGGTGCAATATCTCTACT
TCTGCTTGACGAGGTATTGTTGCCTGTACTTCTTTCTTCTTCTT
CTTGCTGATTGGTTCTATAAGAAATCTAGTATTTTCTTTGAAAC
AGAGTTTTCCCGTGGTTTTCGAACTTGGAGAAAGATTGTTAAGC
TTCTGTATATTCTGCCCAAATTTGTCGGGCCCATGGCATACCGG
AAGAGAGGAGCAAAGCGCGAAAACCTGCCGCAACAGAACGAGAG
ACTGCAAGAAAAAGAGATAGAGAAAGATGTCGACGTAACAATGG
AAAACAAGAATAACAATAGGAAACAACAGCTGTCCGACAAAGTT
CTGTCCCAGAAGGAGGAAATTATCACTGACGCCCAGGACGATAT
TAAAATTGCCGGAGAAATAAAGAAGAGCTCGAAAGAAGAATCTA
AACAGCTGCTCGAAATTCTGAAAACAAAAGAAGACCATCAGAAA
GAGATTCAATATGAAATTTTGCAAAAAACAATACCTACATTTGA
GTCCAAAGAAAGTATCCTCAAGAAGCTTGAAGACATAAGACCGG
AGCAGGCAAAAAAACAGATGAAACTCTTTCGCATTTTCGAGCCA
AAACAGCTCCCTATATATCGCGCCAATGGCGAGAAGGAGCTACG
CAACCGGTGGTACTGGAAGTTGAAAAAAGACACCCTGCCAGATG
GAGATTATGACGTCCGGGAGTATTTCCTCAATCTCTATGATCAG
ATCCTCATCGAAATGCCGGACTATCTGCTCCTCAAGGACATGGC
CGTGGAGAACAAAAATAGCAGAGACGCCGGCAAAGTTGTCGACT
CTGAGACTGCCAATATTTGTGATGCCATCTTCCAGGATGAGGAG
ACCGAGGGAGTCGTCCGTAGATTCATCGCTGATATGCGGCAACA
GGTCCAGGCTGATCGTAACATTGTCAATTACCCTTCCATCCTTC
ACCCTATTGATCATGCATTCAATGAGTATTTTCTTAACCACCAG
TTGGTGGAGCCGCTGAACAATGAGATAATCTTCAATTACATACC
AGAGAGGATAAGGAATGACGTGAATTACATCCTGAACATGGATA
TGAATCTGCCATCTACAGCCAGGTATATCAGGCCAAACTTGTTG
CAGGATAGACTGAATCTTCACGATAATTTTGAGTCCCTGTGGGA
TACCATCACAACATCCAACTACATTCTGGCCAGGTCCGTCGTTC
CCGATTTGAAGGAGAAGGAGCTGGTCTCCACCGAAGCACAGATC
CAGAAAATGAGCCAGGACCTGCAGCTGGAGGCCCTCACTATTCA
GAGCGAGACACAGTTTTTAGCCGGGATTAACAGTCAGGCTGCCA
ATGATTGTTTCAAGACCCTCATAGCCGCCATGCTGTCTCAAAGA
ACCATGTCTTTGGACTTTGTGACCACGAACTATATGAGCCTAAT
CTCCGGAATGTGGCTACTTACAGTGATTCCCAACGATATGTTCC
TCCGGGAGTCACTAGTGGCCTGTGAGCTGGCGATCATCAACACC
ATCGTGTATCCAGCATTCGGAATGCAGAGAATGCATTACCGGAA
TGGCGACCCTCAGACACCCTTCCAGATCGCAGAACAGCAGATCC
AGAATTTCCAGGTGGCGAACTGGCTCCATTTTATTAACAATAAC
AGATTCAGGCAAGTTGTGATTGATGGAGTTCTGAATCAGACTCT
GAACGACAATATACGGAATGGACAGGTCATCAACCAGCTGATGG
AAGCATTGATGCAACTCAGCAGACAGCAGTTCCCCACGATGCCT
GTGGATTACAAACGGAGCATCCAACGGGGCATTCTGCTTCTCTC
CAATAGGCTGGGGCAGCTTGTCGACTTAACCCGACTGGTCTCCT
ATAACTACGAGACGCTAATGGCTTGTGTGACCATGAACATGCAG
CACGTGCAAACCCTGACAACTGAGAAGTTGCAGCTCACTTCTGT
GACTTCGCTTTGTATGTTAATTGGTAACACAACCGTGATTCCGT
CCCCACAGACACTGTTCCACTACTACAACATCAACGTGAATTTC
CACTCCAATTATAATGAGCGGATCAACGACGCCGTCGCCATAAT
TACCGCAGCAAATAGGCTGAATCTTTATCAGAAAAAAATGAAGT
CCATAGTGGAAGACTTTCTGAAACGGCTCCAGATTTTCGACGTA
CCACGAGTGCCTGACGACCAAATGTACAGGCTGAGGGATCGCCT
TCGGCTCTTACCCGTTGAACGGAGACGGCTTGACATATTCAACT
TGATCCTGATGAATATGGAGCAGATCGAACGCGCTTCTGATAAG
ATTGCTCAGGGGGTTATCATCGCATACCGAGATATGCAGCTGGA
ACGCGACGAGATGTACGGATATGTTAATATTGCACGGAATCTTG
ATGGCTACCAGCAAATTAACTTGGAGGAACTCATGCGCACCGGT
GATTACGGACAAATTACGAACATGCTTCTCAACAATCAACCCGT
TGCCCTTGTGGGTGCATTGCCCTTCGTTACGGACTCATCCGTGA
TCAGTCTAATCGCCAAGCTCGACGCAACCGTCTTCGCTCAGATA
GTGAAGCTCAGGAAAGTTGACACACTGAAGCCCATACTGTACAA
AATAAACTCGGATTCCAATGACTTTTACCTTGTGGCCAACTACG
ACTGGATCCCCACAAGTACAACTAAGGTCTACAAACAGGTGCCA
CAACCATTCGACTTTAGAGCCAGCATGCACATGCTGACTTCTAA
CCTTACGTTTACCGTCTACTCTGACCTACTGTCATTTGTTTCAG
CGGACACGGTAGAGCCCATTAACGCAGTCGCATTCGACAATATG
CGAATAATGAACGAGCTTTAAAGGCCTATTTTCTTTAGTTTGAA
TTTACTGTTATTCGGTGTGCATTTCTATGTTTGGTGAGCGGTTT
TCTGTGCTCAGAGTGTGTTTATTTTATGTAATTTAATTTCTTTG
TGAGCTCCTGTTTAGCAGGTCGTCCCTTCAGCAAGGACACAAAA
AGATTTTAATTTTATTAAAAAAAAAAAAAAAAAAGACCGGGAAT
TCGATATCAAGCTTATCGACCTGCAGATCGTTCAAACATTTGGC
AATAAAGTTTCTTAAGATTGAATCCTGTTGCCGGTCTTGCGATG
ATTATCATATAATTTCTGTTGAATTACGTTAAGCATGTAATAAT
TAACATGTAATGCATGACGTTATTTATGAGATGGGTTTTTATGA
TTAGAGTCCCGCAATTATACATTTAATACGCGATAGAAAACAAA
ATATAGCGCGCAAACTAGGATAAATTATCGCGCGCGGTGTCATC
TATGTTACTAGAT
IF-WA_VP6(opt).s1 + 3 c
SEQ ID NO: 7
AAATTTGTCGGGCCCATGGAGGTCCTTTATAGTCTCTCCAAAAC
GCTGA
IF-WA_VP6(opt).s1-4r
SEQ ID NO: 8
ACTAAAGAAAATAGGCCTCTACTTGATCAACATACTCCGGATAG
AGGCCACA
Wa_VP6_DNA_Opt
SEQ ID NO: 9
ATGGAGGTCCTTTATAGTCTCTCCAAAACGCTGAAGGACGCTAG
GGACAAGATCGTGGAGGGTACACTTTATAGCAATGTCAGCGACC
TAATACAGCAGTTTAATCAAATGATCGTTACAATGAATGGGAAT
GATTTCCAAACTGGCGGTATTGGTAATCTGCCCGTGAGGAACTG
GACATTCGATTTCGGCCTGCTGGGCACGACTCTCCTTAATCTCG
ATGCAAATTATGTAGAAAACGCCAGAACGATTATCGAGTACTTT
ATCGATTTCATTGATAACGTTTGTATGGATGAGATGGCCCGCGA
GTCACAACGGAACGGAGTTGCTCCACAGTCCGAGGCCCTTCGGA
AACTCGCCGGCATTAAGTTCAAGCGTATTAATTTCGACAACTCC
TCCGAATATATAGAGAACTGGAACTTGCAGAATCGTCGACAGAG
AACCGGCTTCGTGTTCCATAAACCTAATATCTTTCCGTATAGCG
CCTCATTCACCCTGAATAGGAGTCAGCCCATGCACGACAACCTC
ATGGGTACAATGTGGCTGAATGCGGGGAGTGAAATACAGGTCGC
CGGGTTCGATTACTCCTGTGCCATTAATGCACCCGCAAACATCC
AGCAGTTCGAACATATCGTGCAACTAAGACGGGCTCTCACGACC
GCGACAATTACACTCCTGCCCGACGCCGAGCGCTTCTCCTTTCC
CCGCGTAATCAACTCAGCTGATGGCGCCACCACTTGGTTCTTCA
ACCCTGTTATATTGCGCCCTAACAACGTAGAGGTGGAGTTTCTC
TTAAACGGACAGATCATCAATACCTACCAAGCCAGGTTCGGCAC
GATTATTGCAAGAAATTTCGACGCTATCAGGCTGCTCTTCCAAC
TGATGAGGCCCCCCAATATGACTCCCGCTGTGAACGCTTTGTTT
CCGCAGGCTCAGCCTTTCCAGCACCACGCCACCGTCGGCTTGAC
TCTTCGAATAGAGAGCGCGGTCTGCGAATCAGTGCTGGCAGACG
CCAACGAGACGCTGCTGGCAAACGTTACCGCCGTGCGGCAAGAG
TATGCCATCCCAGTAGGGCCTGTGTTTCCACCCGGCATGAACTG
GACTGAACTAATTACTAACTATAGCCCATCCAGAGAAGACAACT
TGCAGCGGGTCTTCACTGTGGCCTCTATCCGGAGTATGTTGATC
AAGTAG
Wa_VP6_AA
SEQ ID NO: 10
MEVLYSLSKTLKDARDKIVEGTLYSNVSDLIQQFNQMIVTMNGN
DFQTGGIGNLPVRNWTFDFGLLGTTLLNLDANYVENARTHEYFI
DFIDNVCMDEMARESQRNGVAPQSEALRKLAGIKFKRINFDNSS
EYIENWNLQNRRQRTGFVFHKPNIFPYSASFTLNRSQPMHDNLM
GTMWLNAGSEIQVAGFDYSCAINAPANIQQFEHIVQLRRALTTA
TITLLPDAERFSFPRVINSADGATTWFFNPVILRPNNVEVEFLL
NGQIINTYQARFGTIIARNFDAIRLLFQLMRPPNMTPAVNALFP
QAQPFQHHATVGLTLRIESAVCESVLADANETLLANVTAVRQEY
AIPVGPVFPPGMNWTELITNYSPSREDNLQRVFTVASIRSMLIK
Construct 1713 from 2X35S to NOS
SEQ ID NO: 11
GTCAACATGGTGGAGCACGACACACTTGTCTACTCCAAAAATAT
CAAAGATACAGTCTCAGAAGACCAAAGGGCAATTGAGACTTTTC
AACAAAGGGTAATATCCGGAAACCTCCTCGGATTCCATTGCCCA
GCTATCTGTCACTTTATTGTGAAGATAGTGGAAAAGGAAGGTGG
CTCCTACAAATGCCATCATTGCGATAAAGGAAAGGCCATCGTTG
AAGATGCCTCTGCCGACAGTGGTCCCAAAGATGGACCCCCACCC
ACGAGGAGCATCGTGGAAAAAGAAGACGTTCCAACCACGTCTTC
AAAGCAAGTGGATTGATGTGATAACATGGTGGAGCACGACACAC
TTGTCTACTCCAAAAATATCAAAGATACAGTCTCAGAAGACCAA
AGGGCAATTGAGACTTTTCAACAAAGGGTAATATCCGGAAACCT
CCTCGGATTCCATTGCCCAGCTATCTGTCACTTTATTGTGAAGA
TAGTGGAAAAGGAAGGTGGCTCCTACAAATGCCATCATTGCGAT
AAAGGAAAGGCCATCGTTGAAGATGCCTCTGCCGACAGTGGTCC
CAAAGATGGACCCCCACCCACGAGGAGCATCGTGGAAAAAGAAG
ACGTTCCAACCACGTCTTCAAAGCAAGTGGATTGATGTGATATC
TCCACTGACGTAAGGGATGACGCACAATCCCACTATCCTTCGCA
AGACCCTTCCTCTATATAAGGAAGTTCATTTCATTTGGAGAGGT
ATTAAAATCTTAATAGGTTTTGATAAAAGCGAACGTGGGGAAAC
CCGAACCAAACCTTCTTCTAAACTCTCTCTCATCTCTCTTAAAG
CAAACTTCTCTCTTGTCTTTCTTGCGTGAGCGATCTTCAACGTT
GTCAGATCGTGCTTCGGCACCAGTACAACGTTTTCTTTCACTGA
AGCGAAATCAAAGATCTCTTTGTGGACACGTAGTGCGGCGCCAT
TAAATAACGTGTACTTGTCCTATTCTTGTCGGTGTGGTCTTGGG
AAAAGAAAGCTTGCTGGAGGCTGCTGTTCAGCCCCATACATTAC
TTGTTACGATTCTGCTGACTTTCGGCGGGTGCAATATCTCTACT
TCTGCTTGACGAGGTATTGTTGCCTGTACTTCTTTCTTCTTCTT
CTTGCTGATTGGTTCTATAAGAAATCTAGTATTTTCTTTGAAAC
AGAGTTTTCCCGTGGTTTTCGAACTTGGAGAAAGATTGTTAAGC
TTCTGTATATTCTGCCCAAATTTGTCGGGCCCATGGAGGTCCTT
TATAGTCTCTCCAAAACGCTGAAGGACGCTAGGGACAAGATCGT
GGAGGGTACACTTTATAGCAATGTCAGCGACCTAATACAGCAGT
TTAATCAAATGATCGTTACAATGAATGGGAATGATTTCCAAACT
GGCGGTATTGGTAATCTGCCCGTGAGGAACTGGACATTCGATTT
CGGCCTGCTGGGCACGACTCTCCTTAATCTCGATGCAAATTATG
TAGAAAACGCCAGAACGATTATCGAGTACTTTATCGATTTCATT
GATAACGTTTGTATGGATGAGATGGCCCGCGAGTCACAACGGAA
CGGAGTTGCTCCACAGTCCGAGGCCCTTCGGAAACTCGCCGGCA
TTAAGTTCAAGCGTATTAATTTCGACAACTCCTCCGAATATATA
GAGAACTGGAACTTGCAGAATCGTCGACAGAGAACCGGCTTCGT
GTTCCATAAACCTAATATCTTTCCGTATAGCGCCTCATTCACCC
TGAATAGGAGTCAGCCCATGCACGACAACCTCATGGGTACAATG
TGGCTGAATGCGGGGAGTGAAATACAGGTCGCCGGGTTCGATTA
CTCCTGTGCCATTAATGCACCCGCAAACATCCAGCAGTTCGAAC
ATATCGTGCAACTAAGACGGGCTCTCACGACCGCGACAATTACA
CTCCTGCCCGACGCCGAGCGCTTCTCCTTTCCCCGCGTAATCAA
CTCAGCTGATGGCGCCACCACTTGGTTCTTCAACCCTGTTATAT
TGCGCCCTAACAACGTAGAGGTGGAGTTTCTCTTAAACGGACAG
ATCATCAATACCTACCAAGCCAGGTTCGGCACGATTATTGCAAG
AAATTTCGACGCTATCAGGCTGCTCTTCCAACTGATGAGGCCCC
CCAATATGACTCCCGCTGTGAACGCTTTGTTTCCGCAGGCTCAG
CCTTTCCAGCACCACGCCACCGTCGGCTTGACTCTTCGAATAGA
GAGCGCGGTCTGCGAATCAGTGCTGGCAGACGCCAACGAGACGC
TGCTGGCAAACGTTACCGCCGTGCGGCAAGAGTATGCCATCCCA
GTAGGGCCTGTGTTTCCACCCGGCATGAACTGGACTGAACTAAT
TACTAACTATAGCCCATCCAGAGAAGACAACTTGCAGCGGGTCT
TCACTGTGGCCTCTATCCGGAGTATGTTGATCAAGTAGAGGCCT
ATTTTCTTTAGTTTGAATTTACTGTTATTCGGTGTGCATTTCTA
TGTTTGGTGAGCGGTTTTCTGTGCTCAGAGTGTGTTTATTTTAT
GTAATTTAATTTCTTTGTGAGCTCCTGTTTAGCAGGTCGTCCCT
TCAGCAAGGACACAAAAAGATTTTAATTTTATTAAAAAAAAAAA
AAAAAAAGACCGGGAATTCGATATCAAGCTTATCGACCTGCAGA
TCGTTCAAACATTTGGCAATAAAGTTTCTTAAGATTGAATCCTG
TTGCCGGTCTTGCGATGATTATCATATAATTTCTGTTGAATTAC
GTTAAGCATGTAATAATTAACATGTAATGCATGACGTTATTTAT
GAGATGGGTTTTTATGATTAGAGTCCCGCAATTATACATTTAAT
ACGCGATAGAAAACAAAATATAGCGCGCAAACTAGGATAAATTA
TCGCGCGCGGTGTCATCTATGTTACTAGAT
IF-WA_NSP4.s1 + 3c
SEQ ID NO: 12
AAATTTGTCGGGCCCATGGATAAGCTTGCCGACCTCAACTACAC
ATTGAGTG
IF-WA_NSP4.s1-4r
SEQ ID NO: 13
ACTAAAGAAAATAGGCCTTCACATGGATGCAGTCACTTCTGACG
GTTCATATGGA
Wa_NSP4_DNA
SEQ ID NO: 14
ATGGATAAGCTTGCCGACCTCAACTACACATTGAGTGTAATCAC
TTCAATGAATGACACATTGCATTCTATAATTCAAGATCCTGGAA
TGGCGTATTTTCTATATATTGCATCTGTTCTAACAGTTTTGTTC
ACATTACATAAAGCTTCAATTCCAACCATGAAAATAGCATTGAA
AACATCAAAATGTTCATATAAAGTGATTAAATATTGTATAGTCA
CGATCATTAATACTCTTTTAAAATTGGCTGGATATAAAGAGCAG
GTTACTACAAAAGACGAAATTGAGCAACAGATGGACAGAATTGT
GAAAGAGATGAGACGTCAGCTGGAGATGATTGATAAACTAACTA
CTCGTGAAATTGAACAGGTTGAATTGCTTAAACGTATACATGAC
AACCTGATAACTAGACCAGTTGACGTTATAGATATGTCGAAGGA
ATTCAATCAGAAAAACATCAAAACGCTAGATGAATGGGAGAGTG
GAAAAAATCCATATGAACCGTCAGAAGTGACTGCATCCATGTGA
Wa_NSP4_AA
SEQ ID NO: 15
MDKLADLNYTESVITSMNDTLHSIIQDPGMAYFLYIASVETVLF
TLHKASIPTMKIALKTSKCSYKVIKYCIVTIINTLEKLAGYKEQ
VTTKDEIEQQMDRIVKEMRRQLEMIDKETTREIEQVELLKRIHD
NLITRPVDVIDMSKEENQKNIKTEDEWESGKNPYEPSEVTASM
Construct 1706 from 2X35S to NOS
SEQ ID NO: 16
GTCAACATGGTGGAGCACGACACACTTGTCTACTCCAAAAATAT
CAAAGATACAGTCTCAGAAGACCAAAGGGCAATTGAGACTTTTC
AACAAAGGGTAATATCCGGAAACCTCCTCGGATTCCATTGCCCA
GCTATCTGTCACTTTATTGTGAAGATAGTGGAAAAGGAAGGTGG
CTCCTACAAATGCCATCATTGCGATAAAGGAAAGGCCATCGTTG
AAGATGCCTCTGCCGACAGTGGTCCCAAAGATGGACCCCCACCC
ACGAGGAGCATCGTGGAAAAAGAAGACGTTCCAACCACGTCTTC
AAAGCAAGTGGATTGATGTGATAACATGGTGGAGCACGACACAC
TTGTCTACTCCAAAAATATCAAAGATACAGTCTCAGAAGACCAA
AGGGCAATTGAGACTTTTCAACAAAGGGTAATATCCGGAAACCT
CCTCGGATTCCATTGCCCAGCTATCTGTCACTTTATTGTGAAGA
TAGTGGAAAAGGAAGGTGGCTCCTACAAATGCCATCATTGCGAT
AAAGGAAAGGCCATCGTTGAAGATGCCTCTGCCGACAGTGGTCC
CAAAGATGGACCCCCACCCACGAGGAGCATCGTGGAAAAAGAAG
ACGTTCCAACCACGTCTTCAAAGCAAGTGGATTGATGTGATATC
TCCACTGACGTAAGGGATGACGCACAATCCCACTATCCTTCGCA
AGACCCTTCCTCTATATAAGGAAGTTCATTTCATTTGGAGAGGT
ATTAAAATCTTAATAGGTTTTGATAAAAGCGAACGTGGGGAAAC
CCGAACCAAACCTTCTTCTAAACTCTCTCTCATCTCTCTTAAAG
CAAACTTCTCTCTTGTCTTTCTTGCGTGAGCGATCTTCAACGTT
GTCAGATCGTGCTTCGGCACCAGTACAACGTTTTCTTTCACTGA
AGCGAAATCAAAGATCTCTTTGTGGACACGTAGTGCGGCGCCAT
TAAATAACGTGTACTTGTCCTATTCTTGTCGGTGTGGTCTTGGG
AAAAGAAAGCTTGCTGGAGGCTGCTGTTCAGCCCCATACATTAC
TTGTTACGATTCTGCTGACTTTCGGCGGGTGCAATATCTCTACT
TCTGCTTGACGAGGTATTGTTGCCTGTACTTCTTTCTTCTTCTT
CTTGCTGATTGGTTCTATAAGAAATCTAGTATTTTCTTTGAAAC
AGAGTTTTCCCGTGGTTTTCGAACTTGGAGAAAGATTGTTAAGC
TTCTGTATATTCTGCCCAAATTTGTCGGGCCCATGGATAAGCTT
GCCGACCTCAACTACACATTGAGTGTAATCACTTCAATGAATGA
CACATTGCATTCTATAATTCAAGATCCTGGAATGGCGTATTTTC
TATATATTGCATCTGTTCTAACAGTTTTGTTCACATTACATAAA
GCTTCAATTCCAACCATGAAAATAGCATTGAAAACATCAAAATG
TTCATATAAAGTGATTAAATATTGTATAGTCACGATCATTAATA
CTCTTTTAAAATTGGCTGGATATAAAGAGCAGGTTACTACAAAA
GACGAAATTGAGCAACAGATGGACAGAATTGTGAAAGAGATGAG
ACGTCAGCTGGAGATGATTGATAAACTAACTACTCGTGAAATTG
AACAGGTTGAATTGCTTAAACGTATACATGACAACCTGATAACT
AGACCAGTTGACGTTATAGATATGTCGAAGGAATTCAATCAGAA
AAACATCAAAACGCTAGATGAATGGGAGAGTGGAAAAAATCCAT
ATGAACCGTCAGAAGTGACTGCATCCATGTGAAGGCCTATTTTC
TTTAGTTTGAATTTACTGTTATTCGGTGTGCATTTCTATGTTTG
GTGAGCGGTTTTCTGTGCTCAGAGTGTGTTTATTTTATGTAATT
TAATTTCTTTGTGAGCTCCTGTTTAGCAGGTCGTCCCTTCAGCA
AGGACACAAAAAGATTTTAATTTTATTAAAAAAAAAAAAAAAAA
AGACCGGGAATTCGATATCAAGCTTATCGACCTGCAGATCGTTC
AAACATTTGGCAATAAAGTTTCTTAAGATTGAATCCTGTTGCCG
GTCTTGCGATGATTATCATATAATTTCTGTTGAATTACGTTAAG
CATGTAATAATTAACATGTAATGCATGACGTTATTTATGAGATG
GGTTTTTATGATTAGAGTCCCGCAATTATACATTTAATACGCGA
TAGAAAACAAAATATAGCGCGCAAACTAGGATAAATTATCGCGC
GCGGTGTCATCTATGTTACTAGAT
IF(C160)-TrSP + Rtx_VP7(opt).c
SEQ ID NO: 17
TCGTGCTTCGGCACCAGTACAATGGATTATATTATCTATCGTAG
CCTCCTCATCTA
IF-Rtx_VP7(opt).s1-4r
SEQ ID NO: 18
ACTAAAGAAAATAGGCCTCTAAACGCGATAATAGAAGGCTGCTG
AGTTCAGGGA
Rtx_VP7_DNA_Opt
SEQ ID NO: 19
ATGGATTATATTATCTATCGTAGCCTCCTCATCTACGTGGCCCT
TTTTGCCCTGACCAGGGCCCAGAACTATGGCCTGAACTTACCAA
TCACCGGTTCAATGGATACCGTTTACGCTAATTCCACTCAAGAG
GGGATATTTCTGACAAGTACCCTGTGCCTGTATTATCCAACAGA
AGCCTCTACCCAGATCAATGATGGGGAGTGGAAGGATAGTCTCT
CACAGATGTTCCTAACCAAGGGCTGGCCCACCGGTTCCGTCTAC
TTCAAGGAATACTCTAGTATTGTCGACTTCTCAGTTGACCCCCA
GCTTTATTGCGACTACAACCTGGTACTTATGAAATACGACCAGA
ACCTGGAGCTGGATATGTCCGAGCTGGCTGACCTGATCCTCAAT
GAGTGGCTGTGCAACCCCATGGACATCACATTATATTACTACCA
GCAGTCTGGAGAATCCAACAAGTGGATCAGTATGGGCTCAAGTT
GCACCGTGAAGGTGTGTCCCTTGAACACCCAAATGCTGGGCATT
GGTTGTCAGACAACTAATGTGGATTCGTTTGAAATGGTAGCCGA
AAACGAGAAGCTGGCTATAGTGGACGTAGTCGATGGGATTAACC
ACAAGATCAATCTGACTACCACCACTTGTACCATCAGAAACTGT
AAAAAGCTCGGCCCCCGGGAGAACGTCGCCGTGATCCAGGTGGG
GGGGAGCAATGTGCTCGACATTACTGCCGACCCTACCACCAATC
CACAGACGGAACGGATGATGAGAGTCAACTGGAAGAAATGGTGG
CAGGTCTTTTATACCATTGTGGACTACATTAACCAGATTGTGCA
AGTCATGAGTAAACGGTCCAGATCCCTGAACTCAGCAGCCTTCT
ATTATCGCGTTTAG
Rtx_VP7_AA
SEQ ID NO: 20
MDYIIYRSLLIYVALFALTRAQNYGLNLPITGSMDTVYANSTQE
GIFLTSTLCLYYPTEASTQINDGEWKDSLSQMFLTKGWPTGSVY
FKEYSSIVDFSVDPQLYCDYNLVLMKYDQNLELDMSELADLILN
EWLCNPMDITLYYYQQSGESNKWISMGSSCTVKVCPLNTQMLGI
GCQTTNVDSFEMVAENEKLAIVDVVDGINHKINLTTTTCTIRNC
KKLGPRENVAVIQVGGSNVLDITADPTTNPQTERMMRVNWKKWW
QVFYTIVDYINQIVQVMSKRSRSLNSAAFYYRV
Cloning vector 1190 from left to right T-DNA
SEQ ID NO: 21
TGGCAGGATATATTGTGGTGTAAACAAATTGACGCTTAGACAAC
TTAATAACACATTGCGGACGTTTTTAATGTACTGAATTAACGCC
GAATCCCGGGCTGGTATATTTATATGTTGTCAAATAACTCAAAA
ACCATAAAAGTTTAAGTTAGCAAGTGTGTACATTTTTACTTGAA
CAAAAATATTCACCTACTACTGTTATAAATCATTATTAAACATT
AGAGTAAAGAAATATGGATGATAAGAACAAGAGTAGTGATATTT
TGACAACAATTTTGTTGCAACATTTGAGAAAATTTTGTTGTTCT
CTCTTTTCATTGGTCAAAAACAATAGAGAGAGAAAAAGGAAGAG
GGAGAATAAAAACATAATGTGAGTATGAGAGAGAAAGTTGTACA
AAAGTTGTACCAAAATAGTTGTACAAATATCATTGAGGAATTTG
ACAAAAGCTACACAAATAAGGGTTAATTGCTGTAAATAAATAAG
GATGACGCATTAGAGAGATGTACCATTAGAGAATTTTTGGCAAG
TCATTAAAAAGAAAGAATAAATTATTTTTAAAATTAAAAGTTGA
GTCATTTGATTAAACATGTGATTATTTAATGAATTGATGAAAGA
GTTGGATTAAAGTTGTATTAGTAATTAGAATTTGGTGTCAAATT
TAATTTGACATTTGATCTTTTCCTATATATTGCCCCATAGAGTC
AGTTAACTCATTTTTATATTTCATAGATCAAATAAGAGAAATAA
CGGTATATTAATCCCTCCAAAAAAAAAAAACGGTATATTTACTA
AAAAATCTAAGCCACGTAGGAGGATAACAGGATCCCCGTAGGAG
GATAACATCCAATCCAACCAATCACAACAATCCTGATGAGATAA
CCCACTTTAAGCCCACGCATCTGTGGCACATCTACATTATCTAA
ATCACACATTCTTCCACACATCTGAGCCACACAAAAACCAATCC
ACATCTTTATCACCCATTCTATAAAAAATCACACTTTGTGAGTC
TACACTTTGATTCCCTTCAAACACATACAAAGAGAAGAGACTAA
TTAATTAATTAATCATCTTGAGAGAAAATGGAACGAGCTATACA
AGGAAACGACGCTAGGGAACAAGCTAACAGTGAACGTTGGGATG
GAGGATCAGGAGGTACCACTTCTCCCTTCAAACTTCCTGACGAA
AGTCCGAGTTGGACTGAGTGGCGGCTACATAACGATGAGACGAA
TTCGAATCAAGATAATCCCCTTGGTTTCAAGGAAAGCTGGGGTT
TCGGGAAAGTTGTATTTAAGAGATATCTCAGATACGACAGGACG
GAAGCTTCACTGCACAGAGTCCTTGGATCTTGGACGGGAGATTC
GGTTAACTATGCAGCATCTCGATTTTTCGGTTTCGACCAGATCG
GATGTACCTATAGTATTCGGTTTCGAGGAGTTAGTATCACCGTT
TCTGGAGGGTCGCGAACTCTTCAGCATCTCTGTGAGATGGCAAT
TCGGTCTAAGCAAGAACTGCTACAGCTTGCCCCAATCGAAGTGG
AAAGTAATGTATCAAGAGGATGCCCTGAAGGTACTCAAACCTTC
GAAAAAGAAAGCGAGTAAGTTAAAATGCTTCTTCGTCTCCTATT
TATAATATGGTTTGTTATTGTTAATTTTGTTCTTGTAGAAGAGC
TTAATTAATCGTTGTTGTTATGAAATACTATTTGTATGAGATGA
ACTGGTGTAATGTAATTCATTTACATAAGTGGAGTCAGAATCAG
AATGTTTCCTCCATAACTAACTAGACATGAAGACCTGCCGCGTA
CAATTGTCTTATATTTGAACAACTAAAATTGAACATCTTTTGCC
ACAACTTTATAAGTGGTTAATATAGCTCAAATATATGGTCAAGT
TCAATAGATTAATAATGGAAATATCAGTTATCGAAATTCATTAA
CAATCAACTTAACGTTATTAACTACTAATTTTATATCATCCCCT
TTGATAAATGATAGTACACCAATTAGGAAGGAGCATGCTCGCCT
AGGAGATTGTCGTTTCCCGCCTTCAGTTTGCAAGCTGCTCTAGC
CGTGTAGCCAATACGCAAACCGCCTCTCCCCGCGCGTTGGGAAT
TACTAGCGCGTGTCGACAAGCTTGCATGCCGGTCAACATGGTGG
AGCACGACACACTTGTCTACTCCAAAAATATCAAAGATACAGTC
TCAGAAGACCAAAGGGCAATTGAGACTTTTCAACAAAGGGTAAT
ATCCGGAAACCTCCTCGGATTCCATTGCCCAGCTATCTGTCACT
TTATTGTGAAGATAGTGGAAAAGGAAGGTGGCTCCTACAAATGC
CATCATTGCGATAAAGGAAAGGCCATCGTTGAAGATGCCTCTGC
CGACAGTGGTCCCAAAGATGGACCCCCACCCACGAGGAGCATCG
TGGAAAAAGAAGACGTTCCAACCACGTCTTCAAAGCAAGTGGAT
TGATGTGATAACATGGTGGAGCACGACACACTTGTCTACTCCAA
AAATATCAAAGATACAGTCTCAGAAGACCAAAGGGCAATTGAGA
CTTTTCAACAAAGGGTAATATCCGGAAACCTCCTCGGATTCCAT
TGCCCAGCTATCTGTCACTTTATTGTGAAGATAGTGGAAAAGGA
AGGTGGCTCCTACAAATGCCATCATTGCGATAAAGGAAAGGCCA
TCGTTGAAGATGCCTCTGCCGACAGTGGTCCCAAAGATGGACCC
CCACCCACGAGGAGCATCGTGGAAAAAGAAGACGTTCCAACCAC
GTCTTCAAAGCAAGTGGATTGATGTGATATCTCCACTGACGTAA
GGGATGACGCACAATCCCACTATCCTTCGCAAGACCCTTCCTCT
ATATAAGGAAGTTCATTTCATTTGGAGAGGTATTAAAATCTTAA
TAGGTTTTGATAAAAGCGAACGTGGGGAAACCCGAACCAAACCT
TCTTCTAAACTCTCTCTCATCTCTCTTAAAGCAAACTTCTCTCT
TGTCTTTCTTGCGTGAGCGATCTTCAACGTTGTCAGATCGTGCT
TCGGCACCGCGGATGGCGAAAAACGTTGCGATTTTCGGCTTATT
GTTTTCTCTTCTTGTGTTGGTTCCTTCTCAGATCTTCGCCTGCA
GGCTCCTCAGCCAAAACGACACCCCCATCTGTCTATCCACTGGC
CCCTGGATCTGCTGCCCAAACTAACTCCATGGTGACCCTGGGAT
GCCTGGTCAAGGGCTATTTCCCTGAGCCAGTGACAGTGACCTGG
AACTCTGGATCCCTGTCCAGCGGTGTGCACACCTTCCCAGCTGT
CCTGCAGTCTGACCTCTACACTCTGAGCAGCTCAGTGACTGTCC
CCTCCAGCACCTGGCCCAGCGAGACCGTCACCTGCAACGTTGCC
CACCCGGCCAGCAGCACCAAGGTGGACAAGAAAATTGTGCCCAG
GGATTGTGGTTGTAAGCCTTGCATATGTACAGTCCCAGAAGTAT
CATCTGTCTTCATCTTCCCCCCAAAGCCCAAGGATGTGCTCACC
ATTACTCTGACTCCTAAGGTCACGTGTGTTGTGGTAGACATCAG
CAAGGATGATCCCGAGGTCCAGTTCAGCTGGTTTGTAGATGATG
TGGAGGTGCACACAGCTCAGACGCAACCCCGGGAGGAGCAGTTC
AACAGCACTTTCCGCTCAGTCAGTGAACTTCCCATCATGCACCA
GGACTGGCTCAATGGCAAGGAGCGATCGCTCACCATCACCATCA
CCATCACCATCACCATTAAAGGCCTATTTTCTTTAGTTTGAATT
TACTGTTATTCGGTGTGCATTTCTATGTTTGGTGAGCGGTTTTC
TGTGCTCAGAGTGTGTTTATTTTATGTAATTTAATTTCTTTGTG
AGCTCCTGTTTAGCAGGTCGTCCCTTCAGCAAGGACACAAAAAG
ATTTTAATTTTATTAAAAAAAAAAAAAAAAAAGACCGGGAATTC
GATATCAAGCTTATCGACCTGCAGATCGTTCAAACATTTGGCAA
TAAAGTTTCTTAAGATTGAATCCTGTTGCCGGTCTTGCGATGAT
TATCATATAATTTCTGTTGAATTACGTTAAGCATGTAATAATTA
ACATGTAATGCATGACGTTATTTATGAGATGGGTTTTTATGATT
AGAGTCCCGCAATTATACATTTAATACGCGATAGAAAACAAAAT
ATAGCGCGCAAACTAGGATAAATTATCGCGCGCGGTGTCATCTA
TGTTACTAGATCTCTAGAGTCTCAAGCTTGGCGCGCCCACGTGA
CTAGTGGCACTGGCCGTCGTTTTACAACGTCGTGACTGGGAAAA
CCCTGGCGTTACCCAACTTAATCGCCTTGCAGCACATCCCCCTT
TCGCCAGCTGGCGTAATAGCGAAGAGGCCCGCACCGATCGCCCT
TCCCAACAGTTGCGCAGCCTGAATGGCGAATGCTAGAGCAGCTT
GAGCTTGGATCAGATTGTCGTTTCCCGCCTTCAGTTTAAACTAT
CAGTGTTTGACAGGATATATTGGCGGGTAAACCTAAGAGAAAAG
AGCGTTTA
Construct 1199 from 2X35S to NOS
SEQ ID NO: 22
GTCAACATGGTGGAGCACGACACACTTGTCTACTCCAAAAATAT
CAAAGATACAGTCTCAGAAGACCAAAGGGCAATTGAGACTTTTC
AACAAAGGGTAATATCCGGAAACCTCCTCGGATTCCATTGCCCA
GCTATCTGTCACTTTATTGTGAAGATAGTGGAAAAGGAAGGTGG
CTCCTACAAATGCCATCATTGCGATAAAGGAAAGGCCATCGTTG
AAGATGCCTCTGCCGACAGTGGTCCCAAAGATGGACCCCCACCC
ACGAGGAGCATCGTGGAAAAAGAAGACGTTCCAACCACGTCTTC
AAAGCAAGTGGATTGATGTGATAACATGGTGGAGCACGACACAC
TTGTCTACTCCAAAAATATCAAAGATACAGTCTCAGAAGACCAA
AGGGCAATTGAGACTTTTCAACAAAGGGTAATATCCGGAAACCT
CCTCGGATTCCATTGCCCAGCTATCTGTCACTTTATTGTGAAGA
TAGTGGAAAAGGAAGGTGGCTCCTACAAATGCCATCATTGCGAT
AAAGGAAAGGCCATCGTTGAAGATGCCTCTGCCGACAGTGGTCC
CAAAGATGGACCCCCACCCACGAGGAGCATCGTGGAAAAAGAAG
ACGTTCCAACCACGTCTTCAAAGCAAGTGGATTGATGTGATATC
TCCACTGACGTAAGGGATGACGCACAATCCCACTATCCTTCGCA
AGACCCTTCCTCTATATAAGGAAGTTCATTTCATTTGGAGAGGT
ATTAAAATCTTAATAGGTTTTGATAAAAGCGAACGTGGGGAAAC
CCGAACCAAACCTTCTTCTAAACTCTCTCTCATCTCTCTTAAAG
CAAACTTCTCTCTTGTCTTTCTTGCGTGAGCGATCTTCAACGTT
GTCAGATCGTGCTTCGGCACCAGTACAATGGATTATATTATCTA
TCGTAGCCTCCTCATCTACGTGGCCCTTTTTGCCCTGACCAGGG
CCCAGAACTATGGCCTGAACTTACCAATCACCGGTTCAATGGAT
ACCGTTTACGCTAATTCCACTCAAGAGGGGATATTTCTGACAAG
TACCCTGTGCCTGTATTATCCAACAGAAGCCTCTACCCAGATCA
ATGATGGGGAGTGGAAGGATAGTCTCTCACAGATGTTCCTAACC
AAGGGCTGGCCCACCGGTTCCGTCTACTTCAAGGAATACTCTAG
TATTGTCGACTTCTCAGTTGACCCCCAGCTTTATTGCGACTACA
ACCTGGTACTTATGAAATACGACCAGAACCTGGAGCTGGATATG
TCCGAGCTGGCTGACCTGATCCTCAATGAGTGGCTGTGCAACCC
CATGGACATCACATTATATTACTACCAGCAGTCTGGAGAATCCA
ACAAGTGGATCAGTATGGGCTCAAGTTGCACCGTGAAGGTGTGT
CCCTTGAACACCCAAATGCTGGGCATTGGTTGTCAGACAACTAA
TGTGGATTCGTTTGAAATGGTAGCCGAAAACGAGAAGCTGGCTA
TAGTGGACGTAGTCGATGGGATTAACCACAAGATCAATCTGACT
ACCACCACTTGTACCATCAGAAACTGTAAAAAGCTCGGCCCCCG
GGAGAACGTCGCCGTGATCCAGGTGGGGGGGAGCAATGTGCTCG
ACATTACTGCCGACCCTACCACCAATCCACAGACGGAACGGATG
ATGAGAGTCAACTGGAAGAAATGGTGGCAGGTCTTTTATACCAT
TGTGGACTACATTAACCAGATTGTGCAAGTCATGAGTAAACGGT
CCAGATCCCTGAACTCAGCAGCCTTCTATTATCGCGTTTAGAGG
CCTATTTTCTTTAGTTTGAATTTACTGTTATTCGGTGTGCATTT
CTATGTTTGGTGAGCGGTTTTCTGTGCTCAGAGTGTGTTTATTT
TATGTAATTTAATTTCTTTGTGAGCTCCTGTTTAGCAGGTCGTC
CCTTCAGCAAGGACACAAAAAGATTTTAATTTTATTAAAAAAAA
AAAAAAAAAAGACCGGGAATTCGATATCAAGCTTATCGACCTGC
AGATCGTTCAAACATTTGGCAATAAAGTTTCTTAAGATTGAATC
CTGTTGCCGGTCTTGCGATGATTATCATATAATTTCTGTTGAAT
TACGTTAAGCATGTAATAATTAACATGTAATGCATGACGTTATT
TATGAGATGGGTTTTTATGATTAGAGTCCCGCAATTATACATTT
AATACGCGATAGAAAACAAAATATAGCGCGCAAACTAGGATAAA
TTATCGCGCGCGGTGTCATCTATGTTACTAGAT
IF-(160)RVA(G2P5SC2-9)VP7.c
SEQ ID NO: 23
TCGTGCTTCGGCACCAGTACAATGGACTACATTATCTATCGATT
TTTA
IF-RVA(G2P5SC2-9)VP7.r
SEQ ID NO: 24
ACTAAAGAAAATAGGCCTCTACACTCGGTAATAGAAGGCGGCAG
CATCCAGGCTC
Sc2-9_VP7_DNA_Opt
SEQ ID NO: 25
ATGGACTACATTATCTATCGATTTTTATTGGTAATTGTGCTGAT
CTCACCATTCGTCAGGACTCAGAACTACGGGATCTACCTGCCGA
TAACCGGCTCTCTGGATGCAGTGTATACAAATAGCACCTCAGGT
GAGACATTTCTCACAAGCACCCTTTGCCTTTATTACCCAGCAGA
AGCAAAGAATGAAATTAGCGACGATGAGTGGGAGAATACACTTT
CACAGCTGTTTCTCACCAAGGGGTGGCCAACCGGTAGCGTATAC
TTCAAAGACTATAACGACATTACGACCTTTAGTATGAACCCTCA
GCTCTACTGTGACTATAACGTCGTGTTAATGCGCTATGACAATA
CCAGCGAGCTCGACGCCTCTGAGCTGGCTGACCTGATCCTGAAT
GAGTGGTTGTGCAACCCAATGGACATCTCCCTTTACTATTACCA
GCAGTCCTCCGAGAGTAACAAGTGGATTAGCATGGGTACCGATT
GCACTGTAAAGGTGTGTCCCCTGAATACCCAGACTCTCGGAATC
GGTTGCAAAACCACCGACGTGAGCACTTTCGAAATAGTTGCTTC
CTCAGAGAAGCTAGTTATCACAGACGTGGTGAACGGCGTCAACC
ACAAAATCAATATCAGCATCTCCACTTGCACTATTCGAAATTGC
AACAAACTCGGCCCCCGGGAGAACGTGGCCATTATCCAGGTTGG
CGGCCCTAACGCGCTCGACATCACTGCAGATCCAACAACCGTGC
CTCAAATTCAGCGGATTATGAGAATCAATTGGAAAAAGTGGTGG
CAGGTGTTTTATACGGTTGTGGACTATATTAATCAGATCGTACA
GGTGATGAGCAAAAGGAGCAGGAGCCTGGATGCTGCCGCCTTCT
ATTACCGAGTGTAG
Sc2-9_VP7_AA
SEQ ID NO: 26
MDYIIYRELLVIVLISPEVRTQNYGIYLPITGSLDAVYTNSTSG
ETFLTSTLCLYYPAEAKNEISDDEWENTLSQLFLTKGWPTGSVY
FKDYNDITTESMNPQLYCDYNVVLMRYDNTSELDASELADLILN
EWLCNPMDISLYYYQQSSESNKWISMGTDCTVKVCPLNTQTLGI
GCKTTDVSTFEIVASSEKLVITDVVNGVNHKINISISTCTIRNC
NKLGPRENVAIIQVGGPNALDITADPTTVPQIQRIMRINWKKWW
QVFYTVVDYINQIVQVMSKRSRSLDAAAFYYRV
7-1a_Sc2-9_VP7_DNA_Opt
SEQ ID NO: 27
ATGGATTATATTATCTATCGTAGCCTCCTCATCTACGTGGCCCT
TTTTGCCCTGACCAGGGCCCAGAACTATGGCCTGAACTTACCAA
TCACCGGTTCAATGGATACCGTTTACGCTAATTCCACTCAAGAG
GGGATATTTCTGACAAGCACCCTTTGCCTTTATTACCCAGCAGA
AGCAAAGAATGAAATTAGCGACGATGAGTGGGAGAATACACTTT
CACAGCTGTTTCTCACCAAGGGGTGGCCAACCGGTAGCGTATAC
TTCAAAGACTATAACGACATTACGACCTTTAGTATGAACCCTCA
GCTCTACTGTGACTATAACGTCGTGTTAATGCGCTATGACAATA
CCAGCGAGCTCGACGCCTCTGAGCTGGCTGACCTGATCCTGAAT
GAGTGGCTGTGCAACCCCATGGACATCACATTATATTACTACCA
GCAGTCTGGAGAATCCAACAAGTGGATCAGTATGGGCTCAAGTT
GCACCGTGAAGGTGTGTCCCTTGAACACCCAAATGCTGGGCATT
GGTTGTCAGACAACTAATGTGGATTCGTTTGAAATGGTAGCCGA
AAACGAGAAGCTGGCTATAGTGGACGTAGTCGATGGGATTAACC
ACAAGATCAATCTGACTACCACCACTTGTACCATCAGAAACTGT
AAAAAGCTCGGCCCCCGGGAGAACGTCGCCGTGATCCAGGTGGG
GGGGAGCAATGTGCTCGACATTACTGCCGACCCTACCACCAATC
CACAGACGGAACGGATGATGAGAGTCAACTGGAAGAAATGGTGG
CAGGTCTTTTATACCATTGTGGACTACATTAACCAGATTGTGCA
AGTCATGAGTAAACGGTCCAGATCCCTGAACTCAGCAGCCTTCT
ATTATCGCGTTTAG
7-1a_Sc2-9_VP7_AA
SEQ ID NO: 28
MDYIIYRSLLIYVALFALTRAQNYGLNLPITGSMDTVYANSTQE
GIFLTSTLCLYYPAEAKNEISDDEWENTLSQLFLTKGWPTGSVY
FKDYNDITTFSMNPQLYCDYNVVLMRYDNTSELDASELADLILN
EWLCNPMDITLYYYQQSGESNKWISMGSSCTVKVCPLNTQMLGI
GCQTTNVDSFEMVAENEKLAIVDVVDGINHKINLTTTTCTIRNC
KKLGPRENVAVIQVGGSNVLDITADPTTNPQTERMMRVNWKKWW
QVFYTIVDYINQIVQVMSKRSRSLNSAAFYYRV
IF-VP7(3End)Rtx + VP7-1b(G2Sc2-9).r
SEQ ID NO: 29
ACTAAAGAAAATAGGCCTCTAAACGCGATAATAGAAGGCTGCTG
AGTTCAGGGATCTGGACCGTTTGCTCATCACCTGTACGATC
7-1b_Sc2-9_VP7_DNA_Opt
SEQ ID NO: 30
ATGGATTATATTATCTATCGTAGCCTCCTCATCTACGTGGCCCT
TTTTGCCCTGACCAGGGCCCAGAACTATGGCCTGAACTTACCAA
TCACCGGTTCAATGGATACCGTTTACGCTAATTCCACTCAAGAG
GGGATATTTCTGACAAGTACCCTGTGCCTGTATTATCCAACAGA
AGCCTCTACCCAGATCAATGATGGGGAGTGGAAGGATAGTCTCT
CACAGATGTTCCTAACCAAGGGCTGGCCCACCGGTTCCGTCTAC
TTCAAGGAATACTCTAGTATTGTCGACTTCTCAGTTGACCCCCA
GCTTTATTGCGACTACAACCTGGTACTTATGAAATACGACCAGA
ACCTGGAGCTGGATATGTCCGAGCTGGCTGACCTGATCCTCAAT
GAGTGGCTGTGCAACCCCATGGACATCACATTATATTACTACCA
GCAGTCTGGAGAATCCAACAAGTGGATCAGTATGGGCTCAAGTT
GCACCGTGAAGGTGTGTCCCTTGAACACCCAAATGCTGGGCATT
GGTTGTCAGACAACTAATGTGGATTCGTTTGAAATGGTAGCCGA
AAACGAGAAGCTGGCTATAGTGGACGTAGTCGATGGGATTAACC
ACAAGATCAATCTGACTACCACCACTTGTACCATCAGAAACTGT
AAAAAGCTCGGCCCCCGGGAGAACGTGGCCATTATCCAGGTTGG
CGGCCCTAACGCGCTCGACATCACTGCAGATCCAACAACCGTGC
CTCAAATTCAGCGGATTATGAGAATCAATTGGAAAAAGTGGTGG
CAGGTGTTTTATACGGTTGTGGACTATATTAATCAGATCGTACA
GGTGATGAGCAAACGGTCCAGATCCCTGAACTCAGCAGCCTTCT
ATTATCGCGTTTAG
7-1b_Sc2-9_VP7_AA
SEQ ID NO: 31
MDYIIYRSLLIYVALFALTRAQNYGLNLPITGSMDTVYANSTQE
GIFLTSTLCLYYPTEASTQINDGEWKDSLSQMFLTKGWPTGSVY
FKEYSSIVDFSVDPQLYCDYNLVLMKYDQNLELDMSELADLILN
EWLCNPMDITLYYYQQSGESNKWISMGSSCTVKVCPLNTQMLGI
GCQTTNVDSFEMVAENEKLAIVDVVDGINHKINLTTTTCTIRNC
KKLGPRENVAIIQVGGPNALDITADPTTVPQIQRIMRINWKKWW
QVFYTVVDYINQIVQVMSKRSRSLNSAAFYYRV
7-1a + 1b_Sc2-9_VP7_DNA_Opt
SEQ ID NO: 32
ATGGATTATATTATCTATCGTAGCCTCCTCATCTACGTGGCCCT
TTTTGCCCTGACCAGGGCCCAGAACTATGGCCTGAACTTACCAA
TCACCGGTTCAATGGATACCGTTTACGCTAATTCCACTCAAGAG
GGGATATTTCTGACAAGCACCCTTTGCCTTTATTACCCAGCAGA
AGCAAAGAATGAAATTAGCGACGATGAGTGGGAGAATACACTTT
CACAGCTGTTTCTCACCAAGGGGTGGCCAACCGGTAGCGTATAC
TTCAAAGACTATAACGACATTACGACCTTTAGTATGAACCCTCA
GCTCTACTGTGACTATAACGTCGTGTTAATGCGCTATGACAATA
CCAGCGAGCTCGACGCCTCTGAGCTGGCTGACCTGATCCTGAAT
GAGTGGCTGTGCAACCCCATGGACATCACATTATATTACTACCA
GCAGTCTGGAGAATCCAACAAGTGGATCAGTATGGGCTCAAGTT
GCACCGTGAAGGTGTGTCCCTTGAACACCCAAATGCTGGGCATT
GGTTGTCAGACAACTAATGTGGATTCGTTTGAAATGGTAGCCGA
AAACGAGAAGCTGGCTATAGTGGACGTAGTCGATGGGATTAACC
ACAAGATCAATCTGACTACCACCACTTGTACCATCAGAAACTGT
AAAAAGCTCGGCCCCCGGGAGAACGTGGCCATTATCCAGGTTGG
CGGCCCTAACGCGCTCGACATCACTGCAGATCCAACAACCGTGC
CTCAAATTCAGCGGATTATGAGAATCAATTGGAAAAAGTGGTGG
CAGGTGTTTTATACGGTTGTGGACTATATTAATCAGATCGTACA
GGTGATGAGCAAACGGTCCAGATCCCTGAACTCAGCAGCCTTCT
ATTATCGCGTTTAG
7-1a + 1b_Sc2-9_VP7_AA
SEQ ID NO: 33
MDYIIYRSLLIYVALFALTRAQNYGLNLPITGSMDTVYANSTQE
GIFLTSTLCLYYPAEAKNEISDDEWENTLSQLFLTKGWPTGSVY
FKDYNDITTFSMNPQLYCDYNVVLMRYDNTSELDASELADLILN
EWLCNPMDITLYYYQQSGESNKWISMGSSCTVKVCPLNTQMLGI
GCQTTNVDSFEMVAENEKLAIVDVVDGINHKINLTTTTCTIRNC
KKLGPRENVAIIQVGGPNALDITADPTTVPQIQRIMRINWKKWW
QVFYTVVDYINQIVQVMSKRSRSLNSAAFYYRV
IF-(160)RVA(G9P8WI61)VP7.c
SEQ ID NO: 34
TCGTGCTTCGGCACCAGTACAATGGATTTTATCATCTACCGATT
TCTA
IF-RVA(G9P8WI61)VP7.r
SEQ ID NO: 35
ACTAAAGAAAATAGGCCTTCAAACCCGGTAATAGAACGCTGCGG
AGTTT
WI61_VP7_DNA_Opt
SEQ ID NO: 36
ATGGATTTTATCATCTACCGATTTCTATTGTTGATTGTTATCGT
AAGCCCGTTCGTGAAAACGCAGAACTATGGAATCAATCTGCCTA
TTACAGGGAGTATGGATACCGCGTACGCTAATTCTTCACAGCTG
GATACGTTTTTAACCTCCACACTTTGCTTATACTATCCTGCTGA
GGCGAGCACTCAGATTGGAGACACCGAGTGGAAGAACACTCTGA
GCCAGCTATTCCTGACGAAAGGGTGGCCCACAGGCTCTGTGTAT
TTCAAAGAATATACTGACATCGCCTCCTTCAGCATCGATCCACA
GCTCTACTGCGACTATAACGTGGTTTTAATGAAGTATGATTCTA
CACTTAAACTTGACATGTCCGAACTGGCTGACCTGATCCTGAAC
GAGTGGCTGTGCAACCCCATGGACATCACGCTGTATTATTACCA
GCAGACAGACGAAGCCAACAAGTGGATCGCCATGGGACAGAGCT
GTACAATTAAAGTGTGTCCACTCAACACCCAAACTCTCGGTATT
GGGTGCACTACAACCAATACGGCCACTTTTGAGGAGGTGGCGGC
CTCTGAGAAGCTGGTGATTACAGATGTGGTAGACGGCGTGAACC
ACAAACTGGATGTGACGACGACAACGTGTACAATCAGAAACTGT
CGCAAGCTGGGACCTCGGGAAAACGTCGCTATTATACAGGTGGG
AGGGAGCGAAGTGCTAGATATTACGGCAGATCCAACTACAGCCC
CACAGACCGAGAGGATGATGAGGATTAACTGGAAGAAGTGGTGG
CAGGTCTTTTACACCGTCGTGGACTATATTAATCAGATTGTTCA
GGTAATGAGCAAAAGGAGTAGGTCTTTAAACTCCGCAGCGTTCT
ATTACCGGGTTTGA
WI61_VP7_AA
SEQ ID NO: 37
MDFIIYRFLLLIVIVSPFVKTQNYGINLPITGSMDTAYANSSQL
DTFLTSTLCLYYPAEASTQIGDTEWKNTLSQLFLTKGWPTGSVY
FKEYTDIASFSIDPQLYCDYNVVLMKYDSTLKLDMSELADLILN
EWLCNPMDITLYYYQQTDEANKWIAMGQSCTIKVCPLNTQTLGI
GCTTTNTATFEEVAASEKLVITDVVDGVNHKLDVTTTTCTIRNC
RKLGPRENVAIIQVGGSEVLDITADPTTAPQTERMMRINWKKWW
QVFYTVVDYINQIVQVMSKRSRSLNSAAFYYRV
7-1a_WI61_VP7_DNA_Opt
SEQ ID NO: 38
ATGGATTATATTATCTATCGTAGCCTCCTCATCTACGTGGCCCT
TTTTGCCCTGACCAGGGCCCAGAACTATGGCCTGAACTTACCAA
TCACCGGTTCAATGGATACCGTTTACGCTAATTCCACTCAAGAG
GGGATATTTCTGACCTCCACACTTTGCTTATACTATCCTGCTGA
GGCGAGCACTCAGATTGGAGACACCGAGTGGAAGAACACTCTGA
GCCAGCTATTCCTGACGAAAGGGTGGCCCACAGGCTCTGTGTAT
TTCAAAGAATATACTGACATCGCCTCCTTCAGCATCGATCCACA
GCTCTACTGCGACTATAACGTGGTTTTAATGAAGTATGATTCTA
CACTTAAACTTGACATGTCCGAACTGGCTGACCTGATCCTGAAT
GAGTGGCTGTGCAACCCCATGGACATCACATTATATTACTACCA
GCAGTCTGGAGAATCCAACAAGTGGATCAGTATGGGCTCAAGTT
GCACCGTGAAGGTGTGTCCCTTGAACACCCAAATGCTGGGCATT
GGTTGTCAGACAACTAATGTGGATTCGTTTGAAATGGTAGCCGA
AAACGAGAAGCTGGCTATAGTGGACGTAGTCGATGGGATTAACC
ACAAGATCAATCTGACTACCACCACTTGTACCATCAGAAACTGT
AAAAAGCTCGGCCCCCGGGAGAACGTCGCCGTGATCCAGGTGGG
GGGGAGCAATGTGCTCGACATTACTGCCGACCCTACCACCAATC
CACAGACGGAACGGATGATGAGAGTCAACTGGAAGAAATGGTGG
CAGGTCTTTTATACCATTGTGGACTACATTAACCAGATTGTGCA
AGTCATGAGTAAACGGTCCAGATCCCTGAACTCAGCAGCCTTCT
ATTATCGCGTTTAG
7-1a_WI61_VP7_AA
SEQ ID NO: 39
MDYIIYRSLLIYVALFALTRAQNYGLNLPITGSMDTVYANSTQE
GIFLTSTLCLYYPAEASTQIGDTEWKNTLSQLFLTKGWPTGSVY
FKEYTDIASFSIDPQLYCDYNVVLMKYDSTLKLDMSELADLILN
EWLCNPMDITLYYYQQSGESNKWISMGSSCTVKVCPLNTQMLGI
GCQTTNVDSFEMVAENEKLAIVDVVDGINHKINLTTTTCTIRNC
KKLGPRENVAVIQVGGSNVLDITADPTTNPQTERMMRVNWKKWW
QVFYTIVDYINQIVQVMSKRSRSLNSAAFYYRV
7-1b_WI61_VP7_DNA_Opt
SEQ ID NO: 40
ATGGATTATATTATCTATCGTAGCCTCCTCATCTACGTGGCCCT
TTTTGCCCTGACCAGGGCCCAGAACTATGGCCTGAACTTACCAA
TCACCGGTTCAATGGATACCGTTTACGCTAATTCCACTCAAGAG
GGGATATTTCTGACAAGTACCCTGTGCCTGTATTATCCAACAGA
AGCCTCTACCCAGATCAATGATGGGGAGTGGAAGGATAGTCTCT
CACAGATGTTCCTAACCAAGGGCTGGCCCACCGGTTCCGTCTAC
TTCAAGGAATACTCTAGTATTGTCGACTTCTCAGTTGACCCCCA
GCTTTATTGCGACTACAACCTGGTACTTATGAAATACGACCAGA
ACCTGGAGCTGGATATGTCCGAGCTGGCTGACCTGATCCTCAAT
GAGTGGCTGTGCAACCCCATGGACATCACATTATATTACTACCA
GCAGTCTGGAGAATCCAACAAGTGGATCAGTATGGGCTCAAGTT
GCACCGTGAAGGTGTGTCCCTTGAACACCCAAATGCTGGGCATT
GGTTGTCAGACAACTAATGTGGATTCGTTTGAAATGGTAGCCGA
AAACGAGAAGCTGGCTATAGTGGACGTAGTCGATGGGATTAACC
ACAAGATCAATCTGACTACCACCACTTGTACCATCAGAAACTGT
AAAAAGCTCGGCCCCCGGGAGAACGTCGCTATTATACAGGTGGG
AGGGAGCGAAGTGCTAGATATTACGGCAGATCCAACTACAGCCC
CACAGACCGAGAGGATGATGAGGATTAACTGGAAGAAGTGGTGG
CAGGTCTTTTACACCGTCGTGGACTATATTAATCAGATTGTTCA
GGTAATGAGCAAAAGGAGTAGGTCTTTAAACTCCGCAGCGTTCT
ATTACCGGGTTTGA
7-1b_WI61_VP7_AA
SEQ ID NO: 41
MDYIIYRSLLIYVALFALTRAQNYGLNLPITGSMDTVYANSTQE
GIFLTSTLCLYYPTEASTQINDGEWKDSLSQMFLTKGWPTGSVY
FKEYSSIVDFSVDPQLYCDYNLVLMKYDQNLELDMSELADLILN
EWLCNPMDITLYYYQQSGESNKWISMGSSCTVKVCPLNTQMLGI
GCQTTNVDSFEMVAENEKLAIVDVVDGINHKINLTTTTCTIRNC
KKLGPRENVAIIQVGGSEVLDITADPTTAPQTERMMRINWKKWW
QVFYTVVDYINQIVQVMSKRSRSLNSAAFYYRV
7-1a + 1b_WI61_VP7_DNA_Opt
SEQ ID NO: 42
ATGGATTATATTATCTATCGTAGCCTCCTCATCTACGTGGCCCT
TTTTGCCCTGACCAGGGCCCAGAACTATGGCCTGAACTTACCAA
TCACCGGTTCAATGGATACCGTTTACGCTAATTCCACTCAAGAG
GGGATATTTCTGACCTCCACACTTTGCTTATACTATCCTGCTGA
GGCGAGCACTCAGATTGGAGACACCGAGTGGAAGAACACTCTGA
GCCAGCTATTCCTGACGAAAGGGTGGCCCACAGGCTCTGTGTAT
TTCAAAGAATATACTGACATCGCCTCCTTCAGCATCGATCCACA
GCTCTACTGCGACTATAACGTGGTTTTAATGAAGTATGATTCTA
CACTTAAACTTGACATGTCCGAACTGGCTGACCTGATCCTGAAT
GAGTGGCTGTGCAACCCCATGGACATCACATTATATTACTACCA
GCAGTCTGGAGAATCCAACAAGTGGATCAGTATGGGCTCAAGTT
GCACCGTGAAGGTGTGTCCCTTGAACACCCAAATGCTGGGCATT
GGTTGTCAGACAACTAATGTGGATTCGTTTGAAATGGTAGCCGA
AAACGAGAAGCTGGCTATAGTGGACGTAGTCGATGGGATTAACC
ACAAGATCAATCTGACTACCACCACTTGTACCATCAGAAACTGT
AAAAAGCTCGGCCCCCGGGAGAACGTCGCTATTATACAGGTGGG
AGGGAGCGAAGTGCTAGATATTACGGCAGATCCAACTACAGCCC
CACAGACCGAGAGGATGATGAGGATTAACTGGAAGAAGTGGTGG
CAGGTCTTTTACACCGTCGTGGACTATATTAATCAGATTGTTCA
GGTAATGAGCAAAAGGAGTAGGTCTTTAAACTCCGCAGCGTTCT
ATTACCGGGTTTGA
7-1a + 1b_WI61_VP7_AA
SEQ ID NO: 43
MDYIIYRSLLIYVALFALTRAQNYGLNLPITGSMDTVYANSTQE
GIFLTSTLCLYYPAEASTQIGDTEWKNTLSQLFLTKGWPTGSVY
FKEYTDIASFSIDPQLYCDYNVVLMKYDSTLKLDMSELADLILN
EWLCNPMDITLYYYQQSGESNKWISMGSSCTVKVCPLNTQMLGI
GCQTTNVDSFEMVAENEKLAIVDVVDGINHKINLTTTTCTIRNC
KKLGPRENVAIIQVGGSEVLDITADPTTAPQTERMMRINWKKWW
QVFYTVVDYINQIVQVMSKRSRSLNSAAFYYRV
IF-(160)RVA(G3P5WI78-8)VP7.c
SEQ ID NO: 44
TCGTGCTTCGGCACCAGTACAATGGATTTCATTATATACCGCT
TCCTGCTCATC
IF-RVA(G3P5WI78-8)VP7.r
SEQ ID NO: 45
ACTAAAGAAAATAGGCCTTTAAACCCGGTAATAGAATGCGGCC
GAATTCA
WI78-8_VP7_DNA_Opt
SEQ ID NO: 46
ATGGATTTCATTATATACCGCTTCCTGCTCATCATTGTGATACT
TTCTCCCTTGTTGAACGCGCAAAATTACGGGATTAACCTCCCAA
TTACTGGTTCCATGGACACTAGCTACACCAATTCAACCCGGGAG
GAAGTTTTCCTCACGAGCACTCTTTGCCTATATTATCCCACCGA
GGCTGCCACAGAGATCAATGACAATTCCTGGAAAGATACTTTGA
GCCAGCTGTTCCTGACCAAAGGGTGGCCAACCGAGAGTATCTAT
TTTAAAGATTACACCGACATAGCGTCATTTTCTGTTGACCCACA
GCTCTATTGCGACTACAATCTGGTGCTCATGAAATACGACGCAA
CCCTTCAGCTGGACATGTCCGAGCTAGCAGACCTGCTGCTCAAC
GAGTGGCTCTGCAACCCTATGGATATAACGCTGTACTATTACCA
GCAGACAGATGAAGCCAACAAGTGGATTAGTATGGGTTCGAGCT
GCACGATCAAGGTTTGCCCACTGAACACTCAAACCCTCGGTATA
GGTTGTTTGACCACTGACGCGAATACATTTGAGGAAGTGGCCAC
CGCTGAAAAACTTGTGATCACCGACGTCGTGGACGGTGTTAACC
ACAAGCTGAAAGTGACCACCGACACGTGCACGATTCGCAACTGT
AAAAAATTAGGGCCCCGTGAAAACGTGGCTGTGATCCAAGTCGG
AGGGAGTGACGTGCTGGACATTACCGCAGATCCCACAACTGCTC
CACAGACTGAGAGGATGATGAGGGTCAACTGGAAGAAGTGGTGG
CAGGTGTTCTATACGATTGTTGACTACGTCAATCAGATTGTGCA
GGCCATGTCAAAGAGGTCACGATCTCTGAATTCGGCCGCATTCT
ATTACCGGGTTTAA
WI78-8_VP7_AA
SEQ ID NO: 47
MDFIIYRELLIIVILSPLLNAQNYGINLPITGSMDTSYTNSTRE
EVELTSTLCLYYPTEAATEINDNSWKDTLSQLFLTKGWPTESIV
FXDYTDIASFSVDPQLYCDYNLVLMKYDATLQLDMSELADLLLN
EWLCNPMDITLYYYQQTDEANKWISMGSSCTIKVCPLNTQTLGI
GCLTTDANTFEEVATAEKLVITDVVDGVNHKLKVTTDTCTIRNC
KKLGPRENVAVIQVGGSDVLDITADPTTAPQTERMMRVNWKKWW
QVFYTIVDYVNQIVQAMSKRSRSLNSAAFYYRV
IF-VP7(3End)Rtx + VP7-1b(G3P5).r
SEQ ID NO: 48
ACTAAAGAAAATAGGCCTCTAAACGCGATAATAGAAGGCTGCT
GAGTTCAGGGATCTGGAC
CGCTTTGACATGGCCTGCACAATCT
7-1b_WI78-8_VP7_DNA_Opt
SEQ ID NO: 49
ATGGATTATATTATCTATCGTAGCCTCCTCATCTACGTGGCCC
TTTTTGCCCTGACCAGGGCCCAGAACTATGGCCTGAACTTACC
AATCACCGGTTCAATGGATACCGTTTACGCTAATTCCACTCAA
GAGGGGATATTTCTGACAAGTACCCTGTGCCTGTATTATCCAA
CAGAAGCCTCTACCCAGATCAATGATGGGGAGTGGAAGGATAG
TCTCTCACAGATGTTCCTAACCAAGGGCTGGCCCACCGGTTCC
GTCTACTTCAAGGAATACTCTAGTATTGTCGACTTCTCAGTTG
ACCCCCAGCTTTATTGCGACTACAACCTGGTACTTATGAAATA
CGACCAGAACCTGGAGCTGGATATGTCCGAGCTGGCTGACCTG
ATCCTCAATGAGTGGCTGTGCAACCCCATGGACATCACATTAT
ATTACTACCAGCAGTCTGGAGAATCCAACAAGTGGATCAGTAT
GGGCTCAAGTTGCACCGTGAAGGTGTGTCCCTTGAACACCCAA
ATGCTGGGCATTGGTTGTCAGACAACTAATGTGGATTCGTTTG
AAATGGTAGCCGAAAACGAGAAGCTGGCTATAGTGGACGTAGT
CGATGGGATTAACCACAAGATCAATCTGACTACCACCACTTGT
ACCATCAGAAACTGTAAAAAGCTCGGCCCCCGGGAGAACGTGG
CTGTGATCCAAGTCGGAGGGAGTGACGTGCTGGACATTACCGC
AGATCCCACAACTGCTCCACAGACTGAGAGGATGATGAGGGTC
AACTGGAAGAAGTGGTGGCAGGTGTTCTATACGATTGTTGACT
ACGTCAATCAGATTGTGCAGGCCATGTCAAAGCGGTCCAGATC
CCTGAACTCAGCAGCCTTCTATTATCGCGTTTAG
7-1b_WI78-8_VP7_AA
SEQ ID NO: 50
MDYIIYRSLLIYVALFALTRAQNYGLNLPITGSMDTVYANSTQ
EGIFLTSTLCLYYPTEASTQINDGEWKDSLSQMFLTKGWPTGS
VYFKEYSSIVDFSVDPQLYCDYNLVLMKYDQNLELDMSELADL
ILNEWLCNPMDITLYYYQQSGESNKWISMGSSCTVKVCPLNTQ
MLGIGCQTTNVDSFEMVAENEKLAIVDVVDGINHKINLTTTTC
TIRNCKKLGPRENVAVIQVGGSDVLDITADPTTAPQTERMMRV
NWKKWWQVFYTIVDYVNQIVQAMSKRSRSLNSAAFYYRV
7-1a + 1b_WI78-8_VP7_DNA_Opt
SEQ ID NO: 51
ATGGATTATATTATCTATCGTAGCCTCCTCATCTACGTGGCCC
TTTTTGCCCTGACCAGGGCCCAGAACTATGGCCTGAACTTACC
AATCACCGGTTCAATGGATACCGTTTACGCTAATTCCACTCAA
GAGGGGATATTTCTGACGAGCACTCTTTGCCTATATTATCCCA
CCGAGGCTGCCACAGAGATCAATGACAATTCCTGGAAAGATAC
TTTGAGCCAGCTGTTCCTGACCAAAGGGTGGCCAACCGAGAGT
ATCTATTTTAAAGATTACACCGACATAGCGTCATTTTCTGTTG
ACCCACAGCTCTATTGCGACTACAATCTGGTGCTCATGAAATA
CGACGCAACCCTTCAGCTGGACATGTCCGAGCTAGCAGACCTG
CTGCTCAATGAGTGGCTGTGCAACCCCATGGACATCACATTAT
ATTACTACCAGCAGTCTGGAGAATCCAACAAGTGGATCAGTAT
GGGCTCAAGTTGCACCGTGAAGGTGTGTCCCTTGAACACCCAA
ATGCTGGGCATTGGTTGTCAGACAACTAATGTGGATTCGTTTG
AAATGGTAGCCGAAAACGAGAAGCTGGCTATAGTGGACGTAGT
CGATGGGATTAACCACAAGATCAATCTGACTACCACCACTTGT
ACCATCAGAAACTGTAAAAAGCTCGGCCCCCGGGAGAACGTGG
CTGTGATCCAAGTCGGAGGGAGTGACGTGCTGGACATTACCGC
AGATCCCACAACTGCTCCACAGACTGAGAGGATGATGAGGGTC
AACTGGAAGAAGTGGTGGCAGGTGTTCTATACGATTGTTGACT
ACGTCAATCAGATTGTGCAGGCCATGTCAAAGCGGTCCAGATC
CCTGAACTCAGCAGCCTTCTATTATCGCGTTTAG
7-1a + 1b_WI78-8_VP7_AA
SEQ ID NO: 52
MDYIIYRSLLIYVALFALTRAQNYGLNLPITGSMDTVYANSTQ
EGIFLTSTLCLYYPTEAATEINDNSWKDTLSQLFLTKGWPTES
IYEKDYTDIASFSVDPQLYCDYNLVLMKYDATLQLDMSELADL
LLNEWLCNPMDITLYYYQQSGESNKWISMGSSCTVKVCPLNTQ
MLGIGCQTTNVDSFEMVAENEKLAIVDVVDGINHKINLTTTTC
TIRNCKKLGPRENVAVIQVGGSDVLDITADPTTAPQTERMMRV
NWKKWWQVFYTIVDYVNQIVQAMSKRSRSLNSAAFYYRV
IF-(160)RVA(G12P8KDH651)VP7.c
SEQ ID NO: 53
TCGTGCTTCGGCACCAGTACAATGGATTTCATCATCTACCGC
TTCCTCCTA
IF-RVA(G12P8KDH651)VP7.r
SEQ ID NO: 54
ACTAAAGAAAATAGGCCTTCAGATCCTGTAGTAAAAGGCAGC
GGAGTTCAGG
KDH651_VP7_DNA_Opt
SEQ ID NO: 55
ATGGATTTCATCATCTACCGCTTCCTCCTAATAGTAGTGATCAT
CCTGCCCTTCATTAAAGCACAGAACTATGGGATCAACCTGCCCA
TCACAGGCTCTATGGATGCCGCGTACGTGAATTCAACACAACAG
GAAAATTTCATGACCTCCACACTTTGTCTTTACTATCCGAGTAG
CGTGACTACTGAAATCACAGATCCCGATTGGACCAATACCCTGA
GCCAGCTGTTTCTAACCAAGGGATGGCCCGTGAACTCTGTGTAT
TTTAAGAGCTATGCAGATATTTCTTCATTTTCGGTGGACCCCCA
GCTTTATTGCGACTACAACATAGTGCTGATACAGTACCAGAACT
CGCTGGCTTTGGATGTTAGTGAACTGGCTGACCTGATCCTGAAT
GAATGGTTGTGCAACCCTATGGACGTGACACTCTACTACTACCA
ACAGACAGATGAGGCAAACAAGTGGATCTCGATGGGAGAATCTT
GCACAGTCAAAGTCTGCCCCCTCAACACCCAAACCCTGGGTATT
GGATGCACGACTACCGATGTGACAACCTTCGAAGAAGTGGCGAA
TGCCGAGAAACTTGTGATCACCGATGTGGTTGACGGCGTGAACC
ATAAAATTAACATCACCGTCAACACATGTACTATCAGGAATTGC
AAAAAACTCGGTCCAAGGGAGAACGTCGCCATCATACAGGTGGG
GAGTTCAGATGTCATCGATATCACCGCCGACCCCACAACCATCC
CGCAGACCGAGCGAATGATGAGAATCAATTGGAAAAAATGGTGG
CAAGTATTTTACACAGTCGTGGATTATATTAATCAGATCGTGCA
GGTTATGAGCAAAAGGTCAAGAAGCCTGAACTCCGCTGCCTTTT
ACTACAGGATCTGA
KDH651_VP7_AA
SEQ ID NO: 56
MDFIIYRELLIVVIILPFIKAQNYGINLPITGSMDAAYVNSTQ
QENFMTSTLCLYYPSSVTTEITDPDWTNTLSQLFLTKGWPVNS
VYEKSYADISSFSVDPQLYCDYNIVLIQYQNSLALDVSELADL
ILNEWLCNPMDVTLYYYQQTDEANKWISMGESCTVKVCPLNTQ
TLGIGCTTTDVTTFEEVANAEKLVITDVVDGVNHKINITVNTC
TIRNCKKLGPRENVAIIQVGSSDVIDITADPTTIPQTERMMRI
NWKKWWQVFYTVVDYINQIVQVMSKRSRSLNSAAFYYRI
IF-VP7(3End)Rtx + VP7-1b(G12P8).r
SEQ ID NO: 57
ACTAAAGAAAATAGGCCTCTAAACGCGATAATAGAAGGCTGCT
GAGTTCAGGGATCTGGACCGTTTGCTCATAACCTGCACGAT
7-1b_KDH651_VP7_DNA_Opt
SEQ ID NO: 58
ATGGATTATATTATCTATCGTAGCCTCCTCATCTACGTGGCCC
TTTTTGCCCTGACCAGGGCCCAGAACTATGGCCTGAACTTACC
AATCACCGGTTCAATGGATACCGTTTACGCTAATTCCACTCAA
GAGGGGATATTTCTGACCTCCACACTTTGTCTTTACTATCCGA
GTAGCGTGACTACTGAAATCACAGATCCCGATTGGACCAATAC
CCTGAGCCAGCTGTTTCTAACCAAGGGATGGCCCGTGAACTCT
GTGTATTTTAAGAGCTATGCAGATATTTCTTCATTTTCGGTGG
ACCCCCAGCTTTATTGCGACTACAACATAGTGCTGATACAGTA
CCAGAACTCGCTGGCTTTGGATGTTAGTGAACTGGCTGACCTG
ATCCTGAATGAGTGGCTGTGCAACCCCATGGACATCACATTAT
ATTACTACCAGCAGTCTGGAGAATCCAACAAGTGGATCAGTAT
GGGCTCAAGTTGCACCGTGAAGGTGTGTCCCTTGAACACCCAA
ATGCTGGGCATTGGTTGTCAGACAACTAATGTGGATTCGTTTG
AAATGGTAGCCGAAAACGAGAAGCTGGCTATAGTGGACGTAGT
CGATGGGATTAACCACAAGATCAATCTGACTACCACCACTTGT
ACCATCAGAAACTGTAAAAAGCTCGGCCCCCGGGAGAACGTCG
CCATCATACAGGTGGGGAGTTCAGATGTCATCGATATCACCGC
CGACCCCACAACCATCCCGCAGACCGAGCGAATGATGAGAATC
AATTGGAAAAAATGGTGGCAAGTATTTTACACAGTCGTGGATT
ATATTAATCAGATCGTGCAGGTTATGAGCAAACGGTCCAGATC
CCTGAACTCAGCAGCCTTCTATTATCGCGTTTAG
7-1b_KDH651_VP7_AA
SEQ ID NO: 59
MDYIIYRSLLIYVALFALTRAQNYGLNLPITGSMDTVYANSTQ
EGIFLTSTLCLYYPSSVTTEITDPDWTNTLSQLFLTKGWPVNS
VYEKSYADISSFSVDPQLYCDYNIVLIQYQNSLALDVSELADL
ILNEWLCNPMDITLYYYQQSGESNKWISMGSSCTVKVCPLNTQ
MLGIGCQTTNVDSFEMVAENEKLAIVDVVDGINHKINLTTTTC
TIRNCKKLGPRENVAIIQVGSSDVIDITADPTTIPQTERMMRI
NWKKWWQVFYTVVDYINQIVQVMSKRSRSLNSAAFYYRV
7-1a + 1b_KDH651_VP7_DNA_Opt
SEQ ID NO: 60
ATGGATTATATTATCTATCGTAGCCTCCTCATCTACGTGGCCC
TTTTTGCCCTGACCAGGGCCCAGAACTATGGCCTGAACTTACC
AATCACCGGTTCAATGGATACCGTTTACGCTAATTCCACTCAA
GAGGGGATATTTCTGACCTCCACACTTTGTCTTTACTATCCGA
GTAGCGTGACTACTGAAATCACAGATCCCGATTGGACCAATAC
CCTGAGCCAGCTGTTTCTAACCAAGGGATGGCCCGTGAACTCT
GTGTATTTTAAGAGCTATGCAGATATTTCTTCATTTTCGGTGG
ACCCCCAGCTTTATTGCGACTACAACATAGTGCTGATACAGTA
CCAGAACTCGCTGGCTTTGGATGTTAGTGAACTGGCTGACCTG
ATCCTGAATGAGTGGCTGTGCAACCCCATGGACATCACATTAT
ATTACTACCAGCAGTCTGGAGAATCCAACAAGTGGATCAGTAT
GGGCTCAAGTTGCACCGTGAAGGTGTGTCCCTTGAACACCCAA
ATGCTGGGCATTGGTTGTCAGACAACTAATGTGGATTCGTTTG
AAATGGTAGCCGAAAACGAGAAGCTGGCTATAGTGGACGTAGT
CGATGGGATTAACCACAAGATCAATCTGACTACCACCACTTGT
ACCATCAGAAACTGTAAAAAGCTCGGCCCCCGGGAGAACGTCG
CCATCATACAGGTGGGGAGTTCAGATGTCATCGATATCACCGC
CGACCCCACAACCATCCCGCAGACCGAGCGAATGATGAGAATC
AATTGGAAAAAATGGTGGCAAGTATTTTACACAGTCGTGGATT
ATATTAATCAGATCGTGCAGGTTATGAGCAAACGGTCCAGATC
CCTGAACTCAGCAGCCTTCTATTATCGCGTTTAG
7-1a + 1b_KDH651_VP7_AA
SEQ ID NO: 61
MDYIIYRSLLIYVALFALTRAQNYGLNLPITGSMDTVYANST
QEGIFLTSTLCLYYPSSVTTEITDPDWTNTLSQLFLTKGWPV
NSVYEKSYADISSFSVDPQLYCDYNIVLIQYQNSLALDVSEL
ADLILNEWLCNPMDITLYYYQQSGESNKWISMGSSCTVKVCP
LNTQMLGIGCQTTNVDSFEMVAENEKLAIVDVVDGINHKINL
TTTTCTIRNCKKLGPRENVAIIQVGSSDVIDITADPTTIPQT
ERMMRINWKKWWQVFYTVVDYINQIVQVMSKRSRSLNSAAFY
YRV
IF-VP7(G3AAA18522).c
SEQ ID NO: 62
TCGTGCTTCGGCACCAGTACAATGGATTTCATCATATATAGG
TTTCTGTTT
IF-VP7(G3AAA18522).r
SEQ ID NO: 63
ACTAAAGAAAATAGGCCTTTACACCCTATAATAGAATGCAGC
GGAGTTAAGG
G3HCR3_VP7_DNA_opt
SEQ ID NO: 117
ATGGATTTCATCATATATAGGTTTCTGTTTATAATAGTGATT
CTGTCACCTCTACTCAAGGCGCAAAACTATGGCATAAACCTC
CCTATCACCGGCTCAATGGACACCGCCTATGCAAACTCCACG
CAGGAAGAAACTCTGCTGACCAGCACACTCTGCCTCTACTAC
CCGACAGAGGCTGCCACTGAGATCAACGATAATTCTTGGAAA
GACACGTTATCGCAGCTGTTTCTTACTAAGGGCTGGCCCACC
GGTAGTGTCTACTTTAAAGAGTATACCGACATTGCCTCTTTT
AGCGTGGATCCTCAGCTCTACTGTGACTATAACATCGTGTTG
ATGAAGTATGACGCAGCGCTGCAGCTGGATATGAGTGAGCTG
GCCGATTTGATCCTGAATGAGTGGCTGTGTAATCCAATGGAT
ATCACACTCTACTACTACCAGCAGACTGACGAAGCCAACAAG
TGGATCTCTATGGGTTCTAGCTGCACCATCAAAGTGTGCCCC
CTGAACACCCAGACACTGGGCATTGGCTGTCTGACGACAGAT
GTCAGTACCTTCGAGGAGGTGGCGACAACAGAGAAACTGGTG
ATCACCGACGTGGTTGACGGCGTGAACCACAAACTCGACGTG
ACAACTACCACCTGCACCATCCGGAATTGTAAGAAGCTGGGA
CCGAGAGAAAATGTTGCAGTCATCCAGGTAGGAGGCAGTGAT
ATTCTCGACATCACGGCCGACCCGACGACCGCGCCTCAGACA
GAAAGGATGATGCGGATCAATTGGAAGAAGTGGTGGCAGGTG
TTCTACACAGTGGTGGACTACGTTAACCAGATTATTCAGGCT
ATGAGCAAGCGCAGCAGATCCCTTAACTCCGCTGCATTCTAT
TATAGGGTGTAA
G3HCR3_VP7_AA
SEQ ID NO: 77
MDFIIYRELFIIVILSPLLKAQNYGINLPITGSMDTAYANST
QEETLLTSTLCLYYPTEAATEINDNSWKDTLSQLFLTKGWPT
GSVYFKEYTDIASFSVDPQLYCDYNIVLMKYDAALQLDMSEL
ADLILNEWLCNPMDITLYYYQQTDEANKWISMGSSCTIKVCP
LNTQTLGIGCLTTDVSTFEEVATTEKLVITDVVDGVNHKLDV
TTTTCTIRNCKKLGPRENVAVIQVGGSDILDITADPTTAPQT
ERMMRINWKKWWQVFYTVVDYVNQIIQAMSKRSRSLNSAAFY
YRV*
G3HCR3 + 7-1a-1b_G1Rtx_VP7_DNA_opt
SEQ ID NO: 64
ATGGATTTCATCATATATAGGTTTCTGTTTATAATAGTGATT
CTGTCACCTCTACTCAAGGCGCAAAACTATGGCATAAACCTC
CCTATCACCGGCTCAATGGACACCGCCTATGCAAACTCCACG
CAGGAAGAAACTCTGCTGACAAGTACCCTGTGCCTGTATTAT
CCAACAGAAGCCTCTACCCAGATCAATGATGGGGAGTGGAAG
GATAGTCTCTCACAGATGTTCCTAACCAAGGGCTGGCCCACC
GGTTCCGTCTACTTCAAGGAATACTCTAGTATTGTCGACTTC
TCAGTTGACCCCCAGCTTTATTGCGACTACAACCTGGTACTT
ATGAAATACGACCAGAACCTGGAGCTGGATATGTCCGAGCTG
GCTGACCTGATCCTCAATGAGTGGCTGTGTAATCCAATGGAT
ATCACACTCTACTACTACCAGCAGACTGACGAAGCCAACAAG
TGGATCTCTATGGGTTCTAGCTGCACCATCAAAGTGTGCCCC
CTGAACACCCAGACACTGGGCATTGGCTGTCTGACGACAGAT
GTCAGTACCTTCGAGGAGGTGGCGACAACAGAGAAACTGGTG
ATCACCGACGTGGTTGACGGCGTGAACCACAAACTCGACGTG
ACAACTACCACCTGCACCATCCGGAATTGTAAGAAGCTGGGA
CCGAGAGAAAACGTCGCCGTGATCCAGGTGGGGGGGAGCAAT
GTGCTCGACATTACTGCCGACCCTACCACCAATCCACAGACG
GAACGGATGATGAGAGTCAACTGGAAGAAATGGTGGCAGGTC
TTTTATACCATTGTGGACTACATTAACCAGATTGTGCAAGTC
ATGAGTAAACGCAGCAGATCCCTTAACTCCGCTGCATTCTAT
TATAGGGTGTAA
G3HCR3 + 7-1a-1b_G1Rtx_VP7_AA
SEQ ID NO: 65
MDFIIYRELFIIVILSPLLKAQNYGINLPITGSMDTAYANST
QEETLLTSTLCLYYPTEASTQINDGEWKDSLSQMFLTKGWPT
GSVYFKEYSSIVDFSVDPQLYCDYNLVLMKYDQNLELDMSEL
ADLILNEWLCNPMDITLYYYQQTDEANKWISMGSSCTIKVCP
LNTQTLGIGCLTTDVSTFEEVATTEKLVITDVVDGVNHKLDV
TTTTCTIRNCKKLGPRENVAVIQVGGSNVLDITADPTTNPQT
ERMMRVNWKKWWQVFYTIVDYINQIVQVMSKRSRSLNSAAFY
YRV*
G3HCR3 + 7-1a-1b_G2Sc2-9_VP7_DNA_opt
SEQ ID NO: 66
ATGGATTTCATCATATATAGGTTTCTGTTTATAATAGTGATT
CTGTCACCTCTACTCAAGGCGCAAAACTATGGCATAAACCTC
CCTATCACCGGCTCAATGGACACCGCCTATGCAAACTCCACG
CAGGAAGAAACTCTGCTGACAAGCACCCTTTGCCTTTATTAC
CCAGCAGAAGCAAAGAATGAAATTAGCGACGATGAGTGGGAG
AATACACTTTCACAGCTGTTTCTCACCAAGGGGTGGCCAACC
GGTAGCGTATACTTCAAAGACTATAACGACATTACGACCTTT
AGTATGAACCCTCAGCTCTACTGTGACTATAACGTCGTGTTA
ATGCGCTATGACAATACCAGCGAGCTCGACGCCTCTGAGCTG
GCTGACCTGATCCTGAATGAGTGGCTGTGTAATCCAATGGAT
ATCACACTCTACTACTACCAGCAGACTGACGAAGCCAACAAG
TGGATCTCTATGGGTTCTAGCTGCACCATCAAAGTGTGCCCC
CTGAACACCCAGACACTGGGCATTGGCTGTCTGACGACAGAT
GTCAGTACCTTCGAGGAGGTGGCGACAACAGAGAAACTGGTG
ATCACCGACGTGGTTGACGGCGTGAACCACAAACTCGACGTG
ACAACTACCACCTGCACCATCCGGAATTGTAAGAAGCTGGGA
CCGAGAGAAAACGTGGCCATTATCCAGGTTGGCGGCCCTAAC
GCGCTCGACATCACTGCAGATCCAACAACCGTGCCTCAAATT
CAGCGGATTATGAGAATCAATTGGAAAAAGTGGTGGCAGGTG
TTTTATACGGTTGTGGACTATATTAATCAGATCGTACAGGTG
ATGAGCAAACGCAGCAGATCCCTTAACTCCGCTGCATTCTAT
TATAGGGTGTAA
G3HCR3 + 7-1a-1b_G2Sc2-9_VP7_AA
SEQ ID NO: 67
MDFIIYRELFIIVILSPLLKAQNYGINLPITGSMDTAYANST
QEETLLTSTLCLYYPAEAKNEISDDEWENTLSQLFLTKGWPT
GSVYFKDYNDITTESMNPQLYCDYNVVLMRYDNTSELDASEL
ADLILNEWLCNPMDITLYYYQQTDEANKWISMGSSCTIKVCP
LNTQTLGIGCLTTDVSTFEEVATTEKLVITDVVDGVNHKLDV
TTTTCTIRNCKKLGPRENVAIIQVGGPNALDITADPTTVPQI
QRIMRINWKKWWQVFYTVVDYINQIVQVMSKRSRSLNSAAFY
YRV*
G3HCR3 + 7-1a-1b_G4Brb-9_VP7_DNA_opt
SEQ ID NO: 68
ATGGATTTCATCATATATAGGTTTCTGTTTATAATAGTGATT
CTGTCACCTCTACTCAAGGCGCAAAACTATGGCATAAACCTC
CCTATCACCGGCTCAATGGACACCGCCTATGCAAACTCCACG
CAGGAAGAAACTCTGCTGTCTAGCACACTGTGCCTTTACTAT
CCTAGTGAGGCACCGACTCAAATCAGTGATACAGAATGGAAG
GATACACTGTCTCAACTCTTTCTCACCAAGGGATGGCCCACT
GGCTCAGTGTATTTTAATGAATACAGCAACGTTTTGGAGTTC
AGTATTGACCCCAAGCTGTACTGCGACTACAATGTAGTGCTG
ATTCGATTCGCCTCGGGGGAGGAACTTGACGTATCCGAGTTG
GCCGACCTCATCCTGAATGAGTGGCTGTGTAATCCAATGGAT
ATCACACTCTACTACTACCAGCAGACTGACGAAGCCAACAAG
TGGATCTCTATGGGTTCTAGCTGCACCATCAAAGTGTGCCCC
CTGAACACCCAGACACTGGGCATTGGCTGTCTGACGACAGAT
GTCAGTACCTTCGAGGAGGTGGCGACAACAGAGAAACTGGTG
ATCACCGACGTGGTTGACGGCGTGAACCACAAACTCGACGTG
ACAACTACCACCTGCACCATCCGGAATTGTAAGAAGCTGGGA
CCGAGAGAAAACGTTGCCATAATCCAGGTGGGAGGTAGCAAT
ATCCTCGACATAACCGCCGATCCTACGACGTCCCCTCAGACT
GAAAGGATGATGCGAGTCAACTGGAAGAAGTGGTGGCAAGTT
TTCTATACAGTGGTTGACTATATCAACCAAATAGTCAAGGTG
ATGAGTAAACGCAGCAGATCCCTTAACTCCGCTGCATTCTAT
TATAGGGTGTAA
G3HCR3 + 7-1a-1b_G4Brb-9_VP7_AA
SEQ ID NO: 69
MDFIIYRELFIIVILSPLLKAQNYGINLPITGSMDTAYANST
QEETLLSSTLCLYYPSEAPTQISDTEWKDTLSQLFLTKGWPT
GSVYFNEYSNVLEFSIDPKLYCDYNVVLIRFASGEELDVSEL
ADLILNEWLCNPMDITLYYYQQTDEANKWISMGSSCTIKVCP
LNTQTLGIGCLTTDVSTFEEVATTEKLVITDVVDGVNHKLDV
TTTTCTIRNCKKLGPRENVAIIQVGGSNILDITADPTTSPQT
ERMMRVNWKKWWQVFYTVVDYINQIVKVMSKRSRSLNSAAFY
YRV*
G3HCR3 + 7-1a-1b_G9BE2001_VP7_DNA_opt
SEQ ID NO: 70
ATGGATTTCATCATATATAGGTTTCTGTTTATAATAGTGATTCT
GTCACCTCTACTCAAGGCGCAAAACTATGGCATAAACCTCCCTA
TCACCGGCTCAATGGACACCGCCTATGCAAACTCCACGCAGGAA
GAAACTCTGCTGACATCAACCTTGTGCTTGTATTACCCCACTGA
AGCGTCTACTCAGATCGGAGATACCGAGTGGAAAGATACTCTCA
GTCAGCTGTTCCTCACCAAGGGATGGCCAACAGGCTCTGTCTAC
TTTAAAGAGTACACGGACATCGCATCTTTTAGCATCGATCCTCA
GTTATACTGCGACTACAACGTGGTGTTGATGAAATACGACAGCA
CGCTGGAGCTCGACATGTCCGAGCTGGCTGATCTGATTCTCAAT
GAGTGGCTGTGTAATCCAATGGATATCACACTCTACTACTACCA
GCAGACTGACGAAGCCAACAAGTGGATCTCTATGGGTTCTAGCT
GCACCATCAAAGTGTGCCCCCTGAACACCCAGACACTGGGCATT
GGCTGTCTGACGACAGATGTCAGTACCTTCGAGGAGGTGGCGAC
AACAGAGAAACTGGTGATCACCGACGTGGTTGACGGCGTGAACC
ACAAACTCGACGTGACAACTACCACCTGCACCATCCGGAATTGT
AAGAAGCTGGGACCGAGAGAAAACGTGGCTATCGTTCAGGTGGG
CGGTTCCGAGGTTCTCGACATAACGGCTGACCCAACCACCGCCC
CACAGACCGAGAGGATGATGCGCGTGAACTGGAAAAAATGGTGG
CAAGTGTTCTACACTGTGGTGGACTATATCAACCAGATTGTGCA
GGTGATGTCCAAACGCAGCAGATCCCTTAACTCCGCTGCATTCT
ATTATAGGGTGTAA
G3HCR3 + 7-1a-1b_G9BE2001_VP7_AA
SEQ ID NO: 71
MDFIIYRELFIIVILSPLLKAQNYGINLPITGSMDTAYANSTQE
ETLLTSTLCLYYPTEASTQIGDTEWKDTLSQLFLTKGWPTGSVY
FKEYTDIASFSIDPQLYCDYNVVLMKYDSTLELDMSELADLILN
EWLCNPMDITLYYYQQTDEANKWISMGSSCTIKVCPLNTQTLGI
GCLTTDVSTFEEVATTEKLVITDVVDGVNHKLDVTTTTCTIRNC
KKLGPRENVAIVQVGGSEVLDITADPTTAPQTERMMRVNWKKWW
QVFYTVVDYINQIVQVMSKRSRSLNSAAFYYRV*
G3HCR3 + 7-1a-1b_G1 2K12_VP7_DNA_opt
SEQ ID NO: 72
ATGGATTTCATCATATATAGGTTTCTGTTTATAATAGTGATTCT
GTCACCTCTACTCAAGGCGCAAAACTATGGCATAAACCTCCCTA
TCACCGGCTCAATGGACACCGCCTATGCAAACTCCACGCAGGAA
GAAACTCTGCTGACCTCTACCTTGTGTCTGTATTACCCCTCTTC
TGTCACAACAGAGATCACAGATCCTGATTGGACCAATACATTGT
CTCAGCTCTTCATGACCAAAGGGTGGCCTACTAACAGCGTGTAT
TTCAAGTCATATGCGGACATCGCTAGCTTCAGCGTTGATCCACA
GCTCTATTGCGACTACAACATCGTTCTGGTTCAGTACCAAAATT
CCCTGGCCTTAGACGTGTCTGAACTCGCCGACCTGATCCTGAAT
GAGTGGCTGTGTAATCCAATGGATATCACACTCTACTACTACCA
GCAGACTGACGAAGCCAACAAGTGGATCTCTATGGGTTCTAGCT
GCACCATCAAAGTGTGCCCCCTGAACACCCAGACACTGGGCATT
GGCTGTCTGACGACAGATGTCAGTACCTTCGAGGAGGTGGCGAC
AACAGAGAAACTGGTGATCACCGACGTGGTTGACGGCGTGAACC
ACAAACTCGACGTGACAACTACCACCTGCACCATCCGGAATTGT
AAGAAGCTGGGACCGAGAGAAAACGTCGCGATAATACAAGTGGG
TGGCTCTGATGTTATCGATATAACAGCTGATCCCACAACGATTC
CACAGACAGAGCGGATGATGCGGATCAACTGGAAGAAGTGGTGG
CAGGTTTTTTACACCGTGGTCGATTACATCAACCAGATCGTACA
GGTGATGAGCAAGCGCAGCAGATCCCTTAACTCCGCTGCATTCT
ATTATAGGGTGTAA
G3HCR3 + 7-1a-1b_G12K12_VP7_AA
SEQ ID NO: 73
MDFIIYRELFIIVILSPLLKAQNYGINLPITGSMDTAYANSTQE
ETLLTSTLCLYYPSSVTTEITDPDWTNTLSQLFMTKGWPTNSVY
EKSYADIASFSVDPQLYCDYNIVLVQYQNSLALDVSELADLILN
EWLCNPMDITLYYYQQTDEANKWISMGSSCTIKVCPLNTQTLGI
GCLTTDVSTFEEVATTEKLVITDVVDGVNHKLDVTTTTCTIRNC
KKLGPRENVAIIQVGGSDVIDITADPTTIPQTERMMRINWKKWW
QVFYTVVDYINQIVQVMSKRSRSLNSAAFYYRV*
IF-(160)RVA(G4P5BrB-9)VP7.c
SEQ ID NO: 74
TCGTGCTTCGGCACCAGTACAATGGATTATCTGATCTACCGCA
TCACCTTTGTG
IF-RVA(G4P5BrB-9)VP7.r
SEQ ID NO: 75
ACTAAAGAAAATAGGCCTTTAAACTCTGTAGTAAAAGCTACTGG
AGTC
G4BrB-9_VP7_DNA_opt
SEQ ID NO: 76
ATGGATTATCTGATCTACCGCATCACCTTTGTGATTGTCGTTCT
CTCTGTGTTATCAAATGCTCAGAATTACGGCATCAACCTGCCAA
TTACCGGCTCCATGGACACAGCCTACGCTAACTCCACCCAGGAC
AACAACTTTTTGTCTAGCACACTGTGCCTTTACTATCCTAGTGA
GGCACCGACTCAAATCAGTGATACAGAATGGAAGGATACACTGT
CTCAACTCTTTCTCACCAAGGGATGGCCCACTGGCTCAGTGTAT
TTTAATGAATACAGCAACGTTTTGGAGTTCAGTATTGACCCCAA
GCTGTACTGCGACTACAATGTAGTGCTGATTCGATTCGCCTCGG
GGGAGGAACTTGACGTATCCGAGTTGGCCGACCTCATCCTGAAT
GAATGGCTTTGTAATCCTATGGACATTACGCTGTACTATTACCA
GCAGACCGGCGAGGCCAACAAATGGATCTCGATGGGGAGCAGCT
GCACTGTGAAGGTGTGTCCCCTGAACACCCAGACTCTCGGTATC
GGGTGCCAGACAACTGATACCGCAACTTTTGAGACAGTGGCAGA
TAGCGAGAAGCTGGCCCTAATTGATGTGGTGGATAATGTGAACC
ACAAGCTGGACGTAACATCGACAACCTGTACTATCCGAAACTGT
AACAAACTTGGGCCACGAGAGAACGTTGCCATAATCCAGGTGGG
AGGTAGCAATATCCTCGACATAACCGCCGATCCTACGACGTCCC
CTCAGACTGAAAGGATGATGCGAGTCAACTGGAAGAAGTGGTGG
CAAGTTTTCTATACAGTGGTTGACTATATCAACCAAATAGTCAA
GGTGATGAGTAAAAGATCCCGATCCCTAGACTCCAGTAGCTTTT
ACTACAGAGTTTAA
G4BrB-9_VP7_AA
SEQ ID NO: 78
MDYLIYRITEVIVVLSVLSNAQNYGINLPITGSMDTAYANSTQD
NNELSSTLCLYYPSEAPTQISDTEWKDTLSQLFLTKGWPTGSVY
FNEYSNVLEFSIDPKLYCDYNVVLIRFASGEELDVSELADLILN
EWLCNPMDITLYYYQQTGEANKWISMGSSCTVKVCPLNTQTLGI
GCQTTDTATFETVADSEKLALIDVVDNVNHKLDVTSTTCTIRNC
NKLGPRENVAIIQVGGSNILDITADPTTSPQTERMMRVNWKKWW
QVFYTVVDYINQIVKVMSKRSRSLDSSSFYYRV*
G4BrB-9 + 7-1a-1b_G1Rtx_VP7_DNA_opt
SEQ ID NO: 79
ATGGATTATCTGATCTACCGCATCACCTTTGTGATTGTCGTTCT
CTCTGTGTTATCAAATGCTCAGAATTACGGCATCAACCTGCCAA
TTACCGGCTCCATGGACACAGCCTACGCTAACTCCACCCAGGAC
AACAACTTTTTGACAAGTACCCTGTGCCTGTATTATCCAACAGA
AGCCTCTACCCAGATCAATGATGGGGAGTGGAAGGATAGTCTCT
CACAGATGTTCCTAACCAAGGGCTGGCCCACCGGTTCCGTCTAC
TTCAAGGAATACTCTAGTATTGTCGACTTCTCAGTTGACCCCCA
GCTTTATTGCGACTACAACCTGGTACTTATGAAATACGACCAGA
ACCTGGAGCTGGATATGTCCGAGCTGGCTGACCTGATCCTCAAT
GAATGGCTTTGTAATCCTATGGACATTACGCTGTACTATTACCA
GCAGACCGGCGAGGCCAACAAATGGATCTCGATGGGGAGCAGCT
GCACTGTGAAGGTGTGTCCCCTGAACACCCAGACTCTCGGTATC
GGGTGCCAGACAACTGATACCGCAACTTTTGAGACAGTGGCAGA
TAGCGAGAAGCTGGCCCTAATTGATGTGGTGGATAATGTGAACC
ACAAGCTGGACGTAACATCGACAACCTGTACTATCCGAAACTGT
AACAAACTTGGGCCACGAGAGAACGTCGCCGTGATCCAGGTGGG
GGGGAGCAATGTGCTCGACATTACTGCCGACCCTACCACCAATC
CACAGACGGAACGGATGATGAGAGTCAACTGGAAGAAATGGTGG
CAGGTCTTTTATACCATTGTGGACTACATTAACCAGATTGTGCA
AGTCATGAGTAAAAGATCCCGATCCCTAGACTCCAGTAGCTTTT
ACTACAGAGTTTAA
G4BrB-9 + 7-1a-1b_G1Rtx_VP7_AA
SEQ ID NO: 80
MDYLIYRITEVIVVLSVLSNAQNYGINLPITGSMDTAYANSTQD
NNFLTSTLCLYYPTEASTQINDGEWKDSLSQMFLTKGWPTGSVY
FKEYSSIVDFSVDPQLYCDYNLVLMKYDQNLELDMSELADLILN
EWLCNPMDITLYYYQQTGEANKWISMGSSCTVKVCPLNTQTLGI
GCQTTDTATFETVADSEKLALIDVVDNVNHKLDVTSTTCTIRNC
NKLGPRENVAVIQVGGSNVLDITADPTTNPQTERMMRVNWKKWW
QVFYTIVDYINQIVQVMSKRSRSLDSSSFYYRV*
G4BrB-9 + 7-1a-1b_G2Sc2-9_VP7_DNA_opt
SEQ ID NO: 81
ATGGATTATCTGATCTACCGCATCACCTTTGTGATTGTCGTTCT
CTCTGTGTTATCAAATGCTCAGAATTACGGCATCAACCTGCCAA
TTACCGGCTCCATGGACACAGCCTACGCTAACTCCACCCAGGAC
AACAACTTTTTGACAAGCACCCTTTGCCTTTATTACCCAGCAGA
AGCAAAGAATGAAATTAGCGACGATGAGTGGGAGAATACACTTT
CACAGCTGTTTCTCACCAAGGGGTGGCCAACCGGTAGCGTATAC
TTCAAAGACTATAACGACATTACGACCTTTAGTATGAACCCTCA
GCTCTACTGTGACTATAACGTCGTGTTAATGCGCTATGACAATA
CCAGCGAGCTCGACGCCTCTGAGCTGGCTGACCTGATCCTGAAT
GAATGGCTTTGTAATCCTATGGACATTACGCTGTACTATTACCA
GCAGACCGGCGAGGCCAACAAATGGATCTCGATGGGGAGCAGCT
GCACTGTGAAGGTGTGTCCCCTGAACACCCAGACTCTCGGTATC
GGGTGCCAGACAACTGATACCGCAACTTTTGAGACAGTGGCAGA
TAGCGAGAAGCTGGCCCTAATTGATGTGGTGGATAATGTGAACC
ACAAGCTGGACGTAACATCGACAACCTGTACTATCCGAAACTGT
AACAAACTTGGGCCACGAGAGAACGTGGCCATTATCCAGGTTGG
CGGCCCTAACGCGCTCGACATCACTGCAGATCCAACAACCGTGC
CTCAAATTCAGCGGATTATGAGAATCAATTGGAAAAAGTGGTGG
CAGGTGTTTTATACGGTTGTGGACTATATTAATCAGATCGTACA
GGTGATGAGCAAAAGATCCCGATCCCTAGACTCCAGTAGCTTTT
ACTACAGAGTTTAA
G4BrB-9 + 7-1a-1b_G2Sc2-9_VP7_AA
SEQ ID NO: 82
MDYLIYRITEVIVVLSVLSNAQNYGINLPITGSMDTAYANSTQD
NNFLTSTLCLYYPAEAKNEISDDEWENTLSQLFLTKGWPTGSVY
FKDYNDITTFSMNPQLYCDYNVVLMRYDNTSELDASELADLILN
EWLCNPMDITLYYYQQTGEANKWISMGSSCTVKVCPLNTQTLGI
GCQTTDTATFETVADSEKLALIDVVDNVNHKLDVTSTTCTIRNC
NKLGPRENVAIIQVGGPNALDITADPTTVPQIQRIMRINWKKWW
QVFYTVVDYINQIVQVMSKRSRSLDSSSFYYRV*
G4BrB-9 + 7-1a-1b_G3HCR3_VP7_DNA_opt
SEQ ID NO: 83
ATGGATTATCTGATCTACCGCATCACCTTTGTGATTGTCGTTCT
CTCTGTGTTATCAAATGCTCAGAATTACGGCATCAACCTGCCAA
TTACCGGCTCCATGGACACAGCCTACGCTAACTCCACCCAGGAC
AACAACTTTTTGACCAGCACACTCTGCCTCTACTACCCGACAGA
GGCTGCCACTGAGATCAACGATAATTCTTGGAAAGACACGTTAT
CGCAGCTGTTTCTTACTAAGGGCTGGCCCACCGGTAGTGTCTAC
TTTAAAGAGTATACCGACATTGCCTCTTTTAGCGTGGATCCTCA
GCTCTACTGTGACTATAACATCGTGTTGATGAAGTATGACGCAG
CGCTGCAGCTGGATATGAGTGAGCTGGCCGATTTGATCCTGAAT
GAATGGCTTTGTAATCCTATGGACATTACGCTGTACTATTACCA
GCAGACCGGCGAGGCCAACAAATGGATCTCGATGGGGAGCAGCT
GCACTGTGAAGGTGTGTCCCCTGAACACCCAGACTCTCGGTATC
GGGTGCCAGACAACTGATACCGCAACTTTTGAGACAGTGGCAGA
TAGCGAGAAGCTGGCCCTAATTGATGTGGTGGATAATGTGAACC
ACAAGCTGGACGTAACATCGACAACCTGTACTATCCGAAACTGT
AACAAACTTGGGCCACGAGAGAATGTTGCAGTCATCCAGGTAGG
AGGCAGTGATATTCTCGACATCACGGCCGACCCGACGACCGCGC
CTCAGACAGAAAGGATGATGCGGATCAATTGGAAGAAGTGGTGG
CAGGTGTTCTACACAGTGGTGGACTACGTTAACCAGATTATTCA
GGCTATGAGCAAGAGATCCCGATCCCTAGACTCCAGTAGCTTTT
ACTACAGAGTTTAA
G4BrB-9 + 7-1a-1b_G3HCR3_VP7_AA
SEQ ID NO: 84
MDYLIYRITEVIVVLSVLSNAQNYGINLPITGSMDTAYANSTQD
NNFLTSTLCLYYPTEAATEINDNSWKDTLSQLFLTKGWPTGSVY
FKEYTDIASFSVDPQLYCDYNIVLMKYDAALQLDMSELADLILN
EWLCNPMDITLYYYQQTGEANKWISMGSSCTVKVCPLNTQTLGI
GCQTTDTATFETVADSEKLALIDVVDNVNHKLDVTSTTCTIRNC
NKLGPRENVAVIQVGGSDILDITADPTTAPQTERMMRINWKKWW
QVFYTVVDYVNQIIQAMSKRSRSLDSSSFYYRV*
G4BrB-9 + 7-1a-1b_G9BE2001_VP7_DNA_opt
SEQ ID NO: 85
ATGGATTATCTGATCTACCGCATCACCTTTGTGATTGTCGTTCT
CTCTGTGTTATCAAATGCTCAGAATTACGGCATCAACCTGCCAA
TTACCGGCTCCATGGACACAGCCTACGCTAACTCCACCCAGGAC
AACAACTTTTTGACATCAACCTTGTGCTTGTATTACCCCACTGA
AGCGTCTACTCAGATCGGAGATACCGAGTGGAAAGATACTCTCA
GTCAGCTGTTCCTCACCAAGGGATGGCCAACAGGCTCTGTCTAC
TTTAAAGAGTACACGGACATCGCATCTTTTAGCATCGATCCTCA
GTTATACTGCGACTACAACGTGGTGTTGATGAAATACGACAGCA
CGCTGGAGCTCGACATGTCCGAGCTGGCTGATCTGATTCTCAAT
GAATGGCTTTGTAATCCTATGGACATTACGCTGTACTATTACCA
GCAGACCGGCGAGGCCAACAAATGGATCTCGATGGGGAGCAGCT
GCACTGTGAAGGTGTGTCCCCTGAACACCCAGACTCTCGGTATC
GGGTGCCAGACAACTGATACCGCAACTTTTGAGACAGTGGCAGA
TAGCGAGAAGCTGGCCCTAATTGATGTGGTGGATAATGTGAACC
ACAAGCTGGACGTAACATCGACAACCTGTACTATCCGAAACTGT
AACAAACTTGGGCCACGAGAGAACGTGGCTATCGTTCAGGTGGG
CGGTTCCGAGGTTCTCGACATAACGGCTGACCCAACCACCGCCC
CACAGACCGAGAGGATGATGCGCGTGAACTGGAAAAAATGGTGG
CAAGTGTTCTACACTGTGGTGGACTATATCAACCAGATTGTGCA
GGTGATGTCCAAAAGATCCCGATCCCTAGACTCCAGTAGCTTTT
ACTACAGAGTTTAA
G4BrB-9 + 7-1a-1b_G9BE2001_VP7_AA
SEQ ID NO: 86
MDYLIVRITEVIVVLSVLSNAQNYGINLPITGSMDTAYANSTQD
NNFLTSTLCLYYPTEASTQIGDTEWKDTLSQLFLTKGWPTGSVY
FKEYTDIASFSIDPQLYCDYNVVLMKYDSTLELDMSELADLILN
EWLCNPMDITLYYYQQTGEANKWISMGSSCTVKVCPLNTQTLGI
GCQTTDTATFETVADSEKLALIDVVDNVNHKLDVTSTTCTIRNC
NKLGPRENVAIVQVGGSEVLDITADPTTAPQTERMMRVNWKKWW
QVFYTVVDYINQIVQVMSKRSRSLDSSSFYYRV*
G4BrB-9 + 7-1a-1b_Gl2K12_VP7_DNA_opt
SEQ ID NO: 87
ATGGATTATCTGATCTACCGCATCACCTTTGTGATTGTCGTTCT
CTCTGTGTTATCAAATGCTCAGAATTACGGCATCAACCTGCCAA
TTACCGGCTCCATGGACACAGCCTACGCTAACTCCACCCAGGAC
AACAACTTTTTGACCTCTACCTTGTGTCTGTATTACCCCTCTTC
TGTCACAACAGAGATCACAGATCCTGATTGGACCAATACATTGT
CTCAGCTCTTCATGACCAAAGGGTGGCCTACTAACAGCGTGTAT
TTCAAGTCATATGCGGACATCGCTAGCTTCAGCGTTGATCCACA
GCTCTATTGCGACTACAACATCGTTCTGGTTCAGTACCAAAATT
CCCTGGCCTTAGACGTGTCTGAACTCGCCGACCTGATCCTGAAT
GAATGGCTTTGTAATCCTATGGACATTACGCTGTACTATTACCA
GCAGACCGGCGAGGCCAACAAATGGATCTCGATGGGGAGCAGCT
GCACTGTGAAGGTGTGTCCCCTGAACACCCAGACTCTCGGTATC
GGGTGCCAGACAACTGATACCGCAACTTTTGAGACAGTGGCAGA
TAGCGAGAAGCTGGCCCTAATTGATGTGGTGGATAATGTGAACC
ACAAGCTGGACGTAACATCGACAACCTGTACTATCCGAAACTGT
AACAAACTTGGGCCACGAGAGAACGTCGCGATAATACAAGTGGG
TGGCTCTGATGTTATCGATATAACAGCTGATCCCACAACGATTC
CACAGACAGAGCGGATGATGCGGATCAACTGGAAGAAGTGGTGG
CAGGTTTTTTACACCGTGGTCGATTACATCAACCAGATCGTACA
GGTGATGAGCAAGAGATCCCGATCCCTAGACTCCAGTAGCTTTT
ACTACAGAGTTTAA
G4BrB-9 + 7-1a-1b_G12K12_VP7_AA
SEQ ID NO: 88
MDYLIYRITEVIVVLSVLSNAQNYGINLPITGSMDTAYANSTQD
NNFLTSTLCLYYPSSVTTEITDPDWTNTLSQLFMTKGWPTNSVY
EKSVADIASFSVDPQLYCDYNIVLVQYQNSLALDVSELADLILN
EWLCNPMDITLYYYQQTGEANKWISMGSSCTVKVCPLNTQTLGI
GCQTTDTATFETVADSEKLALIDVVDNVNHKLDVTSTTCTIRNC
NKLGPRENVAIIQVGGSDVIDITADPTTIPQTERMMRINWKKWW
QVFYTVVDYINQIVQVMSKRSRSLDSSSFYYRV*
IF-VP7(G9AFJ11215)(opt).c
SEQ ID NO: 89
TCGTGCTTCGGCACCAGTACAATGGATTTCATCATCTACAGGTT
CCTGC
IF-VP7(G9AFJ11215)(opt).r
SEQ ID NO: 90
ACTAAAGAAAATAGGCCTTCACACTCGATAATAGAAGGCGGCTG
AGTTC
G9BE2001_VP7_DNA_opt
SEQ ID NO: 91
ATGGATTTCATCATCTACAGGTTCCTGCTTTTCATCGTTATTGT
GAGCCCCTTTGTGAAGACACAGAACTACGGCATCAACCTCCCAA
TTACCGGTTCGATGGACGCAGCCTACGCAAATTCCTCACAGCAG
GAGACCTTTCTCACATCAACCTTGTGCTTGTATTACCCCACTGA
AGCGTCTACTCAGATCGGAGATACCGAGTGGAAAGATACTCTCA
GTCAGCTGTTCCTCACCAAGGGATGGCCAACAGGCTCTGTCTAC
TTTAAAGAGTACACGGACATCGCATCTTTTAGCATCGATCCTCA
GTTATACTGCGACTACAACGTGGTGTTGATGAAATACGACAGCA
CGCTGGAGCTCGACATGTCCGAGCTGGCTGATCTGATTCTCAAC
GAGTGGCTTTGCAACCCGATGGATATCACCCTGTATTACTATCA
GCAGACCGACGAAGCCAATAAGTGGATTAGCATGGGGCAGTCCT
GCACTATTAAGGTGTGCCCCCTCAATACACAAACCCTCGGCATC
GGCTGCACTACCACCAACACCGCCACTTTTGAGGAGGTGGCTAC
ACGAGAAAAGCTCGTGATCACTGACGTGGTGGACGGCGTGAACC
ACAAGCTGGACGTCACCACCAACACATGTACCATACGCAACTGC
AAGAAGCTGGGACCCAGGGAAAACGTGGCTATCGTTCAGGTGGG
CGGTTCCGAGGTTCTCGACATAACGGCTGACCCAACCACCGCCC
CACAGACCGAGAGGATGATGCGCGTGAACTGGAAAAAATGGTGG
CAAGTGTTCTACACTGTGGTGGACTATATCAACCAGATTGTGCA
GGTGATGTCCAAACGGTCGCGGTCTCTGAACTCAGCCGCCTTCT
ATTATCGAGTGTGA
G9BE2001_VP7_AA
SEQ ID NO: 92
MDFIIYRELLFIVIVSPFVKTQNYGINLPITGSMDAAYANSSQQ
ETFLTSTLCLYYPTEASTQIGDTEWKDTLSQLFLTKGWPTGSVY
FKEYTDIASFSIDPQLYCDYNVVLMKYDSTLELDMSELADLILN
EWLCNPMDITLYYYQQTDEANKWISMGQSCTIKVCPLNTQTLGI
GCTTTNTATFEEVATREKLVITDVVDGVNHKLDVTTNTCTIRNC
KKLGPRENVAIVQVGGSEVLDITADPTTAPQTERMMRVNWKKWW
QVFYTVVDYINQIVQVMSKRSRSLNSAAFYYRV*
G9BE2001 + 7-1a-1b_G1Rtx_VP7_DNA_opt
SEQ ID NO: 93
ATGGATTTCATCATCTACAGGTTCCTGCTTTTCATCGTTATTGT
GAGCCCCTTTGTGAAGACACAGAACTACGGCATCAACCTCCCAA
TTACCGGTTCGATGGACGCAGCCTACGCAAATTCCTCACAGCAG
GAGACCTTTCTCACAAGTACCCTGTGCCTGTATTATCCAACAGA
AGCCTCTACCCAGATCAATGATGGGGAGTGGAAGGATAGTCTCT
CACAGATGTTCCTAACCAAGGGCTGGCCCACCGGTTCCGTCTAC
TTCAAGGAATACTCTAGTATTGTCGACTTCTCAGTTGACCCCCA
GCTTTATTGCGACTACAACCTGGTACTTATGAAATACGACCAGA
ACCTGGAGCTGGATATGTCCGAGCTGGCTGACCTGATCCTCAAC
GAGTGGCTTTGCAACCCGATGGATATCACCCTGTATTACTATCA
GCAGACCGACGAAGCCAATAAGTGGATTAGCATGGGGCAGTCCT
GCACTATTAAGGTGTGCCCCCTCAATACACAAACCCTCGGCATC
GGCTGCACTACCACCAACACCGCCACTTTTGAGGAGGTGGCTAC
ACGAGAAAAGCTCGTGATCACTGACGTGGTGGACGGCGTGAACC
ACAAGCTGGACGTCACCACCAACACATGTACCATACGCAACTGC
AAGAAGCTGGGACCCAGGGAAAACGTCGCCGTGATCCAGGTGGG
GGGGAGCAATGTGCTCGACATTACTGCCGACCCTACCACCAATC
CACAGACGGAACGGATGATGAGAGTCAACTGGAAGAAATGGTGG
CAGGTCTTTTATACCATTGTGGACTACATTAACCAGATTGTGCA
AGTCATGAGTAAACGGTCGCGGTCTCTGAACTCAGCCGCCTTCT
ATTATCGAGTGTGA
G9BE2001 + 7-1a-1b_G1Rtx_VP7_AA
SEQ ID NO: 94
MDFIIYRELLFIVIVSPFVKTQNYGINLPITGSMDAAYANSSQQ
ETFLTSTLCLYYPTEASTQINDGEWKDSLSQMFLTKGWPTGSVY
FKEYSSIVDFSVDPQLYCDYNLVLMKYDQNLELDMSELADLILN
EWLCNPMDITLYYYQQTDEANKWISMGQSCTIKVCPLNTQTLGI
GCTTTNTATFEEVATREKLVITDVVDGVNHKLDVTTNTCTIRNC
KKLGPRENVAVIQVGGSNVLDITADPTTNPQTERMMRVNWKKWW
QVFYTIVDYINQIVQVMSKRSRSLNSAAFYYRV*
G9BE2001 + 7-1a-1b_G2Sc2-9_VP7_DNA_opt
SEQ ID NO: 95
ATGGATTTCATCATCTACAGGTTCCTGCTTTTCATCGTTATTGT
GAGCCCCTTTGTGAAGACACAGAACTACGGCATCAACCTCCCAA
TTACCGGTTCGATGGACGCAGCCTACGCAAATTCCTCACAGCAG
GAGACCTTTCTCACAAGCACCCTTTGCCTTTATTACCCAGCAGA
AGCAAAGAATGAAATTAGCGACGATGAGTGGGAGAATACACTTT
CACAGCTGTTTCTCACCAAGGGGTGGCCAACCGGTAGCGTATAC
TTCAAAGACTATAACGACATTACGACCTTTAGTATGAACCCTCA
GCTCTACTGTGACTATAACGTCGTGTTAATGCGCTATGACAATA
CCAGCGAGCTCGACGCCTCTGAGCTGGCTGACCTGATCCTGAAC
GAGTGGCTTTGCAACCCGATGGATATCACCCTGTATTACTATCA
GCAGACCGACGAAGCCAATAAGTGGATTAGCATGGGGCAGTCCT
GCACTATTAAGGTGTGCCCCCTCAATACACAAACCCTCGGCATC
GGCTGCACTACCACCAACACCGCCACTTTTGAGGAGGTGGCTAC
ACGAGAAAAGCTCGTGATCACTGACGTGGTGGACGGCGTGAACC
ACAAGCTGGACGTCACCACCAACACATGTACCATACGCAACTGC
AAGAAGCTGGGACCCAGGGAAAACGTGGCCATTATCCAGGTTGG
CGGCCCTAACGCGCTCGACATCACTGCAGATCCAACAACCGTGC
CTCAAATTCAGCGGATTATGAGAATCAATTGGAAAAAGTGGTGG
CAGGTGTTTTATACGGTTGTGGACTATATTAATCAGATCGTACA
GGTGATGAGCAAACGGTCGCGGTCTCTGAAC
TCAGCCGCCTTCTATTATCGAGTGTGA
G9BE2001 + 7-1a-1b_G2Sc2-9_VP7_AA
SEQ ID NO: 96
MDFIIYRELLFIVIVSPFVKTQNYGINLPITGSMDAAYANSSQQ
ETFLTSTLCLYYPAEAKNEISDDEWENTLSQLFLTKGWPTGSVY
FKDYNDITTESMNPQLYCDYNVVLMRYDNTSELDASELADLILN
EWLCNPMDITLYYYQQTDEANKWISMGQSCTIKVCPLNTQTLGI
GCTTTNTATFEEVATREKLVITDVVDGVNHKLDVTTNTCTIRNC
KKLGPRENVAIIQVGGPNALDITADPTTVPQIQRIMRINWKKWW
QVFYTVVDYINQIVQVMSKRSRSLNSAAFYYRV*
G9BE2001 + 7-1a-1b_G3HCR3_VP7_DNA_opt
SEQ ID NO: 97
ATGGATTTCATCATCTACAGGTTCCTGCTTTTCATCGTTATTGT
GAGCCCCTTTGTGAAGACACAGAACTACGGCATCAACCTCCCAA
TTACCGGTTCGATGGACGCAGCCTACGCAAATTCCTCACAGCAG
GAGACCTTTCTCACCAGCACACTCTGCCTCTACTACCCGACAGA
GGCTGCCACTGAGATCAACGATAATTCTTGGAAAGACACGTTAT
CGCAGCTGTTTCTTACTAAGGGCTGGCCCACCGGTAGTGTCTAC
TTTAAAGAGTATACCGACATTGCCTCTTTTAGCGTGGATCCTCA
GCTCTACTGTGACTATAACATCGTGTTGATGAAGTATGACGCAG
CGCTGCAGCTGGATATGAGTGAGCTGGCCGATTTGATCCTGAAC
GAGTGGCTTTGCAACCCGATGGATATCACCCTGTATTACTATCA
GCAGACCGACGAAGCCAATAAGTGGATTAGCATGGGGCAGTCCT
GCACTATTAAGGTGTGCCCCCTCAATACACAAACCCTCGGCATC
GGCTGCACTACCACCAACACCGCCACTTTTGAGGAGGTGGCTAC
ACGAGAAAAGCTCGTGATCACTGACGTGGTGGACGGCGTGAACC
ACAAGCTGGACGTCACCACCAACACATGTACCATACGCAACTGC
AAGAAGCTGGGACCCAGGGAAAATGTTGCAGTCATCCAGGTAGG
AGGCAGTGATATTCTCGACATCACGGCCGACCCGACGACCGCGC
CTCAGACAGAAAGGATGATGCGGATCAATTGGAAGAAGTGGTGG
CAGGTGTTCTACACAGTGGTGGACTACGTTAACCAGATTATTCA
GGCTATGAGCAAGCGGTCGCGGTCTCTGAACTCAGCCGCCTTCT
ATTATCGAGTGTGA
G9BE2001 + 7-1a-1b_G3HCR3_VP7_AA
SEQ ID NO: 98
MDFIIYRELLFIVIVSPFVKTQNYGINLPITGSMDAAYANSSQQ
ETFLTSTLCLYYPTEAATEINDNSWKDTLSQLFLTKGWPTGSVY
FKEYTDIASFSVDPQLYCDYNIVLMKYDAALQLDMSELADLILN
EWLCNPMDITLYYYQQTDEANKWISMGQSCTIKVCPLNTQTLGI
GCTTTNTATFEEVATREKLVITDVVDGVNHKLDVTTNTCTIRNC
KKLGPRENVAVIQVGGSDILDITADPTTAPQTERMMRINWKKWW
QVFYTVVDYVNQIIQAMSKRSRSLNSAAFYYRV*
G9BE2001 + 7-1a-1b_G4BrB-9_VP7_DNA_opt
SEQ ID NO: 99
ATGGATTTCATCATCTACAGGTTCCTGCTTTTCATCGTTATTGT
GAGCCCCTTTGTGAAGACACAGAACTACGGCATCAACCTCCCAA
TTACCGGTTCGATGGACGCAGCCTACGCAAATTCCTCACAGCAG
GAGACCTTTCTCTCTAGCACACTGTGCCTTTACTATCCTAGTGA
GGCACCGACTCAAATCAGTGATACAGAATGGAAGGATACACTGT
CTCAACTCTTTCTCACCAAGGGATGGCCCACTGGCTCAGTGTAT
TTTAATGAATACAGCAACGTTTTGGAGTTCAGTATTGACCCCAA
GCTGTACTGCGACTACAATGTAGTGCTGATTCGATTCGCCTCGG
GGGAGGAACTTGACGTATCCGAGTTGGCCGACCTCATCCTGAAC
GAGTGGCTTTGCAACCCGATGGATATCACCCTGTATTACTATCA
GCAGACCGACGAAGCCAATAAGTGGATTAGCATGGGGCAGTCCT
GCACTATTAAGGTGTGCCCCCTCAATACACAAACCCTCGGCATC
GGCTGCACTACCACCAACACCGCCACTTTTGAGGAGGTGGCTAC
ACGAGAAAAGCTCGTGATCACTGACGTGGTGGACGGCGTGAACC
ACAAGCTGGACGTCACCACCAACACATGTACCATACGCAACTGC
AAGAAGCTGGGACCCAGGGAAAACGTTGCCATAATCCAGGTGGG
AGGTAGCAATATCCTCGACATAACCGCCGATCCTACGACGTCCC
CTCAGACTGAAAGGATGATGCGAGTCAACTGGAAGAAGTGGTGG
CAAGTTTTCTATACAGTGGTTGACTATATCAACCAAATAGTCAA
GGTGATGAGTAAACGGTCGCGGTCTCTGAACTCAGCCGCCTTCT
ATTATCGAGTGTGA
G9BE2001 + 7-1a-1b_G4BrB-9_VP7_AA
SEQ ID NO: 100
MDFIIYRELLFIVIVSPFVKTQNYGINLPITGSMDAAYANSSQQ
ETELSSTLCLYYPSEAPTQISDTEWKDTLSQLFLTKGWPTGSVY
FNEYSNVLEFSIDPKLYCDYNVVLIRFASGEELDVSELADLILN
EWLCNPMDITLYYYQQTDEANKWISMGQSCTIKVCPLNTQTLGI
GCTTTNTATFEEVATREKLVITDVVDGVNHKLDVTTNTCTIRNC
KKLGPRENVAIIQVGGSNILDITADPTTSPQTERMMRVNWKKWW
QVFYTVVDYINQIVKVMSKRSRSLNSAAFYYRV*
G9BE2001 + 7-1a-1b_Gl2K12_VP7_DNA_opt
SEQ ID NO: 101
ATGGATTTCATCATCTACAGGTTCCTGCTTTTCATCGTTATTGT
GAGCCCCTTTGTGAAGACACAGAACTACGGCATCAACCTCCCAA
TTACCGGTTCGATGGACGCAGCCTACGCAAATTCCTCACAGCAG
GAGACCTTTCTCACCTCTACCTTGTGTCTGTATTACCCCTCTTC
TGTCACAACAGAGATCACAGATCCTGATTGGACCAATACATTGT
CTCAGCTCTTCATGACCAAAGGGTGGCCTACTAACAGCGTGTAT
TTCAAGTCATATGCGGACATCGCTAGCTTCAGCGTTGATCCACA
GCTCTATTGCGACTACAACATCGTTCTGGTTCAGTACCAAAATT
CCCTGGCCTTAGACGTGTCTGAACTCGCCGACCTGATCCTGAAC
GAGTGGCTTTGCAACCCGATGGATATCACCCTGTATTACTATCA
GCAGACCGACGAAGCCAATAAGTGGATTAGCATGGGGCAGTCCT
GCACTATTAAGGTGTGCCCCCTCAATACACAAACCCTCGGCATC
GGCTGCACTACCACCAACACCGCCACTTTTGAGGAGGTGGCTAC
ACGAGAAAAGCTCGTGATCACTGACGTGGTGGACGGCGTGAACC
ACAAGCTGGACGTCACCACCAACACATGTACCATACGCAACTGC
AAGAAGCTGGGACCCAGGGAAAACGTCGCGATAATACAAGTGGG
TGGCTCTGATGTTATCGATATAACAGCTGATCCCACAACGATTC
CACAGACAGAGCGGATGATGCGGATCAACTGGAAGAAGTGGTGG
CAGGTTTTTTACACCGTGGTCGATTACATCAACCAGATCGTACA
GGTGATGAGCAAGCGGTCGCGGTCTCTGAACTCAGCCGCCTTCT
ATTATCGAGTGTGA
G9BE2001 + 7-1a-1b_Gl2K12_VP7_AA
SEQ ID NO: 102
MDFIIYRELLFIVIVSPFVKTQNYGINLPITGSMDAAYANSSQQ
ETFLTSTLCLYYPSSVTTEITDPDWTNTLSQLFMTKGWPTNSVY
EKSYADIASFSVDPQLYCDYNIVLVQYQNSLALDVSELADLILN
EWLCNPMDITLYYYQQTDEANKWISMGQSCTIKVCPLNTQTLGI
GCTTTNTATFEEVATREKLVITDVVDGVNHKLDVTTNTCTIRNC
KKLGPRENVAIIQVGGSDVIDITADPTTIPQTERMMRINWKKWW
QVFYTVVDYINQIVQVMSKRSRSLNSAAFYYRV*
IF-VP7(G12BAD89095).c
SEQ ID NO: 103
TCGTGCTTCGGCACCAGTACAATGGACTTTATCATATATAGGTT
CCTGCT
IF-VP7(G12BAD89095).r
SEQ ID NO: 104
ACTAAAGAAAATAGGCCTCTAGATCCTGTAGTAGAATGCGGCAG
AATTAA
G12K12_VP7_DNA_opt
SEQ ID NO: 105
ATGGACTTTATCATATATAGGTTCCTGCTCATCGTGGTTGTGAT
GTTGCCATTCATAAAAGCCCAGAACTACGGGATCAACCTGCCCA
TAACAGGATCTATGGACACAGCTTACACCAATTCAACTCAACAA
GAGAATTTCATGACCTCTACCTTGTGTCTGTATTACCCCTCTTC
TGTCACAACAGAGATCACAGATCCTGATTGGACCAATACATTGT
CTCAGCTCTTCATGACCAAAGGGTGGCCTACTAACAGCGTGTAT
TTCAAGTCATATGCGGACATCGCTAGCTTCAGCGTTGATCCACA
GCTCTATTGCGACTACAACATCGTTCTGGTTCAGTACCAAAATT
CCCTGGCCTTAGACGTGTCTGAACTCGCCGACCTGATCCTGAAC
GAATGGCTATGTAACCCAATGGACGTGACCCTGTACTACTACCA
GCAGACCGACGAGGCAAATAAGTGGATCAGCATGGGAGAATCTT
GCACCGTGAAAGTTTGTCCACTGAATACACAGACTCTCGGGATC
GGCTGCACTACTACCGATGTTACCACCTTTGAAGAAGTGGCAAA
CGCCGAGAAGCTTGTCATCACAGATGTAGTTGACGGCGTTAATC
ACAAAATTAATATTACTATGAACACCTGCACGATTAGGAATTGT
AAGAAACTGGGGCCACGCGAAAACGTCGCGATAATACAAGTGGG
TGGCTCTGATGTTATCGATATAACAGCTGATCCCACAACGATTC
CACAGACAGAGCGGATGATGCGGATCAACTGGAAGAAGTGGTGG
CAGGTTTTTTACACCGTGGTCGATTACATCAACCAGATCGTACA
GGTGATGAGCAAGCGTAGCCGGAGCCTTAATTCTGCCGCATTCT
ACTACAGGATCTAG
G12K12_VP7_AA
SEQ ID NO: 106
MDFIIYRELLIVVVMLPFIKAQNYGINLPITGSMDTAYTNSTQQ
ENFMTSTLCLYYPSSVTTEITDPDWTNTLSQLFMTKGWPTNSVY
EKSYADIASFSVDPQLYCDYNIVLVQYQNSLALDVSELADLILN
EWLCNPMDVTLYYYQQTDEANKWISMGESCTVKVCPLNTQTLGI
GCTTTDVTTFEEVANAEKLVITDVVDGVNHKINITMNTCTIRNC
KKLGPRENVAIIQVGGSDVIDITADPTTIPQTERMMRINWKKWW
QVFYTVVDYINQIVQVMSKRSRSLNSAAFYYRI*
G12K12 + 7-1a-1b_G1Rtx_VP7_DNA_opt
SEQ ID NO: 107
ATGGACTTTATCATATATAGGTTCCTGCTCATCGTGGTTGTGAT
GTTGCCATTCATAAAAGCCCAGAACTACGGGATCAACCTGCCCA
TAACAGGATCTATGGACACAGCTTACACCAATTCAACTCAACAA
GAGAATTTCATGACAAGTACCCTGTGCCTGTATTATCCAACAGA
AGCCTCTACCCAGATCAATGATGGGGAGTGGAAGGATAGTCTCT
CACAGATGTTCCTAACCAAGGGCTGGCCCACCGGTTCCGTCTAC
TTCAAGGAATACTCTAGTATTGTCGACTTCTCAGTTGACCCCCA
GCTTTATTGCGACTACAACCTGGTACTTATGAAATACGACCAGA
ACCTGGAGCTGGATATGTCCGAGCTGGCTGACCTGATCCTCAAC
GAATGGCTATGTAACCCAATGGACGTGACCCTGTACTACTACCA
GCAGACCGACGAGGCAAATAAGTGGATCAGCATGGGAGAATCTT
GCACCGTGAAAGTTTGTCCACTGAATACACAGACTCTCGGGATC
GGCTGCACTACTACCGATGTTACCACCTTTGAAGAAGTGGCAAA
CGCCGAGAAGCTTGTCATCACAGATGTAGTTGACGGCGTTAATC
ACAAAATTAATATTACTATGAACACCTGCACGATTAGGAATTGT
AAGAAACTGGGGCCACGCGAAAACGTCGCCGTGATCCAGGTGGG
GGGGAGCAATGTGCTCGACATTACTGCCGACCCTACCACCAATC
CACAGACGGAACGGATGATGAGAGTCAACTGGAAGAAATGGTGG
CAGGTCTTTTATACCATTGTGGACTACATTAACCAGATTGTGCA
AGTCATGAGTAAACGTAGCCGGAGCCTTAATTCTGCCGCATTCT
ACTACAGGATCTAG
G12K12 + 7-1a-1b_G1Rtx_VP7_AA
SEQ ID NO: 108
MDFIIYRELLIVVVMLPFIKAQNYGINLPITGSMDTAYTNSTQQ
ENFMTSTLCLYYPTEASTQINDGEWKDSLSQMFLTKGWPTGSVY
FKEYSSIVDFSVDPQLYCDYNLVLMKYDQNLELDMSELADLILN
EWLCNPMDVTLYYYQQTDEANKWISMGESCTVKVCPLNTQTLGI
GCTTTDVTTFEEVANAEKLVITDVVDGVNHKINITMNTCTIRNC
KKLGPRENVAVIQVGGSNVLDITADPTTNPQTERMMRVNWKKWW
QVFYTIVDYINQIVQVMSKRSRSLNSAAFYYRI*
G12K12 + 7-1a-1b_G2Sc2-9_VP7_DNA_opt
SEQ ID NO: 109
ATGGACTTTATCATATATAGGTTCCTGCTCATCGTGGTTGTGAT
GTTGCCATTCATAAAAGCCCAGAACTACGGGATCAACCTGCCCA
TAACAGGATCTATGGACACAGCTTACACCAATTCAACTCAACAA
GAGAATTTCATGACAAGCACCCTTTGCCTTTATTACCCAGCAGA
AGCAAAGAATGAAATTAGCGACGATGAGTGGGAGAATACACTTT
CACAGCTGTTTCTCACCAAGGGGTGGCCAACCGGTAGCGTATAC
TTCAAAGACTATAACGACATTACGACCTTTAGTATGAACCCTCA
GCTCTACTGTGACTATAACGTCGTGTTAATGCGCTATGACAATA
CCAGCGAGCTCGACGCCTCTGAGCTGGCTGACCTGATCCTGAAC
GAATGGCTATGTAACCCAATGGACGTGACCCTGTACTACTACCA
GCAGACCGACGAGGCAAATAAGTGGATCAGCATGGGAGAATCTT
GCACCGTGAAAGTTTGTCCACTGAATACACAGACTCTCGGGATC
GGCTGCACTACTACCGATGTTACCACCTTTGAAGAAGTGGCAAA
CGCCGAGAAGCTTGTCATCACAGATGTAGTTGACGGCGTTAATC
ACAAAATTAATATTACTATGAACACCTGCACGATTAGGAATTGT
AAGAAACTGGGGCCACGCGAAAACGTGGCCATTATCCAGGTTGG
CGGCCCTAACGCGCTCGACATCACTGCAGATCCAACAACCGTGC
CTCAAATTCAGCGGATTATGAGAATCAATTGGAAAAAGTGGTGG
CAGGTGTTTTATACGGTTGTGGACTATATTAATCAGATCGTACA
GGTGATGAGCAAACGTAGCCGGAGCCTTAATTCTGCCGCATTCT
ACTACAGGATCTAG
G12K12 + 7-1a-1b_G2Sc2-9_VP7_AA
SEQ ID NO: 110
MDFIIYRELLIVVVMLPFIKAQNYGINLPITGSMDTAYTNSTQQ
ENFMTSTLCLYYPAEAKNEISDDEWENTLSQLFLTKGWPTGSVY
FKDYNDITTFSMNPQLYCDYNVVLMRYDNTSELDASELADLILN
EWLCNPMDVTLYYYQQTDEANKWISMGESCTVKVCPLNTQTLGI
GCTTTDVTTFEEVANAEKLVITDVVDGVNHKINITMNTCTIRNC
KKLGPRENVAIIQVGGPNALDITADPTTVPQIQRIMRINWKKWW
QVFYTVVDYINQIVQVMSKRSRSLNSAAFYYRI*
G12K12 + 7-1a-1b_G3HCR3_VP7_DNA_opt
SEQ ID NO: 111
ATGGACTTTATCATATATAGGTTCCTGCTCATCGTGGTTGTGAT
GTTGCCATTCATAAAAGCCCAGAACTACGGGATCAACCTGCCCA
TAACAGGATCTATGGACACAGCTTACACCAATTCAACTCAACAA
GAGAATTTCATGACCAGCACACTCTGCCTCTACTACCCGACAGA
GGCTGCCACTGAGATCAACGATAATTCTTGGAAAGACACGTTAT
CGCAGCTGTTTCTTACTAAGGGCTGGCCCACCGGTAGTGTCTAC
TTTAAAGAGTATACCGACATTGCCTCTTTTAGCGTGGATCCTCA
GCTCTACTGTGACTATAACATCGTGTTGATGAAGTATGACGCAG
CGCTGCAGCTGGATATGAGTGAGCTGGCCGATTTGATCCTGAAC
GAATGGCTATGTAACCCAATGGACGTGACCCTGTACTACTACCA
GCAGACCGACGAGGCAAATAAGTGGATCAGCATGGGAGAATCTT
GCACCGTGAAAGTTTGTCCACTGAATACACAGACTCTCGGGATC
GGCTGCACTACTACCGATGTTACCACCTTTGAAGAAGTGGCAAA
CGCCGAGAAGCTTGTCATCACAGATGTAGTTGACGGCGTTAATC
ACAAAATTAATATTACTATGAACACCTGCACGATTAGGAATTGT
AAGAAACTGGGGCCACGCGAAAATGTTGCAGTCATCCAGGTAGG
AGGCAGTGATATTCTCGACATCACGGCCGACCCGACGACCGCGC
CTCAGACAGAAAGGATGATGCGGATCAATTGGAAGAAGTGGTGG
CAGGTGTTCTACACAGTGGTGGACTACGTTAACCAGATTATTCA
GGCTATGAGCAAGCGTAGCCGGAGCCTTAATTCTGCCGCATTCT
ACTACAGGATCTAG
G12K12 + 7-1a-1b_G3HCR3_VP7_AA
SEQ ID NO: 112
MDFIIYRELLIVVVMLPFIKAQNYGINLPITGSMDTAYTNSTQQ
ENFMTSTLCLYYPTEAATEINDNSWKDTLSQLFLTKGWPTGSVY
FKEYTDIASFSVDPQLYCDYNIVLMKYDAALQLDMSELADLILN
EWLCNPMDVTLYYYQQTDEANKWISMGESCTVKVCPLNTQTLGI
GCTTTDVTTFEEVANAEKLVITDVVDGVNHKINITMNTCTIRNC
KKEGPRENVAVIQVGGSDIEDITADPTTAPQTERMMRINWKKWW
QVFYTVVDYVNQIIQAMSKRSRSLNSAAFYYRI*
G12K12 + 7-1a-1b_G4BrB-9_VP7_DNA_opt
SEQ ID NO: 113
ATGGACTTTATCATATATAGGTTCCTGCTCATCGTGGTTGTGAT
GTTGCCATTCATAAAAGCCCAGAACTACGGGATCAACCTGCCCA
TAACAGGATCTATGGACACAGCTTACACCAATTCAACTCAACAA
GAGAATTTCATGTCTAGCACACTGTGCCTTTACTATCCTAGTGA
GGCACCGACTCAAATCAGTGATACAGAATGGAAGGATACACTGT
CTCAACTCTTTCTCACCAAGGGATGGCCCACTGGCTCAGTGTAT
TTTAATGAATACAGCAACGTTTTGGAGTTCAGTATTGACCCCAA
GCTGTACTGCGACTACAATGTAGTGCTGATTCGATTCGCCTCGG
GGGAGGAACTTGACGTATCCGAGTTGGCCGACCTCATCCTGAAC
GAATGGCTATGTAACCCAATGGACGTGACCCTGTACTACTACCA
GCAGACCGACGAGGCAAATAAGTGGATCAGCATGGGAGAATCTT
GCACCGTGAAAGTTTGTCCACTGAATACACAGACTCTCGGGATC
GGCTGCACTACTACCGATGTTACCACCTTTGAAGAAGTGGCAAA
CGCCGAGAAGCTTGTCATCACAGATGTAGTTGACGGCGTTAATC
ACAAAATTAATATTACTATGAACACCTGCACGATTAGGAATTGT
AAGAAACTGGGGCCACGCGAAAACGTTGCCATAATCCAGGTGGG
AGGTAGCAATATCCTCGACATAACCGCCGATCCTACGACGTCCC
CTCAGACTGAAAGGATGATGCGAGTCAACTGGAAGAAGTGGTGG
CAAGTTTTCTATACAGTGGTTGACTATATCAACCAAATAGTCAA
GGTGATGAGTAAACGTAGCCGGAGCCTTAATTCTGCCGCATTCT
ACTACAGGATCTAG
G12K12 + 7-1a-1b_G4BrB-9_VP7_AA
SEQ ID NO: 114
MDFIIYRFELIVVVMEPFIKAQNYGINEPITGSMDTAYTNSTQQ
ENFMSSTECLYYPSEAPTQISDTEWKDTESQLFETKGWPTGSVY
FNEYSNVLEFSIDPKEYCDYNVVEIRFASGEELDVSELADLIEN
EWECNPMDVTLYYYQQTDEANKWISMGESCTVKVCPENTQTEGI
GCTTTDVTTFEEVANAEKEVITDVVDGVNHKINITMNTCTIRNC
KKEGPRENVAIIQVGGSNIEDITADPTTSPQTERMMRVNWKKWW
QVFYTVVDYINQIVKVMSKRSRSLNSAAFYYRI*
G12K12 + 7-1a-1b_G9BE2001_VP7_DNA_opt
SEQ ID NO: 115
ATGGACTTTATCATATATAGGTTCCTGCTCATCGTGGTTGTGAT
GTTGCCATTCATAAAAGCCCAGAACTACGGGATCAACCTGCCCA
TAACAGGATCTATGGACACAGCTTACACCAATTCAACTCAACAA
GAGAATTTCATGACATCAACCTTGTGCTTGTATTACCCCACTGA
AGCGTCTACTCAGATCGGAGATACCGAGTGGAAAGATACTCTCA
GTCAGCTGTTCCTCACCAAGGGATGGCCAACAGGCTCTGTCTAC
TTTAAAGAGTACACGGACATCGCATCTTTTAGCATCGATCCTCA
GTTATACTGCGACTACAACGTGGTGTTGATGAAATACGACAGCA
CGCTGGAGCTCGACATGTCCGAGCTGGCTGATCTGATTCTCAAC
GAATGGCTATGTAACCCAATGGACGTGACCCTGTACTACTACCA
GCAGACCGACGAGGCAAATAAGTGGATCAGCATGGGAGAATCTT
GCACCGTGAAAGTTTGTCCACTGAATACACAGACTCTCGGGATC
GGCTGCACTACTACCGATGTTACCACCTTTGAAGAAGTGGCAAA
CGCCGAGAAGCTTGTCATCACAGATGTAGTTGACGGCGTTAATC
ACAAAATTAATATTACTATGAACACCTGCACGATTAGGAATTGT
AAGAAACTGGGGCCACGCGAAAACGTGGCTATCGTTCAGGTGGG
CGGTTCCGAGGTTCTCGACATAACGGCTGACCCAACCACCGCCC
CACAGACCGAGAGGATGATGCGCGTGAACTGGAAAAAATGGTGG
CAAGTGTTCTACACTGTGGTGGACTATATCAACCAGATTGTGCA
GGTGATGTCCAAACGTAGCCGGAGCCTTAATTCTGCCGCATTCT
ACTACAGGATCTAG
G12K12 + 7-1a-1b_G9BE2001_VP7_AA
SEQ ID NO: 116
MDFIIYRFELIVVVMEPFIKAQNYGINEPITGSMDTAYTNSTQQ
ENFMTSTECLYYPTEASTQIGDTEWKDTESQLFETKGWPTGSVY
FKEYTDIASFSIDPQLYCDYNVVEMKYDSTEELDMSELADLIEN
EWECNPMDVTLYYYQQTDEANKWISMGESCTVKVCPENTQTEGI
GCTTTDVTTFEEVANAEKEVITDVVDGVNHKINITMNTCTIRNC
KKEGPRENVAIVQVGGSEVEDITADPTTAPQTERMMRVNWKKWW
QVFYTVVDYINQIVQVMSKRSRSENSAAFYYRI*

All citations are hereby incorporated by reference.

1-31. (canceled)

32. A nucleic acid comprising a nucleotide sequence encoding a rotavirus VP7 fusion protein, the sequence comprising a first sequence encoding a 7-1a subdomain, a second sequence encoding a 7-2 domain and a third sequence encoding a 7-1b subdomain; wherein
the sequence of the 7-2 domain is derived from a first rotavirus strain and
the sequence of the 7-1a subdomain, the sequence of the 7-1b subdomain or the sequence of the 7-1a subdomain and the sequence of the 7-1b subdomain are derived from a second rotavirus strain, wherein
the first rotavirus strain is a different rotavirus strain than the second rotavirus strain.

33. The nucleic acid of claim 32, wherein
i) the 7-2 domain and the 7-1b subdomain are derived from a first rotavirus strain and the 7-1a subdomain is derived from a second rotavirus strain;
ii) the 7-2 domain and the 7-1a subdomain are derived from a first rotavirus strain and the 7-1b subdomain is derived from a second rotavirus strain; or
iii) the 7-2 domain is derived from a first rotavirus strain and the 7-1a subdomain and the 7-1b subdomain are derived from a second rotavirus strain.

34. The nucleic acid of claim 32 further encoding a leader peptide and a grip arm, wherein the leader peptide and a grip arm are derived from the first rotavirus strain.

35. The nucleic acid of claim 32, wherein the first rotavirus strain and the second rotavirus strain are selected from any one of rotavirus strain having genotype G1 to G19.

36. The nucleic acid of claim 32, wherein the first rotavirus strain, the second rotavirus strain or the first rotavirus strain and the second rotavirus strain are not rotavirus strain of genotype G4.

37. A rotavirus VP7 fusion protein encoded by the nucleic acid of claim 32.

38. A rotavirus like particle (RLP) comprising the rotavirus VP7 fusion protein of claim 37.

39. The RLP of claim 38, further comprising rotavirus protein VP2 and VP6.

40. A method of producing a rotavirus VP7 fusion protein in a plant, portion of a plant, or a plant cell or increasing yield of production of a rotavirus VP7 fusion protein in a plant, portion of a plant, or a plant cell, the method comprising,
providing a plant, portion of a plant, or a plant cell comprising the nucleic acid of claim 32, or introducing the nucleic acid of claim 32 into the plant, the portion of a plant, or the plant cell, and
incubating the plant, the portion of a plant, or the plant cell under conditions that permit the expression and production of the rotavirus VP7 fusion protein; or
incubating the plant, portion of the plant, or plant cell under conditions that permit expression of the rotavirus VP7 fusion protein encoded by the nucleic acid, thereby producing the rotavirus VP7 fusion protein at a higher yield compared to a plant, portion of the plant, or plant cell expressing native rotavirus VP7 protein.

41. A rotavirus VP7 fusion protein produced by the method of claim 40.

42. A method of producing a rotavirus like particle (RLP) in a plant, portion of a plant or plant cell comprising:
a) providing a plant, portion of a plant or plant cell comprising a first nucleic acid comprising a first regulatory region active in the plant operatively linked to a first nucleotide sequence encoding the rotavirus VP7 fusion protein of claim 37, a second nucleic acid comprising a second regulatory region active in the plant operatively linked to a second nucleotide sequence encoding rotavirus VP2 protein and a third nucleic acid comprising a third regulatory region active in the plant operatively linked to a third nucleotide sequence encoding rotavirus VP6 protein; and
b) incubating the plant, portion of a plant or plant cell under conditions that permit the expression of the first, second and third nucleic acid, thereby producing the RLP; or
a) introducing into the plant, portion of a plant or plant cell
a first nucleic acid comprising a first regulatory region active in the plant operatively linked to a first nucleotide sequence encoding a first rotavirus structural protein selected from one of VP2, VP6 and the rotavirus VP7 fusion protein of claim 37,
a second nucleic acid comprising a second regulatory region active in the plant operatively linked to a second nucleotide sequence encoding a second rotavirus structural protein selected from one of VP2, VP6 and the rotavirus VP7 fusion protein of claim 37, and
a third nucleic acid comprising a third regulatory region active in the plant operatively linked to a third nucleotide sequence encoding a third rotavirus structural protein selected from one of VP2, VP6 and the rotavirus VP7 fusion protein of claim 37,
b) incubating the plant, portion of a plant or plant cell under conditions that permit the expression of the first, second and third nucleic acid, thereby producing the RLP comprising VP2, VP6 and the VP7 fusion protein of claim 37.

43. The method of claim 42, further comprising the steps of
c) harvesting the plant, portion of a plant or plant cell, and
d) extracting and purifying the RLPs from the plant, portion of a plant or plant cell.

44. An RLP produced by the method of claim 42.

45. An antibody or antibody fragment prepared using the RLP of claim 38.

46. The antibody or antibody fragment of claim 45, wherein the antibody or antibody fragment recognizes an epitope of the 7-1a subdomain.

47. A composition for inducing an immune response comprising, an effective dose of the RLP of claim 38, and a pharmaceutically acceptable carrier, adjuvant, vehicle or excipient.

48. A vaccine comprising an effective dose of the RLP of claim 38 for inducing an immune response.

49. A method for inducing immunity to a rotavirus infection in a subject, the method comprising administering the RLP of claim 38 to the subject.

50. A plant, portion of the plant, or the plant cell comprising the nucleic acid of claim 32.

51. The method of claim 40, wherein the method further comprises the step of harvesting the plant, portion of the plant, or plant cell, and purifying the rotavirus VP7 fusion protein.


SEQUENCE LISTING (136 sequences)

SEQ ID NO: 1 - DNA, 48 bp, Artificial Sequence
  Synthetic sequence, Primer IF-WA_VP2(opt).s1+3c
aaatttgtcgggcccatggcataccggaagagaggagcaaagcgcgaa

SEQ ID NO: 2 - DNA, 50 bp, Artificial Sequence
  Synthetic sequence, Primer IF-WA_VP2(opt).s1-4r
actaaagaaaataggcctttaaagctcgttcattattcgcatattgtcga

SEQ ID NO: 3 - DNA, 2673 bp, Artificial Sequence
  Synthetic sequence, PCR template Wa_VP2_DNA_Opt
atggcataccggaagagaggagcaaagcgcgaaaacctgccgcaacagaacgagagactgcaagaaaaagagatagagaaagatgtcgacgtaacaatggaaaacaagaataacaataggaaacaacagctgtccgacaaagttctgtcccagaaggaggaaattatcactgacgcccaggacgatattaaaattgccggagaaataaagaagagctcgaaagaagaatctaaacagctgctcgaaattctgaaaacaaaagaagaccatcagaaagagattcaatatgaaattttgcaaaaaacaatacctacatttgagtccaaagaaagtatcctcaagaagcttgaagacataagaccggagcaggcaaaaaaacagatgaaactctttcgcattttcgagccaaaacagctccctatatatcgcgccaatggcgagaaggagctacgcaaccggtggtactggaagttgaaaaaagacaccctgccagatggagattatgacgtccgggagtatttcctcaatctctatgatcagatcctcatcgaaatgccggactatctgctcctcaaggacatggccgtggagaacaaaaatagcagagacgccggcaaagttgtcgactctgagactgccaatatttgtgatgccatcttccaggatgaggagaccgagggagtcgtccgtagattcatcgctgatatgcggcaacaggtccaggctgatcgtaacattgtcaattacccttccatccttcaccctattgatcatgcattcaatgagtattttcttaaccaccagttggtggagccgctgaacaatgagataatcttcaattacataccagagaggataaggaatgacgtgaattacatcctgaacatggatatgaatctgccatctacagccaggtatatcaggccaaacttgttgcaggatagactgaatcttcacgataattttgagtccctgtgggataccatcacaacatccaactacattctggccaggtccgtcgttcccgatttgaaggagaaggagctggtctccaccgaagcacagatccagaaaatgagccaggacctgcagctggaggccctcactattcagagcgagacacagtttttagccgggattaacagtcaggctgccaatgattgtttcaagaccctcatagccgccatgctgtctcaaagaaccatgtctttggactttgtgaccacgaactatatgagcctaatctccggaatgtggctacttacagtgattcccaacgatatgttcctccgggagtcactagtggcctgtgagctggcgatcatcaacaccatcgtgtatccagcattcggaatgcagagaatgcattaccggaatggcgaccctcagacacccttccagatcgcagaacagcagatccagaatttccaggtggcgaactggctccattttattaacaataacagattcaggcaagttgtgattgatggagttctgaatcagactctgaacgacaatatacggaatggacaggtcatcaaccagctgatggaagcattgatgcaactcagcagacagcagttccccacgatgcctgtggattacaaacggagcatccaacggggcattctgcttctctccaataggctggggcagcttgtcgacttaacccgactggtctcctataactacgagacgctaatggcttgtgtgaccatgaacatgcagcacgtgcaaaccctgacaactgagaagttgcagctcacttctgtgacttcgctttgtatgttaattggtaacacaaccgtgattccgtccccacagacactgttccactactacaacatcaacgtgaatttccactccaattataatgagcggatcaacgacgccgtcgccataattaccgcagcaaataggctgaatctttatcagaaaaaaatgaagtccatagtggaagactttctgaaacggctccagattttcgacgtaccacgagtgcctgacgaccaaatgtacaggctgagggatcgccttcggctcttacccgttgaacggagacggcttgacatattcaacttgatcctgatgaatatggagcagatcgaacgcgcttctgataagattgctcagggggttatcatcgcataccgagatatgcagctggaacgcgacgagatgtacggatatgttaatattgcacggaatcttgatggctaccagcaaattaacttggaggaactcatgcgcaccggtgattacggacaaattacgaacatgcttctcaacaatcaacccgttgcccttgtgggtgcattgcccttcgttacggactcatccgtgatcagtctaatcgccaagctcgacgcaaccgtcttcgctcagatagtgaagctcaggaaagttgacacactgaagcccatactgtacaaaataaactcggattccaatgacttttaccttgtggccaactacgactggatccccacaagtacaactaaggtctacaaacaggtgccacaaccattcgactttagagccagcatgcacatgctgacttctaaccttacgtttaccgtctactctgacctactgtcatttgtttcagcggacacggtagagcccattaacgcagtcgcattcgacaatatgcgaataatgaacgagctttaa

SEQ ID NO: 4 - protein, 890 aa, Artificial Sequence
  Synthetic sequence, Protein Wa_VP2_AA
MAYRKRGAKRENLPQQNERLQEKEIEKDVDVTMENKNNNRKQQLSDKVLSQKEEIITDAQDDIKIAGEIKKSSKEESKQLLEILKTKEDHQKEIQYEILQKTIPTFESKESILKKLEDIRPEQAKKQMKLFRIFEPKQLPIYRANGEKELRNRWYWKLKKDTLPDGDYDVREYFLNLYDQILIEMPDYLLLKDMAVENKNSRDAGKVVDSETANICDAIFQDEETEGVVRRFIADMRQQVQADRNIVNYPSILHPIDHAFNEYFLNHQLVEPLNNEIIFNYIPERIRNDVNYILNMDMNLPSTARYIRPNLLQDRLNLHDNFESLWDTITTSNYILARSVVPDLKEKELVSTEAQIQKMSQDLQLEALTIQSETQFLAGINSQAANDCFKTLIAAMLSQRTMSLDFVTTNYMSLISGMWLLTVIPNDMFLRESLVACELAIINTIVYPAFGMQRMHYRNGDPQTPFQIAEQQIQNFQVANWLHFINNNRFRQVVIDGVLNQTLNDNIRNGQVINQLMEALMQLSRQQFPTMPVDYKRSIQRGILLLSNRLGQLVDLTRLVSYNYETLMACVTMNMQHVQTLTTEKLQLTSVTSLCMLIGNTTVIPSPQTLFHYYNINVNFHSNYNERINDAVAIITAANRLNLYQKKMKSIVEDFLKRLQIFDVPRVPDDQMYRLRDRLRLLPVERRRLDIFNLILMNMEQIERASDKIAQGVIIAYRDMQLERDEMYGYVNIARNLDGYQQINLEELMRTGDYGQITNMLLNNQPVALVGALPFVTDSSVISLIAKLDATVFAQIVKLRKVDTLKPILYKINSDSNDFYLVANYDWIPTSTTKVYKQVPQPFDFRASMHMLTSNLTFTVYSDLLSFVSADTVEPINAVAFDNMRIMNEL

SEQ ID NO: 5 - DNA, 4903 bp, Artificial Sequence
  Synthetic sequence, Cloning vector 1191 from left to right T-DNA
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccgcggatggcgaaaaacgttgcgattttcggcttattgttttctcttcttgtgttggttccttctcagatcttcgcctgcaggctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagcgatcgctcaccatcaccatcaccatcaccatcaccattaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 6 - DNA, 4413 bp, Artificial Sequence
  Synthetic sequence, Construct 1710 from 2X35S to NOS
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcataccggaagagaggagcaaagcgcgaaaacctgccgcaacagaacgagagactgcaagaaaaagagatagagaaagatgtcgacgtaacaatggaaaacaagaataacaataggaaacaacagctgtccgacaaagttctgtcccagaaggaggaaattatcactgacgcccaggacgatattaaaattgccggagaaataaagaagagctcgaaagaagaatctaaacagctgctcgaaattctgaaaacaaaagaagaccatcagaaagagattcaatatgaaattttgcaaaaaacaatacctacatttgagtccaaagaaagtatcctcaagaagcttgaagacataagaccggagcaggcaaaaaaacagatgaaactctttcgcattttcgagccaaaacagctccctatatatcgcgccaatggcgagaaggagctacgcaaccggtggtactggaagttgaaaaaagacaccctgccagatggagattatgacgtccgggagtatttcctcaatctctatgatcagatcctcatcgaaatgccggactatctgctcctcaaggacatggccgtggagaacaaaaatagcagagacgccggcaaagttgtcgactctgagactgccaatatttgtgatgccatcttccaggatgaggagaccgagggagtcgtccgtagattcatcgctgatatgcggcaacaggtccaggctgatcgtaacattgtcaattacccttccatccttcaccctattgatcatgcattcaatgagtattttcttaaccaccagttggtggagccgctgaacaatgagataatcttcaattacataccagagaggataaggaatgacgtgaattacatcctgaacatggatatgaatctgccatctacagccaggtatatcaggccaaacttgttgcaggatagactgaatcttcacgataattttgagtccctgtgggataccatcacaacatccaactacattctggccaggtccgtcgttcccgatttgaaggagaaggagctggtctccaccgaagcacagatccagaaaatgagccaggacctgcagctggaggccctcactattcagagcgagacacagtttttagccgggattaacagtcaggctgccaatgattgtttcaagaccctcatagccgccatgctgtctcaaagaaccatgtctttggactttgtgaccacgaactatatgagcctaatctccggaatgtggctacttacagtgattcccaacgatatgttcctccgggagtcactagtggcctgtgagctggcgatcatcaacaccatcgtgtatccagcattcggaatgcagagaatgcattaccggaatggcgaccctcagacacccttccagatcgcagaacagcagatccagaatttccaggtggcgaactggctccattttattaacaataacagattcaggcaagttgtgattgatggagttctgaatcagactctgaacgacaatatacggaatggacaggtcatcaaccagctgatggaagcattgatgcaactcagcagacagcagttccccacgatgcctgtggattacaaacggagcatccaacggggcattctgcttctctccaataggctggggcagcttgtcgacttaacccgactggtctcctataactacgagacgctaatggcttgtgtgaccatgaacatgcagcacgtgcaaaccctgacaactgagaagttgcagctcacttctgtgacttcgctttgtatgttaattggtaacacaaccgtgattccgtccccacagacactgttccactactacaacatcaacgtgaatttccactccaattataatgagcggatcaacgacgccgtcgccataattaccgcagcaaataggctgaatctttatcagaaaaaaatgaagtccatagtggaagactttctgaaacggctccagattttcgacgtaccacgagtgcctgacgaccaaatgtacaggctgagggatcgccttcggctcttacccgttgaacggagacggcttgacatattcaacttgatcctgatgaatatggagcagatcgaacgcgcttctgataagattgctcagggggttatcatcgcataccgagatatgcagctggaacgcgacgagatgtacggatatgttaatattgcacggaatcttgatggctaccagcaaattaacttggaggaactcatgcgcaccggtgattacggacaaattacgaacatgcttctcaacaatcaacccgttgcccttgtgggtgcattgcccttcgttacggactcatccgtgatcagtctaatcgccaagctcgacgcaaccgtcttcgctcagatagtgaagctcaggaaagttgacacactgaagcccatactgtacaaaataaactcggattccaatgacttttaccttgtggccaactacgactggatccccacaagtacaactaaggtctacaaacaggtgccacaaccattcgactttagagccagcatgcacatgctgacttctaaccttacgtttaccgtctactctgacctactgtcatttgtttcagcggacacggtagagcccattaacgcagtcgcattcgacaatatgcgaataatgaacgagctttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 7 - DNA, 49 bp, Artificial Sequence
  Synthetic sequence, Primer IF-WA_VP6(opt).s1+3c
aaatttgtcgggcccatggaggtcctttatagtctctccaaaacgctga

SEQ ID NO: 8 - DNA, 52 bp, Artificial Sequence
  Synthetic sequence, Primer IF-WA_VP6(opt).s1-4r
actaaagaaaataggcctctacttgatcaacatactccggatagaggccaca

SEQ ID NO: 9 - DNA, 1194 bp, Artificial Sequence
  Synthetic sequence, PCT template Wa_VP6_DNA_Opt
atggaggtcctttatagtctctccaaaacgctgaaggacgctagggacaagatcgtggagggtacactttatagcaatgtcagcgacctaatacagcagtttaatcaaatgatcgttacaatgaatgggaatgatttccaaactggcggtattggtaatctgcccgtgaggaactggacattcgatttcggcctgctgggcacgactctccttaatctcgatgcaaattatgtagaaaacgccagaacgattatcgagtactttatcgatttcattgataacgtttgtatggatgagatggcccgcgagtcacaacggaacggagttgctccacagtccgaggcccttcggaaactcgccggcattaagttcaagcgtattaatttcgacaactcctccgaatatatagagaactggaacttgcagaatcgtcgacagagaaccggcttcgtgttccataaacctaatatctttccgtatagcgcctcattcaccctgaataggagtcagcccatgcacgacaacctcatgggtacaatgtggctgaatgcggggagtgaaatacaggtcgccgggttcgattactcctgtgccattaatgcacccgcaaacatccagcagttcgaacatatcgtgcaactaagacgggctctcacgaccgcgacaattacactcctgcccgacgccgagcgcttctcctttccccgcgtaatcaactcagctgatggcgccaccacttggttcttcaaccctgttatattgcgccctaacaacgtagaggtggagtttctcttaaacggacagatcatcaatacctaccaagccaggttcggcacgattattgcaagaaatttcgacgctatcaggctgctcttccaactgatgaggccccccaatatgactcccgctgtgaacgctttgtttccgcaggctcagcctttccagcaccacgccaccgtcggcttgactcttcgaatagagagcgcggtctgcgaatcagtgctggcagacgccaacgagacgctgctggcaaacgttaccgccgtgcggcaagagtatgccatcccagtagggcctgtgtttccacccggcatgaactggactgaactaattactaactatagcccatccagagaagacaacttgcagcgggtcttcactgtggcctctatccggagtatgttgatcaagtag

SEQ ID NO: 10 - protein, 397 aa, Artificial Sequence
  Synthetic sequence, Protein Wa_VP6_AA
MEVLYSLSKTLKDARDKIVEGTLYSNVSDLIQQFNQMIVTMNGNDFQTGGIGNLPVRNWTFDFGLLGTTLLNLDANYVENARTIIEYFIDFIDNVCMDEMARESQRNGVAPQSEALRKLAGIKFKRINFDNSSEYIENWNLQNRRQRTGFVFHKPNIFPYSASFTLNRSQPMHDNLMGTMWLNAGSEIQVAGFDYSCAINAPANIQQFEHIVQLRRALTTATITLLPDAERFSFPRVINSADGATTWFFNPVILRPNNVEVEFLLNGQIINTYQARFGTIIARNFDAIRLLFQLMRPPNMTPAVNALFPQAQPFQHHATVGLTLRIESAVCESVLADANETLLANVTAVRQEYAIPVGPVFPPGMNWTELITNYSPSREDNLQRVFTVASIRSMLIK

SEQ ID NO: 11 - DNA, 2934 bp, artificial Sequence
  Synthetic sequence, Construct 1713 from 2X35S to NOS
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggaggtcctttatagtctctccaaaacgctgaaggacgctagggacaagatcgtggagggtacactttatagcaatgtcagcgacctaatacagcagtttaatcaaatgatcgttacaatgaatgggaatgatttccaaactggcggtattggtaatctgcccgtgaggaactggacattcgatttcggcctgctgggcacgactctccttaatctcgatgcaaattatgtagaaaacgccagaacgattatcgagtactttatcgatttcattgataacgtttgtatggatgagatggcccgcgagtcacaacggaacggagttgctccacagtccgaggcccttcggaaactcgccggcattaagttcaagcgtattaatttcgacaactcctccgaatatatagagaactggaacttgcagaatcgtcgacagagaaccggcttcgtgttccataaacctaatatctttccgtatagcgcctcattcaccctgaataggagtcagcccatgcacgacaacctcatgggtacaatgtggctgaatgcggggagtgaaatacaggtcgccgggttcgattactcctgtgccattaatgcacccgcaaacatccagcagttcgaacatatcgtgcaactaagacgggctctcacgaccgcgacaattacactcctgcccgacgccgagcgcttctcctttccccgcgtaatcaactcagctgatggcgccaccacttggttcttcaaccctgttatattgcgccctaacaacgtagaggtggagtttctcttaaacggacagatcatcaatacctaccaagccaggttcggcacgattattgcaagaaatttcgacgctatcaggctgctcttccaactgatgaggccccccaatatgactcccgctgtgaacgctttgtttccgcaggctcagcctttccagcaccacgccaccgtcggcttgactcttcgaatagagagcgcggtctgcgaatcagtgctggcagacgccaacgagacgctgctggcaaacgttaccgccgtgcggcaagagtatgccatcccagtagggcctgtgtttccacccggcatgaactggactgaactaattactaactatagcccatccagagaagacaacttgcagcgggtcttcactgtggcctctatccggagtatgttgatcaagtagaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 12 - DNA, 52 bp, Artificial Sequence
  Synthetic sequence, Primer IF-WA_NSP4.s1+3c
aaatttgtcgggcccatggataagcttgccgacctcaactacacattgagtg

SEQ ID NO: 13 - DNA, 55 bp, Artificial Sequence
  Synthetic sequence, Primer IF-WA_NSP4.s1-4r
actaaagaaaataggccttcacatggatgcagtcacttctgacggttcatatgga

SEQ ID NO: 14 - DNA, 528 bp, Artificial Sequence
  Synthetic sequence, PCR template Wa_NSP4_DNA
atggataagcttgccgacctcaactacacattgagtgtaatcacttcaatgaatgacacattgcattctataattcaagatcctggaatggcgtattttctatatattgcatctgttctaacagttttgttcacattacataaagcttcaattccaaccatgaaaatagcattgaaaacatcaaaatgttcatataaagtgattaaatattgtatagtcacgatcattaatactcttttaaaattggctggatataaagagcaggttactacaaaagacgaaattgagcaacagatggacagaattgtgaaagagatgagacgtcagctggagatgattgataaactaactactcgtgaaattgaacaggttgaattgcttaaacgtatacatgacaacctgataactagaccagttgacgttatagatatgtcgaaggaattcaatcagaaaaacatcaaaacgctagatgaatgggagagtggaaaaaatccatatgaaccgtcagaagtgactgcatccatgtga

SEQ ID NO: 15 - protein, 175 aa, Artificial Sequence
  Synthetic sequence, Protein Wa_NSP4_AA
MDKLADLNYTLSVITSMNDTLHSIIQDPGMAYFLYIASVLTVLFTLHKASIPTMKIALKTSKCSYKVIKYCIVTIINTLLKLAGYKEQVTTKDEIEQQMDRIVKEMRRQLEMIDKLTTREIEQVELLKRIHDNLITRPVDVIDMSKEFNQKNIKTLDEWESGKNPYEPSEVTASM

SEQ ID NO: 16 - DNA, 2268 bp, Artificial Sequence
  Synthetic sequence, Construct 1706 from 2X35S to NOS
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggataagcttgccgacctcaactacacattgagtgtaatcacttcaatgaatgacacattgcattctataattcaagatcctggaatggcgtattttctatatattgcatctgttctaacagttttgttcacattacataaagcttcaattccaaccatgaaaatagcattgaaaacatcaaaatgttcatataaagtgattaaatattgtatagtcacgatcattaatactcttttaaaattggctggatataaagagcaggttactacaaaagacgaaattgagcaacagatggacagaattgtgaaagagatgagacgtcagctggagatgattgataaactaactactcgtgaaattgaacaggttgaattgcttaaacgtatacatgacaacctgataactagaccagttgacgttatagatatgtcgaaggaattcaatcagaaaaacatcaaaacgctagatgaatgggagagtggaaaaaatccatatgaaccgtcagaagtgactgcatccatgtgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 17 - DNA, 56 bp, Artificial Sequence
  Synthetic sequence, Primer IF(C160)- TrSP+Rtx_VP7(opt).c
tcgtgcttcggcaccagtacaatggattatattatctatcgtagcctcctcatcta

SEQ ID NO: 18 - DNA, 54 bp, Artificial Sequence
  Synthetic sequence, Primer IF-Rtx_VP7(opt).s1- 4r
actaaagaaaataggcctctaaacgcgataatagaaggctgctgagttcaggga

SEQ ID NO: 19 - DNA, 894 bp, Artificial Sequence
  Synthetic sequence, PCR template Rtx_VP7_DNA_Opt
atggattatattatctatcgtagcctcctcatctacgtggccctttttgccctgaccagggcccagaactatggcctgaacttaccaatcaccggttcaatggataccgtttacgctaattccactcaagaggggatatttctgacaagtaccctgtgcctgtattatccaacagaagcctctacccagatcaatgatggggagtggaaggatagtctctcacagatgttcctaaccaagggctggcccaccggttccgtctacttcaaggaatactctagtattgtcgacttctcagttgacccccagctttattgcgactacaacctggtacttatgaaatacgaccagaacctggagctggatatgtccgagctggctgacctgatcctcaatgagtggctgtgcaaccccatggacatcacattatattactaccagcagtctggagaatccaacaagtggatcagtatgggctcaagttgcaccgtgaaggtgtgtcccttgaacacccaaatgctgggcattggttgtcagacaactaatgtggattcgtttgaaatggtagccgaaaacgagaagctggctatagtggacgtagtcgatgggattaaccacaagatcaatctgactaccaccacttgtaccatcagaaactgtaaaaagctcggcccccgggagaacgtcgccgtgatccaggtgggggggagcaatgtgctcgacattactgccgaccctaccaccaatccacagacggaacggatgatgagagtcaactggaagaaatggtggcaggtcttttataccattgtggactacattaaccagattgtgcaagtcatgagtaaacggtccagatccctgaactcagcagccttctattatcgcgtttag

SEQ ID NO: 20 - protein, 297 aa, Artificial Sequence
  Synthetic sequence, Protein Rtx_VP7_AA
MDYIIYRSLLIYVALFALTRAQNYGLNLPITGSMDTVYANSTQEGIFLTSTLCLYYPTEASTQINDGEWKDSLSQMFLTKGWPTGSVYFKEYSSIVDFSVDPQLYCDYNLVLMKYDQNLELDMSELADLILNEWLCNPMDITLYYYQQSGESNKWISMGSSCTVKVCPLNTQMLGIGCQTTNVDSFEMVAENEKLAIVDVVDGINHKINLTTTTCTIRNCKKLGPRENVAVIQVGGSNVLDITADPTTNPQTERMMRVNWKKWWQVFYTIVDYINQIVQVMSKRSRSLNSAAFYYRV

SEQ ID NO: 21 - DNA, 4540 bp, Artificial Sequence
  Synthetic sequence, Cloning vector 1190 from left to right T-DNA
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccgcggatggcgaaaaacgttgcgattttcggcttattgttttctcttcttgtgttggttccttctcagatcttcgcctgcaggctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagcgatcgctcaccatcaccatcaccatcaccatcaccattaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 22 - DNA, 2277 bp, Artificial Sequence
  Synthetic sequence, Construct 1199 from 2X35S to NOS
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggattatattatctatcgtagcctcctcatctacgtggccctttttgccctgaccagggcccagaactatggcctgaacttaccaatcaccggttcaatggataccgtttacgctaattccactcaagaggggatatttctgacaagtaccctgtgcctgtattatccaacagaagcctctacccagatcaatgatggggagtggaaggatagtctctcacagatgttcctaaccaagggctggcccaccggttccgtctacttcaaggaatactctagtattgtcgacttctcagttgacccccagctttattgcgactacaacctggtacttatgaaatacgaccagaacctggagctggatatgtccgagctggctgacctgatcctcaatgagtggctgtgcaaccccatggacatcacattatattactaccagcagtctggagaatccaacaagtggatcagtatgggctcaagttgcaccgtgaaggtgtgtcccttgaacacccaaatgctgggcattggttgtcagacaactaatgtggattcgtttgaaatggtagccgaaaacgagaagctggctatagtggacgtagtcgatgggattaaccacaagatcaatctgactaccaccacttgtaccatcagaaactgtaaaaagctcggcccccgggagaacgtcgccgtgatccaggtgggggggagcaatgtgctcgacattactgccgaccctaccaccaatccacagacggaacggatgatgagagtcaactggaagaaatggtggcaggtcttttataccattgtggactacattaaccagattgtgcaagtcatgagtaaacggtccagatccctgaactcagcagccttctattatcgcgtttagaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 23 - DNA, 48 bp, Artificial Sequence
  Synthetic sequence, Primer IF-(160)RVA(G2P5SC2- 9)VP7.c
tcgtgcttcggcaccagtacaatggactacattatctatcgattttta

SEQ ID NO: 24 - DNA, 55 bp, Artificial Sequence
  Synthetic sequence, Primer IF-RVA(G2P5SC2- 9)VP7.r
actaaagaaaataggcctctacactcggtaatagaaggcggcagcatccaggctc

SEQ ID NO: 25 - DNA, 894 bp, Artificial Sequence
  Synthetic sequence, PCR template Sc2-9_VP7_DNA_Opt
atggactacattatctatcgatttttattggtaattgtgctgatctcaccattcgtcaggactcagaactacgggatctacctgccgataaccggctctctggatgcagtgtatacaaatagcacctcaggtgagacatttctcacaagcaccctttgcctttattacccagcagaagcaaagaatgaaattagcgacgatgagtgggagaatacactttcacagctgtttctcaccaaggggtggccaaccggtagcgtatacttcaaagactataacgacattacgacctttagtatgaaccctcagctctactgtgactataacgtcgtgttaatgcgctatgacaataccagcgagctcgacgcctctgagctggctgacctgatcctgaatgagtggttgtgcaacccaatggacatctccctttactattaccagcagtcctccgagagtaacaagtggattagcatgggtaccgattgcactgtaaaggtgtgtcccctgaatacccagactctcggaatcggttgcaaaaccaccgacgtgagcactttcgaaatagttgcttcctcagagaagctagttatcacagacgtggtgaacggcgtcaaccacaaaatcaatatcagcatctccacttgcactattcgaaattgcaacaaactcggcccccgggagaacgtggccattatccaggttggcggccctaacgcgctcgacatcactgcagatccaacaaccgtgcctcaaattcagcggattatgagaatcaattggaaaaagtggtggcaggtgttttatacggttgtggactatattaatcagatcgtacaggtgatgagcaaaaggagcaggagcctggatgctgccgccttctattaccgagtgtag

SEQ ID NO: 26 - protein, 297 aa, Artificial Sequence
  Synthetic sequence, Protein Sc2-9_VP7_AA
MDYIIYRFLLVIVLISPFVRTQNYGIYLPITGSLDAVYTNSTSGETFLTSTLCLYYPAEAKNEISDDEWENTLSQLFLTKGWPTGSVYFKDYNDITTFSMNPQLYCDYNVVLMRYDNTSELDASELADLILNEWLCNPMDISLYYYQQSSESNKWISMGTDCTVKVCPLNTQTLGIGCKTTDVSTFEIVASSEKLVITDVVNGVNHKINISISTCTIRNCNKLGPRENVAIIQVGGPNALDITADPTTVPQIQRIMRINWKKWWQVFYTVVDYINQIVQVMSKRSRSLDAAAFYYRV

SEQ ID NO: 27 - DNA, 894 bp, Artificial Sequence
  Synthetic sequence, PCR template 7-1a_Sc2-9_VP7_DNA_Opt
atggattatattatctatcgtagcctcctcatctacgtggccctttttgccctgaccagggcccagaactatggcctgaacttaccaatcaccggttcaatggataccgtttacgctaattccactcaagaggggatatttctgacaagcaccctttgcctttattacccagcagaagcaaagaatgaaattagcgacgatgagtgggagaatacactttcacagctgtttctcaccaaggggtggccaaccggtagcgtatacttcaaagactataacgacattacgacctttagtatgaaccctcagctctactgtgactataacgtcgtgttaatgcgctatgacaataccagcgagctcgacgcctctgagctggctgacctgatcctgaatgagtggctgtgcaaccccatggacatcacattatattactaccagcagtctggagaatccaacaagtggatcagtatgggctcaagttgcaccgtgaaggtgtgtcccttgaacacccaaatgctgggcattggttgtcagacaactaatgtggattcgtttgaaatggtagccgaaaacgagaagctggctatagtggacgtagtcgatgggattaaccacaagatcaatctgactaccaccacttgtaccatcagaaactgtaaaaagctcggcccccgggagaacgtcgccgtgatccaggtgggggggagcaatgtgctcgacattactgccgaccctaccaccaatccacagacggaacggatgatgagagtcaactggaagaaatggtggcaggtcttttataccattgtggactacattaaccagattgtgcaagtcatgagtaaacggtccagatccctgaactcagcagccttctattatcgcgtttag

SEQ ID NO: 28 - protein, 297 aa, Artificial Sequence
  Synthetic sequence, Protein 7-1a_Sc2-9_VP7_AA
MDYIIYRSLLIYVALFALTRAQNYGLNLPITGSMDTVYANSTQEGIFLTSTLCLYYPAEAKNEISDDEWENTLSQLFLTKGWPTGSVYFKDYNDITTFSMNPQLYCDYNVVLMRYDNTSELDASELADLILNEWLCNPMDITLYYYQQSGESNKWISMGSSCTVKVCPLNTQMLGIGCQTTNVDSFEMVAENEKLAIVDVVDGINHKINLTTTTCTIRNCKKLGPRENVAVIQVGGSNVLDITADPTTNPQTERMMRVNWKKWWQVFYTIVDYINQIVQVMSKRSRSLNSAAFYYRV

SEQ ID NO: 29 - DNA, 85 bp, Artificial Sequence
  Synthetic sequence, Primer IF-VP7(3End)Rtx+VP7- 1b(G2SC2-9).r
actaaagaaaataggcctctaaacgcgataatagaaggctgctgagttcagggatctggaccgtttgctcatcacctgtacgatc

SEQ ID NO: 30 - DNA, 894 bp, Artificial Sequence
  Synthetic sequence, Primer 7-1b_Sc2- 9_VP7_DNA_Opt
atggattatattatctatcgtagcctcctcatctacgtggccctttttgccctgaccagggcccagaactatggcctgaacttaccaatcaccggttcaatggataccgtttacgctaattccactcaagaggggatatttctgacaagtaccctgtgcctgtattatccaacagaagcctctacccagatcaatgatggggagtggaaggatagtctctcacagatgttcctaaccaagggctggcccaccggttccgtctacttcaaggaatactctagtattgtcgacttctcagttgacccccagctttattgcgactacaacctggtacttatgaaatacgaccagaacctggagctggatatgtccgagctggctgacctgatcctcaatgagtggctgtgcaaccccatggacatcacattatattactaccagcagtctggagaatccaacaagtggatcagtatgggctcaagttgcaccgtgaaggtgtgtcccttgaacacccaaatgctgggcattggttgtcagacaactaatgtggattcgtttgaaatggtagccgaaaacgagaagctggctatagtggacgtagtcgatgggattaaccacaagatcaatctgactaccaccacttgtaccatcagaaactgtaaaaagctcggcccccgggagaacgtggccattatccaggttggcggccctaacgcgctcgacatcactgcagatccaacaaccgtgcctcaaattcagcggattatgagaatcaattggaaaaagtggtggcaggtgttttatacggttgtggactatattaatcagatcgtacaggtgatgagcaaacggtccagatccctgaactcagcagccttctattatcgcgtttag

SEQ ID NO: 31 - protein, 297 aa, Artificial Sequence
  Synthetic sequence, Protein 7-1b_Sc2-9_VP7_AA
MDYIIYRSLLIYVALFALTRAQNYGLNLPITGSMDTVYANSTQEGIFLTSTLCLYYPTEASTQINDGEWKDSLSQMFLTKGWPTGSVYFKEYSSIVDFSVDPQLYCDYNLVLMKYDQNLELDMSELADLILNEWLCNPMDITLYYYQQSGESNKWISMGSSCTVKVCPLNTQMLGIGCQTTNVDSFEMVAENEKLAIVDVVDGINHKINLTTTTCTIRNCKKLGPRENVAIIQVGGPNALDITADPTTVPQIQRIMRINWKKWWQVFYTVVDYINQIVQVMSKRSRSLNSAAFYYRV

SEQ ID NO: 32 - DNA, 894 bp, Artificial Sequence
  Synthetic sequence, Primer 7-1a+1b_Sc2- 9_VP7_DNA_Opt
atggattatattatctatcgtagcctcctcatctacgtggccctttttgccctgaccagggcccagaactatggcctgaacttaccaatcaccggttcaatggataccgtttacgctaattccactcaagaggggatatttctgacaagcaccctttgcctttattacccagcagaagcaaagaatgaaattagcgacgatgagtgggagaatacactttcacagctgtttctcaccaaggggtggccaaccggtagcgtatacttcaaagactataacgacattacgacctttagtatgaaccctcagctctactgtgactataacgtcgtgttaatgcgctatgacaataccagcgagctcgacgcctctgagctggctgacctgatcctgaatgagtggctgtgcaaccccatggacatcacattatattactaccagcagtctggagaatccaacaagtggatcagtatgggctcaagttgcaccgtgaaggtgtgtcccttgaacacccaaatgctgggcattggttgtcagacaactaatgtggattcgtttgaaatggtagccgaaaacgagaagctggctatagtggacgtagtcgatgggattaaccacaagatcaatctgactaccaccacttgtaccatcagaaactgtaaaaagctcggcccccgggagaacgtggccattatccaggttggcggccctaacgcgctcgacatcactgcagatccaacaaccgtgcctcaaattcagcggattatgagaatcaattggaaaaagtggtggcaggtgttttatacggttgtggactatattaatcagatcgtacaggtgatgagcaaacggtccagatccctgaactcagcagccttctattatcgcgtttag

SEQ ID NO: 33 - protein, 297 aa, Artificial Sequence
  Synthetic sequence, Protein 7-1a+1b_Sc2- 9_VP7_AA
MDYIIYRSLLIYVALFALTRAQNYGLNLPITGSMDTVYANSTQEGIFLTSTLCLYYPAEAKNEISDDEWENTLSQLFLTKGWPTGSVYFKDYNDITTFSMNPQLYCDYNVVLMRYDNTSELDASELADLILNEWLCNPMDITLYYYQQSGESNKWISMGSSCTVKVCPLNTQMLGIGCQTTNVDSFEMVAENEKLAIVDVVDGINHKINLTTTTCTIRNCKKLGPRENVAIIQVGGPNALDITADPTTVPQIQRIMRINWKKWWQVFYTVVDYINQIVQVMSKRSRSLNSAAFYYRV

SEQ ID NO: 34 - DNA, 48 bp, Artificial Sequence
  Synthetic sequence, Primer IF-(160)RVA (G9P8WI61)VP7.c
tcgtgcttcggcaccagtacaatggattttatcatctaccgatttcta

SEQ ID NO: 35 - DNA, 49 bp, Artificial Sequence
  Synthetic sequence, Primer IF-RVA (G9P8WI61)VP7.r
actaaagaaaataggccttcaaacccggtaatagaacgctgcggagttt

SEQ ID NO: 36 - DNA, 894 bp, Artificial Sequence
  Synthetic sequence, Primer WI61_VP7_DNA_Opt
atggattttatcatctaccgatttctattgttgattgttatcgtaagcccgttcgtgaaaacgcagaactatggaatcaatctgcctattacagggagtatggataccgcgtacgctaattcttcacagctggatacgtttttaacctccacactttgcttatactatcctgctgaggcgagcactcagattggagacaccgagtggaagaacactctgagccagctattcctgacgaaagggtggcccacaggctctgtgtatttcaaagaatatactgacatcgcctccttcagcatcgatccacagctctactgcgactataacgtggttttaatgaagtatgattctacacttaaacttgacatgtccgaactggctgacctgatcctgaacgagtggctgtgcaaccccatggacatcacgctgtattattaccagcagacagacgaagccaacaagtggatcgccatgggacagagctgtacaattaaagtgtgtccactcaacacccaaactctcggtattgggtgcactacaaccaatacggccacttttgaggaggtggcggcctctgagaagctggtgattacagatgtggtagacggcgtgaaccacaaactggatgtgacgacgacaacgtgtacaatcagaaactgtcgcaagctgggacctcgggaaaacgtcgctattatacaggtgggagggagcgaagtgctagatattacggcagatccaactacagccccacagaccgagaggatgatgaggattaactggaagaagtggtggcaggtcttttacaccgtcgtggactatattaatcagattgttcaggtaatgagcaaaaggagtaggtctttaaactccgcagcgttctattaccgggtttga

SEQ ID NO: 37 - protein, 297 aa, Artificial Sequence
  Synthetic sequence, Protein WI61_VP7_AA
MDFIIYRFLLLIVIVSPFVKTQNYGINLPITGSMDTAYANSSQLDTFLTSTLCLYYPAEASTQIGDTEWKNTLSQLFLTKGWPTGSVYFKEYTDIASFSIDPQLYCDYNVVLMKYDSTLKLDMSELADLILNEWLCNPMDITLYYYQQTDEANKWIAMGQSCTIKVCPLNTQTLGIGCTTTNTATFEEVAASEKLVITDVVDGVNHKLDVTTTTCTIRNCRKLGPRENVAIIQVGGSEVLDITADPTTAPQTERMMRINWKKWWQVFYTVVDYINQIVQVMSKRSRSLNSAAFYYRV

SEQ ID NO: 38 - DNA, 894 bp, Artificial Sequence
  Synthetic sequence, Primer 7- 1a_WI61_VP7_DNA_Opt
atggattatattatctatcgtagcctcctcatctacgtggccctttttgccctgaccagggcccagaactatggcctgaacttaccaatcaccggttcaatggataccgtttacgctaattccactcaagaggggatatttctgacctccacactttgcttatactatcctgctgaggcgagcactcagattggagacaccgagtggaagaacactctgagccagctattcctgacgaaagggtggcccacaggctctgtgtatttcaaagaatatactgacatcgcctccttcagcatcgatccacagctctactgcgactataacgtggttttaatgaagtatgattctacacttaaacttgacatgtccgaactggctgacctgatcctgaatgagtggctgtgcaaccccatggacatcacattatattactaccagcagtctggagaatccaacaagtggatcagtatgggctcaagttgcaccgtgaaggtgtgtcccttgaacacccaaatgctgggcattggttgtcagacaactaatgtggattcgtttgaaatggtagccgaaaacgagaagctggctatagtggacgtagtcgatgggattaaccacaagatcaatctgactaccaccacttgtaccatcagaaactgtaaaaagctcggcccccgggagaacgtcgccgtgatccaggtgggggggagcaatgtgctcgacattactgccgaccctaccaccaatccacagacggaacggatgatgagagtcaactggaagaaatggtggcaggtcttttataccattgtggactacattaaccagattgtgcaagtcatgagtaaacggtccagatccctgaactcagcagccttctattatcgcgtttag

SEQ ID NO: 39 - protein, 297 aa, Artificial Sequence
  Synthetic sequence, Protein 7-1a_WI61_VP7_AA
MDYIIYRSLLIYVALFALTRAQNYGLNLPITGSMDTVYANSTQEGIFLTSTLCLYYPAEASTQIGDTEWKNTLSQLFLTKGWPTGSVYFKEYTDIASFSIDPQLYCDYNVVLMKYDSTLKLDMSELADLILNEWLCNPMDITLYYYQQSGESNKWISMGSSCTVKVCPLNTQMLGIGCQTTNVDSFEMVAENEKLAIVDVVDGINHKINLTTTTCTIRNCKKLGPRENVAVIQVGGSNVLDITADPTTNPQTERMMRVNWKKWWQVFYTIVDYINQIVQVMSKRSRSLNSAAFYYRV

SEQ ID NO: 40 - DNA, 894 bp, Artificial Sequence
  Synthetic sequence, Primer 7- 1b_WI61_VP7_DNA_Opt
atggattatattatctatcgtagcctcctcatctacgtggccctttttgccctgaccagggcccagaactatggcctgaacttaccaatcaccggttcaatggataccgtttacgctaattccactcaagaggggatatttctgacaagtaccctgtgcctgtattatccaacagaagcctctacccagatcaatgatggggagtggaaggatagtctctcacagatgttcctaaccaagggctggcccaccggttccgtctacttcaaggaatactctagtattgtcgacttctcagttgacccccagctttattgcgactacaacctggtacttatgaaatacgaccagaacctggagctggatatgtccgagctggctgacctgatcctcaatgagtggctgtgcaaccccatggacatcacattatattactaccagcagtctggagaatccaacaagtggatcagtatgggctcaagttgcaccgtgaaggtgtgtcccttgaacacccaaatgctgggcattggttgtcagacaactaatgtggattcgtttgaaatggtagccgaaaacgagaagctggctatagtggacgtagtcgatgggattaaccacaagatcaatctgactaccaccacttgtaccatcagaaactgtaaaaagctcggcccccgggagaacgtcgctattatacaggtgggagggagcgaagtgctagatattacggcagatccaactacagccccacagaccgagaggatgatgaggattaactggaagaagtggtggcaggtcttttacaccgtcgtggactatattaatcagattgttcaggtaatgagcaaaaggagtaggtctttaaactccgcagcgttctattaccgggtttga

SEQ ID NO: 41 - protein, 297 aa, Artificial Sequence
  Synthetic sequence, Protein 7-1b_WI61_VP7_AA
MDYIIYRSLLIYVALFALTRAQNYGLNLPITGSMDTVYANSTQEGIFLTSTLCLYYPTEASTQINDGEWKDSLSQMFLTKGWPTGSVYFKEYSSIVDFSVDPQLYCDYNLVLMKYDQNLELDMSELADLILNEWLCNPMDITLYYYQQSGESNKWISMGSSCTVKVCPLNTQMLGIGCQTTNVDSFEMVAENEKLAIVDVVDGINHKINLTTTTCTIRNCKKLGPRENVAIIQVGGSEVLDITADPTTAPQTERMMRINWKKWWQVFYTVVDYINQIVQVMSKRSRSLNSAAFYYRV

SEQ ID NO: 42 - DNA, 894 bp, Artificial Sequence
  Synthetic sequence, Primer 7- 1a+1b_WI61_VP7_DNA_Opt
atggattatattatctatcgtagcctcctcatctacgtggccctttttgccctgaccagggcccagaactatggcctgaacttaccaatcaccggttcaatggataccgtttacgctaattccactcaagaggggatatttctgacctccacactttgcttatactatcctgctgaggcgagcactcagattggagacaccgagtggaagaacactctgagccagctattcctgacgaaagggtggcccacaggctctgtgtatttcaaagaatatactgacatcgcctccttcagcatcgatccacagctctactgcgactataacgtggttttaatgaagtatgattctacacttaaacttgacatgtccgaactggctgacctgatcctgaatgagtggctgtgcaaccccatggacatcacattatattactaccagcagtctggagaatccaacaagtggatcagtatgggctcaagttgcaccgtgaaggtgtgtcccttgaacacccaaatgctgggcattggttgtcagacaactaatgtggattcgtttgaaatggtagccgaaaacgagaagctggctatagtggacgtagtcgatgggattaaccacaagatcaatctgactaccaccacttgtaccatcagaaactgtaaaaagctcggcccccgggagaacgtcgctattatacaggtgggagggagcgaagtgctagatattacggcagatccaactacagccccacagaccgagaggatgatgaggattaactggaagaagtggtggcaggtcttttacaccgtcgtggactatattaatcagattgttcaggtaatgagcaaaaggagtaggtctttaaactccgcagcgttctattaccgggtttga

SEQ ID NO: 43 - protein, 297 aa, Artificial Sequence
  Synthetic sequence, Protein 7-1a+1b_WI61_VP7_AA
MDYIIYRSLLIYVALFALTRAQNYGLNLPITGSMDTVYANSTQEGIFLTSTLCLYYPAEASTQIGDTEWKNTLSQLFLTKGWPTGSVYFKEYTDIASFSIDPQLYCDYNVVLMKYDSTLKLDMSELADLILNEWLCNPMDITLYYYQQSGESNKWISMGSSCTVKVCPLNTQMLGIGCQTTNVDSFEMVAENEKLAIVDVVDGINHKINLTTTTCTIRNCKKLGPRENVAIIQVGGSEVLDITADPTTAPQTERMMRINWKKWWQVFYTVVDYINQIVQVMSKRSRSLNSAAFYYRV

SEQ ID NO: 44 - DNA, 54 bp, Artificial Sequence
  Synthetic sequence, Primer IF-(160)RVA (G3P5WI78-8)VP7.c
tcgtgcttcggcaccagtacaatggatttcattatataccgcttcctgctcatc

SEQ ID NO: 45 - DNA, 50 bp, Artificial Sequence
  Synthetic sequence, Primer IF-RVA(G3P5WI78- 8)VP7.r
actaaagaaaataggcctttaaacccggtaatagaatgcggccgaattca

SEQ ID NO: 46 - DNA, 894 bp, Artificial Sequence
  Synthetic sequence, Primer WI78-8_VP7_DNA_Opt
atggatttcattatataccgcttcctgctcatcattgtgatactttctcccttgttgaacgcgcaaaattacgggattaacctcccaattactggttccatggacactagctacaccaattcaacccgggaggaagttttcctcacgagcactctttgcctatattatcccaccgaggctgccacagagatcaatgacaattcctggaaagatactttgagccagctgttcctgaccaaagggtggccaaccgagagtatctattttaaagattacaccgacatagcgtcattttctgttgacccacagctctattgcgactacaatctggtgctcatgaaatacgacgcaacccttcagctggacatgtccgagctagcagacctgctgctcaacgagtggctctgcaaccctatggatataacgctgtactattaccagcagacagatgaagccaacaagtggattagtatgggttcgagctgcacgatcaaggtttgcccactgaacactcaaaccctcggtataggttgtttgaccactgacgcgaatacatttgaggaagtggccaccgctgaaaaacttgtgatcaccgacgtcgtggacggtgttaaccacaagctgaaagtgaccaccgacacgtgcacgattcgcaactgtaaaaaattagggccccgtgaaaacgtggctgtgatccaagtcggagggagtgacgtgctggacattaccgcagatcccacaactgctccacagactgagaggatgatgagggtcaactggaagaagtggtggcaggtgttctatacgattgttgactacgtcaatcagattgtgcaggccatgtcaaagaggtcacgatctctgaattcggccgcattctattaccgggtttaa

SEQ ID NO: 47 - protein, 297 aa, Artificial Sequence
  Synthetic sequence, Protein WI78-8_VP7_AA
MDFIIYRFLLIIVILSPLLNAQNYGINLPITGSMDTSYTNSTREEVFLTSTLCLYYPTEAATEINDNSWKDTLSQLFLTKGWPTESIYFKDYTDIASFSVDPQLYCDYNLVLMKYDATLQLDMSELADLLLNEWLCNPMDITLYYYQQTDEANKWISMGSSCTIKVCPLNTQTLGIGCLTTDANTFEEVATAEKLVITDVVDGVNHKLKVTTDTCTIRNCKKLGPRENVAVIQVGGSDVLDITADPTTAPQTERMMRVNWKKWWQVFYTIVDYVNQIVQAMSKRSRSLNSAAFYYRV

SEQ ID NO: 48 - DNA, 86 bp, Artificial Sequence
  Synthetic sequence, Primer IF-VP7(3End)Rtx+VP7- 1b(G3P5).r
actaaagaaaataggcctctaaacgcgataatagaaggctgctgagttcagggatctggaccgctttgacatggcctgcacaatct

SEQ ID NO: 49 - DNA, 894 bp, Artificial Sequence
  Synthetic sequence, Primer 7-1b_WI78- 8_VP7_DNA_Opt
atggattatattatctatcgtagcctcctcatctacgtggccctttttgccctgaccagggcccagaactatggcctgaacttaccaatcaccggttcaatggataccgtttacgctaattccactcaagaggggatatttctgacaagtaccctgtgcctgtattatccaacagaagcctctacccagatcaatgatggggagtggaaggatagtctctcacagatgttcctaaccaagggctggcccaccggttccgtctacttcaaggaatactctagtattgtcgacttctcagttgacccccagctttattgcgactacaacctggtacttatgaaatacgaccagaacctggagctggatatgtccgagctggctgacctgatcctcaatgagtggctgtgcaaccccatggacatcacattatattactaccagcagtctggagaatccaacaagtggatcagtatgggctcaagttgcaccgtgaaggtgtgtcccttgaacacccaaatgctgggcattggttgtcagacaactaatgtggattcgtttgaaatggtagccgaaaacgagaagctggctatagtggacgtagtcgatgggattaaccacaagatcaatctgactaccaccacttgtaccatcagaaactgtaaaaagctcggcccccgggagaacgtggctgtgatccaagtcggagggagtgacgtgctggacattaccgcagatcccacaactgctccacagactgagaggatgatgagggtcaactggaagaagtggtggcaggtgttctatacgattgttgactacgtcaatcagattgtgcaggccatgtcaaagcggtccagatccctgaactcagcagccttctattatcgcgtttag

SEQ ID NO: 50 - protein, 297 aa, Artificial Sequence
  Synthetic sequence, Protein 7-1b_WI78-8_VP7_AA
MDYIIYRSLLIYVALFALTRAQNYGLNLPITGSMDTVYANSTQEGIFLTSTLCLYYPTEASTQINDGEWKDSLSQMFLTKGWPTGSVYFKEYSSIVDFSVDPQLYCDYNLVLMKYDQNLELDMSELADLILNEWLCNPMDITLYYYQQSGESNKWISMGSSCTVKVCPLNTQMLGIGCQTTNVDSFEMVAENEKLAIVDVVDGINHKINLTTTTCTIRNCKKLGPRENVAVIQVGGSDVLDITADPTTAPQTERMMRVNWKKWWQVFYTIVDYVNQIVQAMSKRSRSLNSAAFYYRV

SEQ ID NO: 51 - DNA, 894 bp, Artificial Sequence
  Synthetic sequence, Primer 7-1a+1b_WI78- 8_VP7_DNA_Opt
atggattatattatctatcgtagcctcctcatctacgtggccctttttgccctgaccagggcccagaactatggcctgaacttaccaatcaccggttcaatggataccgtttacgctaattccactcaagaggggatatttctgacgagcactctttgcctatattatcccaccgaggctgccacagagatcaatgacaattcctggaaagatactttgagccagctgttcctgaccaaagggtggccaaccgagagtatctattttaaagattacaccgacatagcgtcattttctgttgacccacagctctattgcgactacaatctggtgctcatgaaatacgacgcaacccttcagctggacatgtccgagctagcagacctgctgctcaatgagtggctgtgcaaccccatggacatcacattatattactaccagcagtctggagaatccaacaagtggatcagtatgggctcaagttgcaccgtgaaggtgtgtcccttgaacacccaaatgctgggcattggttgtcagacaactaatgtggattcgtttgaaatggtagccgaaaacgagaagctggctatagtggacgtagtcgatgggattaaccacaagatcaatctgactaccaccacttgtaccatcagaaactgtaaaaagctcggcccccgggagaacgtggctgtgatccaagtcggagggagtgacgtgctggacattaccgcagatcccacaactgctccacagactgagaggatgatgagggtcaactggaagaagtggtggcaggtgttctatacgattgttgactacgtcaatcagattgtgcaggccatgtcaaagcggtccagatccctgaactcagcagccttctattatcgcgtttag

SEQ ID NO: 52 - protein, 297 aa, Artificial Sequence
  Synthetic sequence, Protein 7-1a+1b_WI78- 8_VP7_AA
MDYIIYRSLLIYVALFALTRAQNYGLNLPITGSMDTVYANSTQEGIFLTSTLCLYYPTEAATEINDNSWKDTLSQLFLTKGWPTESIYFKDYTDIASFSVDPQLYCDYNLVLMKYDATLQLDMSELADLLLNEWLCNPMDITLYYYQQSGESNKWISMGSSCTVKVCPLNTQMLGIGCQTTNVDSFEMVAENEKLAIVDVVDGINHKINLTTTTCTIRNCKKLGPRENVAVIQVGGSDVLDITADPTTAPQTERMMRVNWKKWWQVFYTIVDYVNQIVQAMSKRSRSLNSAAFYYRV

SEQ ID NO: 53 - DNA, 51 bp, Artificial Sequence
  Synthetic sequence, Primer IF-(160)RVA (G12P8KDH651)VP7.c
tcgtgcttcggcaccagtacaatggatttcatcatctaccgcttcctccta

SEQ ID NO: 54 - DNA, 52 bp, Artificial Sequence
  Synthetic sequence, Primer IF-RVA(G12P8KDH651) VP7.r
actaaagaaaataggccttcagatcctgtagtaaaaggcagcggagttcagg

SEQ ID NO: 55 - DNA, 894 bp, Artificial Sequence
  Synthetic sequence, Primer KDH651_VP7_DNA_Opt
atggatttcatcatctaccgcttcctcctaatagtagtgatcatcctgcccttcattaaagcacagaactatgggatcaacctgcccatcacaggctctatggatgccgcgtacgtgaattcaacacaacaggaaaatttcatgacctccacactttgtctttactatccgagtagcgtgactactgaaatcacagatcccgattggaccaataccctgagccagctgtttctaaccaagggatggcccgtgaactctgtgtattttaagagctatgcagatatttcttcattttcggtggacccccagctttattgcgactacaacatagtgctgatacagtaccagaactcgctggctttggatgttagtgaactggctgacctgatcctgaatgaatggttgtgcaaccctatggacgtgacactctactactaccaacagacagatgaggcaaacaagtggatctcgatgggagaatcttgcacagtcaaagtctgccccctcaacacccaaaccctgggtattggatgcacgactaccgatgtgacaaccttcgaagaagtggcgaatgccgagaaacttgtgatcaccgatgtggttgacggcgtgaaccataaaattaacatcaccgtcaacacatgtactatcaggaattgcaaaaaactcggtccaagggagaacgtcgccatcatacaggtggggagttcagatgtcatcgatatcaccgccgaccccacaaccatcccgcagaccgagcgaatgatgagaatcaattggaaaaaatggtggcaagtattttacacagtcgtggattatattaatcagatcgtgcaggttatgagcaaaaggtcaagaagcctgaactccgctgccttttactacaggatctga

SEQ ID NO: 56 - protein, 297 aa, Artificial Sequence
  Synthetic sequence, Protein KDH651_VP7_AA
MDFIIYRFLLIVVIILPFIKAQNYGINLPITGSMDAAYVNSTQQENFMTSTLCLYYPSSVTTEITDPDWTNTLSQLFLTKGWPVNSVYFKSYADISSFSVDPQLYCDYNIVLIQYQNSLALDVSELADLILNEWLCNPMDVTLYYYQQTDEANKWISMGESCTVKVCPLNTQTLGIGCTTTDVTTFEEVANAEKLVITDVVDGVNHKINITVNTCTIRNCKKLGPRENVAIIQVGSSDVIDITADPTTIPQTERMMRINWKKWWQVFYTVVDYINQIVQVMSKRSRSLNSAAFYYRI

SEQ ID NO: 57 - DNA, 84 bp, Artificial Sequence
  Synthetic sequence, Primer IF-VP7(3End)Rtx+VP7- 1b(G12P8).r
actaaagaaaataggcctctaaacgcgataatagaaggctgctgagttcagggatctggaccgtttgctcataacctgcacgat

SEQ ID NO: 58 - DNA, 894 bp, Artificial Sequence
  Synthetic sequence, Primer 7- 1b_KDH651_VP7_DNA_Opt
atggattatattatctatcgtagcctcctcatctacgtggccctttttgccctgaccagggcccagaactatggcctgaacttaccaatcaccggttcaatggataccgtttacgctaattccactcaagaggggatatttctgacctccacactttgtctttactatccgagtagcgtgactactgaaatcacagatcccgattggaccaataccctgagccagctgtttctaaccaagggatggcccgtgaactctgtgtattttaagagctatgcagatatttcttcattttcggtggacccccagctttattgcgactacaacatagtgctgatacagtaccagaactcgctggctttggatgttagtgaactggctgacctgatcctgaatgagtggctgtgcaaccccatggacatcacattatattactaccagcagtctggagaatccaacaagtggatcagtatgggctcaagttgcaccgtgaaggtgtgtcccttgaacacccaaatgctgggcattggttgtcagacaactaatgtggattcgtttgaaatggtagccgaaaacgagaagctggctatagtggacgtagtcgatgggattaaccacaagatcaatctgactaccaccacttgtaccatcagaaactgtaaaaagctcggcccccgggagaacgtcgccatcatacaggtggggagttcagatgtcatcgatatcaccgccgaccccacaaccatcccgcagaccgagcgaatgatgagaatcaattggaaaaaatggtggcaagtattttacacagtcgtggattatattaatcagatcgtgcaggttatgagcaaacggtccagatccctgaactcagcagccttctattatcgcgtttag

SEQ ID NO: 59 - protein, 297 aa, Artificial Sequence
  Synthetic sequence, Protein 7-1b_KDH651_VP7_AA
MDYIIYRSLLIYVALFALTRAQNYGLNLPITGSMDTVYANSTQEGIFLTSTLCLYYPSSVTTEITDPDWTNTLSQLFLTKGWPVNSVYFKSYADISSFSVDPQLYCDYNIVLIQYQNSLALDVSELADLILNEWLCNPMDITLYYYQQSGESNKWISMGSSCTVKVCPLNTQMLGIGCQTTNVDSFEMVAENEKLAIVDVVDGINHKINLTTTTCTIRNCKKLGPRENVAIIQVGSSDVIDITADPTTIPQTERMMRINWKKWWQVFYTVVDYINQIVQVMSKRSRSLNSAAFYYRV

SEQ ID NO: 60 - DNA, 894 bp, Artificial Sequence; identical to SEQ ID NO: 58
  Synthetic sequence, Primer 7- 1a+1b_KDH651_VP7_DNA_Opt

SEQ ID NO: 61 - protein, 297 aa, Artificial Sequence; identical to SEQ ID NO: 59
  Synthetic sequence, Protein 7- 1a+1b_KDH651_VP7_AA

SEQ ID NO: 62 - DNA, 51 bp, Artificial Sequence
  Synthetic sequence, Primer IF-VP7(G3AAA18522).c
tcgtgcttcggcaccagtacaatggatttcatcatatataggtttctgttt

SEQ ID NO: 63 - DNA, 52 bp, Artificial Sequence
  Synthetic sequence, Primer IF-VP7(G3AAA18522).r
actaaagaaaataggcctttacaccctataatagaatgcagcggagttaagg

SEQ ID NO: 64 - DNA, 894 bp, Artificial Sequence
  Synthetic sequence, G3HCR3+7-1a- 1b_G1Rtx_VP7_DNA_opt
atggatttcatcatatataggtttctgtttataatagtgattctgtcacctctactcaaggcgcaaaactatggcataaacctccctatcaccggctcaatggacaccgcctatgcaaactccacgcaggaagaaactctgctgacaagtaccctgtgcctgtattatccaacagaagcctctacccagatcaatgatggggagtggaaggatagtctctcacagatgttcctaaccaagggctggcccaccggttccgtctacttcaaggaatactctagtattgtcgacttctcagttgacccccagctttattgcgactacaacctggtacttatgaaatacgaccagaacctggagctggatatgtccgagctggctgacctgatcctcaatgagtggctgtgtaatccaatggatatcacactctactactaccagcagactgacgaagccaacaagtggatctctatgggttctagctgcaccatcaaagtgtgccccctgaacacccagacactgggcattggctgtctgacgacagatgtcagtaccttcgaggaggtggcgacaacagagaaactggtgatcaccgacgtggttgacggcgtgaaccacaaactcgacgtgacaactaccacctgcaccatccggaattgtaagaagctgggaccgagagaaaacgtcgccgtgatccaggtgggggggagcaatgtgctcgacattactgccgaccctaccaccaatccacagacggaacggatgatgagagtcaactggaagaaatggtggcaggtcttttataccattgtggactacattaaccagattgtgcaagtcatgagtaaacgcagcagatcccttaactccgctgcattctattatagggtgtaa

SEQ ID NO: 65 - protein, 297 aa, Artificial Sequence
  Synthetic sequence, Protein G3HCR3+7-1a- 1b_G1Rtx_VP7_AA
MDFIIYRFLFIIVILSPLLKAQNYGINLPITGSMDTAYANSTQEETLLTSTLCLYYPTEASTQINDGEWKDSLSQMFLTKGWPTGSVYFKEYSSIVDFSVDPQLYCDYNLVLMKYDQNLELDMSELADLILNEWLCNPMDITLYYYQQTDEANKWISMGSSCTIKVCPLNTQTLGIGCLTTDVSTFEEVATTEKLVITDVVDGVNHKLDVTTTTCTIRNCKKLGPRENVAVIQVGGSNVLDITADPTTNPQTERMMRVNWKKWWQVFYTIVDYINQIVQVMSKRSRSLNSAAFYYRV

SEQ ID NO: 66 - DNA, 894 bp, Artificial Sequence
  Synthetic sequence, Primer G3HCR3+7-1a- 1b_G2Sc2-9_VP7_DNA_opt
atggatttcatcatatataggtttctgtttataatagtgattctgtcacctctactcaaggcgcaaaactatggcataaacctccctatcaccggctcaatggacaccgcctatgcaaactccacgcaggaagaaactctgctgacaagcaccctttgcctttattacccagcagaagcaaagaatgaaattagcgacgatgagtgggagaatacactttcacagctgtttctcaccaaggggtggccaaccggtagcgtatacttcaaagactataacgacattacgacctttagtatgaaccctcagctctactgtgactataacgtcgtgttaatgcgctatgacaataccagcgagctcgacgcctctgagctggctgacctgatcctgaatgagtggctgtgtaatccaatggatatcacactctactactaccagcagactgacgaagccaacaagtggatctctatgggttctagctgcaccatcaaagtgtgccccctgaacacccagacactgggcattggctgtctgacgacagatgtcagtaccttcgaggaggtggcgacaacagagaaactggtgatcaccgacgtggttgacggcgtgaaccacaaactcgacgtgacaactaccacctgcaccatccggaattgtaagaagctgggaccgagagaaaacgtggccattatccaggttggcggccctaacgcgctcgacatcactgcagatccaacaaccgtgcctcaaattcagcggattatgagaatcaattggaaaaagtggtggcaggtgttttatacggttgtggactatattaatcagatcgtacaggtgatgagcaaacgcagcagatcccttaactccgctgcattctattatagggtgtaa

SEQ ID NO: 67 - protein, 297 aa, Artificial Sequence
  Synthetic sequence, Protein G3HCR3+7-1a- 1b_G2Sc2-9_VP7_AA
MDFIIYRFLFIIVILSPLLKAQNYGINLPITGSMDTAYANSTQEETLLTSTLCLYYPAEAKNEISDDEWENTLSQLFLTKGWPTGSVYFKDYNDITTFSMNPQLYCDYNVVLMRYDNTSELDASELADLILNEWLCNPMDITLYYYQQTDEANKWISMGSSCTIKVCPLNTQTLGIGCLTTDVSTFEEVATTEKLVITDVVDGVNHKLDVTTTTCTIRNCKKLGPRENVAIIQVGGPNALDITADPTTVPQIQRIMRINWKKWWQVFYTVVDYINQIVQVMSKRSRSLNSAAFYYRV

SEQ ID NO: 68 - DNA, 894 bp, Artificial Sequence
  Synthetic sequence, Primer G3HCR3+7-1a- 1b_G4Brb-9_VP7_DNA_opt
atggatttcatcatatataggtttctgtttataatagtgattctgtcacctctactcaaggcgcaaaactatggcataaacctccctatcaccggctcaatggacaccgcctatgcaaactccacgcaggaagaaactctgctgtctagcacactgtgcctttactatcctagtgaggcaccgactcaaatcagtgatacagaatggaaggatacactgtctcaactctttctcaccaagggatggcccactggctcagtgtattttaatgaatacagcaacgttttggagttcagtattgaccccaagctgtactgcgactacaatgtagtgctgattcgattcgcctcgggggaggaacttgacgtatccgagttggccgacctcatcctgaatgagtggctgtgtaatccaatggatatcacactctactactaccagcagactgacgaagccaacaagtggatctctatgggttctagctgcaccatcaaagtgtgccccctgaacacccagacactgggcattggctgtctgacgacagatgtcagtaccttcgaggaggtggcgacaacagagaaactggtgatcaccgacgtggttgacggcgtgaaccacaaactcgacgtgacaactaccacctgcaccatccggaattgtaagaagctgggaccgagagaaaacgttgccataatccaggtgggaggtagcaatatcctcgacataaccgccgatcctacgacgtcccctcagactgaaaggatgatgcgagtcaactggaagaagtggtggcaagttttctatacagtggttgactatatcaaccaaatagtcaaggtgatgagtaaacgcagcagatcccttaactccgctgcattctattatagggtgtaa

SEQ ID NO: 69 - protein, 297 aa, Artificial Sequence
  Synthetic sequence, G3HCR3+7-1a-1b_G4Brb- 9_VP7_AA
MDFIIYRFLFIIVILSPLLKAQNYGINLPITGSMDTAYANSTQEETLLSSTLCLYYPSEAPTQISDTEWKDTLSQLFLTKGWPTGSVYFNEYSNVLEFSIDPKLYCDYNVVLIRFASGEELDVSELADLILNEWLCNPMDITLYYYQQTDEANKWISMGSSCTIKVCPLNTQTLGIGCLTTDVSTFEEVATTEKLVITDVVDGVNHKLDVTTTTCTIRNCKKLGPRENVAIIQVGGSNILDITADPTTSPQTERMMRVNWKKWWQVFYTVVDYINQIVKVMSKRSRSLNSAAFYYRV

SEQ ID NO: 70 - DNA, 894 bp, Artificial Sequence
  Synthetic sequence, G3HCR3+7-1a- 1b_G9BE2001_VP7_DNA_opt
atggatttcatcatatataggtttctgtttataatagtgattctgtcacctctactcaaggcgcaaaactatggcataaacctccctatcaccggctcaatggacaccgcctatgcaaactccacgcaggaagaaactctgctgacatcaaccttgtgcttgtattaccccactgaagcgtctactcagatcggagataccgagtggaaagatactctcagtcagctgttcctcaccaagggatggccaacaggctctgtctactttaaagagtacacggacatcgcatcttttagcatcgatcctcagttatactgcgactacaacgtggtgttgatgaaatacgacagcacgctggagctcgacatgtccgagctggctgatctgattctcaatgagtggctgtgtaatccaatggatatcacactctactactaccagcagactgacgaagccaacaagtggatctctatgggttctagctgcaccatcaaagtgtgccccctgaacacccagacactgggcattggctgtctgacgacagatgtcagtaccttcgaggaggtggcgacaacagagaaactggtgatcaccgacgtggttgacggcgtgaaccacaaactcgacgtgacaactaccacctgcaccatccggaattgtaagaagctgggaccgagagaaaacgtggctatcgttcaggtgggcggttccgaggttctcgacataacggctgacccaaccaccgccccacagaccgagaggatgatgcgcgtgaactggaaaaaatggtggcaagtgttctacactgtggtggactatatcaaccagattgtgcaggtgatgtccaaacgcagcagatcccttaactccgctgcattctattatagggtgtaa

SEQ ID NO: 71 - protein, 297 aa, Artificial Sequence
  Synthetic sequence, Protein G3HCR3+7-1a- 1b_G9BE2001_VP7_AA
MDFIIYRFLFIIVILSPLLKAQNYGINLPITGSMDTAYANSTQEETLLTSTLCLYYPTEASTQIGDTEWKDTLSQLFLTKGWPTGSVYFKEYTDIASFSIDPQLYCDYNVVLMKYDSTLELDMSELADLILNEWLCNPMDITLYYYQQTDEANKWISMGSSCTIKVCPLNTQTLGIGCLTTDVSTFEEVATTEKLVITDVVDGVNHKLDVTTTTCTIRNCKKLGPRENVAIVQVGGSEVLDITADPTTAPQTERMMRVNWKKWWQVFYTVVDYINQIVQVMSKRSRSLNSAAFYYRV

SEQ ID NO: 72 - DNA, 894 bp, Artificial Sequence
  Synthetic sequence, G3HCR3+7-1a- 1b_G12K12_VP7_DNA_opt
atggatttcatcatatataggtttctgtttataatagtgattctgtcacctctactcaaggcgcaaaactatggcataaacctccctatcaccggctcaatggacaccgcctatgcaaactccacgcaggaagaaactctgctgacctctaccttgtgtctgtattacccctcttctgtcacaacagagatcacagatcctgattggaccaatacattgtctcagctcttcatgaccaaagggtggcctactaacagcgtgtatttcaagtcatatgcggacatcgctagcttcagcgttgatccacagctctattgcgactacaacatcgttctggttcagtaccaaaattccctggccttagacgtgtctgaactcgccgacctgatcctgaatgagtggctgtgtaatccaatggatatcacactctactactaccagcagactgacgaagccaacaagtggatctctatgggttctagctgcaccatcaaagtgtgccccctgaacacccagacactgggcattggctgtctgacgacagatgtcagtaccttcgaggaggtggcgacaacagagaaactggtgatcaccgacgtggttgacggcgtgaaccacaaactcgacgtgacaactaccacctgcaccatccggaattgtaagaagctgggaccgagagaaaacgtcgcgataatacaagtgggtggctctgatgttatcgatataacagctgatcccacaacgattccacagacagagcggatgatgcggatcaactggaagaagtggtggcaggttttttacaccgtggtcgattacatcaaccagatcgtacaggtgatgagcaagcgcagcagatcccttaactccgctgcattctattatagggtgtaa

SEQ ID NO: 73 - protein, 297 aa, Artificial Sequence
  Synthetic sequence, G3HCR3+7-1a- 1b_G12K12_VP7_AA
MDFIIYRFLFIIVILSPLLKAQNYGINLPITGSMDTAYANSTQEETLLTSTLCLYYPSSVTTEITDPDWTNTLSQLFMTKGWPTNSVYFKSYADIASFSVDPQLYCDYNIVLVQYQNSLALDVSELADLILNEWLCNPMDITLYYYQQTDEANKWISMGSSCTIKVCPLNTQTLGIGCLTTDVSTFEEVATTEKLVITDVVDGVNHKLDVTTTTCTIRNCKKLGPRENVAIIQVGGSDVIDITADPTTIPQTERMMRINWKKWWQVFYTVVDYINQIVQVMSKRSRSLNSAAFYYRV

SEQ ID NO: 74 - DNA, 54 bp, Artificial Sequence
  Synthetic sequence, IF-(160)RVA(G4P5BrB-9)VP7.c
tcgtgcttcggcaccagtacaatggattatctgatctaccgcatcacctttgtg

SEQ ID NO: 75 - DNA, 48 bp, Artificial Sequence
  Synthetic sequence, IF-RVA(G4P5BrB-9)VP7.r
actaaagaaaataggcctttaaactctgtagtaaaagctactggagtc

SEQ ID NO: 76 - DNA, 894 bp, Artificial Sequence
  Synthetic sequence, G4BrB-9_VP7_DNA_opt
atggattatctgatctaccgcatcacctttgtgattgtcgttctctctgtgttatcaaatgctcagaattacggcatcaacctgccaattaccggctccatggacacagcctacgctaactccacccaggacaacaactttttgtctagcacactgtgcctttactatcctagtgaggcaccgactcaaatcagtgatacagaatggaaggatacactgtctcaactctttctcaccaagggatggcccactggctcagtgtattttaatgaatacagcaacgttttggagttcagtattgaccccaagctgtactgcgactacaatgtagtgctgattcgattcgcctcgggggaggaacttgacgtatccgagttggccgacctcatcctgaatgaatggctttgtaatcctatggacattacgctgtactattaccagcagaccggcgaggccaacaaatggatctcgatggggagcagctgcactgtgaaggtgtgtcccctgaacacccagactctcggtatcgggtgccagacaactgataccgcaacttttgagacagtggcagatagcgagaagctggccctaattgatgtggtggataatgtgaaccacaagctggacgtaacatcgacaacctgtactatccgaaactgtaacaaacttgggccacgagagaacgttgccataatccaggtgggaggtagcaatatcctcgacataaccgccgatcctacgacgtcccctcagactgaaaggatgatgcgagtcaactggaagaagtggtggcaagttttctatacagtggttgactatatcaaccaaatagtcaaggtgatgagtaaaagatcccgatccctagactccagtagcttttactacagagtttaa

SEQ ID NO: 77 - protein, 297 aa, Artificial Sequence
  Synthetic sequence, G3HCR3_VP7_AA
MDFIIYRFLFIIVILSPLLKAQNYGINLPITGSMDTAYANSTQEETLLTSTLCLYYPTEAATEINDNSWKDTLSQLFLTKGWPTGSVYFKEYTDIASFSVDPQLYCDYNIVLMKYDAALQLDMSELADLILNEWLCNPMDITLYYYQQTDEANKWISMGSSCTIKVCPLNTQTLGIGCLTTDVSTFEEVATTEKLVITDVVDGVNHKLDVTTTTCTIRNCKKLGPRENVAVIQVGGSDILDITADPTTAPQTERMMRINWKKWWQVFYTVVDYVNQIIQAMSKRSRSLNSAAFYYRV

SEQ ID NO: 78 - protein, 297 aa, Artificial sequence
  Synthetic sequence, Protein G4BrB-9_VP7_AA
MDYLIYRITFVIVVLSVLSNAQNYGINLPITGSMDTAYANSTQDNNFLSSTLCLYYPSEAPTQISDTEWKDTLSQLFLTKGWPTGSVYFNEYSNVLEFSIDPKLYCDYNVVLIRFASGEELDVSELADLILNEWLCNPMDITLYYYQQTGEANKWISMGSSCTVKVCPLNTQTLGIGCQTTDTATFETVADSEKLALIDVVDNVNHKLDVTSTTCTIRNCNKLGPRENVAIIQVGGSNILDITADPTTSPQTERMMRVNWKKWWQVFYTVVDYINQIVKVMSKRSRSLDSSSFYYRV

SEQ ID NO: 79 - DNA, 894 bp, Artificial Sequence
  Synthetic sequence, G4BrB-9+7-1a- 1b_G1Rtx_VP7_DNA_opt
atggattatctgatctaccgcatcacctttgtgattgtcgttctctctgtgttatcaaatgctcagaattacggcatcaacctgccaattaccggctccatggacacagcctacgctaactccacccaggacaacaactttttgacaagtaccctgtgcctgtattatccaacagaagcctctacccagatcaatgatggggagtggaaggatagtctctcacagatgttcctaaccaagggctggcccaccggttccgtctacttcaaggaatactctagtattgtcgacttctcagttgacccccagctttattgcgactacaacctggtacttatgaaatacgaccagaacctggagctggatatgtccgagctggctgacctgatcctcaatgaatggctttgtaatcctatggacattacgctgtactattaccagcagaccggcgaggccaacaaatggatctcgatggggagcagctgcactgtgaaggtgtgtcccctgaacacccagactctcggtatcgggtgccagacaactgataccgcaacttttgagacagtggcagatagcgagaagctggccctaattgatgtggtggataatgtgaaccacaagctggacgtaacatcgacaacctgtactatccgaaactgtaacaaacttgggccacgagagaacgtcgccgtgatccaggtgggggggagcaatgtgctcgacattactgccgaccctaccaccaatccacagacggaacggatgatgagagtcaactggaagaaatggtggcaggtcttttataccattgtggactacattaaccagattgtgcaagtcatgagtaaaagatcccgatccctagactccagtagcttttactacagagtttaa

SEQ ID NO: 80 - protein, 297 aa, Artificial Sequence
  Synthetic sequence, G4BrB-9+7-1a- 1b_G1Rtx_VP7_AA
MDYLIYRITFVIVVLSVLSNAQNYGINLPITGSMDTAYANSTQDNNFLTSTLCLYYPTEASTQINDGEWKDSLSQMFLTKGWPTGSVYFKEYSSIVDFSVDPQLYCDYNLVLMKYDQNLELDMSELADLILNEWLCNPMDITLYYYQQTGEANKWISMGSSCTVKVCPLNTQTLGIGCQTTDTATFETVADSEKLALIDVVDNVNHKLDVTSTTCTIRNCNKLGPRENVAVIQVGGSNVLDITADPTTNPQTERMMRVNWKKWWQVFYTIVDYINQIVQVMSKRSRSLDSSSFYYRV

SEQ ID NO: 81 - DNA, 894 bp, Artificial Sequence
  Synthetic sequence, G4BrB-9+7-1a-1b_G2Sc2- 9_VP7_DNA_opt
atggattatctgatctaccgcatcacctttgtgattgtcgttctctctgtgttatcaaatgctcagaattacggcatcaacctgccaattaccggctccatggacacagcctacgctaactccacccaggacaacaactttttgacaagcaccctttgcctttattacccagcagaagcaaagaatgaaattagcgacgatgagtgggagaatacactttcacagctgtttctcaccaaggggtggccaaccggtagcgtatacttcaaagactataacgacattacgacctttagtatgaaccctcagctctactgtgactataacgtcgtgttaatgcgctatgacaataccagcgagctcgacgcctctgagctggctgacctgatcctgaatgaatggctttgtaatcctatggacattacgctgtactattaccagcagaccggcgaggccaacaaatggatctcgatggggagcagctgcactgtgaaggtgtgtcccctgaacacccagactctcggtatcgggtgccagacaactgataccgcaacttttgagacagtggcagatagcgagaagctggccctaattgatgtggtggataatgtgaaccacaagctggacgtaacatcgacaacctgtactatccgaaactgtaacaaacttgggccacgagagaacgtggccattatccaggttggcggccctaacgcgctcgacatcactgcagatccaacaaccgtgcctcaaattcagcggattatgagaatcaattggaaaaagtggtggcaggtgttttatacggttgtggactatattaatcagatcgtacaggtgatgagcaaaagatcccgatccctagactccagtagcttttactacagagtttaa

SEQ ID NO: 82 - protein, 297 aa, Artificial Sequence
  Synthetic sequence, G4BrB-9+7-1a-1b_G2Sc2- 9_VP7_AA
MDYLIYRITFVIVVLSVLSNAQNYGINLPITGSMDTAYANSTQDNNFLTSTLCLYYPAEAKNEISDDEWENTLSQLFLTKGWPTGSVYFKDYNDITTFSMNPQLYCDYNVVLMRYDNTSELDASELADLILNEWLCNPMDITLYYYQQTGEANKWISMGSSCTVKVCPLNTQTLGIGCQTTDTATFETVADSEKLALIDVVDNVNHKLDVTSTTCTIRNCNKLGPRENVAIIQVGGPNALDITADPTTVPQIQRIMRINWKKWWQVFYTVVDYINQIVQVMSKRSRSLDSSSFYYRV

SEQ ID NO: 83 - DNA, 894 bp, Artificial Sequence
  Synthetic sequence, G4BrB-9+7-1a- 1b_G3HCR3_VP7_DNA_opt
atggattatctgatctaccgcatcacctttgtgattgtcgttctctctgtgttatcaaatgctcagaattacggcatcaacctgccaattaccggctccatggacacagcctacgctaactccacccaggacaacaactttttgaccagcacactctgcctctactacccgacagaggctgccactgagatcaacgataattcttggaaagacacgttatcgcagctgtttcttactaagggctggcccaccggtagtgtctactttaaagagtataccgacattgcctcttttagcgtggatcctcagctctactgtgactataacatcgtgttgatgaagtatgacgcagcgctgcagctggatatgagtgagctggccgatttgatcctgaatgaatggctttgtaatcctatggacattacgctgtactattaccagcagaccggcgaggccaacaaatggatctcgatggggagcagctgcactgtgaaggtgtgtcccctgaacacccagactctcggtatcgggtgccagacaactgataccgcaacttttgagacagtggcagatagcgagaagctggccctaattgatgtggtggataatgtgaaccacaagctggacgtaacatcgacaacctgtactatccgaaactgtaacaaacttgggccacgagagaatgttgcagtcatccaggtaggaggcagtgatattctcgacatcacggccgacccgacgaccgcgcctcagacagaaaggatgatgcggatcaattggaagaagtggtggcaggtgttctacacagtggtggactacgttaaccagattattcaggctatgagcaagagatcccgatccctagactccagtagcttttactacagagtttaa

SEQ ID NO: 84 - protein, 297 aa, Artificial Sequence
  Synthetic sequence, G4BrB-9+7-1a- 1b_G3HCR3_VP7_AA
MDYLIYRITFVIVVLSVLSNAQNYGINLPITGSMDTAYANSTQDNNFLTSTLCLYYPTEAATEINDNSWKDTLSQLFLTKGWPTGSVYFKEYTDIASFSVDPQLYCDYNIVLMKYDAALQLDMSELADLILNEWLCNPMDITLYYYQQTGEANKWISMGSSCTVKVCPLNTQTLGIGCQTTDTATFETVADSEKLALIDVVDNVNHKLDVTSTTCTIRNCNKLGPRENVAVIQVGGSDILDITADPTTAPQTERMMRINWKKWWQVFYTVVDYVNQIIQAMSKRSRSLDSSSFYYRV

SEQ ID NO: 85 - DNA, 894 bp, Artificial Sequence
  Synthetic sequence, G4BrB-9+7-1a- 1b_G9BE2001_VP7_DNA_opt
atggattatctgatctaccgcatcacctttgtgattgtcgttctctctgtgttatcaaatgctcagaattacggcatcaacctgccaattaccggctccatggacacagcctacgctaactccacccaggacaacaactttttgacatcaaccttgtgcttgtattaccccactgaagcgtctactcagatcggagataccgagtggaaagatactctcagtcagctgttcctcaccaagggatggccaacaggctctgtctactttaaagagtacacggacatcgcatcttttagcatcgatcctcagttatactgcgactacaacgtggtgttgatgaaatacgacagcacgctggagctcgacatgtccgagctggctgatctgattctcaatgaatggctttgtaatcctatggacattacgctgtactattaccagcagaccggcgaggccaacaaatggatctcgatggggagcagctgcactgtgaaggtgtgtcccctgaacacccagactctcggtatcgggtgccagacaactgataccgcaacttttgagacagtggcagatagcgagaagctggccctaattgatgtggtggataatgtgaaccacaagctggacgtaacatcgacaacctgtactatccgaaactgtaacaaacttgggccacgagagaacgtggctatcgttcaggtgggcggttccgaggttctcgacataacggctgacccaaccaccgccccacagaccgagaggatgatgcgcgtgaactggaaaaaatggtggcaagtgttctacactgtggtggactatatcaaccagattgtgcaggtgatgtccaaaagatcccgatccctagactccagtagcttttactacagagtttaa

SEQ ID NO: 86 - protein, 297 aa, Artificial Sequence
  Synthetic sequence, G4BrB-9+7-1a- 1b_G9BE2001_VP7_AA
MDYLIYRITFVIVVLSVLSNAQNYGINLPITGSMDTAYANSTQDNNFLTSTLCLYYPTEASTQIGDTEWKDTLSQLFLTKGWPTGSVYFKEYTDIASFSIDPQLYCDYNVVLMKYDSTLELDMSELADLILNEWLCNPMDITLYYYQQTGEANKWISMGSSCTVKVCPLNTQTLGIGCQTTDTATFETVADSEKLALIDVVDNVNHKLDVTSTTCTIRNCNKLGPRENVAIVQVGGSEVLDITADPTTAPQTERMMRVNWKKWWQVFYTVVDYINQIVQVMSKRSRSLDSSSFYYRV

SEQ ID NO: 87 - DNA, 894 bp, Artificial Sequence
  Synthetic sequence, G4BrB-9+7-1a- 1b_G12K12_VP7_DNA_opt
atggattatctgatctaccgcatcacctttgtgattgtcgttctctctgtgttatcaaatgctcagaattacggcatcaacctgccaattaccggctccatggacacagcctacgctaactccacccaggacaacaactttttgacctctaccttgtgtctgtattacccctcttctgtcacaacagagatcacagatcctgattggaccaatacattgtctcagctcttcatgaccaaagggtggcctactaacagcgtgtatttcaagtcatatgcggacatcgctagcttcagcgttgatccacagctctattgcgactacaacatcgttctggttcagtaccaaaattccctggccttagacgtgtctgaactcgccgacctgatcctgaatgaatggctttgtaatcctatggacattacgctgtactattaccagcagaccggcgaggccaacaaatggatctcgatggggagcagctgcactgtgaaggtgtgtcccctgaacacccagactctcggtatcgggtgccagacaactgataccgcaacttttgagacagtggcagatagcgagaagctggccctaattgatgtggtggataatgtgaaccacaagctggacgtaacatcgacaacctgtactatccgaaactgtaacaaacttgggccacgagagaacgtcgcgataatacaagtgggtggctctgatgttatcgatataacagctgatcccacaacgattccacagacagagcggatgatgcggatcaactggaagaagtggtggcaggttttttacaccgtggtcgattacatcaaccagatcgtacaggtgatgagcaagagatcccgatccctagactccagtagcttttactacagagtttaa

SEQ ID NO: 88 - protein, 297 aa, Artificial Sequence
  Synthetic sequence, G4BrB-9+7-1a- 1b_G12K12_VP7_AA
MDYLIYRITFVIVVLSVLSNAQNYGINLPITGSMDTAYANSTQDNNFLTSTLCLYYPSSVTTEITDPDWTNTLSQLFMTKGWPTNSVYFKSYADIASFSVDPQLYCDYNIVLVQYQNSLALDVSELADLILNEWLCNPMDITLYYYQQTGEANKWISMGSSCTVKVCPLNTQTLGIGCQTTDTATFETVADSEKLALIDVVDNVNHKLDVTSTTCTIRNCNKLGPRENVAIIQVGGSDVIDITADPTTIPQTERMMRINWKKWWQVFYTVVDYINQIVQVMSKRSRSLDSSSFYYRV

SEQ ID NO: 89 - DNA, 49 bp, Artificial Sequence
  Synthetic sequence, IF-VP7(G9AFJ11215)(opt).c
tcgtgcttcggcaccagtacaatggatttcatcatctacaggttcctgc

SEQ ID NO: 90 - DNA, 49 bp, Artificial Sequence
  Synthetic sequence, IF-VP7(G9AFJ11215)(opt).r
actaaagaaaataggccttcacactcgataatagaaggcggctgagttc

SEQ ID NO: 91 - DNA, 894 bp, Artificial Sequence
  Synthetic sequence, G9BE2001_VP7_DNA_opt
atggatttcatcatctacaggttcctgcttttcatcgttattgtgagcccctttgtgaagacacagaactacggcatcaacctcccaattaccggttcgatggacgcagcctacgcaaattcctcacagcaggagacctttctcacatcaaccttgtgcttgtattaccccactgaagcgtctactcagatcggagataccgagtggaaagatactctcagtcagctgttcctcaccaagggatggccaacaggctctgtctactttaaagagtacacggacatcgcatcttttagcatcgatcctcagttatactgcgactacaacgtggtgttgatgaaatacgacagcacgctggagctcgacatgtccgagctggctgatctgattctcaacgagtggctttgcaacccgatggatatcaccctgtattactatcagcagaccgacgaagccaataagtggattagcatggggcagtcctgcactattaaggtgtgccccctcaatacacaaaccctcggcatcggctgcactaccaccaacaccgccacttttgaggaggtggctacacgagaaaagctcgtgatcactgacgtggtggacggcgtgaaccacaagctggacgtcaccaccaacacatgtaccatacgcaactgcaagaagctgggacccagggaaaacgtggctatcgttcaggtgggcggttccgaggttctcgacataacggctgacccaaccaccgccccacagaccgagaggatgatgcgcgtgaactggaaaaaatggtggcaagtgttctacactgtggtggactatatcaaccagattgtgcaggtgatgtccaaacggtcgcggtctctgaactcagccgccttctattatcgagtgtga

SEQ ID NO: 92 - protein, 297 aa, Artificial Sequence
  Synthetic sequence, G9BE2001_VP7_AA
MDFIIYRFLLFIVIVSPFVKTQNYGINLPITGSMDAAYANSSQQETFLTSTLCLYYPTEASTQIGDTEWKDTLSQLFLTKGWPTGSVYFKEYTDIASFSIDPQLYCDYNVVLMKYDSTLELDMSELADLILNEWLCNPMDITLYYYQQTDEANKWISMGQSCTIKVCPLNTQTLGIGCTTTNTATFEEVATREKLVITDVVDGVNHKLDVTTNTCTIRNCKKLGPRENVAIVQVGGSEVLDITADPTTAPQTERMMRVNWKKWWQVFYTVVDYINQIVQVMSKRSRSLNSAAFYYRV

SEQ ID NO: 93 - DNA, 894 bp, Artificial Sequence
  Synthetic sequence, G9BE2001+7-1a- 1b_G1Rtx_VP7_DNA_opt
atggatttcatcatctacaggttcctgcttttcatcgttattgtgagcccctttgtgaagacacagaactacggcatcaacctcccaattaccggttcgatggacgcagcctacgcaaattcctcacagcaggagacctttctcacaagtaccctgtgcctgtattatccaacagaagcctctacccagatcaatgatggggagtggaaggatagtctctcacagatgttcctaaccaagggctggcccaccggttccgtctacttcaaggaatactctagtattgtcgacttctcagttgacccccagctttattgcgactacaacctggtacttatgaaatacgaccagaacctggagctggatatgtccgagctggctgacctgatcctcaacgagtggctttgcaacccgatggatatcaccctgtattactatcagcagaccgacgaagccaataagtggattagcatggggcagtcctgcactattaaggtgtgccccctcaatacacaaaccctcggcatcggctgcactaccaccaacaccgccacttttgaggaggtggctacacgagaaaagctcgtgatcactgacgtggtggacggcgtgaaccacaagctggacgtcaccaccaacacatgtaccatacgcaactgcaagaagctgggacccagggaaaacgtcgccgtgatccaggtgggggggagcaatgtgctcgacattactgccgaccctaccaccaatccacagacggaacggatgatgagagtcaactggaagaaatggtggcaggtcttttataccattgtggactacattaaccagattgtgcaagtcatgagtaaacggtcgcggtctctgaactcagccgccttctattatcgagtgtga

SEQ ID NO: 94 - protein, 297 aa, Artificial Sequence
  Synthetic sequence, G9BE2001+7-1a- 1b_G1Rtx_VP7_AA
MDFIIYRFLLFIVIVSPFVKTQNYGINLPITGSMDAAYANSSQQETFLTSTLCLYYPTEASTQINDGEWKDSLSQMFLTKGWPTGSVYFKEYSSIVDFSVDPQLYCDYNLVLMKYDQNLELDMSELADLILNEWLCNPMDITLYYYQQTDEANKWISMGQSCTIKVCPLNTQTLGIGCTTTNTATFEEVATREKLVITDVVDGVNHKLDVTTNTCTIRNCKKLGPRENVAVIQVGGSNVLDITADPTTNPQTERMMRVNWKKWWQVFYTIVDYINQIVQVMSKRSRSLNSAAFYYRV

SEQ ID NO: 95 - DNA, 894 bp, Artificial Sequence
  Synthetic sequence, G9BE2001+7-1a-1b_G2Sc2- 9_VP7_DNA_opt
atggatttcatcatctacaggttcctgcttttcatcgttattgtgagcccctttgtgaagacacagaactacggcatcaacctcccaattaccggttcgatggacgcagcctacgcaaattcctcacagcaggagacctttctcacaagcaccctttgcctttattacccagcagaagcaaagaatgaaattagcgacgatgagtgggagaatacactttcacagctgtttctcaccaaggggtggccaaccggtagcgtatacttcaaagactataacgacattacgacctttagtatgaaccctcagctctactgtgactataacgtcgtgttaatgcgctatgacaataccagcgagctcgacgcctctgagctggctgacctgatcctgaacgagtggctttgcaacccgatggatatcaccctgtattactatcagcagaccgacgaagccaataagtggattagcatggggcagtcctgcactattaaggtgtgccccctcaatacacaaaccctcggcatcggctgcactaccaccaacaccgccacttttgaggaggtggctacacgagaaaagctcgtgatcactgacgtggtggacggcgtgaaccacaagctggacgtcaccaccaacacatgtaccatacgcaactgcaagaagctgggacccagggaaaacgtggccattatccaggttggcggccctaacgcgctcgacatcactgcagatccaacaaccgtgcctcaaattcagcggattatgagaatcaattggaaaaagtggtggcaggtgttttatacggttgtggactatattaatcagatcgtacaggtgatgagcaaacggtcgcggtctctgaactcagccgccttctattatcgagtgtga

SEQ ID NO: 96 - protein, 297 aa, Artificial Sequence
  Synthetic sequence, G9BE2001+7-1a-1b_G2Sc2- 9_VP7_AA
MDFIIYRFLLFIVIVSPFVKTQNYGINLPITGSMDAAYANSSQQETFLTSTLCLYYPAEAKNEISDDEWENTLSQLFLTKGWPTGSVYFKDYNDITTFSMNPQLYCDYNVVLMRYDNTSELDASELADLILNEWLCNPMDITLYYYQQTDEANKWISMGQSCTIKVCPLNTQTLGIGCTTTNTATFEEVATREKLVITDVVDGVNHKLDVTTNTCTIRNCKKLGPRENVAIIQVGGPNALDITADPTTVPQIQRIMRINWKKWWQVFYTVVDYINQIVQVMSKRSRSLNSAAFYYRV

SEQ ID NO: 97 - DNA, 894 bp, Artificial Sequence
  Synthetic sequence, G9BE2001+7-1a- 1b_G3HCR3_VP7_DNA_opt
atggatttcatcatctacaggttcctgcttttcatcgttattgtgagcccctttgtgaagacacagaactacggcatcaacctcccaattaccggttcgatggacgcagcctacgcaaattcctcacagcaggagacctttctcaccagcacactctgcctctactacccgacagaggctgccactgagatcaacgataattcttggaaagacacgttatcgcagctgtttcttactaagggctggcccaccggtagtgtctactttaaagagtataccgacattgcctcttttagcgtggatcctcagctctactgtgactataacatcgtgttgatgaagtatgacgcagcgctgcagctggatatgagtgagctggccgatttgatcctgaacgagtggctttgcaacccgatggatatcaccctgtattactatcagcagaccgacgaagccaataagtggattagcatggggcagtcctgcactattaaggtgtgccccctcaatacacaaaccctcggcatcggctgcactaccaccaacaccgccacttttgaggaggtggctacacgagaaaagctcgtgatcactgacgtggtggacggcgtgaaccacaagctggacgtcaccaccaacacatgtaccatacgcaactgcaagaagctgggacccagggaaaatgttgcagtcatccaggtaggaggcagtgatattctcgacatcacggccgacccgacgaccgcgcctcagacagaaaggatgatgcggatcaattggaagaagtggtggcaggtgttctacacagtggtggactacgttaaccagattattcaggctatgagcaagcggtcgcggtctctgaactcagccgccttctattatcgagtgtga

SEQ ID NO: 98 - protein, 297 aa, Artificial Sequence
  Synthetic sequence, G9BE2001+7-1a- 1b_G3HCR3_VP7_AA
MDFIIYRFLLFIVIVSPFVKTQNYGINLPITGSMDAAYANSSQQETFLTSTLCLYYPTEAATEINDNSWKDTLSQLFLTKGWPTGSVYFKEYTDIASFSVDPQLYCDYNIVLMKYDAALQLDMSELADLILNEWLCNPMDITLYYYQQTDEANKWISMGQSCTIKVCPLNTQTLGIGCTTTNTATFEEVATREKLVITDVVDGVNHKLDVTTNTCTIRNCKKLGPRENVAVIQVGGSDILDITADPTTAPQTERMMRINWKKWWQVFYTVVDYVNQIIQAMSKRSRSLNSAAFYYRV

SEQ ID NO: 99 - DNA, 894 bp, Artificial Sequence
  Synthetic sequence, G9BE2001+7-1a-1b_G4BrB- 9_VP7_DNA_opt
atggatttcatcatctacaggttcctgcttttcatcgttattgtgagcccctttgtgaagacacagaactacggcatcaacctcccaattaccggttcgatggacgcagcctacgcaaattcctcacagcaggagacctttctctctagcacactgtgcctttactatcctagtgaggcaccgactcaaatcagtgatacagaatggaaggatacactgtctcaactctttctcaccaagggatggcccactggctcagtgtattttaatgaatacagcaacgttttggagttcagtattgaccccaagctgtactgcgactacaatgtagtgctgattcgattcgcctcgggggaggaacttgacgtatccgagttggccgacctcatcctgaacgagtggctttgcaacccgatggatatcaccctgtattactatcagcagaccgacgaagccaataagtggattagcatggggcagtcctgcactattaaggtgtgccccctcaatacacaaaccctcggcatcggctgcactaccaccaacaccgccacttttgaggaggtggctacacgagaaaagctcgtgatcactgacgtggtggacggcgtgaaccacaagctggacgtcaccaccaacacatgtaccatacgcaactgcaagaagctgggacccagggaaaacgttgccataatccaggtgggaggtagcaatatcctcgacataaccgccgatcctacgacgtcccctcagactgaaaggatgatgcgagtcaactggaagaagtggtggcaagttttctatacagtggttgactatatcaaccaaatagtcaaggtgatgagtaaacggtcgcggtctctgaactcagccgccttctattatcgagtgtga

SEQ ID NO: 100 - protein, 297 aa, Artificial Sequence
  Synthetic sequence, G9BE2001+7-1a-1b_G4BrB- 9_VP7_AA
MDFIIYRFLLFIVIVSPFVKTQNYGINLPITGSMDAAYANSSQQETFLSSTLCLYYPSEAPTQISDTEWKDTLSQLFLTKGWPTGSVYFNEYSNVLEFSIDPKLYCDYNVVLIRFASGEELDVSELADLILNEWLCNPMDITLYYYQQTDEANKWISMGQSCTIKVCPLNTQTLGIGCTTTNTATFEEVATREKLVITDVVDGVNHKLDVTTNTCTIRNCKKLGPRENVAIIQVGGSNILDITADPTTSPQTERMMRVNWKKWWQVFYTVVDYINQIVKVMSKRSRSLNSAAFYYRV

SEQ ID NO: 101 - DNA, 894 bp, Artificial Sequence
  Synthetic sequence, G9BE2001+7-1a- 1b_G12K12_VP7_DNA_opt
atggatttcatcatctacaggttcctgcttttcatcgttattgtgagcccctttgtgaagacacagaactacggcatcaacctcccaattaccggttcgatggacgcagcctacgcaaattcctcacagcaggagacctttctcacctctaccttgtgtctgtattacccctcttctgtcacaacagagatcacagatcctgattggaccaatacattgtctcagctcttcatgaccaaagggtggcctactaacagcgtgtatttcaagtcatatgcggacatcgctagcttcagcgttgatccacagctctattgcgactacaacatcgttctggttcagtaccaaaattccctggccttagacgtgtctgaactcgccgacctgatcctgaacgagtggctttgcaacccgatggatatcaccctgtattactatcagcagaccgacgaagccaataagtggattagcatggggcagtcctgcactattaaggtgtgccccctcaatacacaaaccctcggcatcggctgcactaccaccaacaccgccacttttgaggaggtggctacacgagaaaagctcgtgatcactgacgtggtggacggcgtgaaccacaagctggacgtcaccaccaacacatgtaccatacgcaactgcaagaagctgggacccagggaaaacgtcgcgataatacaagtgggtggctctgatgttatcgatataacagctgatcccacaacgattccacagacagagcggatgatgcggatcaactggaagaagtggtggcaggttttttacaccgtggtcgattacatcaaccagatcgtacaggtgatgagcaagcggtcgcggtctctgaactcagccgccttctattatcgagtgtga

SEQ ID NO: 102 - protein, 297 aa, Artificial Sequence
  Synthetic sequence, G9BE2001+7-1a- 1b_G12K12_VP7_AA
MDFIIYRFLLFIVIVSPFVKTQNYGINLPITGSMDAAYANSSQQETFLTSTLCLYYPSSVTTEITDPDWTNTLSQLFMTKGWPTNSVYFKSYADIASFSVDPQLYCDYNIVLVQYQNSLALDVSELADLILNEWLCNPMDITLYYYQQTDEANKWISMGQSCTIKVCPLNTQTLGIGCTTTNTATFEEVATREKLVITDVVDGVNHKLDVTTNTCTIRNCKKLGPRENVAIIQVGGSDVIDITADPTTIPQTERMMRINWKKWWQVFYTVVDYINQIVQVMSKRSRSLNSAAFYYRV

SEQ ID NO: 103 - DNA, 50 bp, Artificial Sequence
  Synthetic sequence, IF-VP7(G12BAD89095).c
tcgtgcttcggcaccagtacaatggactttatcatatataggttcctgct

SEQ ID NO: 104 - DNA, 50 bp, Artificial Sequence
  Synthetic sequence, IF-VP7(G12BAD89095).r
actaaagaaaataggcctctagatcctgtagtagaatgcggcagaattaa

SEQ ID NO: 105 - DNA, 894 bp, Artificial Sequence
  Synthetic sequence, G12K12_VP7_DNA_opt
atggactttatcatatataggttcctgctcatcgtggttgtgatgttgccattcataaaagcccagaactacgggatcaacctgcccataacaggatctatggacacagcttacaccaattcaactcaacaagagaatttcatgacctctaccttgtgtctgtattacccctcttctgtcacaacagagatcacagatcctgattggaccaatacattgtctcagctcttcatgaccaaagggtggcctactaacagcgtgtatttcaagtcatatgcggacatcgctagcttcagcgttgatccacagctctattgcgactacaacatcgttctggttcagtaccaaaattccctggccttagacgtgtctgaactcgccgacctgatcctgaacgaatggctatgtaacccaatggacgtgaccctgtactactaccagcagaccgacgaggcaaataagtggatcagcatgggagaatcttgcaccgtgaaagtttgtccactgaatacacagactctcgggatcggctgcactactaccgatgttaccacctttgaagaagtggcaaacgccgagaagcttgtcatcacagatgtagttgacggcgttaatcacaaaattaatattactatgaacacctgcacgattaggaattgtaagaaactggggccacgcgaaaacgtcgcgataatacaagtgggtggctctgatgttatcgatataacagctgatcccacaacgattccacagacagagcggatgatgcggatcaactggaagaagtggtggcaggttttttacaccgtggtcgattacatcaaccagatcgtacaggtgatgagcaagcgtagccggagccttaattctgccgcattctactacaggatctag

SEQ ID NO: 106 - protein, 297 aa, Artificial Sequence
  Synthetic sequence, G12K12_VP7_AA
MDFIIYRFLLIVVVMLPFIKAQNYGINLPITGSMDTAYTNSTQQENFMTSTLCLYYPSSVTTEITDPDWTNTLSQLFMTKGWPTNSVYFKSYADIASFSVDPQLYCDYNIVLVQYQNSLALDVSELADLILNEWLCNPMDVTLYYYQQTDEANKWISMGESCTVKVCPLNTQTLGIGCTTTDVTTFEEVANAEKLVITDVVDGVNHKINITMNTCTIRNCKKLGPRENVAIIQVGGSDVIDITADPTTIPQTERMMRINWKKWWQVFYTVVDYINQIVQVMSKRSRSLNSAAFYYRI

SEQ ID NO: 107 - DNA, 894 bp, Artificial Sequence
  Synthetic sequence, G12K12+7-1a- 1b_G1Rtx_VP7_DNA_opt
atggactttatcatatataggttcctgctcatcgtggttgtgatgttgccattcataaaagcccagaactacgggatcaacctgcccataacaggatctatggacacagcttacaccaattcaactcaacaagagaatttcatgacaagtaccctgtgcctgtattatccaacagaagcctctacccagatcaatgatggggagtggaaggatagtctctcacagatgttcctaaccaagggctggcccaccggttccgtctacttcaaggaatactctagtattgtcgacttctcagttgacccccagctttattgcgactacaacctggtacttatgaaatacgaccagaacctggagctggatatgtccgagctggctgacctgatcctcaacgaatggctatgtaacccaatggacgtgaccctgtactactaccagcagaccgacgaggcaaataagtggatcagcatgggagaatcttgcaccgtgaaagtttgtccactgaatacacagactctcgggatcggctgcactactaccgatgttaccacctttgaagaagtggcaaacgccgagaagcttgtcatcacagatgtagttgacggcgttaatcacaaaattaatattactatgaacacctgcacgattaggaattgtaagaaactggggccacgcgaaaacgtcgccgtgatccaggtgggggggagcaatgtgctcgacattactgccgaccctaccaccaatccacagacggaacggatgatgagagtcaactggaagaaatggtggcaggtcttttataccattgtggactacattaaccagattgtgcaagtcatgagtaaacgtagccggagccttaattctgccgcattctactacaggatctag

SEQ ID NO: 108 - protein, 297 aa, Artificial Sequence
  Synthetic sequence, G12K12+7-1a-1b_G1Rtx_VP7_AA
MDFIIYRFLLIVVVMLPFIKAQNYGINLPITGSMDTAYTNSTQQENFMTSTLCLYYPTEASTQINDGEWKDSLSQMFLTKGWPTGSVYFKEYSSIVDFSVDPQLYCDYNLVLMKYDQNLELDMSELADLILNEWLCNPMDVTLYYYQQTDEANKWISMGESCTVKVCPLNTQTLGIGCTTTDVTTFEEVANAEKLVITDVVDGVNHKINITMNTCTIRNCKKLGPRENVAVIQVGGSNVLDITADPTTNPQTERMMRVNWKKWWQVFYTIVDYINQIVQVMSKRSRSLNSAAFYYRI

SEQ ID NO: 109 - DNA, 894 bp, Artificial Sequence
  Synthetic sequence, G12K12+7-1a-1b_G2Sc2- 9_VP7_DNA_opt
atggactttatcatatataggttcctgctcatcgtggttgtgatgttgccattcataaaagcccagaactacgggatcaacctgcccataacaggatctatggacacagcttacaccaattcaactcaacaagagaatttcatgacaagcaccctttgcctttattacccagcagaagcaaagaatgaaattagcgacgatgagtgggagaatacactttcacagctgtttctcaccaaggggtggccaaccggtagcgtatacttcaaagactataacgacattacgacctttagtatgaaccctcagctctactgtgactataacgtcgtgttaatgcgctatgacaataccagcgagctcgacgcctctgagctggctgacctgatcctgaacgaatggctatgtaacccaatggacgtgaccctgtactactaccagcagaccgacgaggcaaataagtggatcagcatgggagaatcttgcaccgtgaaagtttgtccactgaatacacagactctcgggatcggctgcactactaccgatgttaccacctttgaagaagtggcaaacgccgagaagcttgtcatcacagatgtagttgacggcgttaatcacaaaattaatattactatgaacacctgcacgattaggaattgtaagaaactggggccacgcgaaaacgtggccattatccaggttggcggccctaacgcgctcgacatcactgcagatccaacaaccgtgcctcaaattcagcggattatgagaatcaattggaaaaagtggtggcaggtgttttatacggttgtggactatattaatcagatcgtacaggtgatgagcaaacgtagccggagccttaattctgccgcattctactacaggatctag

SEQ ID NO: 110 - protein, 297 aa, Artificial Sequence
  Synthetic sequence, G12K12+7-1a-1b_G2Sc2- 9_VP7_AA
MDFIIYRFLLIVVVMLPFIKAQNYGINLPITGSMDTAYTNSTQQENFMTSTLCLYYPAEAKNEISDDEWENTLSQLFLTKGWPTGSVYFKDYNDITTFSMNPQLYCDYNVVLMRYDNTSELDASELADLILNEWLCNPMDVTLYYYQQTDEANKWISMGESCTVKVCPLNTQTLGIGCTTTDVTTFEEVANAEKLVITDVVDGVNHKINITMNTCTIRNCKKLGPRENVAIIQVGGPNALDITADPTTVPQIQRIMRINWKKWWQVFYTVVDYINQIVQVMSKRSRSLNSAAFYYRI

SEQ ID NO: 111 - DNA, 894 bp, Artificial Sequence
  Synthetic sequence, G12K12+7-1a- 1b_G3HCR3_VP7_DNA_opt
atggactttatcatatataggttcctgctcatcgtggttgtgatgttgccattcataaaagcccagaactacgggatcaacctgcccataacaggatctatggacacagcttacaccaattcaactcaacaagagaatttcatgaccagcacactctgcctctactacccgacagaggctgccactgagatcaacgataattcttggaaagacacgttatcgcagctgtttcttactaagggctggcccaccggtagtgtctactttaaagagtataccgacattgcctcttttagcgtggatcctcagctctactgtgactataacatcgtgttgatgaagtatgacgcagcgctgcagctggatatgagtgagctggccgatttgatcctgaacgaatggctatgtaacccaatggacgtgaccctgtactactaccagcagaccgacgaggcaaataagtggatcagcatgggagaatcttgcaccgtgaaagtttgtccactgaatacacagactctcgggatcggctgcactactaccgatgttaccacctttgaagaagtggcaaacgccgagaagcttgtcatcacagatgtagttgacggcgttaatcacaaaattaatattactatgaacacctgcacgattaggaattgtaagaaactggggccacgcgaaaatgttgcagtcatccaggtaggaggcagtgatattctcgacatcacggccgacccgacgaccgcgcctcagacagaaaggatgatgcggatcaattggaagaagtggtggcaggtgttctacacagtggtggactacgttaaccagattattcaggctatgagcaagcgtagccggagccttaattctgccgcattctactacaggatctag

SEQ ID NO: 112 - protein, 297 aa, Artificial Sequence
  Synthetic sequence, G12K12+7-1a- 1b_G3HCR3_VP7_AA
MDFIIYRFLLIVVVMLPFIKAQNYGINLPITGSMDTAYTNSTQQENFMTSTLCLYYPTEAATEINDNSWKDTLSQLFLTKGWPTGSVYFKEYTDIASFSVDPQLYCDYNIVLMKYDAALQLDMSELADLILNEWLCNPMDVTLYYYQQTDEANKWISMGESCTVKVCPLNTQTLGIGCTTTDVTTFEEVANAEKLVITDVVDGVNHKINITMNTCTIRNCKKLGPRENVAVIQVGGSDILDITADPTTAPQTERMMRINWKKWWQVFYTVVDYVNQIIQAMSKRSRSLNSAAFYYRI

SEQ ID NO: 113 - DNA, 894 bp, Artificial Sequence
  Synthetic sequence, G12K12+7-1a-1b_G4BrB- 9_VP7_DNA_opt
atggactttatcatatataggttcctgctcatcgtggttgtgatgttgccattcataaaagcccagaactacgggatcaacctgcccataacaggatctatggacacagcttacaccaattcaactcaacaagagaatttcatgtctagcacactgtgcctttactatcctagtgaggcaccgactcaaatcagtgatacagaatggaaggatacactgtctcaactctttctcaccaagggatggcccactggctcagtgtattttaatgaatacagcaacgttttggagttcagtattgaccccaagctgtactgcgactacaatgtagtgctgattcgattcgcctcgggggaggaacttgacgtatccgagttggccgacctcatcctgaacgaatggctatgtaacccaatggacgtgaccctgtactactaccagcagaccgacgaggcaaataagtggatcagcatgggagaatcttgcaccgtgaaagtttgtccactgaatacacagactctcgggatcggctgcactactaccgatgttaccacctttgaagaagtggcaaacgccgagaagcttgtcatcacagatgtagttgacggcgttaatcacaaaattaatattactatgaacacctgcacgattaggaattgtaagaaactggggccacgcgaaaacgttgccataatccaggtgggaggtagcaatatcctcgacataaccgccgatcctacgacgtcccctcagactgaaaggatgatgcgagtcaactggaagaagtggtggcaagttttctatacagtggttgactatatcaaccaaatagtcaaggtgatgagtaaacgtagccggagccttaattctgccgcattctactacaggatctag

SEQ ID NO: 114 - protein, 297 aa, Artificial Sequence
  Synthetic sequence, G12K12+7-1a-1b_G4BrB- 9_VP7_AA
MDFIIYRFLLIVVVMLPFIKAQNYGINLPITGSMDTAYTNSTQQENFMSSTLCLYYPSEAPTQISDTEWKDTLSQLFLTKGWPTGSVYFNEYSNVLEFSIDPKLYCDYNVVLIRFASGEELDVSELADLILNEWLCNPMDVTLYYYQQTDEANKWISMGESCTVKVCPLNTQTLGIGCTTTDVTTFEEVANAEKLVITDVVDGVNHKINITMNTCTIRNCKKLGPRENVAIIQVGGSNILDITADPTTSPQTERMMRVNWKKWWQVFYTVVDYINQIVKVMSKRSRSLNSAAFYYRI

SEQ ID NO: 115 - DNA, 894 bp, Artificial Sequence
  Synthetic sequence, G12K12+7-1a- 1b_G9BE2001_VP7_DNA_opt
atggactttatcatatataggttcctgctcatcgtggttgtgatgttgccattcataaaagcccagaactacgggatcaacctgcccataacaggatctatggacacagcttacaccaattcaactcaacaagagaatttcatgacatcaaccttgtgcttgtattaccccactgaagcgtctactcagatcggagataccgagtggaaagatactctcagtcagctgttcctcaccaagggatggccaacaggctctgtctactttaaagagtacacggacatcgcatcttttagcatcgatcctcagttatactgcgactacaacgtggtgttgatgaaatacgacagcacgctggagctcgacatgtccgagctggctgatctgattctcaacgaatggctatgtaacccaatggacgtgaccctgtactactaccagcagaccgacgaggcaaataagtggatcagcatgggagaatcttgcaccgtgaaagtttgtccactgaatacacagactctcgggatcggctgcactactaccgatgttaccacctttgaagaagtggcaaacgccgagaagcttgtcatcacagatgtagttgacggcgttaatcacaaaattaatattactatgaacacctgcacgattaggaattgtaagaaactggggccacgcgaaaacgtggctatcgttcaggtgggcggttccgaggttctcgacataacggctgacccaaccaccgccccacagaccgagaggatgatgcgcgtgaactggaaaaaatggtggcaagtgttctacactgtggtggactatatcaaccagattgtgcaggtgatgtccaaacgtagccggagccttaattctgccgcattctactacaggatctag

SEQ ID NO: 116 - protein, 297 aa, Artificial Sequence
  Synthetic sequence, G12K12+7-1a- 1b_G9BE2001_VP7_AA
MDFIIYRFLLIVVVMLPFIKAQNYGINLPITGSMDTAYTNSTQQENFMTSTLCLYYPTEASTQIGDTEWKDTLSQLFLTKGWPTGSVYFKEYTDIASFSIDPQLYCDYNVVLMKYDSTLELDMSELADLILNEWLCNPMDVTLYYYQQTDEANKWISMGESCTVKVCPLNTQTLGIGCTTTDVTTFEEVANAEKLVITDVVDGVNHKINITMNTCTIRNCKKLGPRENVAIVQVGGSEVLDITADPTTAPQTERMMRVNWKKWWQVFYTVVDYINQIVQVMSKRSRSLNSAAFYYRI

SEQ ID NO: 117 - DNA, 894 bp, Artificial Sequence
  Synthetic sequence, G3HCR3_VP7_DNA_opt
atggatttcatcatatataggtttctgtttataatagtgattctgtcacctctactcaaggcgcaaaactatggcataaacctccctatcaccggctcaatggacaccgcctatgcaaactccacgcaggaagaaactctgctgaccagcacactctgcctctactacccgacagaggctgccactgagatcaacgataattcttggaaagacacgttatcgcagctgtttcttactaagggctggcccaccggtagtgtctactttaaagagtataccgacattgcctcttttagcgtggatcctcagctctactgtgactataacatcgtgttgatgaagtatgacgcagcgctgcagctggatatgagtgagctggccgatttgatcctgaatgagtggctgtgtaatccaatggatatcacactctactactaccagcagactgacgaagccaacaagtggatctctatgggttctagctgcaccatcaaagtgtgccccctgaacacccagacactgggcattggctgtctgacgacagatgtcagtaccttcgaggaggtggcgacaacagagaaactggtgatcaccgacgtggttgacggcgtgaaccacaaactcgacgtgacaactaccacctgcaccatccggaattgtaagaagctgggaccgagagaaaatgttgcagtcatccaggtaggaggcagtgatattctcgacatcacggccgacccgacgaccgcgcctcagacagaaaggatgatgcggatcaattggaagaagtggtggcaggtgttctacacagtggtggactacgttaaccagattattcaggctatgagcaagcgcagcagatcccttaactccgctgcattctattatagggtgtaa

SEQ ID NO: 118 - protein, 12 aa, Artificial Sequence
  Synthetic sequence, Boundary sequence
  misc_feature (4)..(6): Xaa can be any naturally occurring amino acid
STQXXXFLTSTL

SEQ ID NO: 119 - protein, 8 aa, Artificial Sequence
  Synthetic sequence, Boundary sequence
DLILNEWL

SEQ ID NO: 120 - protein, 8 aa, Artificial sequence
  Synthetic sequence, Boundary Sequence
GPRENVAI

SEQ ID NO: 121 - protein, 5 aa, Artificial Sequence
  Synthetic sequence, Boundary Sequence
MSKRS

SEQ ID NO: 122 - DNA, 61 bp, Artificial Sequence
  Synthetic sequence, Expression Enhancer nbGT61
atccagaagtaggaattcttcagtataatctagggttttttgaaaagcaaattgatcgaaa

SEQ ID NO: 123 - DNA, 75 bp, Artificial Sequence
  Synthetic sequence, Expression enhancer nbATL75
atctccaccaccaaaaaccctaatcgcctctccgtttcttcatcagattctcggttctcttcttctacagcaaca

SEQ ID NO: 124 - DNA, 46 bp, Artificial Sequence
  Synthetic sequence, Expression Enhancer nbDJ46
actcaccaagaaaataaacaaattaaagaattttaagaaaaacaag

SEQ ID NO: 125 - DNA, 79 bp, Artificial Sequence
  Synthetic sequence, Expression Enhancer nbCHP79
attctgccctcagttaactaaattatctctctgattaacagtactttctgattttctgtgatttctacaaatctgagac

SEQ ID NO: 126 - DNA, 42 bp, Artificial Sequence
  Synthetic sequence, Expression Enhacer nbEN42
acttttgtatagctccattgaaatagagaaaagaaaatagcc

SEQ ID NO: 127 - DNA, 69 bp, Artificial Sequence
  Synthetic sequence, Expression Enhancer atHSP69
aaattcaaaatttaacacacaaacacaaacacacacaccaaaaaaaacacagaccttaaaaaaataaaa

SEQ ID NO: 128 - DNA, 62 bp, Artificial Sequence
  Synthetic sequence, atGRP62
ataacaaaacaagattttgaagtaaaacataaaagaaaataaaccctaagaatatatcgaaa

SEQ ID NO: 129 - DNA, 65 bp, Artificial Sequence
  Synthetic sequence, Expression Enhancer atPK65
gcaaaaacaaaaataaaaaaaacatcgcacaagaaaataaaagatttgtagaatcaactaagaaa

SEQ ID NO: 130 - DNA, 46 bp, Artificial Sequence
  Synthetic sequence, Expression Enhancer atRP46
agaaacaaaaagaattaaaaaaaaaaaaaaaaaaaagaataaagaa

SEQ ID NO: 131 - DNA, 72 bp, Artificial Sequence
  Synthetic sequence, Expression Enhancer nb30S72
atctttccctcaaaaccctagccgcagtcacttccgtaggtgcttacttcgctgttagtgcaattccaaacc

SEQ ID NO: 132 - DNA, 78 bp, Artificial Sequence
  Synthetic sequence, Expression Enhancer nbMT78
acacaatttgctttagtgattaaactttcttttacaacaaattaaaggtctattatctcccaacaacataagaaaaca

SEQ ID NO: 133 - DNA, 55 bp, Artificial Sequence
  Synthetic sequence, Expression Enhancer nbPV55
aattaaagatcaattcactgtatccctcttctccaaaaaaaactctgctgtagtc

SEQ ID NO: 134 - DNA, 46 bp, Artificial Sequence
  Synthetic sequence, Expression Enhancer nbPPI43
agcacaaatcgtacacagcgaaaacctcactgaaatatttagagag

SEQ ID NO: 135 - DNA, 68 bp, Artificial Sequence
  Synthetic sequence, Expression Enhancer nbPM64
gttcagaaagatttgtttcctctgaaatagttttacagagccagaagaagaaaaagaagaagagagca

SEQ ID NO: 136 - DNA, 86 bp, Artificial Sequence
  Synthetic sequence, Expression Enhancer nbH2A86
actcaacactcaaatcgcaatccaaaagcttcaatttttcctaatacttctctgtattcaagcttcgtaaactttcattcacatca